(19) United States
(12) Patent Application Publication
(10) Pub. No.: US 2003/0119013 A1
(43) Pub. Date: Jun. 26, 2003

(54) Identification of essential genes of Aspergillus fumigatus and methods of use

(76) Inventors: Bo Jiang, Montreal (CA); Daniel Tishkoff, San Diego, CA (US); Carlos Zamudio, La Jolla, CA (US); Alexey M. Eroshkin, San Diego, CA (US); Wenqi Hu, Dollard-des-Ormeaux (CA); Sebastien Lemieux, Montreal (CA)

Correspondence Address: PENNIE AND EDMONDS, 1155 AVENUE OF THE AMERICAS, NEW YORK, NY, 100362711

(21) Appl. No.: 10/128,714

(22) Filed: Apr. 23, 2002

Related U.S. Application Data
(60) Provisional application No. 60/316,362, filed on Aug. 31, 2001.
(60) Provisional application No. 60/303,899, filed on Jul. 9, 2001.
(60) Provisional application No. 60/295,890, filed on Jun. 5, 2001.
(60) Provisional application No. 60/287,066, filed on Apr. 27, 2001.
(60) Provisional application No. 60/285,697, filed on Apr. 23, 2001.

(51) Int. Cl. C12Q 1/68; C07H 21/04; C12N 9/00; C12N 1/18; C12P 21/02; C12N 15/74

(52) U.S. Cl. 435/6; 435/69.1; 435/320.1; 435/254.2; 435/183; 530/350; 536/23.2

(57) ABSTRACT

The present invention provides nucleotide sequences, methods and compositions that enable the experimental determination as to whether any gene in the genome of Aspegillus fumigatus is essential, and whether that gene is required for virulence or pathogenicity. The methods involve the construction of genetic mutants in which a target gene is placed under conditional expression. The identification of essential genes and those genes critical to the development of virulent infections, provides a basis for the development of screens for new drugs against Aspergillus fumigatus.

The present invention further provides Aspergillum fumigatus genes that are essential and are potential targets for drug screening. The nucleotide sequence of the target genes can be used for various drug discovery purposes, such as expression of the recombinant protein, hybridization assay and construction of nucleic acid arrays. The uses of proteins encoded by the essential genes, and genetically engineered cells comprising modified alleles of essential genes in various screening methods are also encompassed by the invention.

### 1. INTRODUCTION

The present invention is directed toward a collection of identified essential genes of Aspergillus fumigatus and methods for identifying and validating gene products as effective targets for therapeutic intervention.

### 2. BACKGROUND OF THE INVENTION

Aspergillus fumigatus is a saprophytic fungus that plays an essential role in recycling environmental carbon and nitrogen. Its natural ecological niche is the soil, wherein it survives and grows on organic debris. Although this species is not the most prevalent fungus in the world, it is one of the most ubiquitous of those with airborne conidia. It sporulates abundantly, with every conidial head producing thousands of conidia. The conidia released into the atmosphere have a diameter small enough (2 to 3 μm) to reach the lung alveoli. Inhalation of conidia by immunocompetent individuals rarely has any adverse effect, since the conidia are eliminated relatively efficiently by innate immune mechanisms. Thus, until recent years, Aspergillus fumigatus was viewed as a weak pathogen responsible for allergic forms of the disease, such as farmer's lung, a clinical condition observed among individuals exposed repeatedly to conidia. Because of the increase in the number of immunosuppressed patients, and the degree of severity of modern immunosuppressive therapies, Aspergillus fumigatus has become the most prevalent airborne fungal pathogen, causing severe and usually fatal invasive infections in immunocompromised hosts.

A fourfold increase in invasive aspergillosis (IA) has been observed in the last 12 years. In 1992, IA was responsible for approximately 30% of fungal infections in patients dying of cancer, and it is estimated that IA occurs in 10 to 25% of all leukemia patients, in whom the mortality rate is 80 to 90%, even when treated. The average incidence of IA is estimated to be 5 to 25% in patients with acute leukemia, 5 to 10% after allogeneic bone marrow transplantation (BMT), and 0.5 to 5% after cytotoxic treatment of blood diseases or autologous BMT and solid-organ transplantation. IA which follows solid-organ transplantation is most common in heart-lung transplant patients (19 to 26%) and is found, in decreasing order, in liver, heart, lung, and kidney recipients (1 to 10%) (Patel and Paya, 1997, Clin. Microbiol. Rev. 10:86-124). IA also occurs in patients with nonhematogenous underlying conditions; it is increasingly reported in AIDS patients (1 to 12%) (Denning et al., 1991, N. Engl. J. Med. 324:654-661) and is also a common infectious complication of chronic granulomatous disease (CGD) (25 to 40%)

Four types of IA have been described (Denning, 1998, Clin, Infect. Dis. 26:781-805): (i) acute or chronic pulmonary aspergillosis, the most common form of IA; (ii) tracheobronchitis and obstructive bronchial disease with various degrees of invasion of the mucosa and cartilage as well as pseudomembrane formation, seen predominantly in AIDS patients; (iii) acute invasive rhinosinusitis; and (iv) disseminated disease commonly involving the brain (10 to 40% in BMT patients) and other organs (for example, the skin, kidneys, heart, and eyes).

Other diseases, such as allergic bronchopulmonary aspergillosis (ABPA), and aspergilloma, involving mycelial growth of Aspergillus fumigatus in the body, also require therapeutic intervention. ABPA is currently the most severe allergic pulmonary complication caused by Aspergillus species. It occurs in patients suffering from atopic asthma or cystic fibrosis. Aspergilloma, commonly referred to as “fungus ball,” occurs in preexisting pulmonary cavities that were caused by tuberculosis, sarcoidosis, or other bullous lung disorders and in chronically obstructed paranasal sinuses.

At present, only amphotericin B (AmB) and itraconazole are available to treat aspergillosis (DePauw, 1997, Eur. J. Clin. Microbiol. Infect. Dis., 16:32-41). In spite of their activity in vitro, the efficacy of these drugs in vivo against Aspergillus fumigatus remains low, and as a consequence, mortality from IA remains high. Despite much work and the development of new drugs, anti-Aspergillus therapy remains inadequate. The overall success rate of AmB therapy for IA is 34%. In addition, most IA cases occur in spite of empirical administration of AmB in response to a fever unresponsive to antibacterial agents. This observation underscores the basic inadequacy of drugs in vivo with activity against A. fumigatus and emphasizes the urgent need for identification of suitable biochemical targets in A. fumigatus and the discovery and development of new antifungal agents active against those biochemical targets.

Identification and validation of a cellular target for drug screening purposes generally involves an experimental demonstration that inactivation of that gene product leaves the cell inviable. Accordingly, a drug active against the same essential gene product expressed by A. fumigatus would be predicted to be an effective therapeutic agent. Similarly, a gene product required for A. fumigatus pathogenicity and virulence is also expected to provide a suitable target for drug screening programs. Target validation in this instance is based upon a demonstration that inactivation of the gene encoding the virulence factor creates a mutant A. fumigatus strain that is shown to be either less pathogenic or, ideally, avirulent, in animal model studies. Identification and validation of drug targets are critical issues for detection and discovery of new drugs because these targets form the basis for high throughput screens within the pharmaceutical industry.

Target discovery has traditionally been a costly, time-consuming process, in which newly-identified genes and gene products have been individually analyzed as potentially-suitable drug targets. However, with the advent of large scale DNA sequence analysis of entire genomes, the gene discovery process has been markedly accelerated. Consequently, new methods and tools are required to analyze this information, first to identify all of the genes of the organism, and then, to discern which genes encode products that will be suitable targets for the discovery of effective, non-toxic drugs.

As A. fumigatus is becoming a major fungal pathogen of humans, there is a growing need for effective, non-toxic therapeutic compounds for clinical use. The present invention takes a genomics approach to identify novel targets for drug screening. The invention provides the nucleotide sequences of essential genes of A. fumigatus, which can be used in high throughput strategies that provide rapid validation and screening of drug targets.

### 3. SUMMARY OF THE INVENTION

The present invention is directed toward the nucleotide sequence of the essential genes of Aspergillus fumigatus, the characterization of the gene products, and the construction of conditional-expression mutants and knock-out mutants of each of those genes. Accordingly, the mutants of the invention provide the experimental determination as to whether the genes are essential, and whether the genes are required for virulence or pathogenicity. The information provided herein forms a basis for the development of high-throughput screens for new drugs against Aspergillus fumigatus.

In one embodiment of the present invention, a set of essential genes of Aspergillus fumigatus which are potential targets for drug screening, is identified. Such genes have been identified by sequence comparisons with Candida albicans genes which have been determined experimentally to be essential for growth, survival, and proliferation of C. albicans. The polynucleotides of the essential genes or virulence genes of a Aspergillus fumigatus (i.e., the target genes) provided by the present invention can be used by various drug discovery purposes. Without limitation, the polynucleotides can be used to express recombinant protein for characterization, screening or therapeutic use; as markers for host tissues in which the pathogenic organisms invade or reside (either permanently or at a particular stage of development or in a disease states); to compare with the DNA sequence of Aspergillus fumigatus to identify duplicated genes or paralogs having the same or similar biochemical activity and/or function; to compare with DNA sequences of other related or distant pathogenic organisms to identify potential orthologous essential or virulence genes; for selecting and making oligomers for attachment to a nucleic acid array for examination of expression patterns; to raise anti-protein antibodies using DNA immunization techniques; as an antigen to raise anti-DNA antibodies or elicit another immune response; and as a therapeutic agent (e.g., antisense molecules). Where the polynucleotide encodes a protein which binds or potentially binds to another protein (such as, for example, in a receptor-ligand interaction), the polynucleotide can also be used in assays to identify polynucleotides encoding the other protein with which binding occurs or to identify inhibitors of the binding interaction.

The polypeptides or proteins encoded by the essential genes (i.e. the target gene products) provided by the present invention can also be used in assays to determine biological activity, including its uses as a member in a panel or an array of multiple proteins for high-throughput screening; to raise antibodies or to elicit immune response; as a reagent (including the labeled reagent) in assays designed to quantitatively determine levels of the protein (or its receptor) in biological fluids; as a marker for host tissues in which the pathogenic organisms invade or reside (either permanently or at a particular stage of development or in a disease states); and, of course, to isolate correlative receptors or ligands (also referred to as binding partners) especially in the case of virulence factors. Where the protein binds or potentially binds to another protein (such as, for example, in a receptor-ligand interaction), the protein can be used to identify the other protein with which binding occurs or to identify inhibitors of the binding interaction. Proteins involved in these binding interactions can also be used to screen for peptide or small molecule inhibitors or agonists of the binding interaction, such as those involved in invasiveness, and pathogenicity of the pathogenic organism.

In another embodiment, the present invention provides Aspergillus fumigatus mutant strains in which an essential gene is modified by the introduction (e.g., by recombination) of a promoter replacement fragment comprising a heterologous promoter, such that the expression of the essential gene is regulated by the heterologous promoter. In one non-limiting example, expression from the heterologous promoter can be regulated by the presence of a transactivator protein comprising a DNA-binding domain and transcription-activation domain. The DNA-binding domain of this transactivator protein recognizes and binds to a sequence in the heterologous promoter and increases transcription of that promoter. The transactivator protein can be produced in the cell by expressing a nucleotide sequence encoding the protein.

In the present invention, the gene modified in Aspergillus fumigatus corresponds to an essential gene, which is required for survival, growth, and proliferation of the strain. In a preferred embodiment, these modifications lead to the production of a rapid cidal phenotype in the mutant organisms. Accordingly, the present invention encompasses collections of Aspergillus fumigatus mutant strains wherein each collection comprises a plurality of strains, each strain containing a different conditional-expression mutant gene.

A collection can be used according to the various methods of the invention, wherein the cells of each strain in the collection are separately subjected to the same manipulation or treatment related to the use. Alternatively, the cells of each strain in a collection are pooled before the manipulation or treatment related to the use. The concept of a collection is also extended to data collection, processing and interpretation where data arising from different strains of fungal cells or a pool of different fungal strains in the collection are handled coordinately as a set.

In another embodiment, the present invention is directed to nucleic acid microarrays which comprise a plurality of defined nucleotide sequences disposed at identifiable positions in an array on a substrate. The defined nucleotide sequences can comprise oligonucleotides complementary to, and capable of hybridizing with, the nucleotide sequences of the essential genes of Aspergillus fumigatus that are required for the survival, growth and proliferation of Aspergillus fumigatus, and/or the unique molecular tags employed to mark each mutant Aspergillus fumigatus strain.

In yet another embodiment of the present invention, conditional-expression mutants of Aspergillus fumigatus, which are constructed according to the methods disclosed herein, are used for the detection of antifungal agents effective against Aspergillus fumigatus. Conditional-expression mutant Aspergillus fumigatus cells of the invention are cultured under differential growth conditions in the presence or absence of a test compound. The growth rates are then compared to indicate whether or not the compound is active against a target gene product encoded by the conditionally-expressed gene. In one aspect of this embodiment, the conditionally-expressed gene is substantially underexpressed to provide cells with enhanced sensitivity to compounds active against the gene product expressed by that geen. Alternatively, the conditionally-expressed gene may be substantially overexpressed to provide Aspergillus fumigatus cells with increased resistance to compounds active against the gene product expressed by the conditional-expression mutant allele of the target gene.

In yet another embodiment of the present invention, the Aspergillus fumigatus strains constructed according to the methods disclosed are used for the screening of therapeutic agents effective for the treatment of non-infectious diseases in a plant or an animal, such as a human. As a consequence of the similarity of a target's amino acid sequence with a plant or animal counterpart active compounds so identified may have therapeutic applications for the treatment of diseases in the plant or animal, in particular, human diseases, such as cancers and immune disorders.

The present invention, in other embodiments, further encompasses the use of transcriptional profiling and proteomics techniques to analyze the expression of essential and/or virulence genes of Aspergillus fumigatus under a variety of conditions, including in the presence of known drugs. The information yielded from such studies can be used to uncover the target and mechanism of known drugs, to discover new drugs that act in a similar fashion to known drugs, and to delineate the interactions between gene products that are essential to survival, growth, and proliferation of Aspergillus fumigatus and that are instrumental to virulence and pathogenicity of Aspergillus fumigatus.

Any or all of these drug discovery utilities are capable of being developed into a kit for commercialization as research products. The kits may comprise polynucleotides and/or polypeptides corresponding to a plurality of A. fumigatus essential genes of the invention, antibodies, and/or other reagents.

### 4. BRIEF DESCRIPTION OF THE DRAWINGS

FIG. 1: shows the growth a transformant of Aspergillus fumigatus in which the essential gene AfErg 8 has been placed under the control of the glucoamylase promoter PglaA, on agar media supplemented with 2% maltose, 2% xylose, or 1% glucose.

### 5. DETAILED DESCRIPTION OF THE INVENTION

### 5.1 Identification of Aspergillus fumigatus Essential Genes

### 5.11 DNA Sequence Analysis of the Aspergillus fumigatus Genome

The nucleotide sequences of Aspergillus fumigatus genomic DNA was obtained by a whole-genome random shotgun DNA sequencing effort. The genomic DNA was prepared from an isolate of Aspergillus fumigatus CEA 10 which was isolated from the infected lung tissue of a human aspergillosis patient. The genomic DNA was sheared mechanically into fragments, enzymatically treated to generate blunt ends, and cloned into E. coli pUC19- and pBR322-based plasmids to form genomic DNA libraries. Average insert sizes of the pUC19-based genomic DNA library clones were about 2 kb and the plasmids were present in high copy numbers in E. coli cells. The other two genomic DNA libraries of pBR322-based clones contain inserts of about 10 kb and about 50 kb respectively. The colonies of genomic clones were transferred robotically to 384-well titre plates; and plasmid DNA templates for dideoxy DNA sequencing reactions were prepared by standard method based on alkaline lysis of cells and isopropanol precipitation of DNA. DNA sequencing reactions were carried out using standard M13 forward and reverse primers and ABI-Prism BigDye terminator chemistry (Applied Biosystems), and analyzed using the capillary array sequencer ABI PRISM 3700 DNA Analyzer (Applied Biosystems). The nucleotide sequences generated were trimmed to discard errors, and assembled to form contigs and scaffolds by the software algorithms developed for sequencing the human genome. For a detailed description of the methodologies of the sequencing reactions and sequence analysis, see Venter et al., 2001, Science 291:1304 and; Myers et al, 2000, Science 287:2196, which are incorporated herein by reference in their entireties.

The present invention provides the nucleotide sequence of essential genes of Aspergillus fumigatus. The essential genes of the invention are identified by comparison of nucleotide sequences of Aspergillus fumigatus genomic DNA and the nucleotide sequences of known essential genes of Candida albicans. Prior to this invention, the nucleotide sequences of these Aspergillus fumigatus genes and their essentially with respect to the survival, growth, and proliferation of Aspergillus fumigatus are not known.

The set of nucleotide sequence data used in the present invention has an estimated 10×coverage of the Aspergillus fumigatus genome. The nucleotide sequences were initially annotated by software programs, such as Genescan and Glimmer M (The Institute of Genome Research), which can identify coding regions, introns, and splice junctions. Further automated and manual curation of the nucleotide sequences were performed to refine and establish precise characterization of the coding regions and other gene features.

The nucleotide sequences of the predicted Aspergillus fumigatus genes were compared with the nucleotide sequences of known essential genes of Candida albicans. Aspergillus fumigatus genes that display a 30% DNA sequence similarity, and/or a 35% similarity of predicted amino acid sequence are identified as essential genes of Aspergillus fumigatus.

The nucleotide sequences of more than six hundred Aspergillus fumigatus essential genes are provided in the attached sequence listing and cross-referenced in Table 1 with the identifiers of their homologs in Candida albicans. To facilitate correlation of the nucleotide sequences of each essential gene with its corresponding amino acid sequence(s) and other related sequences, the sequence identifiers have been organized into a total of eight blocks, each with one thousand SEQ ID numbers. A first series of SEQ ID numbers in four blocks, each of which corresponds to a type of sequence, has 594 sequences with SEQ ID NOs., and 405 SEQ ID NOs. with no sequence, which serve as place holders. Accordingly, the SEQ ID NO. for each of the four related sequences of an essential gene are separated by 1000. For example, SEQ ID NO: 1, 1001, 2001, and 3001, are directed to, respectively, the genomic sequence, the nucleotide sequence of a coding region with introns, the nucleotide sequence of an open reading frame, and the amino acid sequence of a gene product of one essential gene, and in this example, the A. fumigatus essential gene is AfYMR290C. Similarly, a second series of SEQ ID numbers in four blocks, each of which corresponds to a type of sequence, has 603 sequences with SEQ ID NOs., and 397 SEQ ID NOs. with no sequence, which serve as place holders. Accordingly, the SEQ ID NO. for each of the four related sequences of an essential gene are separated by 1000. For example, SEQ ID NO: 5001, 6001, 7001, and 8001, are directed to, respectively, the genomic sequence, the nucleotide sequence of a coding region with introns, the nucleotide sequence of an open reading frame, and the amino acid sequence of a gene product of a variant of the essential Aspergillus fumigatus gene, AfYMR290C.

The features of the nucleotide sequences of the essential genes, the predicted amino acid sequences, nucleic acid arrays, recombinant vectors and expression vectors comprising nucleotide sequences of the Aspergillus fumigatus essential genes are provided and described hereinbelow in Sections 5.2.1, 5.2.2 and 5.2.3. Genetically engineered yeast cells, prokaryotic cells, and cells of higher eukaryotes comprising nucleotide sequences of the Aspergillus fumigatus essential genes are provided and described in Section 5.2.3. Antisense nucleic acid molecules corresponding to the Aspergillus fumigatus essential genes of the invention are provided in Section 5.2.6.

TABLE 1

| Designations of | | | Coding | | Amino Acid |
|---|---|---|---|---|---|
| Aspergillus fumigatus | Designations of | Genomic | Sequence | Open Reading | Sequence |
| Chromosomal | Candida Albicans | Sequence | with Intron(s) | Frame | of Gene Product |
| Sequences | Homologs | SEQ ID NO: | SEQ ID NO: | SEQ ID NO: | SEQ ID NO: |
| AfYMR290C | CaYMR290C | 1 | 1001 | 2001 | 3001 |
| AfYPR034W | CaYPR034W | 2 | 1002 | 2002 | 3002 |
| AfORF6_4497 | CaORF6_4497 | 3 | 1003 | 2003 | 3003 |
| AfYJL008C | CaYJL008C | 4 | 1004 | 2004 | 3004 |
| AfYIL068C | CaYIL068C | 5 | 1005 | 2005 | 3005 |
| AfYHR196W | CaYHR196W | 6 | 1006 | 2006 | 3006 |
| AfYMR197C | CaYMR197C | 7 | 1007 | 2007 | 3007 |
| AfYLR100W | CaYLR100W | 8 | 1008 | 2008 | 3008 |
| AfYDL055C | CaYDL055C | 9 | 1009 | 2009 | 3009 |
| AfYDL043C | CaYDL043C | 10 | 1010 | 2010 | 3010 |
| AfYJL054W | CaYJL054W | 11 | 1011 | 2011 | 3011 |
| AfYHR072W2 | CaYHR072W2 | 12 | 1012 | 2012 | 3012 |
| AfYPR119W | CaYPR119W | 13 | 1013 | 2013 | 3013 |
| AfYDR013W | CaYDR013W | 14 | 1014 | 2014 | 3014 |
| AfYGR255C | CaYGR255C | 15 | 1015 | 2015 | 3015 |
| AfYDR353W | CaYDR353W | 16 | 1016 | 2016 | 3016 |
| AfYNR053C | CaYNR053C | 17 | 1017 | 2017 | 3017 |
| AfYLR222C | CaYLR222C | 18 | 1018 | 2018 | 3018 |
| AfYER025W | CaYER025W | 19 | 1019 | 2019 | 3019 |
| AfYOR272W | CaYOR272W | 20 | 1020 | 2020 | 3020 |
| AfYGL206C | CaYGL206C | 21 | 1021 | 2021 | 3021 |
| AfYMR208W | CaYMR208W | 22 | 1022 | 2022 | 3022 |
| AfYKL019W | CaYKL019W | 23 | 1023 | 2023 | 3023 |
| AfYJR006W | CaYJR006W | 24 | 1024 | 2024 | 3024 |
| AfYIL075C | CaYIL075C | 25 | 1025 | 2025 | 3025 |
| AfYER070W | CaYER070W | 26 | 1026 | 2026 | 3026 |
| AfYMR113W | CaYMR113W | 27 | 1027 | 2027 | 3027 |
| AfYIR011C | CaYIR011C | 28 | 1028 | 2028 | 3028 |
| AfYER012W | CaYER012W | 29 | 1029 | 2029 | 3029 |
| AfORF6_8837 | CaORF6_8837 | 30 | 1030 | 2030 | 3030 |
| AfYJR002W | CaYJR002W | 31 | 1031 | 2031 | 3031 |
| AfYPL043W | CaYPL043W | 32 | 1032 | 2032 | 3032 |
| AfYNL110C | CaYNL110C | 33 | 1033 | 2033 | 3033 |
| AfORF6_4899 | CaORF6_4899 | 34 | 1034 | 2034 | 3034 |
| AfORF6_5199 | CaORF6_5199 | 35 | 1035 | 2035 | 3035 |
| AfYFR052W | CaYFR052W | 36 | 1036 | 2036 | 3036 |
| AfYMR146C | CaYMR146C | 37 | 1037 | 2037 | 3037 |
| AfYGL003C | CaYGL003C | 38 | 1038 | 2038 | 3038 |
| AfYFL008W | CaYFL008W | 39 | 1039 | 2039 | 3039 |
| AfYDL028C | CaYDL028C | 40 | 1040 | 2040 | 3040 |
| AfYHR070W | CaYHR070W | 41 | 1041 | 2041 | 3041 |
| AfYLR115W | CaYLR115W | 42 | 1042 | 2042 | 3042 |
| AfYLR197W | CaYLR197W | 43 | 1043 | 2043 | 3043 |
| AfYOL022C | CaYOL022C | 44 | 1044 | 2044 | 3044 |
| AfYIL062C | CaYIL062C | 45 | 1045 | 2045 | 3045 |
| AfYPR086W | CaYPR086W | 46 | 1046 | 2046 | 3046 |
| AfYPR183W | CaYPR183W | 47 | 1047 | 2047 | 3047 |
| AfYNL126W | CaYNL126W | 48 | 1048 | 2048 | 3048 |
| AfYLR167W | CaYLR167W | 49 | 1049 | 2049 | 3049 |
| AfYJL010C | CaYJL010C | 50 | 1050 | 2050 | 3050 |
| AfYLR026C | CaYLR026C | 51 | 1051 | 2051 | 3051 |
| AfYBR192W | CaYBR192W | 52 | 1052 | 2052 | 3052 |
| AfYDR088C | CaYDR088C | 53 | 1053 | 2053 | 3053 |
| AfYOR280C | CaYOR280C | 54 | 1054 | 2054 | 3054 |
| AfYNL244C | CaYNL244C | 55 | 1055 | 2055 | 3055 |
| AfYER021W | CaYER021W | 56 | 1056 | 2056 | 3056 |
| AfYLR186W | CaYLR186W | 57 | 1057 | 2057 | 3057 |
| AfYDR527W | CaYDR527W | 58 | 1058 | 2058 | 3058 |
| AfYDL017W | CaYDL017W | 59 | 1059 | 2059 | 3059 |
| AfYGR090W | CaYGR090W | 60 | 1060 | 2060 | 3060 |
| AfYKL182W | CaYKL182W | 61 | 1061 | 2061 | 3061 |
| AfYPL231W | CaYPL231W | 62 | 1062 | 2062 | 3062 |
| AfYDR120C | CaYDR120C | 63 | 1063 | 2063 | 3063 |
| AfYBR080C | CaYBR080C | 64 | 1064 | 2064 | 3064 |
| AfYPR082C | CaYPR082C | 65 | 1065 | 2065 | 3065 |
| AfYOR310C | CaYOR310C | 66 | 1066 | 2066 | 3066 |
| AfYDL205C | CaYDL205C | 67 | 1067 | 2067 | 3067 |
| AfYIR022W | CaYIR022W | 68 | 1068 | 2068 | 3068 |
| AfYGR246C | CaYGR246C | 69 | 1069 | 2069 | 3069 |
| AfYCL017C | CaYCL017C | 70 | 1070 | 2070 | 3070 |
| AfYBL023C | CaYBL023C | 71 | 1071 | 2071 | 3071 |
| AfORF6_5147 | CaORF6_5147 | 72 | 1072 | 2072 | 3072 |
| AfYGR209C | CaYGR209C | 73 | 1073 | 2073 | 3073 |
| AfYLR306W | CaYLR306W | 74 | 1074 | 2074 | 3074 |
| AfYLL012W | CaYLL012W | 75 | 1075 | 2075 | 3075 |
| AfYPL217C | CaYPL217C | 76 | 1076 | 2076 | 3076 |
| AfYPR110C | CaYPR110C | 77 | 1077 | 2077 | 3077 |
| AfYMR288W | CaYMR288W | 78 | 1078 | 2078 | 3078 |
| AfYJL074C | CaYJL074C | 79 | 1079 | 2079 | 3079 |
| AfYOR119C | CaYOR119C | 80 | 1080 | 2080 | 3080 |
| AfYNR017W | CaYNR017W | 81 | 1081 | 2081 | 3081 |
| AfYDL060W | CaYDL060W | 82 | 1082 | 2082 | 3082 |
| AfYlL021W | CaYIL021W | 83 | 1083 | 2083 | 3083 |
| AfTRP5 | CaTRP5 | 84 | 1084 | 2084 | 3084 |
| AfYPL203W | CaYPL203W | 85 | 1085 | 2085 | 3085 |
| AfYPL094C | CaYPL094C | 86 | 1086 | 2086 | 3086 |
| AfYBL026W | CaYBL026W | 87 | 1087 | 2087 | 3087 |
| AfYKL210W | CaYKL210W | 88 | 1088 | 2088 | 3088 |
| AfYIL003W | CaYIL003W | 89 | 1089 | 2089 | 3089 |
| AfYDR212W | CaYDR212W | 90 | 1090 | 2090 | 3090 |
| AfYLR002C | CaYLR002C | 91 | 1091 | 2091 | 3091 |
| AfYLR397C | CaYLR397C | 92 | 1092 | 2092 | 3092 |
| AfYGL001C | CaYGL001C | 93 | 1093 | 2093 | 3093 |
| AfYPR041W | CaYPR041W | 94 | 1094 | 2094 | 3094 |
| AfYGR156W | CaYGR156W | 95 | 1095 | 2095 | 3095 |
| AfYPL010W | CaYPL010W | 96 | 1096 | 2096 | 3096 |
| AfYLR259C | CaYLR259C | 97 | 1097 | 2097 | 3097 |
| AfYBR196C | CaYBR196C | 98 | 1098 | 2098 | 3098 |
| AfYJR045C | CaYJR045C | 99 | 1099 | 2099 | 3099 |
| AfYBR202W | CaYBR202W | 100 | 1100 | 2100 | 3100 |
| AfYNL245C | CaYNL245C | 101 | 1101 | 2101 | 3101 |
| AfORF6_4747 | CaORF6_4747 | 102 | 1102 | 2102 | 3102 |
| AfORF6_7375 | CaORF6_7375 | 103 | 1103 | 2103 | 3103 |
| AfYER136W | CaYER136W | 104 | 1104 | 2104 | 3104 |
| AfYPL093W | CaYPL093W | 105 | 1105 | 2105 | 3105 |
| AfYBR159W | CaYBR159W | 106 | 1106 | 2106 | 3106 |
| AfYJL034W | CaYJL034W | 107 | 1107 | 2107 | 3107 |
| AfYDR172W | CaYDR172W | 108 | 1108 | 2108 | 3108 |
| AfYOR157C | CaYOR157C | 109 | 1109 | 2109 | 3109 |
| AfYLR127C | CaYLR127C | 110 | 1110 | 2110 | 3110 |
| AfYMR213W | CaYMR213W | 111 | 1111 | 2111 | 3111 |
| AfYHR019C | CaYHR019C | 112 | 1112 | 2112 | 3112 |
| AfYLR229C | CaYLR229C | 113 | 1113 | 2113 | 3113 |
| AfYGL130W | CaYGL130W | 114 | 1114 | 2114 | 3114 |
| AfYJL002C | CaYJL002C | 115 | 1115 | 2115 | 3115 |
| AfYPR105C | CaYPR105C | 116 | 1116 | 2116 | 3116 |
| AfYBR055C | CaYBR055C | 117 | 1117 | 2117 | 3117 |
| AfYPR175W | CaYPR175W | 118 | 1118 | 2118 | 3118 |
| AfYPL063W | CaYPL063W | 119 | 1119 | 2119 | 3119 |
| AfYFL022C | CaYFL022C | 120 | 1120 | 2120 | 3120 |
| AfYML075C | CaYML075C | 121 | 1121 | 2121 | 3121 |
| AfYNL222W | CaYNL222W | 122 | 1122 | 2122 | 3122 |
| AfYLR086W | CaYLR086W | 123 | 1123 | 2123 | 3123 |
| AfYOL142W | CaYOL142W | 124 | 1124 | 2124 | 3124 |
| AfYHR186C | CaYHR186C | 125 | 1125 | 2125 | 3125 |
| AfYNL287W | CaYNL287W | 126 | 1126 | 2126 | 3126 |
| AfYLL018C | CaYLL018C | 127 | 1127 | 2127 | 3127 |
| AfYAL015C | CaYAL015C | 128 | 1128 | 2128 | 3128 |
| AfYOR335C | CaYOR335C | 129 | 1129 | 2129 | 3129 |
| AfYDL193W | CaYDL193W | 130 | 1130 | 2130 | 3130 |
| AfYML126C | CaYML126C | 131 | 1131 | 2131 | 3131 |
| AfYDR404C | CaYDR404C | 132 | 1132 | 2132 | 3132 |
| AfYML130C | CaYML130C | 133 | 1133 | 2133 | 3133 |
| AfYKR081C | CaYKR081C | 134 | 1134 | 2134 | 3134 |
| AfYER172C | CaYER172C | 135 | 1135 | 2135 | 3135 |
| AfYOL010W | CaYOL010W | 136 | 1136 | 2136 | 3136 |
| AfYPR178W | CaYPR178W | 137 | 1137 | 2137 | 3137 |
| AfORF6_4974 | CaORF6_4974 | 138 | 1138 | 2138 | 3138 |
| AfYGL022W | CaYGL022W | 139 | 1139 | 2139 | 3139 |
| AfYGR280C | CaYGR280C | 140 | 1140 | 2140 | 3140 |
| AfYDL108W | CaYDL108W | 141 | 1141 | 2141 | 3141 |
| AfYOL005C | CaYOL005C | 142 | 1142 | 2142 | 3142 |
| AfYJL194W | CaYJL194W | 143 | 1143 | 2143 | 3143 |
| AfYOR151C | CaYOR151C | 144 | 1144 | 2144 | 3144 |
| AfYCL031C | CaYCL031C | 145 | 1145 | 2145 | 3145 |
| AfYOR210W | CaYOR210W | 146 | 1146 | 2146 | 3146 |
| AfYGL123W | CaYGL123W | 147 | 1147 | 2147 | 3147 |
| AfYOL139C | CaYOL139C | 148 | 1148 | 2148 | 3148 |
| AfORF6_8482 | CaORF6_8482 | 149 | 1149 | 2149 | 3149 |
| AfYKL035W | CaYKL035W | 150 | 1150 | 2150 | 3150 |
| AfORF6_5210 | CaORF6_5210 | 151 | 1151 | 2151 | 3151 |
| AfYOR294W | CaYOR294W | 152 | 1152 | 2152 | 3152 |
| AfYJL001W | CaYJL001W | 153 | 1153 | 2153 | 3153 |
| AfYHR052W | CaYHR052W | 154 | 1154 | 2154 | 3154 |
| AfYNL313C | CaYNL313C | 155 | 1155 | 2155 | 3155 |
| AfYLR249W | CaYLR249W | 156 | 1156 | 2156 | 3156 |
| AfYKL181W | CaYKL181W | 157 | 1157 | 2157 | 3157 |
| AfYML092C | CaYML092C | 158 | 1158 | 2158 | 3158 |
| AfYGL106W | CaYGL106W | 159 | 1159 | 2159 | 3159 |
| AfYLR243W | CaYLR243W | 160 | 1160 | 2160 | 3160 |
| AfYPL151C | CaYPL151C | 161 | 1161 | 2161 | 3161 |
| AfYER125W | CaYER125W | 162 | 1162 | 2162 | 3162 |
| AfYNL006W | CaYNL006W | 163 | 1163 | 2163 | 3163 |
| AfYJL087C | CaYJL087C | 164 | 1164 | 2164 | 3164 |
| AfYBR237W | CaYBR237W | 165 | 1165 | 2165 | 3165 |
| AfYLR117C | CaYLR117C | 166 | 1166 | 2166 | 3166 |
| AfYDR091C | CaYDR091C | 167 | 1167 | 2167 | 3167 |
| AfYGL040C | CaYGL040C | 168 | 1168 | 2168 | 3168 |
| AfYDR454C | CaYDR454C | 169 | 1169 | 2169 | 3169 |
| AfYDR267C | CaYDR267C | 170 | 1170 | 2170 | 3170 |
| AfYGR264C | CaYGR264C | 171 | 1171 | 2171 | 3171 |
| AfYMR308C | CaYMR308C | 172 | 1172 | 2172 | 3172 |
| AfYNL124W | CaYNL124W | 173 | 1173 | 2173 | 3173 |
| AfYDR473C | CaYDR473C | 174 | 1174 | 2174 | 3174 |
| AfYBR154C | CaYBR154C | 175 | 1175 | 2175 | 3175 |
| AfYDL140C | CaYDL140C | 176 | 1176 | 2176 | 3176 |
| AfYHR005C | CaYHR005C | 177 | 1177 | 2177 | 3177 |
| AfYER003C | CaYER003C | 178 | 1178 | 2178 | 3178 |
| AfYBL076C | CaYBL076C | 179 | 1179 | 2179 | 3179 |
| AfYBR265W | CaYBR265W | 180 | 1180 | 2180 | 3180 |
| AfYDL153C | CaYDL153C | 181 | 1181 | 2181 | 3181 |
| AfYHR024C | CaYHR024C | 182 | 1182 | 2182 | 3182 |
| AfYDR062W | CaYDR062W | 183 | 1183 | 2183 | 3183 |
| AfYHR169W | CaYHR169W | 184 | 1184 | 2184 | 3184 |
| AfYBR256C | CaYBR256C | 185 | 1185 | 2185 | 3185 |
| AfYLL011W | CaYLL011W | 186 | 1186 | 2186 | 3186 |
| AfYDL097C | CaYDL097C | 187 | 1187 | 2187 | 3187 |
| AfYCR052W | CaYCR052W | 188 | 1188 | 2188 | 3188 |
| AfYPL169C | CaYPL169C | 189 | 1189 | 2189 | 3189 |
| AfYNL132W | CaYNL132W | 190 | 1190 | 2190 | 3190 |
| AfYNL308C | CaYNL308C | 191 | 1191 | 2191 | 3191 |
| AfYLR060W | CaYLR060W | 192 | 1192 | 2192 | 3192 |
| AfYML093W | CaYML093W | 193 | 1193 | 2193 | 3193 |
| AfORF6_2086-2 | CaORF6_2086-2 | 194 | 1194 | 2194 | 3194 |
| AfYLR029C | CaYLR029C | 195 | 1195 | 2195 | 3195 |
| AfYBR088C | CaYBR088C | 196 | 1196 | 2196 | 3196 |
| AfYKR086W | CaYKR086W | 197 | 1197 | 2197 | 3197 |
| AfYMR015C | CaYMR015C | 198 | 1198 | 2198 | 3198 |
| AfYAL035W | CaYAL035W | 199 | 1199 | 2199 | 3199 |
| AfYJR123W | CaYJR123W | 200 | 1200 | 2200 | 3200 |
| AfYGR245C | CaYGR245C | 201 | 1201 | 2201 | 3201 |
| AfPBS2 | CaPBS2 | 202 | 1202 | 2202 | 3202 |
| AfYFL018C | CaYFL018C | 203 | 1203 | 2203 | 3203 |
| AfYOR206W | CaYOR206W | 204 | 1204 | 2204 | 3204 |
| AfYNR038W | CaYNR038W | 205 | 1205 | 2205 | 3205 |
| AfYML085C | CaYMLO85C | 206 | 1206 | 2206 | 3206 |
| AfYKL125W | CaYKLI25W | 207 | 1207 | 2207 | 3207 |
| AfYLR196W | CaYLR196W | 208 | 1208 | 2208 | 3208 |
| AfYNR035C | CaYNR035C | 209 | 1209 | 2209 | 3209 |
| AfYPR107C | CaYPR107C | 210 | 1210 | 2210 | 3210 |
| AfYNL280C | CaYNL2800 | 211 | 1211 | 2211 | 3211 |
| AfYIL142W | CaYIL142W | 212 | 1212 | 2212 | 3212 |
| AfYCR072C | CaYCR072C | 213 | 1213 | 2213 | 3213 |
| AfYER031C | CaYER031C | 214 | 1214 | 2214 | 3214 |
| AIYOL077C | CaYOL077C | 215 | 1215 | 2215 | 3215 |
| AfYNL088W | CaYNL088W | 216 | 1216 | 2216 | 3216 |
| AfYER113C | CaYER113C | 217 | 1217 | 2217 | 3217 |
| AfYKR062W | CaYKR062W | 218 | 1218 | 2218 | 3218 |
| AfYPL028W | CaYPL028W | 219 | 1219 | 2219 | 3219 |
| AfYKL046C | CaYKL046C | 220 | 1220 | 2220 | 3220 |
| AfYBR142W | CaYBR142W | 221 | 1221 | 2221 | 3221 |
| AfYDR052C | CaYDR052C | 222 | 1222 | 2222 | 3222 |
| AfYLR300W | CaYLR300W | 223 | 1223 | 2223 | 3223 |
| AfYPR010C | CaYPR0l0C | 224 | 1224 | 2224 | 3224 |
| AfORF6_2086 | CaORF6_2086 | 225 | 1225 | 2225 | 3225 |
| AfYDR037W | CaYDR037W | 226 | 1226 | 2226 | 3226 |
| AfYPR108W | CaYPR108W | 227 | 1227 | 2227 | 3227 |
| AfYFR050C | CaYFR050C | 228 | 1228 | 2228 | 3228 |
| AfYBR234C | CaYBR234C | 229 | 1229 | 2229 | 3229 |
| AfYHR174W | CaYHR174W | 230 | 1230 | 2230 | 3230 |
| AfYBR070C | CaYBR070C | 231 | 1231 | 2231 | 3231 |
| AfYGR211W | CaYGR211W | 232 | 1232 | 2232 | 3232 |
| AfYOR095C | CaYOR095C | 233 | 1233 | 2233 | 3233 |
| AfYHR042W | CaYHR042W | 234 | 1234 | 2234 | 3234 |
| AfYJL033W | CaYJL033W | 235 | 1235 | 2235 | 3235 |
| AfYDL031W | CaYDL031W | 236 | 1236 | 2236 | 3236 |
| AfYLR342W | CaYLR342W | 237 | 1237 | 2237 | 3237 |
| AfYDR211W | CaYDR211W | 238 | 1238 | 2238 | 3238 |
| AfYPL160W | CaYPL160W | 239 | 1239 | 2239 | 3239 |
| AfYDR356W | CaYDR356W | 240 | 1240 | 2240 | 3240 |
| AfYFL038C | CaYFL038C | 241 | 1241 | 2241 | 3241 |
| AfYFR002W | CaYFR002W | 242 | 1242 | 2242 | 3242 |
| AfYOR074C | CaYOR074C | 243 | 1243 | 2243 | 3243 |
| AfYCL054W | CaYCL054W | 244 | 1244 | 2244 | 3244 |
| AfYJL026W | CaYJL026W | 245 | 1245 | 2245 | 3245 |
| AfYJL039C | CaYJL039C | 246 | 1246 | 2246 | 3246 |
| AfYML025C | CaYML025C | 247 | 1247 | 2247 | 3247 |
| AfORF6_1934 | CaORF6_1934 | 248 | 1248 | 2248 | 3248 |
| AfYDR361C | CaYDR361C | 249 | 1249 | 2249 | 3249 |
| AfYGL065C | CaYGL065C | 250 | 1250 | 2250 | 3250 |
| AfYNL232W | CaYNL232W | 251 | 1251 | 2251 | 3251 |
| AfYER023W | CaYER023W | 252 | 1252 | 2252 | 3252 |
| AfYBR060C | CaYBR060C | 253 | 1253 | 2253 | 3253 |
| AfYLR378C | CaYLR378C | 254 | 1254 | 2254 | 3254 |
| AfORF6_8025 | CaORF6_8025 | 255 | 1255 | 2255 | 3255 |
| AfYDL007W | CaYDL007W | 256 | 1256 | 2256 | 3256 |
| AfORF6_5739 | CaORF6_5739 | 257 | 1257 | 2257 | 3257 |
| AfYNL277W | CaYNL277W | 258 | 1258 | 2258 | 3258 |
| AfYPR048W | CaYPR048W | 259 | 1259 | 2259 | 3259 |
| AfYHR088W | CaYHR088W | 260 | 1260 | 2260 | 3260 |
| AfYPR016C | CaYPR016C | 261 | 1261 | 2261 | 3261 |
| AfYIL126W | CaYIL126W | 262 | 1262 | 2262 | 3262 |
| AfYLR105C | CaYLR105C | 263 | 1263 | 2263 | 3263 |
| AfYHR072W | CaYHR072W | 264 | 1264 | 2264 | 3264 |
| AfYBR160W | CaYBR160W | 265 | 1265 | 2265 | 3265 |
| AfYBL040C | CaYBL040C | 266 | 1266 | 2266 | 3266 |
| AfYMR240C | CaYMR240C | 267 | 1267 | 2267 | 3267 |
| AfYLR175W | CaYLR175W | 268 | 1268 | 2268 | 3268 |
| AfYLR175W2 | CaYLR175W2 | 269 | 1269 | 2269 | 3269 |
| AfYHR074W | CaYHR074W | 270 | 1270 | 2270 | 3270 |
| AfYPR088C | CaYPR088C | 271 | 1271 | 2271 | 3271 |
| AfYDL030W | CaYDL030W | 272 | 1272 | 2272 | 3272 |
| AfYNL062C | CaYNL062C | 273 | 1273 | 2273 | 3273 |
| AfYDR196C | CaYDR196C | 274 | 1274 | 2274 | 3274 |
| AfYOL038W | CaYOL038W | 275 | 1275 | 2275 | 3275 |
| AfYDL217C | CaYDL217C | 276 | 1276 | 2276 | 3276 |
| AfYOR250C | CaYOR250C | 277 | 1277 | 2277 | 3277 |
| AfYDR167W | CaYDR167W | 278 | 1278 | 2278 | 3278 |
| AfYGL120C | CaYGL120C | 279 | 1279 | 2279 | 3279 |
| AfYHR027C | CaYHR027C | 280 | 1280 | 2280 | 3280 |
| AfYER013W | CaYER013W | 281 | 1281 | 2281 | 3281 |
| AfORF6_569 | CaORF6_569 | 282 | 1282 | 2282 | 3282 |
| AfORF6_6011 | CaORF6_6011 | 283 | 1283 | 2283 | 3283 |
| AfYNL247W | CaYNL247W | 284 | 1284 | 2284 | 3284 |
| AfYJL125C | CaYJL125C | 285 | 1285 | 2285 | 3285 |
| AfYML125C | CaYML125C | 286 | 1286 | 2286 | 3286 |
| AfYJR076C | CaYJR076C | 287 | 1287 | 2287 | 3287 |
| AfYGR070W | CaYGR070W | 288 | 1288 | 2288 | 3288 |
| AfYDL105W | CaYDL105W | 289 | 1289 | 2289 | 3289 |
| AfYHR023W | CaYHR023W | 290 | 1290 | 2290 | 3290 |
| AfLYS4 | CaLYS4 | 291 | 1291 | 2291 | 3291 |
| AfYDR062W2 | CaYDR062W2 | 292 | 1292 | 2292 | 3292 |
| AfYMR203W | CaYMR203W | 293 | 1293 | 2293 | 3293 |
| AfYOL094C | CaYOL094C | 294 | 1294 | 2294 | 3294 |
| AfYDR407C | CaYDR407C | 295 | 1295 | 2295 | 3295 |
| AfYOR287C | CaYOR287C | 296 | 1296 | 2296 | 3296 |
| AfYLL031C | CaYLL031C | 297 | 1297 | 2297 | 3297 |
| AfYPL085W | CaYPL085W | 298 | 1298 | 2298 | 3298 |
| AfYMR260C | CaYMR260C | 299 | 1299 | 2299 | 3299 |
| AfYFL017C | CaYFL017C | 300 | 1300 | 2300 | 3300 |
| AfYMR218C | CaYMR218C | 301 | 1301 | 2301 | 3301 |
| AfYEL026W | CaYEL026W | 302 | 1302 | 2302 | 3302 |
| AfYDL207W | CaYDL207W | 303 | 1303 | 2303 | 3303 |
| AfYNL131W | CaYNL131W | 304 | 1304 | 2304 | 3304 |
| AfYNR026C | CaYNR026C | 305 | 1305 | 2305 | 3305 |
| AfYOR004W | CaYOR004W | 306 | 1306 | 2306 | 3306 |
| AfYBR254C | CaYBR254C | 307 | 1307 | 2307 | 3307 |
| AfYOR232W | CaYOR232W | 308 | 1308 | 2308 | 3308 |
| AfYNR043W | CaYNR043W | 309 | 1309 | 2309 | 3309 |
| AfYOR257W | CaYOR257W | 310 | 1310 | 2310 | 3310 |
| AfYGR060W | CaYGR060W | 311 | 1311 | 2311 | 3311 |
| AfYJR112W | CaYJR112W | 312 | 1312 | 2312 | 3312 |
| AfYPR186C | CaYPR186C | 313 | 1313 | 2313 | 3313 |
| AfYBR079C | CaYBR079C | 314 | 1314 | 2314 | 3314 |
| AfYPR056W | CaYPR056W | 315 | 1315 | 2315 | 3315 |
| AfYDR472W | CaYDR472W | 316 | 1316 | 2316 | 3316 |
| AfYGR172C | CaYGR172C | 317 | 1317 | 2317 | 3317 |
| AfYMR028W | CaYMR028W | 318 | 1318 | 2318 | 3318 |
| AfYMR227C | CaYMR227C | 319 | 1319 | 2319 | 3319 |
| AfYGR029W | CaYGR029W | 320 | 1320 | 2320 | 3320 |
| AfYPR025C | CaYPR025C | 321 | 1321 | 2321 | 3321 |
| AfYOR145C | CaYOR145C | 322 | 1322 | 2322 | 3322 |
| AfYBL041W | CaYBL041W | 323 | 1323 | 2323 | 3323 |
| AfYHR122W | CaYHR122W | 324 | 1324 | 2324 | 3324 |
| AfYPR113W | CaYPR113W | 325 | 1325 | 2325 | 3325 |
| AfYHR143W-A | CaYHR143W-A | 326 | 1326 | 2326 | 3326 |
| AfYDR449C | CaYDR449C | 327 | 1327 | 2327 | 3327 |
| AfYDR016C | CaYDR016C | 328 | 1328 | 2328 | 3328 |
| AfYDR236C | CaYDR236C | 329 | 1329 | 2329 | 3329 |
| AfYKL141W | CaYKL141W | 330 | 1330 | 2330 | 3330 |
| AfYLR078C | CaYLR078C | 331 | 1331 | 2331 | 3331 |
| AfYDR311W | CaYDR311W | 332 | 1332 | 2332 | 3332 |
| AfORF6_3819 | CaORF6_3819 | 333 | 1333 | 2333 | 3333 |
| AfORF6_3864 | CaORF6_3864 | 334 | 1334 | 2334 | 3334 |
| AfORF6_804 | CaORF6_804 | 335 | 1335 | 2335 | 3335 |
| AfORF6_889 | CaORF6_889 | 336 | 1336 | 2336 | 3336 |
| AfYAL033W | CaYAL033W | 337 | 1337 | 2337 | 3337 |
| AfYBL030C | CaYBL030C | 338 | 1338 | 2338 | 3338 |
| AfYBR029C | CaYBR029C | 339 | 1339 | 2339 | 3339 |
| AfYBR123C | CaYBR123C | 340 | 1340 | 2340 | 3340 |
| AfYBR143C | CaYBR143C | 341 | 1341 | 2341 | 3341 |
| AfYBR155W | CaYBR155W | 342 | 1342 | 2342 | 3342 |
| AfYBR198C | CaYBR198C | 343 | 1343 | 2343 | 3343 |
| AfYCL003W | CaYCL003W | 344 | 1344 | 2344 | 3344 |
| AfYCR012W | CaYCR012W | 345 | 1345 | 2345 | 3345 |
| AfYCR057C | CaYCR057C | 346 | 1346 | 2346 | 3346 |
| AfYDL084W | CaYDL084W | 347 | 1347 | 2347 | 3347 |
| AfYDL087C | CaYDL087C | 348 | 1348 | 2348 | 3348 |
| AfYDR002W | CaYDR002W | 349 | 1349 | 2349 | 3349 |
| AfYDR023W | CaYDR023W | 350 | 1350 | 2350 | 3350 |
| AfYDR045C | CaYDR045C | 351 | 1351 | 2351 | 3351 |
| AfYDR054C | CaYDR054C | 352 | 1352 | 2352 | 3352 |
| AfYDR060W | CaYDR060W | 353 | 1353 | 2353 | 3353 |
| AfYDR087C | CaYDR087C | 354 | 1354 | 2354 | 3354 |
| AfYDR226W | CaYDR226W | 355 | 1355 | 2355 | 3355 |
| AfYDR228C | CaYDR228C | 356 | 1356 | 2356 | 3356 |
| AfYDR238C | CaYDR238C | 357 | 1357 | 2357 | 3357 |
| AfYDR299W | CaYDR299W | 358 | 1358 | 2358 | 3358 |
| AfYDR328C | CaYDR328C | 359 | 1359 | 2359 | 3359 |
| AfYDR373W | CaYDR373W | 360 | 1360 | 2360 | 3360 |
| AfYDR390C | CaYDR390C | 361 | 1361 | 2361 | 3361 |
| AfYDR489W | CaYDR489W | 362 | 1362 | 2362 | 3362 |
| AfYEL032W | CaYEL032W | 363 | 1363 | 2363 | 3363 |
| AfYEL055C | CaYEL055C | 364 | 1364 | 2364 | 3364 |
| AfYER006W | CaYER006W | 365 | 1365 | 2365 | 3365 |
| AfYER036C | CaYER036C | 366 | 1366 | 2366 | 3366 |
| AfYFL045C | CaYFL045C | 367 | 1367 | 2367 | 3367 |
| AfYGL008C | CaYGL008C | 368 | 1368 | 2368 | 3366 |
| AfYGL048C | CaYGL048C | 369 | 1369 | 2369 | 3369 |
| AfYGL097W | CaYGL097W | 370 | 1370 | 2370 | 3370 |
| AfYGL112C | CaYGL112C | 371 | 1371 | 2371 | 3371 |
| AfYGL201C | CaYGL201C | 372 | 1372 | 2372 | 3372 |
| AfYGL207W | CaYGL207W | 373 | 1373 | 2373 | 3373 |
| AfYGL225W | CaYGL225W | 374 | 1374 | 2374 | 3374 |
| AfYGL245W | CaYGL245W | 375 | 1375 | 2375 | 3375 |
| AfYGR047C | CaYGR047C | 376 | 1376 | 2376 | 3376 |
| AfYGR048W | CaYGR048W | 377 | 1377 | 2377 | 3377 |
| AfYGR083C | CaYGR083C | 378 | 1378 | 2378 | 3378 |
| AfYGR185C | CaYGR185C | 379 | 1379 | 2379 | 3379 |
| AfYGR218W | CaYGR218W | 380 | 1380 | 2380 | 3380 |
| AfYGR267C | CaYGR267C | 381 | 1381 | 2381 | 3381 |
| AfYHR005C-A | CaYHR005C-A | 382 | 1382 | 2382 | 3382 |
| AfYHR072W-A | CaYHR072W-A | 383 | 1383 | 2383 | 3383 |
| AfYHR166C | CaYHR166C | 384 | 1384 | 2384 | 3384 |
| AfYHR188C | CaYHR188C | 385 | 1385 | 2385 | 3385 |
| AfYIL022W | CaYIL022W | 386 | 1386 | 2386 | 3386 |
| AfYIL109C | CaYIL109C | 387 | 1387 | 2387 | 3387 |
| AfYJL109C | CaYJL109C | 388 | 1388 | 2388 | 3388 |
| AfYJL111W | CaYJL111W | 389 | 1389 | 2389 | 3389 |
| AfYJL167W | CaYJL167W | 390 | 1390 | 2390 | 3390 |
| AfYJR064W | CaYJR064W | 391 | 1391 | 2391 | 3391 |
| AfYJR065C | CaYJR065C | 392 | 1392 | 2392 | 3392 |
| AfYKL013C | CaYKL013C | 393 | 1393 | 2393 | 3393 |
| AfYKL045W | CaYKL045W | 394 | 1394 | 2394 | 3394 |
| AfYKL104C | CaYKL104C | 395 | 1395 | 2395 | 3395 |
| AfYKL193C | CaYKL193C | 396 | 1396 | 2396 | 3396 |
| AfYLR088W | CaYLR088W | 397 | 1397 | 2397 | 3397 |
| AfYLR129W | CaYLR129W | 398 | 1398 | 2398 | 3398 |
| AfYLR274W | CaYLR274W | 399 | 1399 | 2399 | 3399 |
| AfYLR291C | CaYLR291C | 400 | 1400 | 2400 | 3400 |
| AfYLR293C | CaYLR293C | 401 | 1401 | 2401 | 3401 |
| AfYML064C | CaYML064C | 402 | 1402 | 2402 | 3402 |
| AfYMR049C | CaYMR049C | 403 | 1403 | 2403 | 3403 |
| AfYMR055C | CaYMR055C | 404 | 1404 | 2404 | 3404 |
| AfYMR131C | CaYMR131C | 405 | 1405 | 2405 | 3405 |
| AfYMR220W | CaYMR220W | 406 | 1406 | 2406 | 3406 |
| AfYMR309C | CaYMR309C | 407 | 1407 | 2407 | 3407 |
| AfYNL061W | CaYNL061W | 408 | 1408 | 2408 | 3408 |
| AfYNL113W | CaYNL113W | 409 | 1409 | 2409 | 3409 |
| AfYNL163C | CaYNL163C | 410 | 1410 | 2410 | 3410 |
| AfYNL178W | CaYNL178W | 411 | 1411 | 2411 | 3411 |
| AfYNL181W | CaYNL181W | 412 | 1412 | 2412 | 3412 |
| AfYNL263C | CaYNL263C | 413 | 1413 | 2413 | 3413 |
| AfYNR046W | CaYNR046W | 414 | 1414 | 2414 | 3414 |
| AfYOL034W | CaYOL034W | 415 | 1415 | 2415 | 3415 |
| AfYOR056C | CaYOR056C | 416 | 1416 | 2416 | 3416 |
| AfYOR057W | CaYOR057W | 417 | 1417 | 2417 | 3417 |
| AfYOR117W | CaYOR117W | 418 | 1418 | 2418 | 3418 |
| AfYOR207C | CaYOR207C | 419 | 1419 | 2419 | 3419 |
| AfYOR224C | CaYOR224C | 420 | 1420 | 2420 | 3420 |
| AfYOR259C | CaYOR259C | 421 | 1421 | 2421 | 3421 |
| AfYOR261C | CaYOR261C | 422 | 1422 | 2422 | 3422 |
| AfYOR262W | CaYOR262W | 423 | 1423 | 2423 | 3423 |
| AfYPL117C | CaYPL117C | 424 | 1424 | 2424 | 3424 |
| AfYPL122C | CaYPL122C | 425 | 1425 | 2425 | 3425 |
| AfYPL218W | CaYPL218W | 426 | 1426 | 2426 | 3426 |
| AfYPL235W | CaYPL235W | 427 | 1427 | 2427 | 3427 |
| AfYPR103W | CaYPR103W | 428 | 1428 | 2428 | 3428 |
| AfYPR112C | CaYPR112C | 429 | 1429 | 2429 | 3429 |
| AfYPR165W | CaYPR165W | 430 | 1430 | 2430 | 3430 |
| AfPRO1 | CaPRO1 | 431 | 1431 | 2431 | 3431 |
| AfYBL050W | CaYBL050W | 432 | 1432 | 2432 | 3432 |
| AfYDL029W | CaYDL029W | 433 | 1433 | 2433 | 3433 |
| AfYDR397C | CaYDR397C | 434 | 1434 | 2434 | 3434 |
| AfYDR460W | CaYDR460W | 435 | 1435 | 2435 | 3435 |
| AfYER094C | CaYER094C | 436 | 1436 | 2436 | 3436 |
| AfYER171W | CaYER171W | 437 | 1437 | 2437 | 3437 |
| AfYGL091C | CaYGL091C | 438 | 1438 | 2438 | 3438 |
| AfYGR074W | CaYGR074W | 439 | 1439 | 2439 | 3439 |
| AfYGR103W | CaYGR103W | 440 | 1440 | 2440 | 3440 |
| AfYGR253C | CaYGR253C | 441 | 1441 | 2441 | 3441 |
| AfYHR170W | CaYHR170W | 442 | 1442 | 2442 | 3442 |
| AfYJR017C | CaYJR017C | 443 | 1443 | 2443 | 3443 |
| AfYLR103C | CaYLR103C | 444 | 1444 | 2444 | 3444 |
| AfYNL075W | CaYNL075W | 445 | 1445 | 2445 | 3445 |
| AfYPL131W | CaYPL131W | 446 | 1446 | 2446 | 3446 |
| AfORF6_1717 | CaORF6_1717 | 447 | 1447 | 2447 | 3447 |
| AfORF6_2193 | CaORF6_2193 | 448 | 1448 | 2448 | 3448 |
| AfORF6_2398 | CaORF6_2398 | 449 | 1449 | 2449 | 3449 |
| AfORF6_4499 | CaORF6_4499 | 450 | 1450 | 2450 | 3450 |
| AfORF6_5520 | CaORF6_5520 | 451 | 1451 | 2451 | 3451 |
| AfORF6_7629 | CaORF6_7629 | 452 | 1452 | 2452 | 3452 |
| AfORF6_7847 | CaORF6_7847 | 453 | 1453 | 2453 | 3453 |
| AfORF6_8362 | CaORF6_8362 | 454 | 1454 | 2454 | 3454 |
| AfORF6_8377 | CaORF6_8377 | 455 | 1455 | 2455 | 3455 |
| AfORF6_8461 | CaORF6_8461 | 456 | 1456 | 2456 | 3456 |
| AfORF6_8607 | CaORF6_8607 | 457 | 1457 | 2457 | 3457 |
| AfORF6_8654 | CaORF6_8654 | 458 | 1458 | 2458 | 3458 |
| AfYBL020W | CaYBL020W | 459 | 1459 | 2459 | 3459 |
| AfYBL097W | CaYBL097W | 460 | 1460 | 2460 | 3460 |
| AfYBR002C | CaYBR002C | 461 | 1461 | 2461 | 3461 |
| AfYBR011C | CaYBR011C | 462 | 1462 | 2462 | 3462 |
| AfYBR087W | CaYBR087W | 463 | 1463 | 2463 | 3463 |
| AfYBR135W | CaYBR135W | 464 | 1464 | 2464 | 3464 |
| AfYBR243C | CaYBR243C | 465 | 1465 | 2465 | 3465 |
| AfYCL059C | CaYCL059C | 466 | 1466 | 2466 | 3466 |
| AfYDL102W | CaYDL102W | 467 | 1467 | 2467 | 3467 |
| AfYDL132W | CaYDL132W | 468 | 1468 | 2468 | 3468 |
| AfYDL141W | CaYDL141W | 469 | 1469 | 2469 | 3469 |
| AfYDL143W | CaYDL143W | 470 | 1470 | 2470 | 3470 |
| AfYDL145C | CaYDL145C | 471 | 1471 | 2471 | 3471 |
| AfYDL147W | CaYDL147W | 472 | 1472 | 2472 | 3472 |
| AfYDL195W | CaYDL195W | 473 | 1473 | 2473 | 3473 |
| AfYDL208W | CaYDL208W | 474 | 1474 | 2474 | 3474 |
| AfYDR170C | CaYDR170C | 475 | 1475 | 2475 | 3475 |
| AfYDR188W | CaYDR188W | 476 | 1476 | 2476 | 3476 |
| AfYDR189W | CaYDR189W | 477 | 1477 | 2477 | 3477 |
| AfYDR190C | CaYDR190C | 478 | 1478 | 2478 | 3478 |
| AfYDR235W | CaYDR235W | 479 | 1479 | 2479 | 3479 |
| AfYDR246W | CaYDR246W | 480 | 1480 | 2480 | 3480 |
| AfYDR324C | CaYDR324C | 481 | 1481 | 2481 | 3481 |
| AfYDR341C | CaYDR341C | 482 | 1482 | 2482 | 3482 |
| AfYDR365C | CaYDR365C | 483 | 1483 | 2483 | 3483 |
| AfYDR376W | CaYDR376W | 484 | 1484 | 2484 | 3484 |
| AfYDR394W | CaYDR394W | 485 | 1485 | 2485 | 3485 |
| AfYDR429C | CaYDR429C | 486 | 1486 | 2486 | 3486 |
| AfYER007W | CaYER007W | 487 | 1487 | 2487 | 3487 |
| AfYER048W-A | CaYER048W-A | 488 | 1488 | 2488 | 3488 |
| AfYER082C | CaYER082C | 489 | 1489 | 2489 | 3489 |
| AfYER148W | CaYER148W | 490 | 1490 | 2490 | 3490 |
| AfYFL002C | CaYFL002C | 491 | 1491 | 2491 | 3491 |
| AfYFR004W | CaYFR004W | 492 | 1492 | 2492 | 3492 |
| AfYFR031C | CaYFR031C | 493 | 1493 | 2493 | 3493 |
| AfYFR037C | CaYFR037C | 494 | 1494 | 2494 | 3494 |
| AfYGL011C | CaYGL011C | 495 | 1495 | 2495 | 3495 |
| AfYGL068W | CaYGL068W | 496 | 1496 | 2496 | 3496 |
| AfYGL103W | CaYGL103W | 497 | 1497 | 2497 | 3497 |
| AfYGR094W | CaYGR094W | 498 | 1498 | 2498 | 3498 |
| AfYHL015W | CaYHL015W | 499 | 1499 | 2499 | 3499 |
| AfYHR007C | CaYHR007C | 500 | 1500 | 2500 | 3500 |
| AfYHR020W | CaYHR020W | 501 | 1501 | 2501 | 3501 |
| AfYHR090C | CaYHR090C | 502 | 1502 | 2502 | 3502 |
| AfYHR148W | CaYHR148W | 503 | 1503 | 2503 | 3503 |
| AfYHR165C | CaYHR165C | 504 | 1504 | 2504 | 3504 |
| AfYHR190W | CaYHR190W | 505 | 1505 | 2505 | 3505 |
| AfYIL046W | CaYIL046W | 506 | 1506 | 2506 | 3506 |
| AfYIL078W | CaYIL078W | 507 | 1507 | 2507 | 3507 |
| AfYIR008C | CaYIR008C | 508 | 1508 | 2508 | 3508 |
| AfYJL014W | CaYJL014W | 509 | 1509 | 2509 | 3509 |
| AfYJL050W | CaYJL050W | 510 | 1510 | 2510 | 3510 |
| AfYJL069C | CaYJL069C | 511 | 1511 | 2511 | 3511 |
| AfYJL081C | CaYJL081C | 512 | 1512 | 2512 | 3512 |
| AfYJL104W | CaYJL104W | 513 | 1513 | 2513 | 3513 |
| AfYJL143W | CaYJL143W | 514 | 1514 | 2514 | 3514 |
| AfYJL153C | CaYJL153C | 515 | 1515 | 2515 | 3515 |
| AfYJL203W | CaYJL203W | 516 | 1516 | 2516 | 3516 |
| AfYJR007W | CaYJR007W | 517 | 1517 | 2517 | 3517 |
| AfYJR063W | CaYJR063W | 518 | 1518 | 2518 | 3518 |
| AfYJR072C | CaYJR072C | 519 | 1519 | 2519 | 3519 |
| AfYKL058W | CaYKL058W | 520 | 1520 | 2520 | 3520 |
| AfYKL060C | CaYKL060C | 521 | 1521 | 2521 | 3521 |
| AfYKL145W | CaYKL145W | 522 | 1522 | 2522 | 3522 |
| AfYKR068C | CaYKR068C | 523 | 1523 | 2523 | 3523 |
| AfYKR079C | CaYKR079C | 524 | 1524 | 2524 | 3524 |
| AfYLR078C | CaYLR078C | 525 | 1525 | 2525 | 3525 |
| AfYLR116W | CaYLR116W | 526 | 1526 | 2526 | 3526 |
| AfYLR153C | CaYLR153C | 527 | 1527 | 2527 | 3527 |
| AfYLR163C | CaYLR163C | 528 | 1528 | 2528 | 3528 |
| AfYLR208W | CaYLR208W | 529 | 1529 | 2529 | 3529 |
| AfYLR272C | CaYLR272C | 530 | 1530 | 2530 | 3530 |
| AfYLR276C | CaYLR276C | 531 | 1531 | 2531 | 3531 |
| AfYLR277C | CaYLR277C | 532 | 1532 | 2532 | 3532 |
| AfYLR336C | CaYLR336C | 533 | 1533 | 2533 | 3533 |
| AfYLR347C | CaYLR347C | 534 | 1534 | 2534 | 3534 |
| AfYLR355C | CaYLR355C | 535 | 1535 | 2535 | 3535 |
| AfYLR383W | CaYLR383W | 536 | 1536 | 2536 | 3536 |
| AfYML069W | CaYML069W | 537 | 1537 | 2537 | 3537 |
| AfYMR093W | CaYMR093W | 538 | 1538 | 2538 | 3538 |
| AfYMR235C | CaYMR235C | 539 | 1539 | 2539 | 3539 |
| AfYNL102W | CaYNL102W | 540 | 1540 | 2540 | 3540 |
| AfYNL189W | CaYNL189W | 541 | 1541 | 2541 | 3541 |
| AfYNL240C | CaYNL240C | 542 | 1542 | 2542 | 3542 |
| AfYNR050C | CaYNR050C | 543 | 1543 | 2543 | 3543 |
| AfYOL027C | CaYOL027C | 544 | 1544 | 2544 | 3544 |
| AfYOL097C | CaYOL097C | 545 | 1545 | 2545 | 3545 |
| AfYOL102C | CaYOL102C | 546 | 1546 | 2546 | 3546 |
| AfYOR048C | CaYOR048C | 547 | 1547 | 2547 | 3547 |
| AfYOR063W | CaYOR063W | 548 | 1548 | 2548 | 3548 |
| AfYOR116C | CaYOR116C | 549 | 1549 | 2549 | 3549 |
| AfYOR159C | CaYOR159C | 550 | 1550 | 2550 | 3550 |
| AfYOR204W | CaYOR204W | 551 | 1551 | 2551 | 3551 |
| AfYOR217W | CaYOR217W | 552 | 1552 | 2552 | 3552 |
| AfYOR341W | CaYOR341W | 553 | 1553 | 2553 | 3553 |
| AfYPL076W | CaYPL076W | 554 | 1554 | 2554 | 3554 |
| AfYPL211W | CaYPL211W | 555 | 1555 | 2555 | 3555 |
| AfYPL242C | CaYPL242C | 556 | 1556 | 2556 | 3556 |
| AfYPL266W | CaYPL266W | 557 | 1557 | 2557 | 3557 |
| AfYPR019W | CaYPR019W | 558 | 1558 | 2558 | 3558 |
| AfYPR176C | CaYPR176C | 559 | 1559 | 2559 | 3559 |
| AfYLR355C | CaYLR355C | 560 | 1560 | 2560 | 3560 |
| AfYGR083C | CaYGR083C | 561 | 1561 | 2561 | 3561 |
| AfYHR172W | CaYHR172W | 562 | 1562 | 2562 | 3562 |
| AfYOL130W | CaYOL130W | 563 | 1563 | 2563 | 3563 |
| AfYJL143W | CaYJL143W | 564 | 1564 | 2564 | 3564 |
| AfYNL039W | CaYNL039W | 565 | 1565 | 2565 | 3565 |
| AfYPR187W | CaYPR187W | 566 | 1566 | 2566 | 3566 |
| AfYPR144C | CaYPR144C | 567 | 1567 | 2567 | 3567 |
| AfYGR002C | CaYGR002C | 568 | 1568 | 2568 | 3568 |
| AfYKL059C | CaYKL059C | 569 | 1569 | 2569 | 3569 |
| AfYGR009C | CaYGR009C | 570 | 1570 | 2570 | 3570 |
| AfYGR186W | CaYGR186W | 571 | 1571 | 2571 | 3571 |
| AfORF6_1498 | CaORF6_1498 | 572 | 1572 | 2572 | 3572 |
| AfORF6_3819 | CaORF6_3819 | 573 | 1573 | 2573 | 3573 |
| AfORF6_4463 | CaORF6_4463 | 574 | 1574 | 2574 | 3574 |
| AfORF6_6069 | CaORF6_6069 | 575 | 1575 | 2575 | 3575 |
| AfORF6_6140 | CaORF6_6140 | 576 | 1576 | 2576 | 3576 |
| AfORF6_6390 | CaORF6_6390 | 577 | 1577 | 2577 | 3577 |
| AfORF6_6660 | CaORF6_6660 | 578 | 1578 | 2578 | 3578 |
| AfORF6_6664 | CaORF6_6664 | 579 | 1579 | 2579 | 3579 |
| AfORF6_6808 | CaORF6_6808 | 580 | 1580 | 2580 | 3580 |
| AfORF6_6933 | CaORF6_6933 | 581 | 1581 | 2581 | 3581 |
| AfORF6_6939 | CaORF6_6939 | 582 | 1582 | 2582 | 3582 |
| AfORF6_7203 | CaORF6_7203 | 583 | 1583 | 2583 | 3583 |
| AfORF6_8654 | CaORF6_8654 | 584 | 1584 | 2584 | 3584 |
| AfYBR038W | CaYBR038W | 585 | 1585 | 2585 | 3585 |
| AfYER059W | CaYER059W | 586 | 1586 | 2586 | 3586 |
| AfYGL233W | CaYGL233W | 587 | 1587 | 2587 | 3587 |
| AfYNL048W | CaYNL048W | 588 | 1588 | 2588 | 3588 |
| AfYNL221C | CaYNL221C | 589 | 1589 | 2589 | 3589 |
| AfYOL066C | CaYOL066C | 590 | 1590 | 2590 | 3590 |
| AfORF6_3026 | CaORF6_3026 | 591 | 1591 | 2591 | 3591 |
| AfORF6_4005 | CaORF6_4005 | 592 | 1592 | 2592 | 3592 |
| AfYNL256W | CaYNL256W | 593 | 1593 | 2593 | 3593 |
| AfYPL128C | CaYPL128C | 594 | 1594 | 2594 | 3594 |
| AfHIS3 | | 4001 | | | |
| AfHIS3 Prom. Replace | | 4002 | | | |
| AfHIS3 5′ primer | | 4003 | | | |
| AfHIS3-pyrG primer | | 4004 | | | |
| pyrG-PglaA 5′ | | 4005 | | | |
| pyrG-PglaA 3′ | | 4006 | | | |
| pyrG-AfHIS3 | | 4007 | | | |
| AfHIS3 3′ primer | | 4008 | | | |
| AfALB1 | | 4009 | | | |
| AfALB1 prom. Replace | | 4010 | | | |
| AfALB1 5′ primer | | 4011 | | | |
| AfALB1-pyrG | | 4012 | | | |
| AnpyrG upstream | | 4013 | | | |
| AnpyrG downstream | | 4014 | | | |
| pyrG-AfALB1 | | 4015 | | | |
| AfALB1 3′ primer | | 4016 | | | |
| AfPYROA ORF | | 4017 | | | |
| AfPYROA ORF | | 4018 | | | |
| AfPYROA genomic | | 4019 | | | |
| AfPYROA Prom Replac | | 4020 | | | |
| AfPYROA 5′ primer | | 4021 | | | |
| AfPYROA-pyrG | | 4022 | | | |
| pyrG-AfPYROA | | 4023 | | | |
| AfPYROA 3′ primer | | 4024 | | | |
| AfERG11 | | 4025 | | | |
| AfERG11 | | 4026 | | | |
| AfERG11beta | | 4027 | | | |
| AfERG11beta | | 4028 | | | |
| AfALG7 genomic | | 4029 | | | |
| AfALG7 ORF | | 4030 | | | |
| AfALG7 ORF | | 4031 | | | |
| AfAAD14 genomic | | 4032 | | | |
| AfAAD14 ORF | | 4033 | | | |
| AfAAD14 ORF | | 4034 | | | |
| AfEF-2 ORF + 5′ | | 4035 | | | |
| AfEF-2 ORF | | 4036 | | | |
| AfEF-2 ORF | | 4037 | | | |
| AfErg8 Prom Replac | | 4038 | | | |
| AfYMR290C | CaYMR290C | 5001 | 6001 | 7001 | 8001 |
| AfYPR034W | CaYPR034W | 5002 | 6002 | 7002 | 8002 |
| AfORF6_4497 | CaORF6_4497 | 5003 | 6003 | 7003 | 8003 |
| AfYJL008C | CaYJL008C | 5004 | 6004 | 7004 | 8004 |
| AfYIL068C | CaYIL068C | 5005 | 6005 | 7005 | 8005 |
| AfYHR196W | CaYHR196W | 5006 | 6006 | 7006 | 8006 |
| AfYMR197C | CaYMR197C | 5007 | 6007 | 7007 | 8007 |
| AfYLR100W | CaYLR100W | 5008 | 6008 | 7008 | 8008 |
| AfYDL055C | CaYDL055C | 5009 | 6009 | 7009 | 8009 |
| AfYDL043C | CaYDL043C | 5010 | 6010 | 7010 | 8010 |
| AfYJL054W | CaYJL054W | 5011 | 6011 | 7011 | 8011 |
| AfYHR072W2 | CaYHR072W2 | 5012 | 6012 | 7012 | 8012 |
| AfYPR119W | CaYPR119W | 5013 | 6013 | 7013 | 8013 |
| AfYDR013W | CaYDR013W | 5014 | 6014 | 7014 | 8014 |
| AfYGR255C | CaYGR255C | 5015 | 6015 | 7015 | 8015 |
| AfYDR353W | CaYDR353W | 5016 | 6016 | 7016 | 8016 |
| AfYNR053C | CaYNR053C | 5017 | 6017 | 7017 | 8017 |
| AfYLR222C | CaYLR222C | 5018 | 6018 | 7018 | 8018 |
| AfYER025W | CaYER025W | 5019 | 6019 | 7019 | 8019 |
| AfYOR272W | CaYOR272W | 5020 | 6020 | 7020 | 8020 |
| AfYGL206C | CaYGL206C | 5021 | 6021 | 7021 | 8021 |
| AfYMR208W | CaYMR208W | 5022 | 6022 | 7022 | 8022 |
| AfYKL019W | CaYKL019W | 5023 | 6023 | 7023 | 8023 |
| AfYJR006W | CaYJR006W | 5024 | 6024 | 7024 | 8024 |
| AfYIL075C | CaYIL075C | 5025 | 6025 | 7025 | 8025 |
| AfYER070W | CaYER070W | 5026 | 6026 | 7026 | 8026 |
| AfYMR113W | CaYMR113W | 5027 | 6027 | 7027 | 8027 |
| AfYIR011C | CaYIR011C | 5028 | 6028 | 7028 | 8028 |
| AfYER012W | CaYER012W | 5029 | 6029 | 7029 | 8029 |
| AfORF6_8837 | CaORF6_8837 | 5030 | 6030 | 7030 | 8030 |
| AfYJR002W | CaYJR002W | 5031 | 6031 | 7031 | 8031 |
| AfYPL043W | CaYPL043W | 5032 | 6032 | 7032 | 8032 |
| AfYNL110C | CaYNL110C | 5033 | 6033 | 7033 | 8033 |
| AfORF6_4899 | CaORF6_4899 | 5034 | 6034 | 7034 | 8034 |
| AfORF6_5199 | CaORF6_5199 | 5035 | 6035 | 7035 | 8035 |
| AfYFR052W | CaYFR052W | 5036 | 6036 | 7036 | 8036 |
| AfYMR146C | CaYMR146C | 5037 | 6037 | 7037 | 8037 |
| AfYGL003C | CaYGL003C | 5038 | 6038 | 7038 | 8038 |
| AfYFL008W | CaYFL008W | 5039 | 6039 | 7039 | 8039 |
| AfYDL028C | CaYDL028C | 5040 | 6040 | 7040 | 8040 |
| AfYHR070W | CaYHR070W | 5041 | 6041 | 7041 | 8041 |
| AfYLR115W | CaYLR115W | 5042 | 6042 | 7042 | 8042 |
| AfYLR197W | CaYLR197W | 5043 | 6043 | 7043 | 8043 |
| AfYOL022C | CaYOL022C | 5044 | 6044 | 7044 | 8044 |
| AfYIL062C | CaYIL062C | 5045 | 6045 | 7045 | 8045 |
| AfYPR086W | CaYPR086W | 5046 | 6046 | 7046 | 8046 |
| AfYPR183W | CaYPR183W | 5047 | 6047 | 7047 | 8047 |
| AfYNL126W | CaYNL126W | 5048 | 6048 | 7048 | 8048 |
| AfYLR167W | CaYLR167W | 5049 | 6049 | 7049 | 8049 |
| AfYJL010C | CaYJL010C | 5050 | 6050 | 7050 | 8050 |
| AfYLR026C | CaYLR026C | 5051 | 6051 | 7051 | 8051 |
| AfYBR192W | CaYBR192W | 5052 | 6052 | 7052 | 8052 |
| AfYDR088C | CaYDR088C | 5053 | 6053 | 7053 | 8053 |
| AfYOR280C | CaYOR280C | 5054 | 6054 | 7054 | 8054 |
| AfYNL244C | CaYNL244C | 5055 | 6055 | 7055 | 8055 |
| AfYER021W | CaYER021W | 5056 | 6056 | 7056 | 8056 |
| AfYLR186W | CaYLR186W | 5057 | 6057 | 7057 | 8057 |
| AfYDR527W | CaYDR527W | 5058 | 6058 | 7058 | 8058 |
| AfYDL017W | CaYDL017W | 5059 | 6059 | 7059 | 8059 |
| AfYGR090W | CaYGR090W | 5060 | 6060 | 7060 | 8060 |
| AfYKL182W | CaYKL182W | 5061 | 6061 | 7061 | 8061 |
| AfYPL231W | CaYPL231W | 5062 | 6062 | 7062 | 8062 |
| AfYDR120C | CaYDR120C | 5063 | 6063 | 7063 | 8063 |
| AfYBR080C | CaYBR080C | 5064 | 6064 | 7064 | 8064 |
| AfYPR082C | CaYPR082C | 5065 | 6065 | 7065 | 8065 |
| AfYOR310C | CaYOR310C | 5066 | 6066 | 7066 | 8066 |
| AfYDL205C | CaYDL205C | 5067 | 6067 | 7067 | 8067 |
| AfYIR022W | CaYIR022W | 5068 | 6068 | 7068 | 8068 |
| AfYGR246C | CaYGR246C | 5069 | 6069 | 7069 | 8069 |
| AfYCL017C | CaYCL017C | 5070 | 6070 | 7070 | 8070 |
| AfYBL023C | CaYBL023C | 5071 | 6071 | 7071 | 8071 |
| AfORF6_5147 | CaORF6_5147 | 5072 | 6072 | 7072 | 8072 |
| AfYGR209C | CaYGR209C | 5073 | 6073 | 7073 | 8073 |
| AfYLR306W | CaYLR306W | 5074 | 6074 | 7074 | 8074 |
| AfYLL012W | CaYLL012W | 5075 | 6075 | 7075 | 8075 |
| AfYPL217C | CaYPL217C | 5076 | 6076 | 7076 | 8076 |
| AfYPR110C | CaYPR110C | 5077 | 6077 | 7077 | 8077 |
| AfYMR288W | CaYMR288W | 5078 | 6078 | 7078 | 8078 |
| AfYJL074C | CaYJL074C | 5079 | 6079 | 7079 | 8079 |
| AfYOR119C | CaYOR119C | 5080 | 6080 | 7080 | 8080 |
| AfYNR017W | CaYNR017W | 5081 | 6081 | 7081 | 8081 |
| AfYDL060W | CaYDL060W | 5082 | 6082 | 7082 | 8082 |
| AfYIL021W | CaYIL021W | 5083 | 6083 | 7083 | 8083 |
| AfTRP5 | CaTRP5 | 5084 | 6084 | 7084 | 8084 |
| AfYPL203W | CaYPL203W | 5085 | 6085 | 7085 | 8085 |
| AfYPL094C | CaYPL094C | 5086 | 6086 | 7086 | 8086 |
| AfYBL026W | CaYBL026W | 5087 | 6087 | 7087 | 8087 |
| AfYKL210W | CaYKL210W | 5088 | 6088 | 7088 | 8088 |
| AfYIL003W | CaYIL003W | 5089 | 6089 | 7089 | 8089 |
| AfYDR212W | CaYDR212W | 5090 | 6090 | 7090 | 8090 |
| AfYLR002C | CaYLR002C | 5091 | 6091 | 7091 | 8091 |
| AfYLR397C | CaYLR397C | 5092 | 6092 | 7092 | 8092 |
| AfYGL001C | CaYGL001C | 5093 | 6093 | 7093 | 8093 |
| AfYPR041W | CaYPR041W | 5094 | 6094 | 7094 | 8094 |
| AfYGR156W | CaYGR156W | 5095 | 6095 | 7095 | 8095 |
| AfYPL010W | CaYPL010W | 5096 | 6096 | 7096 | 8096 |
| AfYLR259C | CaYLR259C | 5097 | 6097 | 7097 | 8097 |
| AfYBR196C | CaYBR196C | 5098 | 6098 | 7098 | 8098 |
| AfYJR045C | CaYJR045C | 5099 | 6099 | 7099 | 8099 |
| AfYBR202W | CaYBR202W | 5100 | 6100 | 7100 | 8100 |
| AfYNL245C | CaYNL245C | 5101 | 6101 | 7101 | 8101 |
| AfORF6_4747 | CaORF6_4747 | 5102 | 6102 | 7102 | 8102 |
| AfORF6_7375 | CaORF6_7375 | 5103 | 6103 | 7103 | 8103 |
| AfYER136W | CaYER136W | 5104 | 6104 | 7104 | 8104 |
| AfYPL093W | CaYPL093W | 5105 | 6105 | 7105 | 8105 |
| AfYBR159W | CaYBR159W | 5106 | 6106 | 7106 | 8106 |
| AfYJL034W | CaYJL034W | 5107 | 6107 | 7107 | 8107 |
| AfYDR172W | CaYDR172W | 5108 | 6108 | 7108 | 8108 |
| AfYOR157C | CaYOR157C | 5109 | 6109 | 7109 | 8109 |
| AfYLR127C | CaYLR127C | 5110 | 6110 | 7110 | 8110 |
| AfYMR213W | CaYMR213W | 5111 | 6111 | 7111 | 8111 |
| AfYHR019C | CaYHR019C | 5112 | 6112 | 7112 | 8112 |
| AfYLR229C | CaYLR229C | 5113 | 6113 | 7113 | 8113 |
| AfYGL130W | CaYGL130W | 5114 | 6114 | 7114 | 8114 |
| AfYJL002C | CaYJL002C | 5115 | 6115 | 7115 | 8115 |
| AfYPR105C | CaYPR105C | 5116 | 6116 | 7116 | 8116 |
| AfYBR055C | CaYBR055C | 5117 | 6117 | 7117 | 8117 |
| AfYPR175W | CaYPR175W | 5118 | 6118 | 7118 | 8118 |
| AfYPL063W | CaYPL063W | 5119 | 6119 | 7119 | 8119 |
| AfYFL022C | CaYFL022C | 5120 | 6120 | 7120 | 8120 |
| AfYML075C | CaYML075C | 5121 | 6121 | 7121 | 8121 |
| AfYNL222W | CaYNL222W | 5122 | 6122 | 7122 | 8122 |
| AfYLR086W | CaYLR086W | 5123 | 6123 | 7123 | 8123 |
| AfYOL142W | CaYOL142W | 5124 | 6124 | 7124 | 8124 |
| AfYHR186C | CaYHR186C | 5125 | 6125 | 7125 | 8125 |
| AfYNL287W | CaYNL287W | 5126 | 6126 | 7126 | 8126 |
| AfYLL018C | CaYLL018C | 5127 | 6127 | 7127 | 8127 |
| AfYAL015C | CaYAL015C | 5128 | 6128 | 7128 | 8128 |
| AfYOR335C | CaYOR335C | 5129 | 6129 | 7129 | 8129 |
| AfYDL193W | CaYDL193W | 5130 | 6130 | 7130 | 8130 |
| AfYML126C | CaYML126C | 5131 | 6131 | 7131 | 8131 |
| AfYDR404C | CaYDR404C | 5132 | 6132 | 7132 | 8132 |
| AfYML130C | CaYML130C | 5133 | 6133 | 7133 | 8133 |
| AfYKR081C | CaYKR081C | 5134 | 6134 | 7134 | 8134 |
| AfYER172C | CaYER172C | 5135 | 6135 | 7135 | 8135 |
| AfYOL010W | CaYOL010W | 5136 | 6136 | 7136 | 8136 |
| AfYPR178W | CaYPR178W | 5137 | 6137 | 7137 | 8137 |
| AfORF6_4974 | CaORF6_4974 | 5138 | 6138 | 7138 | 8138 |
| AfYGL022W | CaYGL022W | 5139 | 6139 | 7139 | 8139 |
| AfYGR280C | CaYGR280C | 5140 | 6140 | 7140 | 8140 |
| AfYDL108W | CaYDL108W | 5141 | 6141 | 7141 | 8141 |
| AfYOL005C | CaYOL005C | 5142 | 6142 | 7142 | 8142 |
| AfYJL194W | CaYJL194W | 5143 | 6143 | 7143 | 8143 |
| AfYOR151C | CaYOR151C | 5144 | 6144 | 7144 | 8144 |
| AfYCL031C | CaYCL031C | 5145 | 6145 | 7145 | 8145 |
| AfYOR210W | CaYOR210W | 5146 | 6146 | 7146 | 8146 |
| AfYGL123W | CaYGL123W | 5147 | 6147 | 7147 | 8147 |
| AfYOL139C | CaYOL139C | 5148 | 6148 | 7148 | 8148 |
| AfORF6_8482 | CaORF6_8482 | 5149 | 6149 | 7149 | 8149 |
| AfYKL035W | CaYKL035W | 5150 | 6150 | 7150 | 8150 |
| AfORF6_5210 | CaORF6_5210 | 5151 | 6151 | 7151 | 8151 |
| AfYOR294W | CaYOR294W | 5152 | 6152 | 7152 | 8152 |
| AfYJL001W | CaYJL001W | 5153 | 6153 | 7153 | 8153 |
| AfYHR052W | CaYHR052W | 5154 | 6154 | 7154 | 8154 |
| AfYNL313C | CaYNL313C | 5155 | 6155 | 7155 | 8155 |
| AfYLR249W | CaYLR249W | 5156 | 6156 | 7156 | 8156 |
| AfYKL181W | CaYKL181W | 5157 | 6157 | 7157 | 8157 |
| AfYML092C | CaYML092C | 5158 | 6158 | 7158 | 8158 |
| AfYGL106W | CaYGL106W | 5159 | 6159 | 7159 | 8159 |
| AfYLR243W | CaYLR243W | 5160 | 6160 | 7160 | 8160 |
| AfYPL151C | CaYPL151C | 5161 | 6161 | 7161 | 8161 |
| AfYER125W | CaYER125W | 5162 | 6162 | 7162 | 8162 |
| AfYNL006W | CaYNL006W | 5163 | 6163 | 7163 | 8163 |
| AfYJL087C | CaYJL087C | 5164 | 6164 | 7164 | 8164 |
| AfYBR237W | CaYBR237W | 5165 | 6165 | 7165 | 8165 |
| AfYLR117C | CaYLR117C | 5166 | 6166 | 7166 | 8166 |
| AfYDR091C | CaYDR091C | 5167 | 6167 | 7167 | 8167 |
| AfYGL040C | CaYGL040C | 5168 | 6168 | 7168 | 8168 |
| AfYDR454C | CaYDR454C | 5169 | 6169 | 7169 | 8169 |
| AfYDR267C | CaYDR267C | 5170 | 6170 | 7170 | 8170 |
| AfYGR264C | CaYGR264C | 5171 | 6171 | 7171 | 8171 |
| AfYMR308C | CaYMR308C | 5172 | 6172 | 7172 | 8172 |
| AfYNL124W | CaYNL124W | 5173 | 6173 | 7173 | 8173 |
| AfYDR473C | CaYDR473C | 5174 | 6174 | 7174 | 8174 |
| AfYBR154C | CaYBR154C | 5175 | 6175 | 7175 | 8175 |
| AfYDL140C | CaYDL140C | 5176 | 6176 | 7176 | 8176 |
| AfYHR005C | CaYHR005C | 5177 | 6177 | 7177 | 8177 |
| AfYER003C | CaYER003C | 5178 | 6178 | 7178 | 8178 |
| AfYBL076C | CaYBL076C | 5179 | 6179 | 7179 | 8179 |
| AfYBR265W | CaYBR265W | 5180 | 6180 | 7180 | 8180 |
| AfYDL153C | CaYDL153C | 5181 | 6181 | 7181 | 8181 |
| AfYHR024C | CaYHR024C | 5182 | 6182 | 7182 | 8182 |
| AfYDR062W | CaYDR062W | 5183 | 6183 | 7183 | 8183 |
| AfYHR169W | CaYHR169W | 5184 | 6184 | 7184 | 8184 |
| AfYBR256C | CaYBR256C | 5185 | 6185 | 7185 | 8185 |
| AfYLL011W | CaYLL011W | 5186 | 6186 | 7186 | 8186 |
| AfYDL097C | CaYDL097C | 5187 | 6187 | 7187 | 8187 |
| AfYCR052W | CaYCR052W | 5188 | 6188 | 7188 | 8188 |
| AfYPL169C | CaYPL169C | 5189 | 6189 | 7189 | 8189 |
| AfYNL132W | CaYNL132W | 5190 | 6190 | 7190 | 8190 |
| AfYNL308C | CaYNL308C | 5191 | 6191 | 7191 | 8191 |
| AfYLR060W | CaYLR060W | 5192 | 6192 | 7192 | 8192 |
| AfYML093W | CaYML093W | 5193 | 6193 | 7193 | 8193 |
| AfORF6_2086-2 | CaORF6_2086-2 | 5194 | 6194 | 7194 | 8194 |
| AfYLR029C | CaYLR029C | 5195 | 6195 | 7195 | 8195 |
| AfYBR088C | CaYBR088C | 5196 | 6196 | 7196 | 8196 |
| AfYKR086W | CaYKR086W | 5197 | 6197 | 7197 | 8197 |
| AfYMR015C | CaYMR015C | 5198 | 6198 | 7198 | 8198 |
| AfYAL035W | CaYAL035W | 5199 | 6199 | 7199 | 8199 |
| AfYJR123W | CaYJR123W | 5200 | 6200 | 7200 | 8200 |
| AfYGR245C | CaYGR245C | 5201 | 6201 | 7201 | 8201 |
| AfPBS2 | CaPBS2 | 5202 | 6202 | 7202 | 8202 |
| AfYFL018C | CaYFL018C | 5203 | 6203 | 7203 | 8203 |
| AfYOR206W | CaYOR206W | 5204 | 6204 | 7204 | 8204 |
| AfYNR038W | CaYNR038W | 5205 | 6205 | 7205 | 8205 |
| AfYML085C | CaYML085C | 5206 | 6206 | 7206 | 8206 |
| AfYKL125W | CaYKL125W | 5207 | 6207 | 7207 | 8207 |
| AfYLR196W | CaYLR196W | 5208 | 6208 | 7208 | 8208 |
| AfYNR035C | CaYNR035C | 5209 | 6209 | 7209 | 8209 |
| AfYPR107C | CaYPR107C | 5210 | 6210 | 7210 | 8210 |
| AfYNL280C | CaYNL280C | 5211 | 6211 | 7211 | 8211 |
| AfYIL142W | CaYIL142W | 5212 | 6212 | 7212 | 8212 |
| AfYCR072C | CaYCR072C | 5213 | 6213 | 7213 | 8213 |
| AfYER031C | CaYER031C | 5214 | 6214 | 7214 | 8214 |
| AfYOL077C | CaYOL077C | 5215 | 6215 | 7215 | 8215 |
| AfYNL088W | CaYNL088W | 5216 | 6216 | 7216 | 8216 |
| AfYER113C | CaYER113C | 5217 | 6217 | 7217 | 8217 |
| AfYKR062W | CaYKR062W | 5218 | 6218 | 7218 | 8218 |
| AfYPL028W | CaYPL028W | 5219 | 6219 | 7219 | 8219 |
| AfYKL046C | CaYKL046C | 5220 | 6220 | 7220 | 8220 |
| AfYBR142W | CaYBR142W | 5221 | 6221 | 7221 | 8221 |
| AfYDR052C | CaYDR052C | 5222 | 6222 | 7222 | 8222 |
| AfYLR300W | CaYLR300W | 5223 | 6223 | 7223 | 8223 |
| AfYPR010C | CaYPR010C | 5224 | 6224 | 7224 | 8224 |
| AfORF6_2086 | CaORF6_2086 | 5225 | 6225 | 7225 | 8225 |
| AfYDR037W | CaYDR037W | 5226 | 6226 | 7226 | 8226 |
| AfYPR108W | CaYPR108W | 5227 | 6227 | 7227 | 8227 |
| AfYFR050C | CaYFR050C | 5228 | 6228 | 7228 | 8228 |
| AfYBR234C | CaYBR234C | 5229 | 6229 | 7229 | 8229 |
| AfYHR174W | CaYHR174W | 5230 | 6230 | 7230 | 8230 |
| AfYBR070C | CaYBR070C | 5231 | 6231 | 7231 | 8231 |
| AfYGR211W | CaYGR211W | 5232 | 6232 | 7232 | 8232 |
| AfYOR095C | CaYOR095C | 5233 | 6233 | 7233 | 8233 |
| AfYHR042W | CaYHR042W | 5234 | 6234 | 7234 | 8234 |
| AfYJL033W | CaYJL033W | 5235 | 6235 | 7235 | 8235 |
| AfYDL031W | CaYDL031W | 5236 | 6236 | 7236 | 8236 |
| AfYLR342W | CaYLR342W | 5237 | 6237 | 7237 | 8237 |
| AfYDR211W | CaYDR211W | 5238 | 6238 | 7238 | 8238 |
| AfYPL160W | CaYPL160W | 5239 | 6239 | 7239 | 8239 |
| AfYDR356W | CaYDR356W | 5240 | 6240 | 7240 | 8240 |
| AfYFL038C | CaYFL038C | 5241 | 6241 | 7241 | 8241 |
| AfYFR002W | CaYFR002W | 5242 | 6242 | 7242 | 8242 |
| AfYOR074C | CaYOR074C | 5243 | 6243 | 7243 | 8243 |
| AfYCL054W | CaYCL054W | 5244 | 6244 | 7244 | 8244 |
| AfYJL026W | CaYJL026W | 5245 | 6245 | 7245 | 8245 |
| AfYJL039C | CaYJL039C | 5246 | 6246 | 7246 | 8246 |
| AfYML025C | CaYML025C | 5247 | 6247 | 7247 | 8247 |
| AfORF6_1934 | CaORF6_1934 | 5248 | 6248 | 7248 | 8248 |
| AfYDR361C | CaYDR361C | 5249 | 6249 | 7249 | 8249 |
| AfYGL065C | CaYGL065C | 5250 | 6250 | 7250 | 8250 |
| AfYNL232W | CaYNL232W | 5251 | 6251 | 7251 | 8251 |
| AfYER023W | CaYER023W | 5252 | 6252 | 7252 | 8252 |
| AfYBR060C | CaYBR060C | 5253 | 6253 | 7253 | 8253 |
| AfYLR378C | CaYLR378C | 5254 | 6254 | 7254 | 8254 |
| AfORF6_8025 | CaORF6_8025 | 5255 | 6255 | 7255 | 8255 |
| AfYDL007W | CaYDL007W | 5256 | 6256 | 7256 | 8256 |
| AfORF6_5739 | CaORF6_5739 | 5257 | 6257 | 7257 | 8257 |
| AfYNL277W | CaYNL277W | 5258 | 6258 | 7258 | 8258 |
| AfYPR048W | CaYPR048W | 5259 | 6259 | 7259 | 8259 |
| AfYHR088W | CaYHR088W | 5260 | 6260 | 7260 | 8260 |
| AfYPR016C | CaYPR016C | 5261 | 6261 | 7261 | 8261 |
| AfYIL126W | CaYIL126W | 5262 | 6262 | 7262 | 8262 |
| AfYLR105C | CaYLR105C | 5263 | 6263 | 7263 | 8263 |
| AfYHR072W | CaYHR072W | 5264 | 6264 | 7264 | 8264 |
| AfYBR160W | CaYBR160W | 5265 | 6265 | 7265 | 8265 |
| AfYBL040C | CaYBL040C | 5266 | 6266 | 7266 | 8266 |
| AfYMR240C | CaYMR240C | 5267 | 6267 | 7267 | 8267 |
| AfYLR175W | CaYLR175W | 5268 | 6268 | 7268 | 8268 |
| AfYLR175W2 | CaYLR175W2 | 5269 | 6269 | 7269 | 8269 |
| AfYHR074W | CaYHR074W | 5270 | 6270 | 7270 | 8270 |
| AfYPR088C | CaYPR088C | 5271 | 6271 | 7271 | 8271 |
| AfYDL030W | CaYDL030W | 5272 | 6272 | 7272 | 8272 |
| AfYNL062C | CaYNL062C | 5273 | 6273 | 7273 | 8273 |
| AfYDR196C | CaYDR196C | 5274 | 6274 | 7274 | 8274 |
| AfYOL038W | CaYOL038W | 5275 | 6275 | 7275 | 8275 |
| AfYDL217C | CaYDL217C | 5276 | 6276 | 7276 | 8276 |
| AfYOR250C | CaYOR250C | 5277 | 6277 | 7277 | 8277 |
| AfYDR167W | CaYDR167W | 5278 | 6278 | 7278 | 8278 |
| AfYGL120C | CaYGL120C | 5279 | 6279 | 7279 | 8279 |
| AfYHR027C | CaYHR027C | 5280 | 6280 | 7280 | 8280 |
| AfYER013W | CaYER013W | 5281 | 6281 | 7281 | 8281 |
| AfORF6_569 | CaORF6_569 | 5282 | 6282 | 7282 | 8282 |
| AfORF6_6011 | CaORF6_6011 | 5283 | 6283 | 7283 | 8283 |
| AfYNL247W | CaYNL247W | 5284 | 6284 | 7284 | 8284 |
| AfYJL125C | CaYJL125C | 5285 | 6285 | 7285 | 8285 |
| AfYML125C | CaYML125C | 5286 | 6286 | 7286 | 8286 |
| AfYJR076C | CaYJR076C | 5287 | 6287 | 7287 | 8287 |
| AfYGR070W | CaYGR070W | 5288 | 6288 | 7288 | 8288 |
| AfYDL105W | CaYDL105W | 5289 | 6289 | 7289 | 8289 |
| AfYHR023W | CaYHR023W | 5290 | 6290 | 7290 | 8290 |
| AfLYS4 | CaLYS4 | 5291 | 6291 | 7291 | 8291 |
| AfYDR062W2 | CaYDR062W2 | 5292 | 6292 | 7292 | 8292 |
| AfYMR203W | CaYMR203W | 5293 | 6293 | 7293 | 8293 |
| AfYOL094C | CaYOL094C | 5294 | 6294 | 7294 | 8294 |
| AfYDR407C | CaYDR407C | 5295 | 6295 | 7295 | 8295 |
| AfYOR287C | CaYOR287C | 5296 | 6296 | 7296 | 8296 |
| AfYLL031C | CaYLL031C | 5297 | 6297 | 7297 | 8297 |
| AfYPL085W | CaYPL085W | 5298 | 6298 | 7298 | 8298 |
| AfYMR260C | CaYMR260C | 5299 | 6299 | 7299 | 8299 |
| AfYFL017C | CaYFL017C | 5300 | 6300 | 7300 | 8300 |
| AfYMR218C | CaYMR218C | 5301 | 6301 | 7301 | 8301 |
| AfYEL026W | CaYEL026W | 5302 | 8302 | 7302 | 8302 |
| AfYDL207W | CaYDL207W | 5303 | 5303 | 7303 | 8303 |
| AfYNL131W | CaYNL131W | 5304 | 6304 | 7304 | 8304 |
| AfYNR026C | CaYNR026C | 5305 | 6305 | 7305 | 8305 |
| AfYOR004W | CaYOR004W | 5306 | 6306 | 7306 | 8306 |
| AfYBR254C | CaYBR254C | 5307 | 6307 | 7307 | 8307 |
| AfYOR232W | CaYOR232W | 5308 | 6308 | 7308 | 8308 |
| AfYNR043W | CaYNR043W | 5309 | 6309 | 7309 | 8309 |
| AfYOR257W | CaYOR257W | 5310 | 6310 | 7310 | 8310 |
| AfYGR060W | CaYGR060W | 5311 | 6311 | 7311 | 8311 |
| AfYJR112W | CaYJR112W | 5312 | 6312 | 7312 | 8312 |
| AfYPR186C | CaYPR186C | 5313 | 6313 | 7313 | 8313 |
| AfYBR079C | CaYBR079C | 5314 | 6314 | 7314 | 8314 |
| AfYPR056W | CaYPR056W | 5315 | 6315 | 7315 | 8315 |
| AfYDR472W | CaYDR472W | 5316 | 6316 | 7316 | 8316 |
| AfYGR172C | CaYGR172C | 5317 | 6317 | 7317 | 8317 |
| AfYMR028W | CaYMR028W | 5318 | 6318 | 7318 | 8318 |
| AfYMR227C | CaYMR227C | 5319 | 6319 | 7319 | 8319 |
| AfYGR029W | CaYGR029W | 5320 | 6320 | 7320 | 8320 |
| AfYPR025C | CaYPR025C | 5321 | 6321 | 7321 | 8321 |
| AfYOR145C | CaYOR145C | 5322 | 6322 | 7322 | 8322 |
| AfYBL041W | CaYBL041W | 5323 | 6323 | 7323 | 8323 |
| AfYHR122W | CaYHR122W | 5324 | 6324 | 7324 | 8324 |
| AfYPR113W | CaYPR113W | 5325 | 6325 | 7325 | 8325 |
| AfYHR143W-A | CaYHR143W-A | 5326 | 6326 | 7326 | 8326 |
| AfYDR449C | CaYDR449C | 5327 | 6327 | 7327 | 8327 |
| AfYDR016C | CaYDR016C | 5328 | 6328 | 7328 | 8328 |
| AfYDR236C | CaYDR236C | 5329 | 6329 | 7329 | 8329 |
| AfYKL141W | CaYKL141W | 5330 | 6330 | 7330 | 8330 |
| AfYLR078C | CaYLR078C | 5331 | 6331 | 7331 | 8331 |
| AfYDR311W | CaYDR311W | 5332 | 6332 | 7332 | 8332 |
| AfORF6_3819 | CaORF6_3819 | 5333 | 6333 | 7333 | 8333 |
| AfORF6_3864 | CaORF6_3864 | 5334 | 6334 | 7334 | 8334 |
| AfORF6_804 | CaORF6_804 | 5335 | 6335 | 7335 | 8335 |
| AfORF6_889 | CaORF6_889 | 5336 | 6336 | 7336 | 8336 |
| AfYAL033W | CaYAL033W | 5337 | 6337 | 7337 | 8337 |
| AfYBL030C | CaYBL030C | 5338 | 6338 | 7338 | 8338 |
| AfYBR029C | CaYBR029C | 5339 | 6339 | 7339 | 8339 |
| AfYBR123C | CaYBR123C | 5340 | 6340 | 7340 | 8340 |
| AfYBR143C | CaYBR143C | 5341 | 6341 | 7341 | 8341 |
| AfYBR155W | CaYBR155W | 5342 | 6342 | 7342 | 8342 |
| AfYBR198C | CaYBR198C | 5343 | 6343 | 7343 | 8343 |
| AfYCL003W | CaYCL003W | 5344 | 6344 | 7344 | 8344 |
| AfYCR012W | CaYCR012W | 5345 | 6345 | 7345 | 8345 |
| AfYCR057C | CaYCR057C | 5346 | 6346 | 7346 | 8346 |
| AfYDL084W | CaYDL084W | 5347 | 6347 | 7347 | 8347 |
| AfYDL087C | CaYDL087C | 5348 | 6348 | 7348 | 8348 |
| AfYDR002W | CaYDR002W | 5349 | 6349 | 7349 | 8349 |
| AfYDR023W | CaYDR023W | 5350 | 6350 | 7350 | 8350 |
| AfYDR045C | CaYDR045C | 5351 | 6351 | 7351 | 8351 |
| AfYDR054C | CaYDR054C | 5352 | 6352 | 7352 | 8352 |
| AfYDR060W | CaYDR060W | 5353 | 6353 | 7353 | 8353 |
| AfYDR087C | CaYDR087C | 5354 | 6354 | 7354 | 8354 |
| AfYDR226W | CaYDR226W | 5355 | 6355 | 7355 | 8355 |
| AfYDR228C | CaYDR228C | 5356 | 6356 | 7356 | 8356 |
| AfYDR238C | CaYDR238C | 5357 | 6357 | 7357 | 8357 |
| AfYDR299W | CaYDR299W | 5358 | 6358 | 7358 | 8358 |
| AfYDR328C | CaYDR328C | 5359 | 6359 | 7359 | 8359 |
| AfYDR373W | CaYDR373W | 5360 | 6360 | 7360 | 8360 |
| AfYDR390C | CaYDR390C | 5361 | 6361 | 7361 | 8361 |
| AfYDR489W | CaYDR489W | 5362 | 6362 | 7362 | 8362 |
| AfYEL032W | CaYEL032W | 5363 | 6363 | 7363 | 8363 |
| AfYEL055C | CaYEL055C | 5364 | 6364 | 7364 | 8364 |
| AfYER006W | CaYER006W | 5365 | 6365 | 7365 | 8365 |
| AfYER036C | CaYER036C | 5366 | 6366 | 7366 | 8366 |
| AfYFL045C | CaYFL045C | 5367 | 6367 | 7367 | 8367 |
| AfYGL008C | CaYGL008C | 5368 | 6368 | 7368 | 8368 |
| AfYGL048C | CaYGL048C | 5369 | 6369 | 7369 | 8369 |
| AfYGL097W | CaYGL097W | 5370 | 6370 | 7370 | 8370 |
| AfYGL112C | CaYGL112C | 5371 | 6371 | 7371 | 8371 |
| AfYGL201C | CaYGL201C | 5372 | 6372 | 7372 | 8372 |
| AfYGL207W | CaYGL207W | 5373 | 6373 | 7373 | 8373 |
| AfYGL225W | CaYGL225W | 5374 | 6374 | 7374 | 8374 |
| AfYGL245W | CaYGL245W | 5375 | 6375 | 7375 | 8375 |
| AfYGR047C | CaYGR047C | 5376 | 6376 | 7376 | 8376 |
| AfYGR048W | CaYGR048W | 5377 | 6377 | 7377 | 8377 |
| AfYGR083C | CaYGR083C | 5378 | 6378 | 7378 | 8378 |
| AfYGR185C | CaYGR185C | 5379 | 6379 | 7379 | 8379 |
| AfYGR218W | CaYGR218W | 5380 | 6380 | 7380 | 8380 |
| AfYGR267C | CaYGR267C | 5381 | 6381 | 7381 | 8381 |
| AfYHR005C-A | CaYHR005C-A | 5382 | 6382 | 7382 | 8382 |
| AfYHR072W-A | CaYHR072W-A | 5383 | 6383 | 7383 | 8383 |
| AfYHR166C | CaYHR166C | 5384 | 6384 | 7384 | 8384 |
| AfYHR188C | CaYHR188C | 5385 | 6385 | 7385 | 8385 |
| AfYIL022W | CaYIL022W | 5386 | 6386 | 7386 | 8386 |
| AfYIL109C | CaYIL109C | 5387 | 6387 | 7387 | 8387 |
| AfYJL109C | CaYJL109C | 5388 | 6388 | 7388 | 8388 |
| AfYJL111W | CaYJL111W | 5389 | 6389 | 7389 | 8389 |
| AfYJL167W | CaYJL167W | 5390 | 6390 | 7390 | 8390 |
| AfYJR064W | CaYJR064W | 5391 | 6391 | 7391 | 8391 |
| AfYJR065C | CaYJR065C | 5392 | 6392 | 7392 | 8392 |
| AfYKL013C | CaYKL013C | 5393 | 6393 | 7393 | 8393 |
| AfYKL045W | CaYKL045W | 5394 | 6394 | 7394 | 8394 |
| AfYKL104C | CaYKL104C | 5395 | 6395 | 7395 | 8395 |
| AfYKL193C | CaYKL193C | 5396 | 6396 | 7396 | 8396 |
| AfYLR088W | CaYLR088W | 5397 | 6397 | 7397 | 8397 |
| AfYLR129W | CaYLR129W | 5398 | 6398 | 7398 | 8398 |
| AfYLR274W | CaYLR274W | 5399 | 6399 | 7399 | 8399 |
| AfYLR291C | CaYLR291C | 5400 | 6400 | 7400 | 8400 |
| AfYLR293C | CaYLR293C | 5401 | 6401 | 7401 | 8401 |
| AfYML064C | CaYML064C | 5402 | 6402 | 7402 | 8402 |
| AfYMR049C | CaYMR049C | 5403 | 6403 | 7403 | 8403 |
| AfYMR055C | CaYMR055C | 5404 | 6404 | 7404 | 8404 |
| AfYMR131C | CaYMR131C | 5405 | 6405 | 7405 | 8405 |
| AfYMR220W | CaYMR220W | 5406 | 6406 | 7406 | 8406 |
| AfYMR309C | CaYMR309C | 5407 | 6407 | 7407 | 8407 |
| AfYNL061W | CaYNL061W | 5408 | 6408 | 7408 | 8408 |
| AfYNL113W | CaYNL113W | 5409 | 6409 | 7409 | 8409 |
| AfYNL163C | CaYNL163C | 5410 | 6410 | 7410 | 8410 |
| AfYNL178W | CaYNL178W | 5411 | 6411 | 7411 | 8411 |
| AfYNL181W | CaYNL181W | 5412 | 6412 | 7412 | 8412 |
| AfYNL263C | CaYNL263C | 5413 | 6413 | 7413 | 8413 |
| AfYNR046W | CaYNR046W | 5414 | 6414 | 7414 | 8414 |
| AfYOL034W | CaYOL034W | 5415 | 6415 | 7415 | 8415 |
| AfYOR056C | CaYOR056C | 5416 | 6416 | 7416 | 8416 |
| AfYOR057W | CaYOR057W | 5417 | 6417 | 7417 | 8417 |
| AfYOR117W | CaYOR117W | 5418 | 6418 | 7418 | 8418 |
| AfYOR207C | CaYOR207C | 5419 | 6419 | 7419 | 8419 |
| AfYOR224C | CaYOR224C | 5420 | 6420 | 7420 | 8420 |
| AfYOR259C | CaYOR259C | 5421 | 6421 | 7421 | 8421 |
| AfYOR261C | CaYOR261C | 5422 | 6422 | 7422 | 8422 |
| AfYOR262W | CaYOR262W | 5423 | 6423 | 7423 | 8423 |
| AfYPL117C | CaYPL117C | 5424 | 6424 | 7424 | 8424 |
| AfYPL122C | CaYPL122C | 5425 | 6425 | 7425 | 8425 |
| AfYPL218W | CaYPL218W | 5426 | 6426 | 7426 | 8426 |
| AfYPL235W | CaYPL235W | 5427 | 6427 | 7427 | 8427 |
| AfYPR103W | CaYPR103W | 5428 | 6428 | 7428 | 8428 |
| AfYPR112C | CaYPR112C | 5429 | 6429 | 7429 | 8429 |
| AfYPR165W | CaYPR165W | 5430 | 6430 | 7430 | 8430 |
| AfPRO1 | CaPRO1 | 5431 | 6431 | 7431 | 8431 |
| AfYBL050W | CaYBL050W | 5432 | 6432 | 7432 | 8432 |
| AfYDL029W | CaYDL029W | 5433 | 6433 | 7433 | 8433 |
| AfYDR397C | CaYDR397C | 5434 | 6434 | 7434 | 8434 |
| AfYDR460W | CaYDR460W | 5435 | 6435 | 7435 | 8435 |
| AfYER094C | CaYER094C | 5436 | 6436 | 7436 | 8436 |
| AfYER171W | CaYER171W | 5437 | 6437 | 7437 | 8437 |
| AfYGL091C | CaYGL091C | 5438 | 6438 | 7438 | 8438 |
| AfYGR074W | CaYGR074W | 5439 | 6439 | 7439 | 8439 |
| AfYGR103W | CaYGR103W | 5440 | 6440 | 7440 | 8440 |
| AfYGR253C | CaYGR253C | 5441 | 6441 | 7441 | 8441 |
| AfYHR170W | CaYHR170W | 5442 | 6442 | 7442 | 8442 |
| AfYJR017C | CaYJR017C | 5443 | 6443 | 7443 | 8443 |
| AfYLR103C | CaYLR103C | 5444 | 6444 | 7444 | 8444 |
| AfYNL075W | CaYNL075W | 5445 | 6445 | 7445 | 8445 |
| AfYPL131W | CaYPL131W | 5446 | 6446 | 7446 | 8446 |
| AfORF6_1717 | CaORF6_1717 | 5447 | 6447 | 7447 | 8447 |
| AfORF6_2193 | CaORF6_2193 | 5448 | 6448 | 7448 | 8448 |
| AfORF6_2398 | CaORF6_2398 | 5449 | 6449 | 7449 | 8449 |
| AfORF6_4499 | CaORF6_4499 | 5450 | 6450 | 7450 | 8450 |
| AfORF6_5520 | CaORF6_5520 | 5451 | 6451 | 7451 | 8451 |
| AfORF6_7629 | CaORF6_7629 | 5452 | 6452 | 7452 | 8452 |
| AfORF6_7847 | CaORF6_7847 | 5453 | 6453 | 7453 | 8453 |
| AfORF6_8362 | CaORF6_8362 | 5454 | 6454 | 7454 | 8454 |
| AfORF6_8377 | CaORF6_8377 | 5455 | 6455 | 7455 | 8455 |
| AfORF6_8461 | CaORF6_8461 | 5456 | 6456 | 7456 | 8456 |
| AfORF6_8607 | CaORF6_8607 | 5457 | 6457 | 7457 | 8457 |
| AfORF6_8654 | CaORF6_8654 | 5458 | 6458 | 7458 | 8458 |
| AfYBL020W | CaYBL020W | 5459 | 6459 | 7459 | 8459 |
| AfYBL097W | CaYBL097W | 5460 | 6460 | 7460 | 8460 |
| AfYBR002C | CaYBR002C | 5461 | 6461 | 7461 | 8461 |
| AfYBR011C | CaYBR011C | 5462 | 6462 | 7462 | 8462 |
| AfYBR087W | CaYBR087W | 5463 | 6463 | 7463 | 8463 |
| AfYBR135W | CaYBR135W | 5464 | 6464 | 7464 | 8464 |
| AfYBR243C | CaYBR243C | 5465 | 6465 | 7465 | 8465 |
| AfYCL059C | CaYCL059C | 5466 | 6466 | 7466 | 8466 |
| AfYDL102W | CaYDL102W | 5467 | 6467 | 7467 | 8467 |
| AfYDL132W | CaYDL132W | 5468 | 6468 | 7468 | 8468 |
| AfYDL141W | CaYDL141W | 5469 | 6469 | 7469 | 8469 |
| AfYDL143W | CaYDL143W | 5470 | 6470 | 7470 | 8470 |
| AfYDL145C | CaYDL145C | 5471 | 6471 | 7471 | 8471 |
| AfYDL147W | CaYDL147W | 5472 | 6472 | 7472 | 8472 |
| AfYDL195W | CaYDL195W | 5473 | 6473 | 7473 | 8473 |
| AfYDL208W | CaYDL208W | 5474 | 6474 | 7474 | 8474 |
| AfYDR170C | CaYDR170C | 5475 | 6475 | 7475 | 8475 |
| AfYDR188W | CaYDR188W | 5476 | 6476 | 7476 | 8476 |
| AfYDR189W | CaYDR189W | 5477 | 6477 | 7477 | 8477 |
| AfYDR190C | CaYDR190C | 5478 | 6478 | 7478 | 8478 |
| AfYDR235W | CaYDR235W | 5479 | 6479 | 7479 | 8479 |
| AfYDR246W | CaYDR246W | 5480 | 6480 | 7480 | 8480 |
| AfYDR324C | CaYDR324C | 5481 | 6481 | 7481 | 8481 |
| AfYDR341C | CaYDR341C | 5482 | 6482 | 7482 | 8482 |
| AfYDR365C | CaYDR365C | 5483 | 6483 | 7483 | 8483 |
| AfYDR376W | CaYDR376W | 5484 | 6484 | 7484 | 8484 |
| AfYDR394W | CaYDR394W | 5485 | 6485 | 7485 | 8485 |
| AfYDR429C | CaYDR429C | 5486 | 6486 | 7486 | 8486 |
| AfYER007W | CaYER007W | 5487 | 6487 | 7487 | 8487 |
| AfYER048W-A | CaYER048W-A | 5488 | 6488 | 7488 | 8488 |
| AfYER082C | CaYER082C | 5489 | 6489 | 7489 | 8489 |
| AfYER148W | CaYER148W | 5490 | 6490 | 7490 | 8490 |
| AfYFL002C | CaYFL002C | 5491 | 6491 | 7491 | 8491 |
| AfYFR004W | CaYFR004W | 5492 | 6492 | 7492 | 8492 |
| AfYFR031C | CaYFR031C | 5493 | 6493 | 7493 | 8493 |
| AfYFR037C | CaYFR037C | 5494 | 6494 | 7494 | 8494 |
| AfYGL011C | CaYGL011C | 5495 | 6495 | 7495 | 8495 |
| AfYGL068W | CaYGL068W | 5496 | 6496 | 7496 | 8496 |
| AfYGL103W | CaYGL103W | 5497 | 6497 | 7497 | 8497 |
| AfYGR094W | CaYGR094W | 5498 | 6498 | 7498 | 8498 |
| AfYHL015W | CaYHL015W | 5499 | 6499 | 7499 | 8499 |
| AfYHR007C | CaYHR007C | 5500 | 6500 | 7500 | 8500 |
| AfYHR020W | CaYHR020W | 5501 | 6501 | 7501 | 8501 |
| AfYHR090C | CaYHR090C | 5502 | 6502 | 7502 | 8502 |
| AfYHR148W | CaYHR148W | 5503 | 6503 | 7503 | 8503 |
| AfYHR165C | CaYHR165C | 5504 | 6504 | 7504 | 8504 |
| AfYHR190W | CaYHR190W | 5505 | 6505 | 7505 | 8505 |
| AfYIL046W | CaYIL046W | 5506 | 6506 | 7506 | 8506 |
| AfYIL078W | CaYIL078W | 5507 | 6507 | 7507 | 8507 |
| AfYIR008C | CaYIR008C | 5508 | 6508 | 7508 | 8508 |
| AfYJL014W | CaYJL014W | 5509 | 6509 | 7509 | 8509 |
| AfYJL050W | CaYJL050W | 5510 | 6510 | 7510 | 8510 |
| AfYJL069C | CaYJL069C | 5511 | 6511 | 7511 | 8511 |
| AfYJL081C | CaYJL081C | 5512 | 6512 | 7512 | 8512 |
| AfYJL104W | CaYJL104W | 5513 | 6513 | 7513 | 8513 |
| AfYJL143W | CaYJL143W | 5514 | 6514 | 7514 | 8514 |
| AfYJL153C | CaYJL153C | 5515 | 6515 | 7515 | 8515 |
| AfYJL203W | CaYJL203W | 5516 | 6516 | 7516 | 8516 |
| AfYJR007W | CaYJR007W | 5517 | 6517 | 7517 | 8517 |
| AfYJR063W | CaYJR063W | 5518 | 6518 | 7518 | 8518 |
| AfYJR072C | CaYJR072C | 5519 | 6519 | 7519 | 8519 |
| AfYKL058W | CaYKL058W | 5520 | 6520 | 7520 | 8520 |
| AfYKL060C | CaYKL060C | 5521 | 6521 | 7521 | 8521 |
| AfYKL145W | CaYKL145W | 5522 | 6522 | 7522 | 8522 |
| AfYKR068C | CaYKR068C | 5523 | 6523 | 7523 | 8523 |
| AfYKR079C | CaYKR079C | 5524 | 6524 | 7524 | 8524 |
| AfYLR078C | CaYLR078C | 5525 | 6525 | 7525 | 8525 |
| AfYLR116W | CaYLR116W | 5526 | 6526 | 7526 | 8526 |
| AfYLR153C | CaYLR153C | 5527 | 6527 | 7527 | 8527 |
| AfYLR163C | CaYLR163C | 5528 | 6528 | 7528 | 8528 |
| AfYLR208W | CaYLR208W | 5529 | 6529 | 7529 | 8529 |
| AfYLR272C | CaYLR272C | 5530 | 6530 | 7530 | 8530 |
| AfYLR276C | CaYLR276C | 5531 | 6531 | 7531 | 8531 |
| AfYLR277C | CaYLR277C | 5532 | 6532 | 7532 | 8532 |
| AfYLR336C | CaYLR336C | 5533 | 6533 | 7533 | 8533 |
| AfYLR347C | CaYLR347C | 5534 | 6534 | 7534 | 8534 |
| AfYLR355C | CaYLR355C | 5535 | 6535 | 7535 | 8535 |
| AfYLR383W | CaYLR383W | 5536 | 6536 | 7536 | 8536 |
| AfYML069W | CaYML069W | 5537 | 6537 | 7537 | 8537 |
| AfYMR093W | CaYMR093W | 5538 | 6538 | 7538 | 8538 |
| AfYMR235C | CaYMR235C | 5539 | 6539 | 7539 | 8539 |
| AfYNL102W | CaYNL102W | 5540 | 6540 | 7540 | 8540 |
| AfYNL189W | CaYNL189W | 5541 | 6541 | 7541 | 8541 |
| AfYNL240C | CaYNL240C | 5542 | 6542 | 7542 | 8542 |
| AfYNR050C | CaYNR050C | 5543 | 6543 | 7543 | 8543 |
| AfYOL027C | CaYOL027C | 5544 | 6544 | 7544 | 8544 |
| AfYOL097C | CaYOL097C | 5545 | 6545 | 7545 | 8545 |
| AfYOL102C | CaYOL102C | 5546 | 6546 | 7546 | 8546 |
| AfYOR048C | CaYOR048C | 5547 | 6547 | 7547 | 8547 |
| AfYOR063W | CaYOR063W | 5548 | 6548 | 7548 | 8548 |
| AfYOR116C | CaYOR116C | 5549 | 6549 | 7549 | 8549 |
| AfYOR159C | CaYOR159C | 5550 | 6550 | 7550 | 8550 |
| AfYOR204W | CaYOR204W | 5551 | 6551 | 7551 | 8551 |
| AfYOR217W | CaYOR217W | 5552 | 6552 | 7552 | 8552 |
| AfYOR341W | CaYOR341W | 5553 | 6553 | 7553 | 8553 |
| AfYPL076W | CaYPL076W | 5554 | 6554 | 7554 | 8554 |
| AfYPL211W | CaYPL211W | 5555 | 6555 | 7555 | 8555 |
| AfYPL242C | CaYPL242C | 5556 | 6556 | 7556 | 8556 |
| AfYPL266W | CaYPL266W | 5557 | 6557 | 7557 | 8557 |
| AfYPR019W | CaYPR019W | 5558 | 6558 | 7558 | 8558 |
| AfYPR176C | CaYPR176C | 5559 | 6559 | 7559 | 8559 |
| AfYLR355C | CaYLR355C | 5560 | 6560 | 7560 | 8560 |
| AfYGR083C | CaYGR083C | 5561 | 6561 | 7561 | 8561 |
| AfYHR172W | CaYHR172W | 5562 | 6562 | 7562 | 8562 |
| AfYOL130W | CaYOL130W | 5563 | 6563 | 7563 | 8563 |
| AfYJL143W | CaYJL143W | 5564 | 6564 | 7564 | 8564 |
| AfYNL039W | CaYNL039W | 5565 | 6565 | 7565 | 8565 |
| AfYPR187W | CaYPR187W | 5566 | 6566 | 7566 | 8566 |
| AfYPR144C | CaYPR144C | 5567 | 6567 | 7567 | 8567 |
| AfYGR002C | CaYGR002C | 5568 | 6568 | 7568 | 8568 |
| AfYKL059C | CaYKL059C | 5569 | 6569 | 7569 | 8569 |
| AfYGR009C | CaYGR009C | 5570 | 6570 | 7570 | 8570 |
| AfYGR186W | CaYGR186W | 5571 | 6571 | 7571 | 8571 |
| AfORF6_1498 | CaORF6_1498 | 5572 | 6572 | 7572 | 8572 |
| AfORF6_3819 | CaORF6_3819 | 5573 | 6573 | 7573 | 8573 |
| AfORF6_4463 | CaORF6_4463 | 5574 | 6574 | 7574 | 8574 |
| AfORF6_6069 | CaORF6_6069 | 5575 | 6575 | 7575 | 8575 |
| AfORF6_6140 | CaORF6_6140 | 5576 | 6576 | 7576 | 8576 |
| AfORF6_6390 | CaORF6_6390 | 5577 | 6577 | 7577 | 8577 |
| AfORF6_6660 | CaORF6_6660 | 5578 | 6578 | 7578 | 8578 |
| AfORF6_6664 | CaORF6_6664 | 5579 | 6579 | 7579 | 8579 |
| AfORF6_6808 | CaORF6_6808 | 5580 | 6580 | 7580 | 8580 |
| AfORF6_6933 | CaORF6_6933 | 5581 | 6581 | 7581 | 8581 |
| AfORF6_6939 | CaORF6_6939 | 5582 | 6582 | 7582 | 8582 |
| AfORF6_7203 | CaORF6_7203 | 5583 | 6583 | 7583 | 8583 |
| AfORF6_8654 | CaORF6_8654 | 5584 | 6584 | 7584 | 8584 |
| AfYBR038W | CaYBR038W | 5585 | 6585 | 7585 | 8585 |
| AfYER059W | CaYER059W | 5586 | 6586 | 7586 | 8586 |
| AfYGL233W | CaYGL233W | 5587 | 6587 | 7587 | 8587 |
| AfYNL048W | CaYNL048W | 5588 | 6588 | 7588 | 8588 |
| AfYNL221C | CaYNL221C | 5589 | 6589 | 7589 | 8589 |
| AfYOL066C | CaYOL066C | 5590 | 6590 | 7590 | 8590 |
| AfORF6_3026 | CaORF6_3026 | 5591 | 6591 | 7591 | 8591 |
| AfORF6_4005 | CaORF6_4005 | 5592 | 6592 | 7592 | 8592 |
| AfYNL256W | CaYNL256W | 5593 | 6593 | 7593 | 8593 |
| AfYPL128C | CaYPL128C | 5594 | 6594 | 7594 | 8594 |
| AfYAL041W | CaYAL041W | 5595 | 6595 | 7595 | 8595 |
| AfYFL024C | CaYFL024C | 5596 | 6596 | 7596 | 8596 |
| AfYJL041W | CaYJL041W | | | 7597 | 8597 |
| AfYMR314W | CaYMR314W | | | 7598 | 8598 |
| AfYKL004W | CaYKL004W | | | 7599 | 8599 |
| AfYFL005W | CaYFL005W | | | 7600 | 8600 |
| AfYDL126C | CaYDL126C | | | 7601 | 8601 |
| AfYAR007C | CaYAR007C | | 6602 | 7602 | 8602 |
| AfYMR043W | CaYMR043W | | 6603 | 7603 | 8603 |

### 5.1.2. Essentiality of Aspergillus fumigatus Gene Sequences

In one embodiment of the invention, based on the high degree of sequence conservation displayed between the Aspergillus fumigatus genes of the invention and their homologs in Candida albicans, it is predicted that these Aspergillus fumigatus genes perform biological functions similar to their homologous Candida albicans counterparts. Accordingly, the homologous Aspergillus fumigatus genes of the invention are predicted to be essential to the survival or growth of Aspergillus fumigatus.

The essentiality of each of the Candida albicans genes used in the sequence homology analysis has been demonstrated experimentally by creating such conditional-expression mutants. Since C. albicans is an obligate diploid organism which comprises two alleles of each gene, one allele of the gene is disrupted or knocked out and the expression of the other allele is placed under the control of a heterologous promoter. The creation and testing of conditional-expression mutants of the C. albicans essential genes are described in copending U.S. patent application Ser. Nos. 60/259,128 and 09/792,024, respectively filed Dec. 29, 2000, and Feb. 20, 2001, which are both incorporated herein by reference in their entireties.

An Aspergillus fumigatus gene is considered essential when survival, growth, and proliferation and/or viability of an Aspergillus fumigatus strain is substantially coupled to or dependent on the expression of the gene. An essential function for a cell depends in part on the genotype of the cell and in part the cell's environment. Multiple genes are required for some essential function, for example, energy metabolism, etc. biosynthesis of cell structure, replication and repair of genetic material, etc. Thus, the expression of many genes in an organism are essential for its survival and/or growth. A deletion of or mutation in such a gene resulting in a loss or reduction of its expression and/or biological activity can lead to a loss or reduction of viability or growth of the fungus under normal growth conditions. A deletion of or mutation in an essential gene can cause the Aspergillus fumigatus cells to perish, stop growing, or display a severe growth defect. The reduction or loss of function of an Aspergillus fumigatus essential gene can result in cell numbers or growth rate that are in the range of 50%, 40%, 30%, 20%, 10%, 5%, or 1% of that of a wild type Aspergillus fumigatus under similar conditions. Many essential genes in Aspergillus fumigatus are expected to contribute to the virulence and/or pathogenicity of the organism. Accordingly, when the virulence and/or pathogenicity of an Aspergillus fumigatus strain to a defined host, or to a defined set of cells from a host, is associated with the conditional expression of the mutant gene, that essential gene may also be referred to as a “virulence gene” of Aspergillus fumigatus.

The essentiality of a gene can be demonstrated by knocking out (insertionally inactivating or deleting) the target gene in Aspergillus fumigatus and observing the phenotype of the resulting mutant Aspergillus fumigatus under normal growth conditions and other permissive growth conditions. However, in gene disruption experiments, the observation that a knock-out (e.g. by insertional inactivation or deletion of the target gene) cannot be generated for a gene, cannot, per se, support the conclusion that the gene is an essential gene. Rather, a direct demonstration of expression of the gene in question that is coupled with viability of the cell carrying that gene, is required for the unambiguous confirmation that the gene in question is essential. Accordingly, an essential Aspergillus fumigatus gene can be placed under the control of a regulatable, heterologous promoter such that a range of expression level of the essential gene in the mutant cell can be obtained. Such levels of expression include negligible or very low expression levels, enabling an evaluation of the phenotype of such a genetically engineered conditional-expression Aspergillus fumigatus mutant when grown under normal growth conditions and other permissive growth conditions. A loss or reduction of viability or growth of the conditional-expression mutant confirms the essentiality of the Aspergillus fumigatus gene.

According to the methods of the invention, a collection of conditional-expression mutants of Aspergillus fumigatus can be generated in which the dosage of specific genes can be modulated, such that their functions in survival, growth, proliferation and/or pathogenicity can be investigated. The information accrued from such investigations allows the identification and validation of individual gene products as potential drug targets. The present invention further provides methods of use of the genetic mutants either individually or as a collection in drug screening and for investigating the mechanisms of drug action.

In another embodiment of the invention, each of the essential genes of the invention represents a potential drug target in Aspergillus fumigatus that is used individually or as part of a collection, in various methods of screening for drugs active against Aspergillus fumigatus and other Aspergillus fungi. Depending on the objective of the drug screening program and the target disease, the essential genes of the invention can be classified and divided into subsets based on the structural features, functional properties, and expression profile of the gene products. The gene products encoded by the essential genes within each subset may share similar biological activity, similar intracellular localization, structural homology, and/or sequence homology. Subsets may also be created based on the homology or similarity in sequence to other organisms in a similar or distant taxonomic group, e.g. homology to Saccharomyces cerevisiae, Schizosaccharomyces pombe genes, or to human genes, or a complete lack of sequence similarity or homology to genes of other organisms, such as S. cerevisiae, S. pombe, or human. Subsets may also be created based on the display of cidal terminal phenotype or static terminal phenotype by the respective Aspergillus fumigatus mutants. Such subsets, referred to as essential gene sets which can be conveniently investigated as a group in a drug screening program, are provided by the present invention. In a particular embodiment, mutants that display a rapid cidal terminal phenotype are preferred. Moreover, since the products encoded by Aspergillus fumigatus genes of the invention are involved in biochemical pathways essential to the fungus, analysis of these genes and their encode products facilitates elucidation of such pathways, thereby identifying additional drug targets. Therefore, the present invention provides a systematic and efficient method for drug target identification and validation. The approach is based on genomics information as well as the biological function of individual genes.

Various methods of use of the Aspergillus fumigatus nucleotide sequences of the invention in drug target identification and drug screening as described in Section 5.4. Methods of making the gene products of the Aspergillus fumigatus nucleotide sequences and fragments thereof in prokaryotes, yeasts, and higher eukaryotes, and methods for making antibodies that bind specifically to the gene products and fragments thereof are also encompassed and described in Sections 5.2.3 and 5.2.4.

In yet another embodiment, Aspergillus fumigatus essential genes can be genetically engineered to be expressed in complementation studies with specific strains of mutant yeast cells, such as Saccharomyces cerevisiae and Candida albicans mutant cells, that display a loss or reduction of function of the corresponding homologous essential gene. In this manner, an Aspergillus fumigatus essential gene can be used in complementation studies in a Candida albicans or Saccharomyces cerevisiae mutant cell in order to elucidate and establish the structure and function of the gene product of the homologous Aspergillus fumigatus essential gene.

In a further embodiment, Aspergillus fumigatus essential gene sequences can also be used to facilitate the creation of a mutant strain of Aspergillus fumigatus, wherein the Aspergillus fumigatus essential gene is replaced with the homologous Candida albicans gene. Such Aspergillus fumigatus mutants can be especially useful as Candida albicans is an obligate diploid which contains two alleles of every essential gene, and thus requires two molecular events to create a knockout mutant in Candida albicans. This Aspergillus fumigatus mutant allows the expression of a Candida albicans essential gene in the cellular background of another pathogen which does not display the respective essential gene function, and can be useful in evaluating the action of potential drug candidates against Candida albicans.

Moreover, the present invention provides specifically the use of this information of essentiality to identify orthologs of these essential genes in a non-pathogenic yeast, such as Saccharomyces cerevisiae, and the use of these orthologs in drug screening methods. Although the nucleotide sequence of the orthologs of these essential genes in Saccharomyces cerevisiae may be known, in certain instances, it was not appreciated that these Saccharomyces cerevisiae genes can be useful for discovering drugs against pathogenic fungi, such as Aspergillus fumigatus.

Furthermore, because of the sequence conservation between gene products of the essential genes of Aspergillus fumigatus and Candida albicans, the structure of the gene product of the Aspergillus fumigatus essential gene can also be used to aid in the rational design of a drug against the homologous Candida albicans gene product. Thus, in various embodiments, the Aspergillus fumigatus essential genes can be used in developing drugs that act against Candida albicans or other pathogenic fungi. Fungistatic or fungicidal compounds developed by such methods may have a broad host range.

The biological function of the gene products encoded by the Aspergillus fumigatus essential genes of the invention can be predicted by the function of their corresponding homologs in Candida albicans, and/or Saccharomyces cerevisiae. Accordingly, the Aspergillus fumigatus genes of the invention may have one or more of the following biological functions:

Metabolism: amino-acid metabolism, amino-acid biosynthesis, assimilatory reduction of sulfur and biosynthesis of the serine family, regulation of amino-acid metabolism, amino-acid transport, amino-acid degradation (catabolism), other amino-acid metabolism activities, nitrogen and sulphur metabolism, nitrogen and sulphur utilization, regulation of nitrogen and sulphur utilization, nitrogen and sulphur transport, nucleotide metabolism, purine-ribonucleotide metabolism, pyrimidine-ribonucleotide metabolism, deoxyribonucleotide metabolism, metabolism of cyclic and unusual nucleotides, regulation of nucleotide metabolism, polynucleotide degradation, nucleotide transport, other nucleotide-metabolism activities, phosphate metabolism, phosphate utilization, regulation of phosphate utilization, phosphate transport, other phosphate metabolism activities, C-compound and carbohydrate metabolism, C-compound and carbohydrate utilization, regulation of C-compound and carbohydrate utilization, C-compound, carbohydrate transport, other carbohydrate metabolism activities, lipid, fatty-acid and isoprenoid metabolism, lipid, fatty-acid and isoprenoid biosynthesis, phospholipid biosynthesis, glycolipid biosynthesis, breakdown of lipids, fatty acids and isoprenoids, lipid, fatty-acid and isoprenoid utilization, regulation of lipid, fatty-acid and isoprenoid biosynthesis, lipid and fatty-acid transport, lipid and fatty-acid binding, other lipid, fatty-acid and isoprenoid metabolism activities, metabolism of vitamins, cofactors, and prosthetic groups, biosynthesis of vitamins, cofactors, and prosthetic groups, utilization of vitamins, cofactors, and prosthetic groups, regulation of vitamins, cofactors, and prosthetic groups, transport of vitamins, cofactors, and prosthetic groups, other vitamin, cofactor, and prosthetic group activities, secondary metabolism, metabolism of primary metabolic sugars derivatives, biosynthesis of glycosides, biosynthesis of secondary products derived from primary amino acids, biosynthesis of amines.

Energy: glycolysis and gluconeogenesis, pentose-phosphate pathway, tricarboxylic-acid pathway, electron transport and membrane-associated energy conservation, accessory proteins of electron transport and membrane-associated energy conservation, other electron transport and membrane-associated energy conservation proteins, respiration, fermentation, metabolism of energy reserves (glycogen, trehalose), glyoxylate cycle, oxidation of fatty acids, other energy generation activities.

Cell Growth, Cell Division and DNA Synthesis: cell growth, budding, cell polarity and filament formation, pheromone response, mating-type determination, sex-specific proteins, sporulation and germination, meiosis, DNA synthesis and replication, recombination and DNA repair, cell cycle control and mitosis, cell cycle check point proteins, cytokinesis, other cell growth, cell division and DNA synthesis activities.

Transcription: rRNA transcription, rRNA synthesis, rRNA processing, other rRNA-transcription activities, tRNA transcription, tRNA synthesis, tRNA processing, tRNA modification, other tRNA-transcription activities, mRNA transcription, mRNA synthesis, general transcription activities, transcriptional control, chromatin modification, mRNA processing (splicing), mRNA processing (5′-, 3′-end processing, mRNA degradation), 3′-end processing, mRNA degradation, other mRNA-transcription activities, RNA transport, other transcription activities.

Protein Synthesis: ribosomal proteins, translation, translational control, tRNA-synthetases, other protein-synthesis activities.

Protein Destination: protein folding and stabilization, protein targeting, sorting and translocation, protein modification, modification with fatty acids (e.g. myristylation, palmitylation, famesylation), modification by phosphorylation, dephosphorylation, modification by acetylation, other protein modifications, assembly of protein complexes, proteolysis, cytoplasmic and nuclear degradation, lysosomal and vacuolar degradation, other proteolytic degradation, other proteolytic proteins, other protein-destination activities.

Transport Facilitation: channels/pores, ion channels, ion transporters, metal ion transporters (Cu, Fe, etc.), other cation transporters (Na, K, Ca, NH₄, etc.), anion transporters (Cl, SO₄, PO₄, etc.), C-compound and carbohydrate transporters, other C-compound transporters, amino-acid transporters, peptide-transporters, lipid transporters, purine and pyrimidine transporters, allantoin and allantoate transporters, transport ATPases, ABC transporters, drug transporters, other transport facilitators Cellular Transport and Transport Mechanisms: nuclear transport, mitochondrial transport, vesicular transport (Golgi network, etc.), peroxisomal transport, vacuolar transport, extracellular transport (secretion), cellular import, cytoskeleton-dependent transport, transport mechanism, other transport mechanisms, other intracellular-transport activities.

Cellular Biogenesis: biogenesis of cell wall (cell envelope), biogenesis of plasma membrane, biogenesis of cytoskeleton, biogenesis of endoplasmatic reticulum, biogenesis of Golgi, biogenesis of intracellular transport vesicles, nuclear biogenesis, biogenesis of chromosome structure, mitochondrial biogenesis, peroxisomal biogenesis, endosomal biogenesis, vacuolar and lysosomal biogenesis, other cellular biogenesis activities.

Cellular Communication/signal Transduction: intracellular communication, unspecified signal transduction, second messenger formation, regulation of G-protein activity, key kinases, other unspecified signal transduction activities, morphogenesis, G-proteins, regulation of G-protein activity, key kinases, other morphogenetic activities, osmosensing, receptor proteins, mediator proteins, key kinases, key phosphatases, other osmosensing activities, nutritional response pathway, receptor proteins, second messenger formation, G-proteins, regulation of G-protein activity, key kinases, key phosphatases, other nutritional-response activities, pheromone response generation, receptor proteins, G-proteins, regulation of G-protein activity, key kinases, key phosphatases, other pheromone response activities, other signal-transduction activities.

Cell Rescue, Defense, Cell Death and Ageing: stress response, DNA repair, other DNA repair, detoxificaton, detoxification involving cytochrome P450, other detoxification, cell death, ageing, degradation of exogenous polynucleotides, other cell rescue activities.

Ionic Homeostasis: homeostasis of cations, homeostasis of metal ions, homeostasis of protons, homeostasis of other cations, homeostasis of anions, homeostasis of sulfates, homeostasis of phosphate, homeostasis of chloride, homeostasis of other anions.

Cellular Organization (proteins are localized to the corresponding organelle): organization of cell wall, organization of plasma membrane, organization of cytoplasm, organization of cytoskeleton, organization of centrosome, organization of endoplasmatic reticulum, organization of Golgi, organization of intracellular transport vesicles, nuclear organization, organization of chromosome structure, mitochondrial organization, peroxisomal organization, endosomal organization, vacuolar and lysosomal organization, inner membrane organization, extracellular/secretion proteins.

### 5.2. Essential Genes of Aspergillus fumigatus

### 5.2.1 Nucleic Acid Molecules, Vectors, and Host Cells

Described herein are the nucleic acid molecules of the invention which encompass a collection of essential genes of Aspergillus fumigatus.

As used herein, the terms “gene” and “recombinant gene” refer to nucleic acid molecules or polynucleotides comprising a nucleotide sequence encoding a polypeptide or a biologically active ribonucleic acid (RNA). The term can further include nucleic acid molecules comprising upstream, downstream, and/or intron nucleotide sequences. The term “open reading frame (ORF),” means a series of nucleotide triplets coding for amino acids without any termination codons and the triplet sequence is translatable into protein using the codon usage information appropriate for a particular organism.

As used herein, the term “target gene” refers to an essential gene useful in the invention, especially in the context of drug screening. Since it is expected that some genes will contribute to virulence and be essential to the survival of the organism, the terms “target essential gene” and “target virulence gene” will be used where it is appropriate. The target genes of the invention may be partially characterized, fully characterized, or validated as a drug target, by methods known in the art and/or methods taught hereinbelow. As used herein, the term “target organism” refers to a pathogenic organism, the essential and/or virulence genes of which are useful in the invention.

The term “nucleotide sequence” refers to a heteropolymer of nucleotides, including but not limited to ribonucleotides and deoxyribonucleotides, or the sequence of these nucleotides. The terms “nucleic acid” and “polynucleotide” are also used interchangeably herein to refer to a heteropolymer of nucleotides, which may be unmodified or modified DNA or RNA. For example, polynucleotides can be single-stranded or double-stranded DNA, DNA that is a mixture of single-stranded and double-stranded regions, hybrid molecules comprising DNA and RNA with a mixture of single-stranded and double-stranded regions. In addition, the polynucleotide can be composed of triple-stranded regions comprising DNA, RNA, or both. A polynucleotide can also contain one or modified bases, or DNA or RNA backbones modified for nuclease resistance or other reasons. Generally, nucleic acid segments provided by this invention can be assembled from fragments of the genome and short oligonucleotides, or from a series of oligonucleotides, or from individual nucleotides, to provide a synthetic nucleic acid.

The term “recombinant,” when used herein to refer to a polypeptide or protein, means that a polypeptide or protein is derived from recombinant (e. g., microbial or mammalian) expression systems. “Microbial” refers to recombinant polypeptides or proteins made in bacterial or fungal (e.g., yeast) expression systems. As a product, “recombinant microbial” defines a polypeptide or protein essentially unaccompanied by associated native glycosylation. Polypeptides or proteins expressed in most bacterial cultures, e. g., E. coli, will be free of glycosylation modifications; polypeptides or proteins expressed in yeast will be glycosylated.

The term “expression vehicle or vector” refers to a plasmid or phage or virus, for expressing a polypeptide from a nucleotide sequence. An expression vehicle can comprise a transcriptional unit, also referred to as an expression construct, comprising an assembly of (1) a genetic element or elements having a regulatory role in gene expression, for example, promoters or enhancers, (2) a structural or coding sequence which is transcribed into mRNA and translated into protein, and which is operably linked to the elements of (1); and (3) appropriate transcription initiation and termination sequences. “Operably linked” refers to a link in which the regulatory regions and the DNA sequence to be expressed are joined and positioned in such a way as to permit transcription, and ultimately, translation. In the case of Candida albicans, due to its unusual codon usage, modification of a coding sequence derived from other organisms may be necessary to ensure a polypeptide having the expected amino acid sequence is produced in this organism. Structural units intended for use in yeast or eukaryotic expression systems preferably include a leader sequence enabling extracellular secretion of translated protein by a host cell. Alternatively, where a recombinant protein is expressed without a leader or transport sequence, it may include an N-terminal methionine residue. This residue may or may not be subsequently cleaved from the expressed recombinant protein to provide a final product.

The term “recombinant host cells” means cultured cells which have stably integrated a recombinant transcriptional unit into chromosomal DNA or carry stably the recombinant transcriptional unit extrachromosomally. Recombinant host cells as defined herein will express heterologous polypeptides or proteins, and RNA encoded by the DNA segment or synthetic gene in the recombinant transcriptional unit. This term also means host cells which have stably integrated a recombinant genetic element or elements having a regulatory role in gene expression, for example, promoters or enhancers. Recombinant expression systems as defined herein will express RNA, polypeptides or proteins endogenous to the cell upon induction of the regulatory elements linked to the endogenous DNA segment or gene to be expressed. The cells can be prokaryotic or eukaryotic.

The term “polypeptide” refers to the molecule formed by joining amino acids to each other by peptide bonds, and may contain amino acids other than the twenty commonly used gene-encoded amino acids. The term “active polypeptide” refers to those forms of the polypeptide which retain the biologic and/or immunologic activities of any naturally occurring polypeptide. The term “naturally occurring polypeptide” refers to polypeptides produced by cells that have not been genetically engineered and specifically contemplates various polypeptides arising from post-translational modifications of the polypeptide including, but not limited to, proteolytic processing, acetylation, carboxylation, glycosylation, phosphorylation, lipidation and acylation.

The term “isolated” as used herein refers to a nucleic acid or polypeptide separated from at least one macromolecular component (e.g., nucleic acid or polypeptide) present with the nucleic acid or polypeptide in its natural source. In one embodiment, the polynucleotide or polypeptide is purified such that it constitutes at least 95% by weight, more preferably at least 99.8% by weight, of the indicated biological macromolecules present (but water, buffers, and other small molecules, especially molecules having a molecular weight of less than 1000 daltons, can be present).

In one embodiment, the present invention provides the identities of more than six hundred essential genes. Table 1 lists the sequence identifiers of the genomic nucleotide sequences and coding region of these genes that are essential in Aspergillus fumigatus and that share a high degree of sequence conservation with the known essential genes of Candida albicans. The genomic sequences including sequences upstream and downstream of the coding regions, the reading frames, the positions of exons and introns of these genes are not previously known. For each genetic locus, Table 1 provides stretches of the genomic sequence encompassing the Aspergillus fumigatus gene as well as approximately 1 kb of nucleotide sequence both upstream and downstream of the gene (SEQ ID NO.: 1-594 and 5001-5603). The coding sequence, including introns (SEQ ID NO.: 1001-1594 and 6001-6603), the coding region (SEQ ID NO.: 2001-2594 and 7001-7603) and the endoded protein (SEQ ID NO.: 3001-3594 and 8001-8603), derived from each genetic locus are also provided in Table 1. In many cases where a genetic locus can provide more than one open reading frame, the nucleotide sequences of the alternative open reading frame and intron(s), and the amino acid sequence of the alternative gene product are also listed in Table 1. In Table 1, two sets of genomic DNA sequences are also provided for each genetic locus (SEQ ID NO.: 1-594 and 5001-5603) to reflect the fact that the genomic sequences provided in Table 1 include approximately 1 kb of nucleotide sequence before and after each variant coding region. The alternative coding regions or open reading frames may be caused by the use of alternative start and stop codons and/or different messenger RNA splicing patterns. In preferred embodiments, the nucleotide sequences of the essential genes set forth in SEQ ID NO: 7001-7603 and the amino acid sequences of the essential gene products set forth in SEQ ID NO: 8001-8603 are used in accordance to the invention.

The fact that these genes are essential to the growth and/or survival of Aspergillus fumigatus was not known until the inventors' discovery. Thus, the uses of these gene sequences and their gene products are encompassed by the present invention. Accordingly, SEQ ID NO: 2001-2594, and 7001-7603, each identifies a nucleotide sequence of the opening reading frame (ORF) of an identified essential gene. The genomic sequences of the essential genes including sequences upstream and downstream of the coding regions are set forth in SEQ ID NOs: 1-594 and 5001-5603. The genomic sequences of the essential genes including intron sequences are set forth in SEQ ID NO: 1001-1594 and 6001-6603. The predicted amino acid sequence of the identified essential genes are set forth in SEQ ID NO: 3001-3594 and 8001-8603, which are obtained by conceptual translation of the nucleotide sequences of SEQ ID NO: 2001-2594 and 7001-7603, respectively. Also encompassed are gene products, such as splice variants, that are encoded by the genomic sequences of SEQ ID NO: 1-594, 5001-5603, 1001-1594, and 6001-6603, and their nucleotide sequences and amino acid sequences.

The DNA sequences were generated by sequencing reactions and may contain minor errors which may exist as misidentified nucleotides, insertions, and/or deletions. However, such minor errors, if present, in the sequence database should not disturb the identification of the ORF as an essential gene of the invention. Since sequences of the ORFs are provided herein and can be used to uniquely identify the corresponding gene in the Aspergillus fumigatus genome, one can readily obtain a clone of the gene corresponding to the ORFs by any of several art-known methods, repeat the sequencing and correct the minor error(s). The disclosure of the ORFs or a portion thereof essentially provides the complete gene by uniquely identifying the coding sequence in question, and providing sufficient guidance to obtain the complete cDNA or genomic sequence.

Moreover, minor sequence errors, genetic polymorphisms, and variation in splicing do not affect the construction of conditional-expression Aspergillus fumigatus mutant strains and the uses of those strains, since these methods do not require absolute sequence identity between the chromosomal DNA sequences and the sequences of the gene in the primers or recombinant DNA. In some instances, the correct reading frame of the Aspergillus fumigatus gene can be identified by comparing its overall amino acid sequence with known Saccharomyces cerevisiae, Candida albicans and/or C. neoformans sequences. Accordingly, the present invention encompasses Aspergillus fumigatus genes which correspond to the ORFs identified in the invention, polypeptides encoded by Aspergillus fumigatus genes which correspond to the ORFs identified in the invention, and the various uses of the polynucleotides and polypeptides of the genes which correspond to the ORFs of the invention. As used herein in referring to the relationship between a specified nucleotide sequence and a gene, the term “corresponds” or “corresponding” indicates that the specified sequence effectively identifies the gene. In general, correspondence is substantial sequence identity barring minor errors in sequencing, allelic variations and/or variations in splicing. Correspondence can be a transcriptional relationship between the gene sequence and the mRNA or a portion thereof which is transcribed from that gene. This correspondence is present also between portions of an mRNA which is not translated into polypeptide and DNA sequence of the gene.

To identify and characterize the essential genes of the invention, computer algorithms are employed to perform searches in computer databases and comparative analysis, and the results of such analyses are stored in or displayed on a computer. Such computerized tools for analyzing sequence information are very useful in determining the relatedness of structure of genes and gene products with respect to other genes and gene products in the same species or a different species, and may provide putative functions to novel genes and their products. Biological information such as nucleotide and amino acid sequences are coded and represented as streams of data in a computer. As used here, the term “computer” includes but is not limited to personal computers, data terminals, computer workstations, networks, computerized storage and retrieval systems, and graphical displays for presentation of sequence information, and results of analyses. Typically, a computer comprises a data entry means, a display means, a programmable processing unit, and a data storage means. A “computer readable medium” can be used to store information such as sequence data, lists, and databases, and includes but is not limited to computer memory, magnetic storage devices, such as floppy discs and magnetic tapes, optical-magnetic storage devices, and optical storage devices, such as compact discs. Accordingly, the present invention also encompass a computer or a computer readable medium that comprises at least one nucleotide sequence selected from the group consisting of SEQ ID NO: 1001-1594, 6001-6603, 2001-2594, and 7001-7603, or at least one amino acid sequence selected from the group consisting of SEQ ID NO: 3001-3361 and 8001-8603. In preferred embodiments, the sequences are curated and stored in a form with links to other annotations and biological information associated with the sequences. It is also an object of the invention to provide one or more computers programmed with instructions to perform sequence homology searching, sequence alignment, structure prediction and model construction, using the nucleotide sequences of the invention, preferably one or more nucleotide sequences selected from the group consisting of SEQ ID NO: 1001-1594, 6001-6603, 2001-2594, 7001-7603, and/or one or more amino acid sequence selected from the group consisting of SEQ ID NO: 3001-3594 and 8001-8603. Devices, including computers, that comprise, and that can transmit and distribute the nucleotide and/or amino acid sequences of the invention are also contemplated. Also encompassed by the present invention are the uses of one or more nucleotide sequences selected from the group consisting of SEQ ID NO: 1001-1594, 6001-6603, 2001-2361, 7001-7603, and/or one or more amino acid sequence selected from the group consisting of SEQ ID NO: 3001-3361 and 8001-8603, in computer-assisted methods for identifying homologous sequences in public and private sequence databases, in computer-assisted methods for providing putative functional characteristics of a gene product based on structural homology with other gene products with known function(s), and in computer-assisted methods of constructing a model of the gene product. In one specific embodiment, the invention encompasses a method assisted by a computer for identifying a putatively essential gene of a fungus, comprising detecting sequence homology between a fungal nucleotide sequence or fungal amino acid sequence with at least one nucleotide sequence selected from the group consisting of SEQ ID NO: 1001-1594, 6001-6603, 2001-2594, and 7001-7603, or at least one amino acid sequence selected from the group consisting of SEQ ID NO: 3001-3361 and 8001-8603.

The essential genes listed in Table 1 can be obtained using cloning methods well known to those of skill in the art, and include but are not limited to the use of appropriate probes to detect the genes within an appropriate cDNA or gDNA (genomic DNA) library (See, for example, Sambrook et al., 1989, Molecular Cloning: A Laboratory Manual, Cold Spring Harbor Laboratories, which is incorporated herein by reference in its entirety). Probes for the sequences identified herein can be synthesized based on the DNA sequences disclosed herein in SEQ ID NO:1-594, 5001-5603, 1001-1594, 6001-6603, 2001-2594, and 7001-7603. As used herein, “target gene” (e.g. essential and/or virulence gene) refers to (a) a gene containing at least one of the DNA sequences and/or fragments thereof that are set forth in SEQ ID NO: 2001-2594; (b) any DNA sequence or fragment thereof that encodes the amino acid sequence that are set forth in SEQ ID NO: 3001-3594 and 8001-8603, as well as the gene product encoded by genomic SEQ ID NO: 1-594, 5001-5603, 1001-1594, and 6001-6603, as expressed by Aspergillus fumigatus; (c) any DNA sequence that hybridizes to the complement of the nucleotide sequences set forth in SEQ ID NO: 1-594, 5001-5603, 1001-1594, 6001-6603, 2001-2594, and 7001-7603 under stringent conditions, e.g., hybridization to filter-bound DNA in 6×sodium chloride/sodium citrate (SSC) at about 45° C. followed by one or more washes in 0.2×SSC/0.1% SDS at about 50-65° C., or under highly stringent conditions, e.g., hybridization to filter-bound nucleic acid in 6×SSC at about 45° C. followed by one or more washes in 0.1×SSC/0.2% SDS at about 68° C., or under other hybridization conditions which are apparent to those of skill in the art (see, for example, Ausubel, F. M. et al., eds., 1989, Current Protocols in Molecular Biology, Vol. 1, Green Publishing Associates, Inc. and John Wiley & Sons, Inc., New York, at pp. 6.3.1-6.3.6 and 2.10.3). Preferably, the polynucleotides that hybridize to the complements of the DNA sequences disclosed herein encode gene products, e.g., gene products that are functionally equivalent to a gene product encoded by a target gene. As described above, target gene sequences include not only degenerate nucleotide sequences that encode the amino acid sequences of SEQ ID NO: 3001-3594 and 8001-8603, as well as the gene product encoded by genomic SEQ ID NO: 1-594, 5001-5603, 1001-1594, and 6001-6603, as expressed in Aspergillus fumigatus, but also degenerate nucleotide sequences that when translated in organisms other than Aspergillus fumigatus, would yield a polypeptide comprising one of the amino acid sequences of SEQ ID NO: 3001-3594 and 8001-8603, as well as the gene product encoded by genomic SEQ ID NO: 1-594, 5001-5603, 1001-1594, and 6001-6603, as expressed by Aspergillus fumigatus, or a fragment thereof. One of skill in the art would know how to select the appropriate codons or modify the nucleotide sequences of SEQ ID NO: 2001-2594 and 7001-7603, when using the target gene sequences in Aspergillus fumigatus or in other organisms. Moreover, the term “target gene” encompasses genes that are naturally occurring in Saccharomyces cerevisiae, or Candida albicans or variants thereof, that share extensive nucleotide sequence homology with Aspergillus fumigatus genes having one of the DNA sequences that are set forth in SEQ ID NO: 2001-2594 and 7001-7603, i.e., the orthologs in Saccharomyces cerevisiae or in Candida albicans. It is contemplated that methods for drug screening that can be applied to Aspergillus fumigatus genes can also be applied to orthologs of the same genes in the non-pathogenic Saccharomyces cerevisiae and in the pathogenic Candida albicans. Thus, the screening methods of the invention are applicable to target genes that, depending on the objective of the screen, may or may not include genes of Saccharomyces cerevisiae or Candida albicans origin.

In another embodiment, the invention also encompasses the following polynucleotides, host cells expressing such polynucleotides and the expression products of such nucleotides: (a) polynucleotides that encode portions of target gene product that corresponds to its functional domains, and the polypeptide products encoded by such nucleotide sequences, and in which, in the case of receptor-type gene products, such domains include, but are not limited to signal sequences, extracellular domains (ECD), transmembrane domains (TM) and cytoplasmic domains (CD); (b) polynucleotides that encode mutants of a target gene product, in which all or part of one of its domains is deleted or altered, and which, in the case of receptor-type gene products, such mutants include, but are not limited to, mature proteins in which the signal sequence is cleaved, soluble receptors in which all or a portion of the TM is deleted, and nonfunctional receptors in which all or a portion of CD is deleted; and (d) polynucleotides that encode fusion proteins containing a target gene product or one of its domains fused to another polypeptide.

The invention also includes polynucleotides, preferably DNA molecules, that hybridize to, and are therefore the complements of, the DNA sequences of the target gene sequences. Such hybridization conditions can be highly stringent or less highly stringent, as described above and known in the art. The nucleic acid molecules of the invention that hybridize to the above described DNA sequences include oligodeoxynucleotides (“oligos”) which hybridize to the target gene under highly stringent or stringent conditions. In general, for oligos between 14 and 70 nucleotides in length the melting temperature (Tm) is calculated using the formula:

Tm(° C.)=81.5+16.6(log[monovalent cations (molar)]+0.41 (% G+C)−(500/N)

where N is the length of the probe. If the hybridization is carried out in a solution containing formamide, the melting temperature may be calculated using the equation:

Tm(° C.)=81.5+16.6 (log[monovalent cations (molar)])+0.41 (% G+C)−(0.61) (% formamide)−(500/N).

where N is the length of the probe. In general, hybridization is carried out at about 20-25 degrees below Tm (for DNA-DNA hybrids) or about 10-15 degrees below Tm (for RNA-DNA hybrids). Other exemplary highly stringent conditions may refer, e.g., to washing in 6×SSC/0.05% sodium pyrophosphate at 37° C. (for 14-base oligos), 48° C. (for 17-base oligos), 55° C. (for 20-base oligos), and 60° C. (for 23-base oligos).

These nucleic acid molecules can encode or act as target gene antisense molecules, useful, for example, in target gene regulation and/or as antisense primers in amplification reactions of target gene nucleotide sequences. Further, such sequences can be used as part of ribozyme and/or triple helix sequences, also useful for target gene regulation. Still further, such molecules can be used as components of diagnostic methods whereby the presence of the pathogen can be detected. The uses of these nucleic acid molecules are discussed in detail below.

Fragments of the target genes of the invention can be at least 10 nucleotides in length. In alternative embodiments, the fragments can be about 20, 30, 40, 50, 60, 70, 80, 90, 100, 200, 300, 400, 500, 1000, 1500, 2000, 2500, 3000, 3500, 4000, 4500, 5000 or more contiguous nucleotides in length. Alternatively, the fragments can comprise nucleotide sequences that encode at least 10, 20, 30, 40, 50, 100, 150, 200, 250, 300, 350, 400, 450 or more contiguous amino acid residues of the target gene products. Fragments of the target genes of the invention can also refer to exons or introns of the above described nucleic acid molecules, as well as portions of the coding regions of such nucleic acid molecules that encode functional domains such as signal sequences, extracellular domains (ECD), transmembrane domains (TM) and cytoplasmic domains (CD).

In another embodiment, the present invention is directed toward the regulatory regions that are found upstream and downstream of the coding sequences disclosed herein, which are readily determined and isolated from the genomic sequences provided herein. Included within such regulatory regions are, inter alia, promoter sequences, upstream activator sequences, as well as binding sites for regulatory proteins that modulate the expression of the genes identified herein.

In another embodiment, the present invention encompasses nucleic acid molecules comprising nucleotide sequences of introns of the essential genes of the invention. The nucleotide sequences of one or more introns of each essential gene, where present, are provided by the segment(s) of nucleotide sequences that are present in the genomic sequences (SEQ ID NO: 1001-1361 and 6001-6603) and that are absent in the corresponding open reading frame sequences (SEQ ID NO: 2001-2361 and 7001-7603, respectively). Nucleic acid molecules comprising these intron sequences or fragments thereof, although not separately provided in the sequence listing, are encompassed, and are useful for a variety of purposes, for example, as oligonucleotide primers for isolating individual exons by polymerase chain reaction or as a diagnostic tool for identifying and/or detecting a strain of A. fumigatus.

In addition to the above enumerated uses, the nucleotide sequences of essential genes of Aspergillus fumigatus have the following specific utilities:

The nucleotide sequences of the invention can be used as genetic markers and/or sequence markers to aid the development of a genetic or sequence map of the Aspergillus fumigatus genome. The nucleotide sequences and corresponding gene products of the invention can also be used to detect the presence of Aspergillus fumigatus. Hybridization and antibody-based methods well known in the art can be used to determine the presence and concentration of the nucleotide sequences and corresponding gene products of the invention.

The nucleotide sequences can also be used to make the corresponding gene products which can be used individually or in combination as an immunogen or a subunit vaccine to elicit a protective immune response in animals or subjects at high risk of developing a clinical condition, such as those that are under continual exposure of high levels of Aspergillus fumigatus conidia.

In yet another embodiment, the invention also encompasses (a) DNA vectors that contain a nucleotide sequence comprising any of the foregoing coding sequences of the target gene and/or their complements (including antisense); (b) DNA expression vectors that contain a nucleotide sequence comprising any of the foregoing coding sequences operably linked with a regulatory element that directs the expression of the coding sequences; and (c) genetically engineered host cells that contain any of the foregoing coding sequences of the target gene operably linked with a regulatory element that directs the expression of the coding sequences in the host cell. Vectors, expression constructs, expression vectors, and genetically engineered host cells containing the coding sequences of homologous target genes of other species (excluding Saccharomyces cerevisiae) are also contemplated. Also contemplated are genetically engineered host cells containing mutant alleles in homologous target genes of the other species. As used herein, regulatory elements include but are not limited to inducible and non-inducible promoters, enhancers, operators and other elements known to those skilled in the art that drive and regulate expression. Such regulatory elements include but are not limited to the lac system, the tip system, the tet system and other antibiotic-based repression systems (e.g. PIP), the TAC system, the TRC system, the major operator and promoter regions of phage A, the control regions of fd coat protein, and the fungal promoters for 3-phosphoglycerate kinase, acid phosphatase, the yeast mating pheromone responsive promoters (e.g. STE2 and STE3), and promoters isolated from genes involved in carbohydrate metabolism (e.g. GAL promoters), phosphate-responsive promoters (e.g. PHO5), or amino acid metabolism (e.g. MET genes). The invention includes fragments of any of the DNA vector sequences disclosed herein.

A variety of techniques can be utilized to further characterize the identified essential genes and virulence genes. First, the nucleotide sequence of the identified genes can be used to reveal homologies to one or more known sequence motifs which can yield information regarding the biological function of the identified gene product. Computer programs well known in the art can be employed to identify such relationships. Second, the sequences of the identified genes can be used, utilizing standard techniques such as in situ hybridization, to place the genes onto chromosome maps and genetic maps which can be correlated with similar maps constructed for another organism, e.g., Saccharomyces cerevisiae or Candida albicans. The information obtained through such characterizations can suggest relevant methods for using the polynucleotides and polypeptides for discovery of drugs against Aspergillus fumigatus and other pathogens.

Methods for performing the uses listed above are well known to those skilled in the art. References disclosing such methods include without limitation “Molecular Cloning: A Laboratory Manual,” 2d ed., Cold Spring Harbor Laboratory Press, Sambrook, J., E. F. Fritsch and T. Maniatis eds., 1989, and “Methods in Enzymology: Guide to Molecular Cloning Techniques,” Academic Press, Berger, S. L. and A. R. Kimmel eds., 1987. Basic molecular biology techniques, such as transformation and gene disruption via homologous recombination, have been developed for Aspergillus species, including Aspergillus fumigatus. Selectable markers are available for genetic manipulation in Aspergillus fumigatus which include genes conferring antibiotic resistance to hygromycin B and phleomycin, and the auxotrophic marker, Aspergillus niger PYRG which complements orotidine-5′-phosphate decarboxylase mutant alleles.

### 5.2.2 Identification of Homologs of Aspergillus fumigatus Essential Genes

The invention also provides biological and computational methods, and reagents that allow the isolation and identification of genes that are homologous to the identified essential genes of Aspergillus fumigatus. The identities and uses of such homologous genes are also encompassed by the present invention.

The methods for drug target identification and validation disclosed herein can be directly applied to other haploid pathogenic fungi. Deuteromycetous fungi, i.e. those lacking a sexual cycle and classical genetics represent the majority of human fungal pathogens. Aspergillus fumigatus is a medically-significant member of this phylum, which, more strictly, includes members of the Ascomycota and the Basidiomycota. Additional pathogenic deuteromycetous fungi to which the present methods may be extended, include Aspergillus flavus, Aspergillus niger, and Coccidiodes immitis. In those instances in which a pathogenic fungus is diploid and lacks a haploid life cycle, one allele is knocked out and the second allele is conditionally expressed as disclosed herein.

In the same way medically relevant fungal pathogens are suitable for a rational drug target discovery using the present invention, so too may plant fungal pathogens and animal pathogens be examined to identify novel drug targets for agricultural and veterinary purposes. The quality and yield of many agricultural crops including fruits, nuts, vegetables, rice, soybeans, oats, barley and wheat are significantly reduced by plant fungal pathogens. Examples include the wheat fungal pathogens causing leaf blotch (Septoria tritici, glume blotch (Septoria nodorum), various wheat rusts (Puccinia recondita, Puccinia graminis); powdery mildew (various species), and stem/stock rot (Fusarium spp.). Other particularly destructive examples of plant pathogens include, Phytophthora infestans, the causative agent of the Irish potato famine, the Dutch elm disease causing ascomycetous fungus, Ophiostoma ulmi, the corn smut causing pathogen, Ustilago maydis, the rice-blast-causing pathogen Magnapurtla grisea, Peronospora parasitica (Century et al., Proc Natl Acad Sci U S A 1995 Jul 3;92(14):6597-601); Cladosporium fulvum (leaf mould pathogen of tomato); Fusarium graminearum, Fusarium culmorum, and Fusarium avenaceum, (wheat, Abramson et al., J. Food Prot 2001 Aug;64(8):1220-5); Alternaria brassicicola (broccoli; Mora et al., Appl Microbiol Biotechnol 2001 Apr;55(3):306-10); Alternaria tagetica (Gamboa-Angulo et al., J. Agric Food Chem 2001 Mar;49(3):1228-32); the cereal pathogen Bipolaris sorokiniana (Apoga et al., FEMS Microbiol Lett 2001 Apr 13;197(2):145-50); the rice seedling blast fungus Pyricularia grisea (Lee et al., Mol Plant Microbe Interact 2001 Apr;14(4):527-35); the anther smut fungus Microbotryum violaceum (Bucheli et al.,: Mol Ecol 2001 Feb;10(2):285-94); Verticillium longisporum comb. Nov (wilt of oilseed rape, Karapapa et al., Curr Microbiol 2001 Mar;42(3):217-24); Aspergillus flavus infection of cotton bolls (Shieh et al., Appl Environ Microbiol 1997 Sep;63(9):3548-52; the eyespot pathogen Tapesia yallundae (Wood et al., FEMS Microbiol Lett 2001 Mar 15;196(2):183-7); Phytophthora cactorum strain P381 (strawberry leaf necrosis, Orsomando et al., J. Biol Chem 2001 Jun 15;276(24):21578-84); Sclerotinia sclerotiorum, an ubiquitous necrotrophic fungus (sunflowers, Poussereau et al., Microbiology 2001 Mar;147(Pt 3):717-35 26); pepper plant/cranberry, anthracnose fungus Colletotrichum gloeosporioides (Kim et al., Mol Plant Microbe Interact 2001 Jan;14(1):80-5); Nectria haematococca (pea plants, Han et al., Plant J. 2001 Feb;25(3):305-14); Cochliobolus heterostrophus (Monke et al., Mol Gen Genet 1993 Oct;241(1-2):73-80), Glomerella cingulata (Rodriquez et al., Gene 1987;54(1):73-81) obligate pathogen Bremia lactucae (lettuce downy mildew; Judelson et a]., Mol Plant Microbe Interact 1990 Jul-Aug;3(4):225-32) Rhynchosporium secalis (Rohe et al., Curr Genet 1996 May;29(6):587-90), Gibberella pulicaris (Fusarium sambucinum), Leptosphaeria maculans (Farman et al., Mol Gen Genet 1992 Jan;231(2):243-7), Cryphonectria parasitica and Mycosphaerella fjiensis and Mycosphaerella musicola, the causal agents of black and yellow Sigatoka, respectively, and Mycosphaerella eumusae, which causes Septoria leaf spot of banana (banana & plantain, Balint-Kurti et al., FEMS Microbiol Lett 2001 Feb 5;195(1):9-15). The emerging appearance of fungicidal-resistant plant pathogens and increasing reliance on monoculture practices, clearly indicate a growing need for novel and improved fungicidal compounds. Accordingly, the present invention encompasses identification and validation of drug targets in pathogens and parasites of plants and livestock. Table 2 lists exemplary groups of haploid and diploid fungi of medical, agricultural, or commercial value.

TABLE 2
Exemplary Haploid and Diploid Fungi

| | | |
|---|---|---|
| | | General Commercial |
| Animal pathogens: | Plant Pathogens: | Significance |

Ascomycota

| | | |
|---|---|---|
| Aspergillus fumigatus | Alternaria solanii | Aspergillus niger |
| Alternaria spp | Gaeumannomyces | Schizosaccharomyces |
| | graminis | pombe |
| Blastomyces dermatidis | Cercospora | Pichia pastoris |
| | zeae-maydis | |
| Candida spp including | Botrytis cinerea | Hansenula |
| | | polymorpha |
| Candida dublinensis | Claviceps purpurea | Ashbya gossipii |
| Candida glabrata | Corticum rolfsii | Aspergillus nidulans |
| Candida krusei | Endothia parasitica | Trichoderma reesei |
| Candida lustaniae | Sclerotinia | Aureobasidium |
| | sclerotiorum | pullulans |
| Candida parapsilopsis | Erysiphe gramini | Yarrowia lipolytica |
| Candida tropicalis | Erysiphe triticii | Candida utilis |
| Coccidioides immitis | Fusarium spp. | Kluveromyces lactis |
| Exophalia dermatiditis | Magnaporthe grisea | |
| Fusarium oxysporum | Plasmopara viticola | |
| Histoplasma capsulatum | Penicillium digitatum | |
| Pneumocystis carinii | Ophiostoma ulmi | |
| | Rhizoctonia species | |
| | including oryzae | |
| | Septoria species | |
| | including | |
| | Septoria avenae | |
| | Septoria nodorum | |
| | Septoria passerinii | |
| | Septoria triticii | |
| | Venturia inequalis | |
| | Verticillium dahliae | |
| | Verticillium | |
| | albo-atrum | |

Basidiomycota

| | | |
|---|---|---|
| Cryptococcus | Puccinia spp including | Agaricus campestris |
| neoformans | | |
| Trichosporon beigelii | Puccinia coronata | Phanerochaete |
| | | chrysosporium |
| | Puccinia graminis | Gloeophyllum trabeum |
| | Puccinia recondita | Trametes versicolor |
| | Puccinia striiformis | |
| | Tilletia spp including | |
| | Tilletia caries | |
| | Tilletia controversa | |
| | Tilletia indica | |
| | Tilletia tritici | |
| | Tilletiafoetida | |
| | Ustilago maydis | |
| | Ustilago hordeii | |

Zygomycota

Absidia corymbifera
Mucor rouxii
Rhizomucor pusillus
Rhizopus arrhizus

Thus, in addition to the nucleotide sequences of Aspergillus fumigatus, described above, homologs or orthologs of these target gene sequences, as can be present in other species, can be identified and isolated by molecular biological techniques well known in the art, and without undue experimentation, used in the methods of the invention. For example, homologous target genes in Aspergillus flavus, Aspergillus niger, Coccidiodes immitis, Cryptococcus neoformans, Histoplasma capsulatum, Phytophthora infestans, Puccinia seconditii, Pneumocystis carinii, or any species falling within the genera of any of the above species. Other yeasts in the genera of Candida, including Candida albicans, Saccharomyces, Schizosaccharomyces, Sporobolomyces, Torulopsis, Trichosporon, Tricophyton, Dermatophytes, Microsproum, Wickerhamia, Ashbya, Blastomyces, Candida, Citeromyces, Crebrothecium, Cryptococcus, Debaryomyces, Endomycopsis, Geotrichum, Hansenula, Kloeckera, Kluveromyces, Lipomyces, Pichia, Rhodosporidium, Rhodotorula, and Yarrowia are also contemplated. Also included are homologs of these target gene sequences which can be identified in and isolated from animal fungal pathogens such as Aspergillus niger, Aspergillus flavis, Candida albicans, Candida tropicalis, Candida parapsilopsis, Candida krusei, Cryptococcus neoformans, Coccidioides immitis, Exophalia dermatiditis, Fusarium oxysporum, Histoplasma capsulatum, Phneumocystis carinii, Trichosporon beigelii, Rhizopus arrhizus, Mucor rouxii, Rhizomucor pusillus, or Absidia corymbigera, or the plant fungal pathogens, such as Alternaria solanii, Botrytis cinerea, Erysiphe graminis, Magnaporthe grisea, Puccinia recodita, Sclerotinia sclerotiorum, Septoria triticii, Tilletia controversa, Ustilago maydis, Venturia inequalis, Verticullium dahliae or any species falling within the genera of any of the above species.

Accordingly, the present invention provides nucleotide sequences that are hybridizable to the polynucleotides of the target genes, and that are of a species other than Saccharomyces cerevisiae and Candida albicans. In one embodiment, the present invention encompasses an isolated nucleic acid comprising a nucleotide sequence that is at least 50% identical to a nucleotide sequence selected from the group consisting of SEQ ID NO.: 1-594, 5001-5603, 1001-1594, 6001-6603, 2001-2594, and 7001-7603. In another embodiment, the present invention encompasses an isolated nucleic acid comprising a nucleotide sequence that hybridizes under medium stringency conditions to a second nucleic acid that consists of a nucleotide sequence selected from the group consisting of SEQ ID NO: 1-594, 5001-5603, 1001-1594, 6001-6603, 2001-2594, 7001-7603.

In yet another embodiment, the present invention includes an isolated nucleic acid comprising a nucleotide sequence that encodes a polypeptide the amino acid sequence of which is at least 50% identical to an amino acid sequence selected from the group consisting of SEQ ID No.: 3001-3594 and 8001-8603, as well as the gene product encoded by genomic SEQ ID NO: 1-594, 5001-5603, 1001-1594, and 6001-6603, as expressed by Aspergillus fumigatus, wherein the polypeptide is that of a species other than Saccharomyces cerevisiae, Candida albicans, and Aspergillus fumigatus. Although the nucleotide sequences and amino acid sequences of homologs or orthologs of such genes in Saccharomyces cerevisiae is mostly published, as well as those homologs or orthologs of such genes in Candida albicans which is available as database version 6 assembled by the Candida albicans Sequencing Project and is accessible by internet at the web sites of Stanford University and University of Minnesota (See http://www-sequence.stanford.edu:8080/ and http://alces.med.umn.edu/Candida.html), uses of many of such homologs or orthologs in S. cerevisae or in Candida albicans in drug screening are not known and are thus specifically provided by the invention. To use such nucleotide and/or amino acid sequences of Candida albicans or Saccharomyces cerevisiae, public databases, such as Stanford Genomic Resources (www-genome.stanford.edu), Munich Information Centre for Protein Sequences (www.mips.biochem.mpg.de), or Proteome (www.proteome.com) may be used to identify and retrieve the sequences. In cases where the ortholog or homolog of a Aspergillus fumigatus gene in Candida albicans or Saccharomyces cerevisiae is known, the name of the Saccharomyces cerevisiae and/or Candida albicans gene is indicated in Table I. Orthologs of Saccharomyces cerevisiae or Candida albicans can also be identified by hybridization assays using nucleic acid probes consisting of any one of the nucleotide sequences of SEQ ID NO: 1-594, 5001-5603, 1001 -1594, 6001-6603, 2001-2594 or 7001-7603.

The nucleotide sequences of the invention still further include nucleotide sequences that have at least 40%, 45%, 55%, 60%, 65%, 70%, 75%, 80%, 85%, 90%, 95%, 98% or more nucleotide sequence identity to the nucleotide sequences set forth in SEQ ID NO: 1-594, 5001-5603, 1001-1594, 6001-6603, 2001-2594, and 7001-7603. The nucleotide sequences of the invention also include nucleotide sequences that encode polypeptides having at least 25%, 30%, 40%, 50%, 55%, 60%, 65%, 70%, 75%, 80%, 85%, 90%, 95%, 98% or higher amino acid sequence identity or similarity to the amino acid sequences set forth in SEQ ID NO: 3001-3594 and 8001-8603, as well as the gene product encoded by genomic SEQ ID NO: 1-594, 5001-5603, 1001-1594, 6001-6603, as expressed by Aspergillus fumigatus. Such nucleotide sequences may exclude S. cerevisiae and/or C. albicans sequences that are known.

Nucleotide sequences that have at least 40%, 45%, 55%, 60%, 65%, 70%, 75%, 80%, 85%, 90%, 95%, 98% or more nucleotide sequence identity to the nucleotide sequences set forth in SEQ ID NO: 1-594, 5001-5603, 1001-1594, 6001-6603, 2001-2594, and 7001-7603, can be generated by DNA shuffling, as described by Stemmer (Stemmer 1994 Proc. Natl. Acad. Sci. USA 91: 10747-51) and as disclosed in U.S. Pat. Nos. 6,323,030 B1, 6,372,497 B1, and 6,365,408 B1, each of which is hereby incorporated by reference in its entirety. In one non-limiting aspect of DNA shuffling, a DNA molecule is digested, e.g. with DNase I to provide a pool of DNA fragments. These random fragments are subjected to repeated cycles of annealing in the presence of DNA polymerase. Homology between fragments provides extendable priming sites, which generate recombinant fragments when the individual fragments are from different genes. Shuffling therefore can be carried out with a mixture of DNA fragments including, e.g. a DNA fragment encoding an Aspergillus fumigatus gene as disclosed herein as well as one or more DNA fragments encoding a homolog of the Aspergillus fumigatus gene. DNA fragments encoding such homologs can be isolated from other fungal species such as, but not limited to: Aspergillus flavus, Aspergillus niger, Coccidiodes immitis, Cryptococcus neoformans, Histoplasma capsulatum, Phytophthora infestans, Puccinia seconditii, Pneumocystis carinii, or any species falling within the genera of any of the above species. Moreover, DNA fragments encoding homologs of an Aspergillus fumigatus gene disclosed herein, which can be subjected to DNA shuffling, can also be isolated from other yeasts in the genera of Candida, including Candida albicans, Saccharomyces, Schizosaccharomyces, Sporobolomyces, Torulopsis, Trichosporon, Tricophyton, Dermatophytes, Microsproum, Wickerhamia, Ashbya, Blastomyces, Candida, Citeromyces, Crebrothecium, Cryptococcus, Debaryomyces, Endomycopsis, Geotrichum, Hansenula, Kloeckera, Kluveromyces, Lipomyces, Pichia, Rhodosporidium, Rhodotorula, and Yarrowia are also contemplated. Still further, DNA fragments useful for DNA shuffling, which encode homologs of Aspergillus fumigatus genes disclosed herein, can be identified in and isolated from animal fungal pathogens such as Aspergillus niger, Aspergillus flavis, Candida albicans, Candida tropicalis, Candida parapsilopsis, Candida krusei, Cryptococcus neoformans, Coccidioides immitis, Exophalia dermatiditis, Fusarium oxysporum, Histoplasma capsulatum, Phneumocystis carinii, Trichosporon beigeli, Rhizopus arrhizus, Mucor rouxii, Rhizomucor pusillus, or Absidia corymbigera, or the plant fungal pathogens, such as Alternaria solanii, Botrytis cinerea, Erysiphe graminis, Magnaporthe grisea, Puccinia recodita, Sclerotinia sclerotiorum, Septoria triticii, Tilletia controversa, Ustilago maydis, Venturia inequalis, Verticullium dahliae or any species falling within the genera of any of the above species.

Similarly, DNA shuffling, as described above, may also be used to construct nucleotide sequences that encode polypeptides having at least 25%, 30%, 40%, 50%, 55%, 60%, 65%, 70%, 75%, 80%, 85%, 90%, 95%, 98% or higher amino acid sequence identity or similarity to the amino acid sequences set forth in SEQ ID NO: 3001-3594 and 8001-8603, as well as the gene product encoded by genomic SEQ ID NO: 1-594, 5001-5603, 1001-1594, and 6001-6603, as expressed by Aspergillus fumigatus. Such nucleotide sequences may exclude S. cerevisiae and/or C. albicans sequences that are known.

To determine the percent identity of two amino acid sequences or of two nucleotide sequences, the sequences are aligned for optimal comparison purposes (e.g., gaps can be introduced in the sequence of a first amino acid or nucleotide sequence for optimal alignment with a second amino acid or nucleotide sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The determination of percent identity between two sequences can also be accomplished using a mathematical algorithm. A preferred, non-limiting example of a mathematical algorithm utilized for the comparison of two sequences is the algorithm of Karlin and Altschul (1990) Proc. Natl. Acad. Sci. U.S.A. 87:2264-2268, modified as in Karlin and Altschul (1993) Proc. Natl. Acad. Sci. U.S.A. 90:5873-5877. Such an algorithm is incorporated into the NBLAST and XBLAST programs of Altschul et al., 1990, J. Mol. Biol. 215: 403-0. BLAST nucleotide searches can be performed with the NBLAST nucleotide program parameters set, e.g., for score=100, wordlength=12 to obtain nucleotide sequences homologous to a nucleic acid molecules of the present invention. BLAST protein searches can be performed with the XBLAST program parameters set, e.g., to score-50, wordlength=3 to obtain amino acid sequences homologous to a protein molecule of the present invention. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., http://www.ncbi.nlm.nih.gov). Another preferred, non-limiting example of a mathematical algorithm utilized for the comparison of sequences is the algorithm of Myers and Miller, (1988) CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

To isolate homologous target genes, the Aspergillus fumigatus target gene sequence described above can be labeled and used to screen a cDNA library constructed from mRNA obtained from the organism of interest. Hybridization conditions should be of a lower stringency when the cDNA library was derived from an organism different from the type of organism from which the labeled sequence was derived. cDNA screening can also identify clones derived from alternatively spliced transcripts in the same or different species. Alternatively, the labeled fragment can be used to screen a genomic library derived from the organism of interest, again, using appropriately stringent conditions. Low stringency conditions will be well known to those of skill in the art, and will vary predictably depending on the specific organisms from which the library and the labeled sequences are derived. For guidance regarding such conditions see, for example, Sambrook et al., 1989, Molecular Cloning, A Laboratory Manual, Cold Spring Harbor Press, N.Y.; and Ausubel et al., 1989, Current Protocols in Molecular Biology, (Green Publishing Associates and Wiley Interscience, N.Y.).

Further, a homologous target gene sequence can be isolated by performing a polymerase chain reaction (PCR) using two degenerate oligonucleotide primer pools designed on the basis of amino acid sequences within the target gene of interest. The template for the reaction can be cDNA obtained by reverse transcription of mRNA prepared from the organism of interest. The PCR product can be subcloned and sequenced to ensure that the amplified sequences represent the sequences of a homologous target gene sequence.

The PCR fragment can then be used to isolate a full length cDNA clone by a variety of methods well known to those of ordinary skill in the art. Alternatively, the labeled fragment can be used to screen a genomic library.

PCR technology can also be utilized to isolate full length cDNA sequences. For example, RNA can be isolated, following standard procedures, from an organism of interest. A reverse transcription reaction can be performed on the RNA using an oligonucleotide primer specific for the most 5′ end of the amplified fragment for the priming of first strand synthesis. The resulting RNA/DNA hybrid can then be “tailed” with guanines using a standard terminal transferase reaction, the hybrid can be digested with RNAase H, and second strand synthesis can then be primed with a poly-C primer. Thus, cDNA sequences upstream of the amplified fragment can easily be isolated. For a review of cloning strategies which can be used, see e.g., Sambrook et al., 1989, Molecular Cloning, A Laboratory Manual, Cold Springs Harbor Press, N.Y.; and Ausubel et al., 1989, Current Protocols in Molecular Biology, (Green Publishing Associates and Wiley Interscience, N.Y.).

Additionally, an expression library can be constructed utilizing DNA isolated from or cDNA synthesized from the organism of interest. In this manner, gene products made by the homologous target gene can be expressed and screened using standard antibody screening techniques in conjunction with antibodies raised against the Candida albicans gene product, as described, below. (For screening techniques, see, for example, Harlow, E. and Lane, eds., 1988, “Antibodies: A Laboratory Manual,” Cold Spring Harbor Press, Cold Spring Harbor). Library clones detected via their reaction with such labeled antibodies can be purified and subjected to sequence analysis by well known methods.

Alternatively, homologous target genes or polypeptides may be identified by searching a database to identify sequences having a desired level of homology to a target gene or polypeptide involved in proliferation, virulence or pathogenicity. A variety of such databases are available to those skilled in the art, including GenBank and GenSeq. In various embodiments, the databases are screened to identify nucleic acids with at least 97%, at least 95%, at least 90%, at least 85%, at least 80%, at least 70%, at least 60%, at least 50%, or at least 40% identity to a target nucleotide sequence, or a portion thereof. In other embodiments, the databases are screened to identify polypeptides having at least 99%, at least 95%, at least 90%, at least 85%, at least 80%, at least 70%, at least 60%, at least 50%, at least 40% or at least 25% identity or similarity to a polypeptide involved in proliferation, virulence or pathogenicity or a portion thereof.

Alternatively, functionally homologous target sequences or polypeptides may be identified by creating mutations that have phenotypes by removing or altering the function of a gene. This can be done for one or all genes in a given fungal species including, for example: Saccharomyces cerevisiae, Candida albicans, and Aspergillus fumigatus. Having mutants in the genes of one fingal species offers a method to identify functionally similar genes or related genes (orthologs) in another species, or functionally similar genes in the same species (paralogs), by use of a functional complementation test.

A library of gene or cDNA copies of messenger RNA of genes can be made from a given species, e.g. Aspergillus fumigatus, and the library cloned into a vector permitting expression (for example, with the Aspergillus fumigatus, Aspergillus nidulans promoters or Saccharomyces cerevisiae promoters) of the genes in a second species, e.g. Saccharomyces cerevisiae or Candida albicans. Such a library is referred to as a “heterologous library.” Transformation of the Aspergillus fumigatus heterologous library into a defined mutant of Saccharomyces cerevisiae or Candida albicans that is functionally deficient with respect to the identified gene, and screening or selecting for a gene in the heterologous library that restores phenotypic function in whole or in part of the mutational defect is said to be “heterologous functional complementation” and in this example, permits identification of gene in Aspergillus fumigatus that are functionally related to the mutated gene in Saccharomyces cerevisiae or Candida albicans. Inherent in this functional-complementation method, is the ability to restore gene function without the requirement for sequence similarity of nucleic acids or polypeptides; that is, this method permits interspecific identification of genes with conserved biological function, even where sequence similarity comparisons fail to reveal or suggest such conservation.

In those instances in which the gene to be tested is an essential gene, a number of possibilities exist regarding performing heterologous functional complementation tests. The mutation in the essential gene can be a conditional allele, including but not limited to, a temperature-sensitive allele, an allele conditionally expressed from a regulatable promoter, or an allele that has been rendered the mRNA transcript or the encoded gene product conditionally unstable. Alternatively, the strain carrying a mutation in an essential gene can be propagated using a copy of the native gene (a wild type copy of the gene mutated from the same species) on a vector comprising a marker that can be selected against, permitting selection for those strains carrying few or no copies of the vector and the included wild type allele. A strain constructed in this manner is transformed with the heterologous library, and those clones in which a heterologous gene can functionally complement the essential gene mutation, are selected on medium non-permissive for maintenance of the plasmid carrying the wild type gene.

A heterologous functional complementation test is not restricted to the exchange of genetic information between Aspergillus fumigatus, Candida albicans and Saccharomyces cerevisiae; functional complementation tests can be performed, as described above, using any pair of fungal species. For example, the CRE1 gene of the fungus Sclerotininia sclerotiorum can functionally complement the creAD30 mutant of the CREA gene of Aspergillus nidulans (see Vautard et al. 1999, “The glucose repressor gene CRE1 from Selerotininia sclerotiorum is functionally related to CREA from Aspergillus niger but not to the Mig proteins from Saccharomyces cerevisiae,” FEBS Lett. 453: 54-58).

In yet another embodiment, where the source of nucleic acid deposited on a gene expression array and the source of the nucleic acid probe being hybridized to the array are from two different species of organisms, the results allow rapid identification of homologous genes in the two species.

### 5.2.3 Products Encoded by Aspergillus fumigatus Essential Genes

The target gene products used and encompassed in the methods and compositions of the present invention include those gene products (e.g., RNA or proteins) that are encoded by the target essential gene sequences as described above, such as, the target gene sequences set forth in SEQ ID NO: 2001-2594 and 7001-7603. When expressed in an organism which does not use the universal genetic code, protein products of the target genes having the amino acid sequences of SEQ ID NO: 3001-3594 and 8001-8603, as well as the gene product encoded by genomic SEQ ID NO: 1-594, 5001-5603, 1001-1594, and 6001-6603, as expressed by Aspergillus fumigatus, may be encoded by nucleotide sequences that conform to the known codon usage in the organism. One of skill in the art would know the modifications that are necessary to accommodate for a difference in codon usage, e.g., that of Candida albicans.

In addition, however, the methods and compositions of the invention also use and encompass proteins and polypeptides that represent functionally equivalent gene products. Such functionally equivalent gene products include, but are not limited to, natural variants of the polypeptides comprising or consisting essentially of an amino acid sequence set forth in SEQ ID NO: 3001-3594 and 8001-8603, as well as the gene product encoded by genomic SEQ I) NO: 1-594, 5001-5603, 1001-1594, and 6001-6603, as expressed by Aspergillus fumigatus.

Such equivalent target gene products can contain, e.g., deletions, additions or substitutions of amino acid residues within the amino acid sequences encoded by the target gene sequences described above, but which result in a silent change, thus producing a functionally equivalent target gene product. Amino acid substitutions can be made on the basis of similarity in polarity, charge, solubility, hydrophobicity, hydrophilicity and/or the amphipathic nature of the residues involved. For example, nonpolar (i.e., hydrophobic) amino acid residues can include alanine (Ala or A), leucine (Leu or L), isoleucine (Ile or I), valine (Val or V), proline (Pro or P), phenylalanine (Phe or F), tryptophan (Trp or W) and methionine (Met or M); polar neutral amino acid residues can include glycine (Gly or G), serine (Ser or S), threonine (Thr or T), cysteine (Cys or C), tyrosine (Tyr or Y), asparagine (Asn or N) and glutamine (Gln or Q); positively charged (i.e., basic) amino acid residues can include arginine (Arg or R), lysine (Lys or K) and histidine (His or H); and negatively charged (i.e., acidic) amino acid residues can include aspartic acid (Asp or D) and glutamic acid (Glu or E).

“Functionally equivalent,” as the term is utilized herein, refers to a polypeptide capable of exhibiting a substantially similar in vivo activity as the Aspergillus fumigatus target gene product encoded by one or more of the target gene sequences described in Table 2. Alternatively, when utilized as part of assays described hereinbelow, the term “functionally equivalent” can refer to peptides or polypeptides that are capable of interacting with other cellular or extracellular molecules in a manner substantially similar to the way in which the corresponding portion of the target gene product would interact with such other molecules. Preferably, the functionally equivalent target gene products of the invention are also the same size or about the same size as a target gene product encoded by one or more of the target gene sequences described in Table I.

Peptides and polypeptides corresponding to one or more domains of the target gene products (e.g., signal sequence, TM, ECD, CD, or ligand-binding domains), truncated or deleted target gene products (e.g., polypeptides in which one or more domains of a target gene product are deleted) and fusion target gene proteins (e.g., proteins in which a full length or truncated or deleted target gene product, or a peptide or polypeptide corresponding to one or more domains of a target gene product is fused to an unrelated protein) are also within the scope of the present invention. Such peptides and polypeptides (also referred to as chimeric protein or polypeptides) can be readily designed by those skilled in the art on the basis of the target gene nucleotide and amino acid sequences listed in Table I. Exemplary fusion proteins can include, but are not limited to, epitope tag-fusion proteins which facilitate isolation of the target gene product by affinity chromatography using reagents that binds the epitope. Other exemplary fusion proteins include fusions to any amino acid sequence that allows, e.g., the fusion protein to be anchored to a cell membrane, thereby allowing target gene polypeptides to be exhibited on a cell surface; or fusions to an enzyme (e.g., β-galactosidase encoded by the LAC4 gene of Kluyveronmyces lactis (Leuker et al., 1994, Mol. Gen. Genet., 245:212-217)), to a fluorescent protein (e.g., from Renilla reniformis (Srikantha et al., 1996, J. Bacteriol. 178:121-129), or to a 20 luminescent protein which can provide a marker function. Accordingly, the invention provides a fusion protein comprising a fragment of a first polypeptide fused to a second polypeptide, said fragment of the first polypeptide consisting of at least 6 consecutive residues of an amino acid sequence selected from one of SEQ ID NO: 3001-3594 and 8001-8603.

Other modifications of the target gene product coding sequences described above can be made to generate polypeptides that are better suited, e.g., for expression, for scale up, etc. in a chosen host cell. For example, cysteine residues can be deleted or substituted with another amino acid in order to eliminate disulfide bridges.

The target gene products of the invention preferably comprise at least as many contiguous amino acid residues as are necessary to represent an epitope fragment (that is, for the gene products to be recognized by an antibody directed to the target gene product). For example, such protein fragments or peptides can comprise at least about 8 contiguous amino acid residues from a full length differentially expressed or pathway gene product. In alternative embodiments, the protein fragments and peptides of the invention can comprise about 6, 10, 20, 30, 40, 50, 60, 70, 80, 90, 100, 150, 200, 250, 300, 350, 400, 450 or more contiguous amino acid residues of a target gene product.

The target gene products used and encompassed in the methods and compositions of the present invention also encompass amino acid sequences encoded by one or more of the above-described target gene sequences of the invention wherein domains often encoded by one or more exons of those sequences, or fragments thereof, have been deleted. The target gene products of the invention can still further comprise post translational modifications, including, but not limited to, glycosylations, acetylations and myristylations.

The target gene products of the invention can be readily produced, e.g., by synthetic techniques or by methods of recombinant DNA technology using techniques that are well known in the art. Thus, methods for preparing the target gene products of the invention are discussed herein. First, the polypeptides and peptides of the invention can be synthesized or prepared by techniques well known in the art. See, for example, Creighton, 1983, Proteins: Structures and Molecular Principles, W. H. Freeman and Co., N.Y., which is incorporated herein by reference in its entirety. Peptides can, for example, be synthesized on a solid support or in solution.

Alternatively, recombinant DNA methods which are well known to those skilled in the art can be used to construct expression vectors containing target gene protein coding sequences such as those set forth in SEQ ID NO: 2001-2594 and 7001-7603, and appropriate transcriptional/translational control signals. These methods include, for example, in vitro recombinant DNA techniques, synthetic techniques and in vivo recombination/genetic recombination. See, for example, the techniques described in Sambrook et al., 1989, Molecular Cloning: A Laboratory Manual, Cold Spring Harbor Press, Cold Spring Harbor, N.Y., Pla et al., Yeast 12:1677-1702 (1996), which are incorporated by reference herein in their entireties, and Ausubel, 1989, supra. Alternatively, RNA capable of encoding target gene protein sequences can be chemically synthesized using, for example, synthesizers. See, for example, the techniques described in Oligonucleotide Synthesis, 1984, Gait, M. J. ed., IRL Press, Oxford, which is incorporated by reference herein in its entirety.

A variety of host-expression vector systems can be utilized to express the target gene coding sequences of the invention. Such host-expression systems represent vehicles by which the coding sequences of interest can be produced and subsequently purified, but also represent cells which can, when transformed or transfected with the appropriate nucleotide coding sequences, exhibit the target gene protein of the invention in situ. These include but are not limited to microorganisms such as bacteria (e.g., E. coli, B. subtilis) transformed with recombinant bacteriophage DNA, plasmid DNA or cosmid DNA expression vectors containing target gene protein coding sequences; yeast (e.g., Saccharomyces, Schizosaccarhomyces, Neurospora, Aspergillus, Candida, Pichia) transformed with recombinant yeast expression vectors containing the target gene protein coding sequences; insect cell systems infected with recombinant virus expression vectors (e.g., baculovirus) containing the target gene protein coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing target gene protein coding sequences; or mammalian cell systems (e.g. COS, CHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5K promoter). If necessary, the nucleotide sequences of coding regions may be modified according to the codon usage of the host such that the translated product has the correct amino acid sequence.

In bacterial systems, a number of expression vectors can be advantageously selected depending upon the use intended for the target gene protein being expressed For example, when a large quantity of such a protein is to be produced, for the generation of antibodies or to screen peptide libraries, for example, vectors which direct the expression of high levels of fusion protein products that are readily purified can be desirable. Such vectors include, but are not limited, to the E. coli expression vector pUR278 (Ruther et al., 1983, EMBO J. 2:1791), in which the target gene protein coding sequence can be ligated individually into the vector in frame with the lacZ coding region so that a fusion protein is produced; pIN vectors (Inouye & Inouye, 1985, Nucleic Acids Res. 13:3101-3109; Van Heeke & Schuster, 1989, J. Biol. Chem. 264:5503-5509); and the like. pGEX vectors can also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned target gene protein can be released from the GST moiety.

When a target gene is to be expressed in mammalian host cells, a number of viral-based expression systems can be utilized. In cases where an adenovirus is used as an expression vector, the target gene coding sequence of interest can be ligated to an adenovirus transcription/translation control complex, e.g., the late promoter and tripartite leader sequence. This chimeric gene can then be inserted in the adenovirus genome by ill vitro or in vivo recombination. Insertion in a non-essential region of the viral genome (e.g., region E1 or E3) will result in a recombinant virus that is viable and capable of expressing target gene protein in infected hosts, (e.g., See Logan & Shenk, 1984, Proc. Natl. Acad. Sci. USA 81:3655-3659). Specific initiation signals can also be required for efficient translation of inserted target gene coding sequences. These signals include the ATG initiation codon and adjacent sequences. In cases where an entire target gene, including its own initiation codon and adjacent sequences, is inserted into the appropriate expression vector, no additional translational control signals can be needed. However, in cases where only a portion of the target gene coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see Bittner et al., 1987, Methods in Enzymol. 153:516-544).

In addition, a host cell strain can be chosen which modulates the expression of the inserted sequences, or modifies and processes the gene product in the specific fashion desired. Such modifications (e.g., glycosylation) and processing (e.g., cleavage) of protein products can be important for the function of the protein. Different host cells have characteristic and specific mechanisms for the post-translational processing and modification of proteins. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product can be used.

For long-term, high-yield production of recombinant proteins, stable expression is preferred. For example, cell lines which stably express the target gene protein can be engineered. Host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells can be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which express the target gene protein. Such engineered cell lines can be particularly useful in screening and evaluation of compounds that affect the endogenous activity of the target gene protein.

A number of selection systems can be used, including but not limited to the herpes simplex virus thymidine kinase (Wigler et al, 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy et al., 1980, Cell 22:817) genes can be employed in tk⁻, hgprt⁻ or aprt⁻ cells, respectively. Also, antimetabolite resistance can be used as the basis of selection for dhfr, which confers resistance to methotrexate (Wigler et al., 1980, Proc. Natl. Acad. Sci. USA 77:3567; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin et al., 1981, J. Mol. Biol. 150:1); and hygro, which confers resistance to hygromycin (Santerre et al., 1984, Gene 30:147) genes.

Alternatively, any fusion protein may be readily purified by utilizing an antibody specific for the fusion protein being expressed. For example, a system described by Janknecht et al. allows for the ready purification of non-denatured fusion proteins expressed in human cells lines (Janknecht et al., 1991, Proc. Natl. Acad. Sci. USA 88: 8972-8976). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺ nitriloacetic acid-agarose columns and histidine-tagged proteins are selectively eluted with imidazole-containing buffers. Fusions at the carboxy terminal of the target gene product are also contemplated.

When used as a component in assay systems such as those described herein, the target gene protein can be labeled, either directly or indirectly, to facilitate detection of a complex formed between the target gene protein and a test substance. Any of a variety of suitable labeling systems can be used including but not limited to radioisotopes such as ¹²⁵I; enzyme labeling systems that generate a detectable colorimetric signal or light when exposed to substrate; and fluorescent labels.

Indirect labeling involves the use of a protein, such as a labeled antibody, which specifically binds to either a target gene product. Such antibodies include but are not limited to polyclonal antibodies, monoclonal antibodies (mAbs), human, humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above.

Following expression of the target gene protein encoded by the identified target nucleotide sequence, the protein is purified. Protein purification techniques are well known in the art. Proteins encoded and expressed from identified exogenous nucleotide sequences can be partially purified using precipitation techniques, such as precipitation with polyethylene glycol. Alternatively, epitope tagging of the protein can be used to allow simple one step purification of the protein. In addition, chromatographic methods such as ion-exchange chromatography, gel filtration, use of hydroxyapaptite columns, immobilized reactive dyes, chromatofocusing, and use of high-performance liquid chromatography, may also be used to purify the protein. Electrophoretic methods such as one-dimensional gel electrophoresis, high-resolution two-dimensional polyacrylamide electrophoresis, isoelectric focusing, and others are contemplated as purification methods. Also, affinity chromatographic methods, comprising solid phase bound- antibody, ligand presenting columns and other affinity chromatographic matrices are contemplated as purification methods in the present invention.

In addition, the purified target gene products, fragments thereof, or derivatives thereof may be administered to an individual in a pharmaceutically acceptable carrier to induce an immune response against the protein or polypeptide. Preferably, the immune response is a protective immune response which protects the individual. Methods for determining appropriate dosages of the protein (including use of adjuvants) and pharmaceutically acceptable carriers are familiar to those skilled in the art.

### 5.2.4 Isolation and Use of Antibodies Recognizing Products Encoded by Aspergillus fumigatus Essential Genes

Described herein are methods for the production of antibodies capable of specifically recognizing epitopes of one or more of the target gene products described above. Such antibodies can include, but are not limited to, polyclonal antibodies, monoclonal antibodies (mAbs), human, humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above.

For the production of antibodies to a target gene or gene product, various host animals can be immunized by injection with a target gene protein, or a portion thereof. Such host animals can include but are not limited to rabbits, mice, and rats, to name but a few. Various adjuvants can be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum. Accordingly, the invention provides a method of eliciting an immune response in an animal, comprising introducing into the animal an immunogenic composition comprising an isolated polypeptide, the amino acid sequence of which comprises at least 6 consecutive residues of one of SEQ ID NOs: 3001-3594 or 8001-8603, as well as the gene products, such as splice variants, that are encoded by genomic sequences, SEQ ID NOs: 1-594, 5001-5603, 1001-1594, 6001-6603, as expressed by Aspergillus fumigatus.

Polyclonal antibodies are heterogeneous populations of antibody molecules derived from the sera of animals immunized with an antigen, such as target gene product, or an antigenic functional derivative thereof. For the production of polyclonal antibodies, host animals such as those described above, can be immunized by injection with differentially expressed or pathway gene product supplemented with adjuvants as also described above. The antibody titer in the immunized animal can be monitored over time by standard techniques, such as with an enzyme linked immunosorbent assay (ELISA) using immobilized polypeptide. If desired, the antibody molecules can be isolated from the animal (e.g., from the blood) and further purified by well-known techniques, such as protein A chromatography to obtain the IgG fraction.

Monoclonal antibodies, which are homogeneous populations of antibodies to a particular antigen, can be obtained by any technique which provides for the production of antibody molecules by continuous cell lines in culture. These include, but are not limited to the hybridoma technique of Kohler and Milstein, (1975, Nature 256:495-497; and U.S. Pat. No. 4,376,110), the human B-cell hybridoma technique (Kosbor et al., 1983, Immunology Today 4:72; Cole et al., 1983, Proc. Natl. Acad. Sci. USA 80:2026-2030), and the EBV-hybridoma technique (Cole et al., 1985, Monoclonal Antibodies And Cancer Therapy, Alan R. Liss, Inc., pp. 77-96). Such antibodies can be of any immunoglobulin class including IgG, IgM, IgE, IgA, IgD and any subclass thereof. The hybridoma producing the mAb of this invention can be cultivated in vitro or in vivo. Production of high titers of mAbs in vivo makes this the presently preferred method of production.

Alternative to preparing monoclonal antibody-secreting hybridomas, a monoclonal antibody directed against a polypeptide of the invention can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al (1991) Bio/Technology 9:1370-1372; Hayetal. (1992) Hum. Antibod. Hybridomas 3:81-85; Huse et al. (1989) Science 246:1275-1281; Griffiths et al. (1993) EMBO J. 12:725-734.

Additionally, recombinant antibodies, such as chimeric and humanized monoclonal antibodies, comprising both human and non-human portions, which can be made using standard recombinant DNA techniques, are within the scope of the invention. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816397, which are incorporated herein by reference in their entirety.) Humanized antibodies are antibody molecules from non-human species having one or more complementarily determining regions (CDRs) from the non-human species and a framework region from a human immunoglobulin molecule. (See, e.g., Queen, U.S. Pat. No. 5,585,089, which is incorporated herein by reference in its entirety.) Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

Completely human antibodies are particularly desirable for therapeutic treatment of human patients. Such antibodies can be produced using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al. (1994) Bio/technology 12:899-903).

Antibody fragments which recognize specific epitopes can be generated by known techniques. For example, such fragments include but are not limited to: the F(ab′)₂ fragments which can be produced by pepsin digestion of the antibody molecule and the Fab fragments which can be generated by reducing the disulfide bridges of the F(ab′)₂ fragments. Alternatively, Fab expression libraries can be constructed (Huse et al., 1989, Science 246:1275-1281) to allow rapid and easy identification of monoclonal Fab fragments with the desired specificity.

Antibodies of the present invention may also be described or specified in terms of their binding affinity to a target gene product. Preferred binding affinities include those with a dissociation constant or Kd less than 5×10⁻⁶ M, 10⁻⁶M, 5×10⁻⁷M, 10⁻⁷M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻M.

Antibodies directed against a target gene product or fragment thereof can be used to detect the a target gene product in order to evaluate the abundance and pattern of expression of the polypeptide under various environmental conditions, in different morphological forms (mycelium, yeast, spores) and stages of an organism's life cycle. Antibodies directed against a target gene product or fragment thereof can be used diagnostically to monitor levels of a target gene product in the tissue of an infected host as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Further, antibodies directed against a target gene product or fragment thereof can be used therapeutically to treat an infectious disease by preventing infection, and/or inhibiting growth of the pathogen. Antibodies can also be used to modify a biological activity of a target gene product. Antibodies to gene products related to virulence or pathogenicity can also be used to prevent infection and alleviate one or more symptoms associated with infection by the organism. To facilitate or enhance its therapeutic effect, an antibody (or fragment thereof) may be conjugated to a therapeutic moiety such as a toxin or fungicidal agent. Techniques for conjugating a therapeutic moiety to antibodies are well known, see, e.g., Thorpe et al., “The Preparation And Cytotoxic Properties Of Antibody-Toxin Conjugates,” Immunol. Rev., 62:119-58 (1982).

An antibody with or without a therapeutic moiety conjugated to it can be used as a therapeutic that is administered alone or in combination with chemotherapeutic agents.

### 5.2.5 Modulation of Essential Aspergillus fumigatus Gene Expression Using Ribozymes

Ribozymes are enzymatic RNA molecules capable of catalyzing the specific cleavage of RNA (For a review see, for example Rossi, J., 1994, Current Biology 4:469-471). The mechanism of ribozyme action involves sequence specific hybridization of the ribozyme molecule to complementary target RNA, followed by a endonucleolytic cleavage. The composition of ribozyme molecules must include one or more sequences complementary to the target gene mRNA, and must include the well known catalytic sequence responsible for mRNA cleavage. For this sequence, see U.S. Pat. No. 5,093,246, which is incorporated by reference herein in its entirety. As such, within the scope of the invention are engineered hammerhead motif ribozyme molecules that specifically and efficiently catalyze endonucleolytic cleavage of RNA sequences encoding target gene proteins.

Ribozyme molecules designed to catalytically cleave specific target gene mRNA transcripts can also be used to prevent translation of target gene mRNA and expression of target genes. While ribozymes that cleave mRNA at site specific recognition sequences can be used to destroy target gene mRNAs, the use of hammerhead ribozymes is preferred. Hammerhead ribozymes cleave mRNAs at locations dictated by flanking regions that form complementary base pairs with the target gene mRNA. The sole requirement is that the target mRNA have the following sequence of two bases: 5′-UG-3′. The construction and production of hammerhead ribozymes is well known in the art and is described more fully in Haseloff and Gerlach, 1988, Nature, 334:585-591. Preferably the ribozyme is engineered so that the cleavage recognition site is located near the 5′ end of the target gene mRNA; i.e., to increase efficiency and minimize the intracellular accumulation of non-functional mRNA transcripts.

The ribozymes of the present invention also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al., 1984, Science, 224:574-578; Zaug and Cech, 1986, Science, 231:470-475; Zaug, et al, 1986, Nature, 324:429-433; published International patent application No. WO 88/04300 by University Patents Inc.; Been and Cech, 1986, Cell, 47:207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in a target gene.

As in the antisense approach, the ribozymes can be composed of modified oligonucleotides (e.g. for improved stability, targeting, etc.) and should be delivered to cells which express the target gene in vivo. Because ribozymes unlike antisense molecules, are catalytic, a lower intracellular concentration is required for efficiency. Multiple ribozyme molecules directed against different target genes can also be used in combinations, sequentially or simultaneously.

Anti-sense RNA and DNA, ribozyme, and triple helix molecules of the invention can be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules can be generated by in vitro and in vivo transcription of DNA sequences encoding the antisense RNA molecule. Such DNA sequences can be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines. These nucleic acid constructs can be administered selectively to the desired cell population via a delivery complex.

Various well-known modifications to the DNA molecules can be introduced as a means of increasing intracellular stability and half-life. Possible modifications include, but are not limited to, the addition of flanking sequences of ribo- or deoxy- nucleotides to the 5′ and/or 3′ ends of the molecule or the use of phosphorothioate or 2′ O-methyl rather than phosphodiesterase linkages within the oligodeoxyribonucleotide backbone.

### 5.2.6 Modulation of Essential Aspergillus fumigatus Gene Expression Using Antisense Molecules

The use of antisense molecules as inhibitors of gene expression may be a specific, genetically based therapeutic approach (for a review, see Stein, in Ch. 69, Section 5 “Cancer: Principle and Practice of Oncology”, 4th ed., ed. by DeVita et al., J. B. Lippincott, Philadelphia 1993). The present invention provides the therapeutic or prophylactic use of nucleic acids of at least six nucleotides that are antisense to a target essential or virulence gene or a portion thereof in the target organism. An “antisense” target nucleic acid as used herein refers to a nucleic acid capable of hybridizing to a portion of a target gene RNA (preferably mRNA) by virtue of some sequence complementarity. The invention further provides pharmaceutical compositions comprising an effective amount of the antisense nucleic acids of the invention in a pharmaceutically acceptable carrier, as described infra.

In another embodiment, the invention is directed to methods for inhibiting the expression of a target gene in an organism of interest, such as Aspergillus fumigatus, either in vitro, ex vivo, or in vivo, comprising providing the cell with an effective amount of a composition comprising an antisense nucleic acid of the invention. Multiple antisense polynucleotides hybridizable to different target genes may be used in combinations, sequentially or simultaneously.

In another embodiment, the present invention is directed toward methods for modulating expression of an essential gene which has been identified by the methods described supra, in which an antisense RNA molecule, which inhibits translation of mRNA transcribed from an essential gene, is expressed from a regulatable promoter. In one aspect of this embodiment, the antisense RNA molecule is expressed in a conditional-expression Aspergillus fumigatus mutant strain. In other aspects of this embodiment, the antisense RNA molecule is expressed in a wild-type strain of Aspergillus or another haploid or diploid pathogenic organism, including animal fugal pathogens such as Aspergillus niger, Aspergillus flavis, Candida albicans, Candida tropicalis, Candida parapsilopsis, Candida krusei, Cryptococcus neoformans, Coccidioides immitis, Exophalia dermatiditis, Fusarium oxysporum, Histoplasma capsulatum, Phneumocystis carinii, Trichosporon beigelii, Rhizopus arrhizus, Mucor rouxii, Rhizomucor pusillus, or Absidia corymbigera, or the plant fungal pathogens, such as Botrytis cinerea, Erysiphe graminis, Magnaporthe grisea, Puccinia recodita, Septoria triticii, Tilletia controversa, Ustilago maydiss, or any species falling within the genera of any of the above species.

The nucleic acid molecule comprising an antisense nucleotide sequence of the invention may be complementary to a coding and/or noncoding region of a target gene mRNA. The antisense molecules will bind to the complementary target gene mRNA transcripts and reduce or prevent translation. Absolute complementarity, although preferred, is not required. A sequence “complementary” to a portion of an RNA, as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. One skilled in the art can ascertain a tolerable degree of mismatch by use of standard procedures to determine the melting point of the hybridized complex.

Nucleic acid molecules that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have recently been shown to be effective at inhibiting translation of mRNAs as well. See generally, Wagner, R., 1994, Nature 372:333-335.

Nucleic acid molecules comprising nucleotide sequences complementary to the 5′ untranslated region of the mRNA can include the complement of the AUG start codon. Antisense nucleic acid molecules complementary to mRNA coding regions are less efficient inhibitors of translation but could be used in accordance with the invention. Whether designed to hybridize to the 5′-, 3′- or coding region of target gene mRNA, antisense nucleic acids should be at least six nucleotides in length, and are preferably oligonucleotides ranging from 6 to about 50 nucleotides in length. In specific aspects, the oligonucleotide is at least 10 nucleotides, at least 17 nucleotides, at least 25 nucleotides, at least 50 nucleotides, or at least 200 nucleotides.

Regardless of the choice of target gene sequence, it is preferred that in vitro studies are first performed to quantitate the ability of the antisense molecule to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The antisense molecule can be DNA or RNA or chimeric mixtures or derivatives or modified versions thereof, single-stranded or double-stranded. The antisense molecule can be modified at the base moiety, sugar moiety, or phosphate backbone, for example, to improve stability of the molecule, hybridization, etc. The antisense molecule may include other appended groups such as peptides (e.g., for targeting cell receptors in vivo), hybridization-triggered cleavage agents. (See, e.g., Krol et al., 1988, BioTechniques 6:958-976) or intercalating agents. (See, e.g., Zon, 1988, Pharm. Res. 5:539-549). To this end, the antisense molecule may be conjugated to another molecule, e.g., a peptide, hybridization triggered cross-linking agent, transport agent, hybridization-triggered cleavage agent, etc.

The antisense molecule may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense molecule may also comprise at least one modified sugar moiety selected from the group including but not limited to arabinose, 2-fluoroarabinose, xylulose, and hexose.

In yet another embodiment, the antisense molecule comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense molecule is an α-anomeric oligonucleotide. An α-anomeric oligonucleotide forms specific double-stranded hybrids with complementary RNA in which, contrary to the usual β-units, the strands run parallel to each other (Gautier et al., 1987, Nucl. Acids Res. 15:6625-6641). The oligonucleotide is a 2′-0-methylribonucleotide (Inoue et al., 1987, Nucl. Acids Res. 15:6131-6148), or a chimeric RNA-DNA analogue (Inoue et al., 1987, FEBS Lett. 215:327-330).

Antisense molecules of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad. Sci. U.S.A. 85:7448-7451), etc.

While antisense nucleotides complementary to the coding region of a target gene could be used, those complementary to the transcribed untranslated region are also preferred.

Pharmaceutical compositions of the invention comprising an effective amount of an antisense nucleic acid in a pharmaceutically acceptable carrier, can be administered to a subject infected with the pathogen of interest.

The amount of antisense nucleic acid which will be effective in the treatment of a particular disease caused by the pathogen will depend on the site of the infection or condition, and can be determined by standard techniques. Where possible, it is desirable to determine the antisense cytotoxicity of the pathogen to be treated in vitro, and then in useful animal model systems prior to testing and use in humans.

A number of methods have been developed for delivering antisense DNA or RNA to cells; e.g., antisense molecules can be injected directly into the tissue site in which the pathogens are residing, or modified antisense molecules, designed to target the desired cells (e.g., antisense molecule linked to peptides or antibodies that specifically bind receptors or antigens expressed on the pathogen's cell surface) can be administered systemically. Antisense molecules can be delivered to the desired cell population via a delivery complex. In a specific embodiment, pharmaceutical compositions comprising antisense nucleic acids of the target genes are administered via biopolymers (e.g., poly-β-1→4-N-acetylglucosamine polysaccharide), liposomes, microparticles, or microcapsules. In various embodiments of the invention, it may be useful to use such compositions to achieve sustained release of the antisense nucleic acids. In a specific embodiment, it may be desirable to utilize liposomes targeted via antibodies to specific identifiable pathogen antigens (Leonetti et al., 1990, Proc. Natl. Acad. Sci. U.S.A. 87:2448-2451; Renneisen et al., 1990, J. Biol. Chem. 265:16337-16342).

### 5.2.7 Construction of Aspergillus fumigatus Strains Carrying Mutant Essential Genes

In one embodiment of the present invention, each of the essential genes of the invention is placed under the control of the heterologous promoter, the activity of which is regulatable. Where the gene is essential, elimination of expression of that gene will be lethal or severely crippling for growth. Therefore, in the present invention, a heterologous promoter is used to provide a range of levels of expression of a target. Depending on the conditions, the gene may be under-expressed, over-expressed, or expressed at a level comparable to that observed when the target gene is linked to its native promoter. A heterologous promoter is a promoter from a different gene from the same pathogenic organism, or it can be a promoter from a different species. In one embodiment of the invention, the heterologous, regulatable promoter is the Aspergillus niger Pgla A promoter. Transcription from the Pgla A promoter is stimulated in the presence of maltose and repressed in the presence of xylose. Accordingly, replacement of the promoter regions of the target essential gene with the Pgla A, enables regulation of the expression of the target gene by growing the Aspergillus fumigatus host carrying the modified gene in the presence of maltose and/or xylose (see the Example disclosed in Section 6.2, infra).

The process can be repeated for a desired subset of the genes such that a collection of conditional-expression mutant Aspergillus fumigatus strains is generated wherein each strain comprises a different, conditionally-expressed gene. A preferred embodiment for the construction of conditional-expression mutants of Aspergillus fumigatus strains, uses the following, non-limiting method.

PCR amplification of a dominant selectable marker so as to include about 65 bp of flanking sequence identical to the sequence 5′ and 3′ of the Aspergillus fumigatus gene to be disrupted, allows construction of a gene disruption cassette for any given Aspergillus fumigatus gene.

Where a knock-out mutant of a target Aspergillus fumigatus gene is desired, it may be constructed generally according to the method of Baudin et al. et al. (1993, Nucleic Acids Research 21:3329-30), whereby a gene disruption event can be obtained following transformation of an Aspergillus fumigatus strain with the PCR-amplified gene disruption cassette and selection for drug resistant transformants or prototrophic isolates that have precisely replaced the wild type gene with the dominant selectable marker. Such mutant strains can be selected for growth in the presence of a drug, or the absence of a nutritional requirement such as but not limited to uracil. The disrupted gene is non-functional, and expression from this gene is nil. (See the Examples provided in Sections 6.3 and 6.4 infra).

In another embodiment of the present invention, essential genes of Aspergillus fumigatus are conditionally expressed by replacing the native promoter with a conditional-expression promoter, such as the tetracycline-regulatable promoter system that is developed initially for Saccharomyces cerevisiae but which can be modified for use in Aspergillus fumigatus (See Gari et al., 1997, Yeast 13:837-848; and Nagahashi et al., 1997, Mol. Gen. Genet. 255:372-375).

In this embodiment, conditional expression is achieved by first constructing a transactivation fusion protein comprising the E. coli TetR tetracycline repressor domain or DNA binding domain (amino acids 1-207) fused to the transcription activation domain of Saccharomyces cerevisiae GAL4 (amino acids 785-881) or HAP4 (amino acids 424-554). The nucleotide sequences encoding the transactivation fusion proteins of E. coli TetR (amino acids 1-207) plus Saccharomyces cerevisiae GAL4 (amino acids 785-881), and of E. coli TetR (amino acids 1-207) plus Saccharomyces cerevisiae HAP4 (amino acids 424-554), is encompassed by the present invention. Accordingly, the invention provides Aspergillus fumigatus cells that comprise a nucleotide sequence encoding a transactivation fusion protein expressible in the cells, wherein the transactivation fusion protein comprises a DNA binding domain and a transcription activation domain.

Expression of the transactivation fusion protein in Aspergillus fumigatus is achieved by providing, in one non-limiting example, an Aspergillus niger glucoamylase promoter, PGLA A. However, it will be appreciated that any regulatory regions, promoters and terminators, that are functional in Aspergillus fumigatus can be used to express the fusion protein. Thus, a nucleic acid molecule comprising a promoter functional in Aspergillus fumigatus, the coding region of a transactivation fusion protein, and a terminator functional in Aspergillus fumigatus, are encompassed by the present invention. Such a nucleic acid molecule can be a plasmid, a cosmid, a transposon, or a mobile genetic element. In a preferred embodiment, the TetR-Gal4 or TetR-Hap4 transactivators are stably integrated into a Aspergillus fumigatus strain, by using a suitable auxotrophic marker for selection of the desired integrant.

In this embodiment, the promoter replacement fragment comprises a nucleotide sequence encoding a heterologous promoter that comprises at least one copy of a nucleotide sequence recognized by the DNA binding domain of the transactivation fusion protein, whereby binding of the transactivation fusion protein to the heterologous promoter increases transcription from that promoter. The heterologous tetracycline promoters initially developed for Saccharomyces cerevisiae gene expression contains a variable number of copies of the tetracycline operator sequence, i.e., 2, 4, or 7 copies. The tetracycline promoter is subcloned adjacent to, e.g., a PYRG selectable marker, in the orientation favoring tetracycline promoter-dependent regulation when placed immediately upstream the open reading frame of the target gene. PCR amplification of the PYRG-Tet promoter cassette incorporates approximately 65 bp of flanking sequence homologous to the regulatory region to be replaced, that is, the region from around nucleotide positions −200 and −1 (relative to the start codon) of the target gene, thereby producing a conditional-expression promoter replacement fragment for transformation. When transformed into a Aspergillus fumigatus homologous recombination between the promoter replacement fragment and the upstream regulatory region of the target gene generates a strain in which the wild type regulatory region is replaced by the conditionally regulated tetracycline promoter. Transformants are selected as uracil prototrophs and verified by Southern blot and PCR analysis.

In this particular embodiment, the promoter is induced in the absence of tetracycline, and repressed by the presence of tetracycline. Analogs of tetracycline, including but not limited to chlortetracycline, demeclocycline, doxycycline, meclocycline, methocycline, minocycline hydrochloride, anhydrotetracycline, and oxytetracycline, can also be used to repress the expression of the conditional-expression mutant of the Aspergillus fumigatus target gene.

The present invention also encompasses the use of alternative variants of the tetracycline promoter system, based upon a mutated tetracycline repressor (tetR) molecule, designated tetR′, which is activated (i.e. binds to its cognate operator sequence) by binding of the antibiotic effector molecule to promote expression, and is repressed (i.e. does not bind to the operator sequence) in the absence of the antibiotic effectors, when the tetR′ is used instead of, or in addition to, the wild-type tetR. For example, analysis of the essentiality of a Aspergillus fumigatus gene could be performed using tetR′ instead of tetR in cases where repression is desired under conditions which lack the presence of tetracycline, such as shut off of a gene participating in drug transport (e.g. Aspergillus fumigatus homologs of the CaCDR1, CaPDR5, or CaMDR1 genes of Candida albicans). Also, the present methods could be adapted to incorporate both the tetR and tetR′ molecules in a dual activator/repressor system where tetR is fused to an activator domain and tetR′ is fused to a general repressor (e.g. the Aspergillus fumigatus homologs of the Candida albicans genes CaSsr6 and CaTup1) to enhance or further repress expression in the presence of the antibiotic effector molecules (Belli et al., 1998, Nucl Acid Res 26:942-947 which is incorporated herein by reference). These methods of providing conditional expression are also contemplated. By repeating this process whereby the wild type promoter for an Aspergillus fumigatus gene is replaced with a conditionally-expressed heterologous promoter, for a preferred subset of genes of Aspergillus fumigatus, or its entire genome, a collection or a complete set of conditional-expression mutant strains of Aspergillus fumigatus is obtained.

In another embodiment of the invention, the method is also applied to other haploid pathogenic fungi by modifying the target gene via recombination of the allele with a promoter replacement fragment comprising a nucleotide sequence encoding a heterologous promoter, such that the expression of the gene is conditionally regulated by the heterologous promoter. A preferred subset of genes comprises genes that share substantial nucleotide sequence homology with target genes of other organisms, e.g., Candida albicans and Saccharomyces cerevisiae. For example, this method of the invention may be applied to other haploid fungal pathogens including, but not limited to, animal fugal pathogens such as Aspergillus niger, Aspergillus favis, Candida glabrata, Cryptococcus neoformans, Coccidioides immitis, Exophalia dermatiditis, Fusarium oxysporum, Histoplasma capsulatum, Phneumocystis carinii, Trichosporon beigeli, Rhizopus arrhizus, Mucor rouxii, Rhizomucor pusillus, or Absidia corymbigera, or the plant fungal pathogens, such as Botrytis cinerea, Erysiphe graminis, Magnaporthe grisea, Puccinia recodita, Septoria triticii, Tilletia controversa, Ustilago maydis, or any species falling within the genera of any of the above species.

The means to achieve conditional expression are not restricted to the tetracycline promoter system and can be performed using other conditional promoters. Such conditional promoter may, for example, be regulated by a repressor which repress transcription from the promoter under particular condition or by a transactivator which increases transcription from the promoter, such as, when in the presence of an inducer. For example, as noted above, the Aspergillus niger glucoamylase promoter is not transcribed in Aspergillus fumigatus in the presence of xylose but has a high level of expression in cells grown maltose. Alternative promoters that could functionally replace the tetracycline promoter include but are not limited to other antibiotic-based regulatable promoter systems (e.g., pristinamycin-induced promoter or PIP) as well as the Aspergillus fumigatus homologs of Candida albicans conditionally-regulated promoters such as MET25, MAL2, PHO5, GAL1,10, STE2, or STE3.

Relatively few endogenous regulatable promoters have been identified or characterized in Aspergillus fumigatus although a number of endogenous and heterologous, inducible, promoters have been successfully employed for production of proteins in Aspergillus niger (Van den Hondel et al (1991) In: MORE GENE MANIPULATIONS IN FUNGI J. W. Bennett, L. Lasure, Eds. (Academic Press. Orlando, Fla.). One regulated promoter used for heterologous gene expression in Aspergillus is the glaA (glucoamylase) promoter. Transcription from the glaA promoter is induced by starch, maltose, or maltodextrin, and strongly repressed by xylose (Verdoes et al. (1994) Gene 146(2):159-65; Fowler et al. (1990) Curr Genet, 18(6):537-45). As measured using the E. coli uidA reporter in Aspergillus niger, expression of the glaA promoter is 100 fold induced in the presence of maltose as compared to xylose (Verdoes et al. (1994) Gene 146(2):159-65). This regulation appears to be at the level of transcription (Verdoes et al. (1994) Gene 146(2):159-65; Fowler et al. (1990) Curr Genet, 18(6):537-45).

An example of a heterologous promoter recognized in Aspergillus ndulans is the xylP promoter Penicillium chrysogenum, which retains conditional expression in Aspergillus ndulans (Zadra et al. (2000) Appl Environ Microbiol, 66(11): 4810-16). An advantage provided by the use of heterologous promoters during promoter-replacement strain constructions is that homology between the promoter-replacement cassette and non-target genomic sequences is minimized.

Promoters demonstrated to provide tightly-regulated, conditional expression of Aspergillus nidulans genes may be incorporated into the promoter-replacement cassettes of the present invention and used to demonstrate the essentiality of Aspergillus fumigatus genes. Therefore, in certain embodiments of the invention, promoters derived from the A. nidulans genes, alcA and aldA could be incorporated into promoter-replacement cassettes. The alcA and aldA genes encode alcohol and aldehyde dehydrogenases respectively and expression of both of these genes is tightly controlled by carbon source (Flipphi et al. (2001) J Biol Chem., 276(10): 6950-58). These genes are repressed in the presence of preferred carbon sources, such as glucose and lactose, and they are induced if either ethanol or 2-butanone is the sole carbon source. Other regulatable promoters useful in the promoter-replacement strategies of the present invention include, but are not limited to, promoters derived from the A. nidulans. facC and gabA genes (Stemple et al. 1998 J. Bacteriol. 180(23): 6242-6251; Espeso et al (2000), Molecular and Cellular Biology 20(10): 3355-3363. Expression of the facC gene,which encodes a camitine acetyltransferase, is induced by acetate and fatty acids but repressed by glucose (Stemple et al. 1998 J. Bacteriol. 180(23): 6242-6251). Expression of the gabA gene, which encodes a γ-aminobutyrate permease, is induced under acid conditions but repressed under alkaline growth conditions (Espeso et al (2000), Molecular and Cellular Biology 20(10): 3355-3363). In addition, regulatable promoters including, but not limited to those derived from the Neurospora crassa copper-metallothionein and ornithinedecarboxylase gene (ODC) may be employed in the promoter replacement methods used for establishing gene essentiality in Aspergillus fumigatus. The N. crassa copper-metallothionein is conditionally regulated according to level of copper in the medium (Schilling et al. 1992 Current Genetics 22(3):197-203; and ODC is repressed by spermidine (Williams et al. 1992, Molecular and Cellular Biology 12(1): 347-359; Hoyt et al. 2000, Molecular and Cellular Biology 20(80): 760-773). Another heterologous promoter useful in the present invention for promoter-replacement analysis of the essentiality of Aspergillus fumigatus genes is the xylP promoter from Penicillium chrysogenum. Expression of the xylP gene, which encodes a endoxylanase, is induced in the presence of xylan or xylose, but is strongly repressed by glucose (Zadra et al. (2000) Appl Environ Microbiol, 66(11): 4810-16). Accordingly, promoter-replacement cassettes can be constructed using copper-metallothionein, ODC, alcA, aldA, facC, gabA, or xylP promoter sequences identified in N. crassa, A. nidulans and P. chrysogenum respectively, or by using Aspergillus fumigatus promoters isolated genes homologous thereto.

Essentiality of the gene being tested may be determined by comparing growth of the promoter replacement strain under the specific conditions that induce or repress the chosen condtional promoter.

In other embodiments of the present invention, an endogenous, regulatable Aspergillus fumigatus, promoter may be used to determine gene essentiality in Aspergillus fumigatus. For example, promoters regulating the expression of niiA, niaD or crnA may be used for promoter replacement in A. fumigatus. Each of these genes is part of the nitrate-assimilation gene cluster in A. fumigatus. The nitrate-assimilation cluster is conserved in A. nidulans and is tightly regulated according to nitrogen sources available (Cove 1979 Biol Rev Camb Philos Soc 54(3): 291-327; Kinghorn JR. GENETICAL, BIOCHEMICAL, AND STRUCTURAL ORGANIZATION OF ASPERGILLUS NIDULANS cRNA-NIIA-NIAD GENE CLUSTER. In: Wray J L, Kinghorn J R (eds) Molecular and genetic aspects of nitrate assimilation in Aspergillus nidulans. Oxford University Press, Oxford, pp 69-87 (1989); Johnstone et al 1990 Gene 90(2): 181-92). This catabolic pathway, as in A. nidulans, contains niiA, niaD, and crnA genes that encode a nitrite reductase, nitrate reductase, and nitrate transporter respectively (Amaar et al. 1998 Curr Genet 33(3): 206-15). These three genes are coordinately induced under conditions where primary nitrogen sources, which include ammonia, glutamine or glutamate are absent, but secondary nitrogen sources such as nitrate, purines, amino acids and/or amides are available for growth. Under such inducing conditions, cmA induction facilitates nitrogen uptake from the environment. Expression of nitrate reductase encoded by niaD then converts nitrate to nitrite, which is then converted to ammonium by the nitrite reductase encoded by niiA. Northern analysis indicates that each of the three nitrate-assimilation genes are induced in the presence of nitrate and dramatically repressed by ammonium (Amaar et al. 1998 Curr Genet 33(3): 206-15). Therefore essentiality of a gene tested may be determined by comparing growth of the promoter replacement strain on medium containing nitrate (inducing condition) versus growth on a medium in which ammonium is the sole nitrogen source (repressing conditions).

In still further embodiments, the present invention is directed toward the use of of additional regulatable, endogenous promoters of Aspergillus fumigauts for the construction of promoter replacement cassettes for the conditional expression of genes in Aspergillus fumigatus for determining gene essentiality, including, but not limited to ADH1, GAL1-10, MAL2, MET3, MET25, PCK1, and PHO5 (www.stanford.edu/Saccharomyces). In each of these examples, regulation of these promoters is demonstrated to be under tight control in S. cerevisisae. Orthologues of each of these genes have been identified in Aspergillus fumigatus. Consequently, promoter sequences of each gene identified in the Aspergillus fumigatus orthologs may be used to construct promoter replacement cassettes by standard molecular biology methods. Orthologues of each of the above-listed, regulated S. cerevisiae, genes that are found in the species closely related to A. fumigatus, including but not restricted to A. niger, A. nidulans, and A. parasiticus, may also exhibit conserved regulation in Aspergillus fumigatus and would, therefore, also be suitable for promoter replacement-based essential gene determination in Aspergillus fumigatus. Similarly, promoters including but not restricted to the A. niger glaA, and the A. nidulans alcA and aldA promoters that are identified in other Aspergilli, including A. fumigatus, could be used in the methods of the present invention. For example, a gene family comprising three homologues of the A. niger glaA gene has been identified in Aspergillus fumigatus. Promoters regulating each of these AfglaA genes may be used for promoter replacement methods for determination of gene essentiality. Transformation and precise promoter replacement using the replacement cassettes containing the regulatable promoter may then be carried out in A. fumigatus to establish conditional expression of any gene whose growth phenotype is sought. Gene essentiality is determined by comparing growth under conditions that specifically induce or repress the conditionally-expressed promoter used in constructing the A. fumigatus promoter replacement strain.

Specific applications of the present method, used to construct modified alleles of the target genes Aspergillus fumigatus HIS3 and Aspergillus fumigatus genes are provided in Sections 6.2 and 6.10, infra.

In other embodiments of the invention, conditional expression is achieved by means other than the reliance of conditional promoters. For example, conditional expression could be achieved by the replacement of the wild type allele with temperature sensitive alleles derived in vitro, and their phenotype would then be analyzed at the nonpermissive temperature. In a related approach, insertion of a ubiquitination signal into the a gene to destabilize the encoded gene product during activation conditions can be adopted to examine phenotypic effects resulting from gene inactivation. Collectively, these examples demonstrate the manner in which Aspergillus fumigatus genes can be disrupted and conditionally regulated using the methods disclosed herein.

In an alternative embodiment of the present invention, a constitutive promoter regulated by an excisable transactivator can be used. The promoter is placed upstream to a target gene to repress expression to the basal level characteristic of the promoter. For example, in a fungal cell, a heterologous promoter containing lexA operator elements may be used in combination with a fusion protein composed of the lexA DNA binding domain and any transcriptional activator domain (e.g. GAL4, HAP4, VP16) to provide constitutive expression of a target gene. Counterselection mediated by 5-FOA can be used to select those cells which have excised the gene encoding the fusion protein. This procedure enables an examination of the phenotype associated with repression of the target gene to the basal level of expression provided by the lexA heterologous promoter in the absence of a functional transcription activator. The conditional-expression Aspergillus fumigatus mutant strains generated by this approach can be used for drug target validation as described in detail in the sections below. In this system, the low basal level expression associated with the heterologous promoter is critical. Thus, it is preferable that the basal level of expression of the promoter is low to make this alternative shut-off system more useful for target validation.

Alternatively, conditional expression of a target gene can be achieved without the use of a transactivator containing a DNA binding, transcriptional activator domain. A cassette could be assembled to contain a heterologous constitutive promoter downstream of, for example, the PYRG selectable marker, which is flanked with a direct repeat containing homologous sequences to the 5′ portion of the target gene. Additional homologous sequences upstream of the target, when added to this cassette would facilitate homologous recombination and replacement of the native promoter withe above-described heterologous promoter cassette immediately upstream of the start codon of the target gene or open reading frame. Conditional expression is achieved by selecting uracil prototrophic strains, by using the appropriate media, which have integrated the heterologous constitutive promoter and PYRG marker and examining the growth of the resulting strain versus a wild type strain grown under identical conditions. Subsequent selection of 5-FOA resistant strains provides isolates which have lost the PYRG marker and heterologous, constitutive promoter, allowing a comparison between the growth of the resulting strain lacking a promoter for expression of the target gene and the growth of a wild type strain cultured under identical conditions.

### 5.3 Identification and Validation of Essential Genes

### 5.3.1 Target Genes

Target discovery has traditionally been a costly, time-consuming process, in which newly-identified genes and gene products have been individually analyzed as potentially-suitable drug targets. DNA sequence analysis of entire genomes has markedly accelerated the gene discovery process. Consequently, new methods and tools are required to analyze this information, first to identify all of the genes of the organism, and then, to discern which genes encode products that will be suitable targets for the discovery of effective, non-toxic drugs. Gene discovery through sequence analysis alone does not validate either known or novel genes as drug targets. Elucidation of the function of a gene from the underlying and a determination of whether or not that gene is essential still present substantial obstacles to the identification of appropriate drug targets.

As noted above, Aspergillus fumigatus is a major fungal pathogen of humans. An absence of identified specific, sensitive, and unique drug targets in this organism has hampered the development of effective, non-toxic compounds for clinical use. The recent completion of an extensive DNA sequence analysis of the Aspergillus fumigatus genome is rejuvenating efforts to identify new antifungal drug targets. Nevertheless, two primary obstacles to the exploitation of this information for the development of useful drug targets remain: the paucity of suitable markers for genetic manipulations in Aspergillus fumigatus and the inherent difficulty in establishing whether a specific gene encodes an essential product.

Several strategies are available to produce single or multiple mutants of Aspergillus fumigatus. The classic method involves the disruption of the gene of interest by the insertion of an antibiotic resistance gene. Two genes, one conferring resistance to hygromycin and one conferring resistance to phleomycin, have been commonly used (Mattern et al., 1988, Fungal Genet. Newsl. 35:25; Punt et al., 1987, Gene 56:117-124). They are placed under the control of either the GPD promoter or the TRP C terminator of A. nidulans or the promoter and terminator of the gene subjected to disruption. Disruption is usually made in a nitrate reductase-deficient genetic background to obviate external contamination. However, these systems can lead to only two successive mutations. To compensate for this disadvantage, a PYRG blaster has been developed (d'Enfert, 1996, Curr. Genet. 30:76-82). This system is very similar to the URA blaster previously developed in Saccharomyces cerevisiae and Candida albicans. The system consists of the Aspergillus niger PYRG gene flanked by a direct repeat that encodes the neomycin phosphotransferase of Tn5. This cassette may also include flanking sequences corresponding to a target gene to be replace or insertionally inactivated. The PYRG cassette is inserted by gene replacement or ectopic insertion into the genome following transformation of a uridine/uracil-autotrophic PYRG strain, creating a mutant Aspergillus fumigatus as a result of the insertion or replacement. Excision of the cassette, including the Aspergillus niger PYRG gene, is selected in the presence of 5-fluoroorotic acid, provides a A. fumigatus uridine/uracil auxotroph which retains the mutant phenotype since one copy of the direct repeat remains at the site of insertion of the PYRG blaster cassette. Selection for uridine/uracil prototrophy can be used again to disrupt another gene. Transformation can be performed with protoplasts or by electroporation (Brown et. al, 1998 Mol. Gen. Genet. 259:327-335; Werdner et al., 1998, Curr. Genet. 33:378-385). Where the PYRG blaster cassette carries flanking sequences corresponding to the gene to be replaced, precise replacement of that gene by homologous recombination can be obtained. Putative transformants are selected as uracil prototrophs and their identity and chromosomal structure confirmed by Southern blot and PCR analyses.

However, mutants in which an essential gene has been deleted or insertionally inactivated (collectively referred to herein as “knock-out mutants) will not be viable. Accordingly, the PYRG blaster method will not provide an unequivocal result, establishing the essential nature of the target gene since alternative explanations, including poor growth of a viable mutant strain, may be equally likely for the negative results obtained. Moreover, in those instances in which a target gene is duplicated or there exists a paralog encoding a gene product having the same biochemical function as the target gene, the PYRG blaster method would not provide an unambiguous result. Accordingly, such an approach is too labor-intensive to be suitable for genome-wide analyses.

Finally, the PYRG blaster method precludes direct demonstration of gene essentiality. Therefore, one is unable to critically evaluate the terminal phenotype characteristic of essential target genes. Consequently, establishing whether inactivation of a validated drug target gene results in cell death (i.e., a cidal terminal phenotype) versus growth inhibition (i.e., a static terminal phenotype) is not possible with current approaches, despite the value such information would provide in prioritizing drug targets for suitability in drug development.

Clearly, since current gene disruption methods are labor intensive and largely refractile to a high throughput strategy for target validation, there is a need for effective methods and tools for unambiguous, rapid, and accurate identification of essential genes in Aspergillus fumigatus. The present invention overcomes these limitations in current drug discovery approaches by providing Aspergillus fumigatus genes, the nucleotide sequence of those genes, the identification of the encoded gene products, thereby enabling high throughput strategies that provide rapid identification, validation, and prioritization of drug targets, and consequently, accelerate drug screening.

### 5.3.2 Validation of Genes Encoding Drug Targets

Target gene validation refers to the process by which a gene product is identified as suitable for use in screening methods or assays in order to find modulators of the function or structure of that gene product. Criteria used for validation of a gene product as a target for drug screening, however, may be varied depending on the desired mode of action that the compounds sought will have, as well as the host to be protected.

In one aspect of the present invention, conditional-expression Aspergillus fumigatus mutant strains having modified essential genes can be used directly for drug screening. In another aspect, the initial set of essential genes is further characterized using, for example, nucleotide sequence comparisons, to identify a subset of essential genes which include only those genes specific to fungi—that is, a subset of genes encoding essential genes products which do not have homologs in a host of the pathogen, such as humans. Modulators, and preferably inhibitors, of such a subset of genes in a fungal pathogen of humans would be predicted to be much less likely to have toxic side effects when used to treat humans.

Similarly, other subsets of the larger essential gene set could be defined to include only those conditional-expression Aspergillus fumigatus mutant strains carrying modified genes that do not have a homologous sequence in one or more host (e.g., mammalian) species to allow the detection of compounds expected to be used in veterinary applications. In addition, using other homology criteria, a subset of conditional-expression Aspergillus fumigatus mutant strains is identified and used for the detection of anti-fungal compounds active against agricultural pathogens, inhibiting targets that do not have homologs in the crop to be protected.

Current Aspergillus fumigauts gene disruption strategies identify nonessential genes and permit the inference that other genes are essential, based on a failure to generate a null mutant. The null phenotype of a drug target predicts the absolute efficaciousness of the “perfect” drug acting on this target. For example, the difference between a cidal (cell death) versus static (inhibitory growth) null terminal phenotype for a particular drug target.

For example, in Candida albicans, gene disruption of CaERG11, the drug target of fluconazole, is presumed to be essential based on the failure to construct a homozygous CaERG11 deletion strain using the URA blaster method. However, direct evaluation of its null phenotype being cidal or static could not be performed in the pathogen, and only after the discovery of fluconazole was it possible to biochemically determine both the drug, and presumably the drug target to be static rather than as cidal. Despite the success fluconazole enjoys in the marketplace, its fungistatic mode of action contributes to its primary limitation, i.e., drug resistance after prolonged treatment. Therefore, for the first time, the ability to identify and evaluate cidal null phenotypes for validated drug targets within the pathogen as provided by the invention, now enables directed strategies to identifying antifungal drugs that specifically display a fungicidal mode of action.

Using a single conditional-expression Aspergillus fumigatus mutant strain or a desired collection of conditional-expression Aspergillus fumigatus mutant strains comprising essential genes, one or more target genes can be directly evaluated as displaying either a cidal or static null phenotype. This is determined by first incubating conditional-expression Aspergillus fumigatus mutant strains under repressing conditions for the conditional expression of the modified gene for varying lengths of time in liquid culture, and measuring the percentage of viable cells following plating a defined number of cells onto growth conditions which relieve repression. The percentage of viable cells that remain after return to non-repressing conditions reflects either a cidal (low percent survival) or static (high percent survival) phenotype. Alternatively, vital dyes such as methylene blue or propidium iodide could be used to quantify percent viability of cells for a particular strain under repressing versus inducing conditions. As known fungicidal drug targets are included in the conditional-expression Aspergillus fumigatus mutant strains strain collection, direct comparisons can be made between this standard fungicidal drug target and novel targets comprising the drug target set. In this way each member of the target set can be immediately ranked and prioritized against an industry standard cidal drug target to select appropriate drug targets and screening assays for the identification of the most rapid-acting cidal compounds. Accordingly, in preferred embodiments, mutations of the essential genes of the invention confer to the cells a rapid cidal phenotype.

In one embodiment of the invention, as described infra in Section 6.2, the promoter of the target gene is replaced with the Aspergillus niger glucoamylase promoter, Pgla A. The Aspergillus niger Pgla A promoter is induced in the presence of maltose, repressed in the presence of xylose, and exhibits intermediate levels of expression in cells grown in the presence of glucose or mixtures of maltose and xylose. Therefore, by adjusting the level of maltose and/or xylose in the growth medium, the amount of transcription from the target gene is titrated. The nucleotide sequence of the glucoamylase promoter of Aspergillus niger, PglaA, has been characterized (Verdoes et al., Gene 145:179-187 (1994), which is incorporated by reference in its entirety), and the nucleotide sequence of PglaA may be obtained from publically available databases, such as EMBL Data Library Accession No. Z30918.

### 5.4 SCREENING ASSAYS

The following assays are designed to identify compounds that bind to target gene products, bind to other cellular proteins that interact with the target gene product, and to compounds that interfere with the interaction of the target gene product with other cellular proteins. Compounds identified via such methods can include compounds which modulate the activity of a polypeptide encoded by a target gene of the invention (that is, increase or decrease its activity, relative to activity observed in the absence of the compound). Alternatively, compounds identified via such methods can include compounds which modulate the expression of the polynucleotide (that is, increase or decrease expression relative to expression levels observed in the absence of the compound), or increase or decrease the stability of the expressed product encoded by that polynucleotide. Compounds, such as compounds identified via the methods of the invention, can be tested using standard assays well known to those of skill in the art for their ability to modulate activity/expression.

Accordingly, the present invention provides a method for identifying an antimycotic compound comprising screening a plurality of compounds to identify a compound that modulates the activity or level of a gene product, said gene product being encoded by a nucleotide sequence selected from the group consisting of SEQ ID NOs: 2001-2594 and 7001-7603, as well as the gene product encoded by genomic SEQ ID NOs: 1-594, 5001-5603,1001-1594, 6001-6603, as expressed by Aspergillus fumigatus, or a nucleotide sequence that is naturally occurring in Saccharomyces cerevisiae or Candida albicans and that is the ortholog of a gene having a nucleotide sequence selected from the group consisting of SEQ ID NOs: 2001-2594 and 7001-7603.

### 5.4.1 In Vitro Screening Assays

In vitro systems are designed to identify compounds capable of binding the target gene products of the invention. Compounds identified in this manner are useful, for example, in modulating the activity of wild type and/or mutant target gene products, are useful in elucidating the biological function of target gene products, are utilized in screens for identifying other compounds that disrupt normal target gene product interactions, or are useful themselves for the disruption of such interactions.

The principle of the assays used to identify compounds that bind to the target gene product involves preparing a reaction mixture comprising the target gene product and the test compound under conditions and for a time sufficient to allow the two components to interact and bind, thus forming a complex which is removed and/or detected within the reaction mixture. These assays are conducted in a variety of ways. For example, one method involves anchoring target gene product or the test substance onto a solid phase and detecting target gene product/test compound complexes anchored, via the intermolecular binding reaction, to the solid phase at the end of the reaction. In one embodiment of such a method, the target gene product is anchored onto a solid surface, and the test compound, which is not anchored, is labeled, either directly or indirectly.

In practice, microtiter plates are conveniently utilized as the solid phase. The anchored component is immobilized by non-covalent or covalent attachments. Non-covalent attachment can be accomplished by simply coating the solid surface with a solution of the protein and drying the coated surface. Alternatively, an immobilized antibody, preferably a monoclonal antibody, specific for the protein to be immobilized is used to anchor the protein to the solid surface. The surfaces are prepared in advance and stored.

In order to conduct the assay, the nonimmobilized component is added to the coated surface containing the anchored component. After the reaction is complete, unreacted components are removed (e. g., by washing) under conditions such that any complexes formed will remain immobilized on the solid surface. The detection of complexes anchored on the solid surface is accomplished in a number of ways. Where the previously nonimmobilized component is pre-labeled, the detection of label immobilized on the surface indicates that complexes were formed. Where the previously nonimmobilized component is not pre-labeled, an indirect label is used to detect complexes anchored on the surface; e.g., using a labeled antibody specific for the previously nonimmobilized component (the antibody, in turn, is directly labeled or indirectly labeled with a labeled anti-Ig antibody).

Alternatively, a reaction is conducted in a liquid phase, the reaction products are separated from unreacted components, and complexes are detected; e.g., using an immobilized antibody specific for the target gene product or for the test compound, to anchor complexes formed in solution, and a second labeled antibody, specific for the other component of the complex to allow detection of anchored complexes.

### 5.4.1.1 Assays For Proteins That Interact With A Target Gene Product

Any method suitable for detecting protein-protein interactions can be employed for identifying novel target protein-cellular or extracellular protein interactions.

The target gene products of the invention interact, in vivo, with one or more cellular or extracellular macromolecules, such as proteins. Such macromolecules include, but are not limited to, nucleic acid molecules and proteins identified via methods such as those described above. For purposes of this discussion, such cellular and extracellular macromolecules are referred to herein as “binding partners.” Compounds that disrupt such interactions can be useful in regulating the activity of the target gene protein, especially mutant target gene proteins. Such compounds include, but are not limited to molecules such as antibodies, peptides, and the like, as described.

The basic principle of the assay systems used to identify compounds that interfere with the interaction between the target gene product and its cellular or extracellular binding partner or partners involves preparing a reaction mixture containing the target gene product and the binding partner under conditions and for a time sufficient to allow the two to interact and bind, thus forming a complex. In order to test a compound for inhibitory activity, the reaction mixture is prepared in the presence and absence of the test compound.

The test compound is initially included in the reaction mixture, or added at a time subsequent to the addition of target gene product and its cellular or extracellular binding partner. Control reaction mixtures are incubated without the test compound. The formation of complexes between the target gene protein and the cellular or extracellular binding partner is then detected. The formation of a complex in the control reaction, but not in the reaction mixture containing the test compound, indicates that the compound interferes with the interaction of the target gene protein and the interactive binding partner. Additionally, complex formation within reaction mixtures containing the test compound and normal target gene protein can also be compared to complex formation within reaction mixtures containing the test compound and a mutant target gene protein. This comparison can be important in those cases wherein it is desirable to identify compounds that disrupt intermolecular interactions involving mutant but not normal target gene proteins.

The assay for compounds that interfere with the interaction of the target gene products and binding partners is conducted in either a heterogeneous or a homogeneous format. Heterogeneous assays involve anchoring either the target gene product or the binding partner onto a solid phase and detecting complexes anchored on the solid phase at the end of the reaction. In homogeneous assays, the entire reaction is carried out in a liquid phase. In either approach, the order of addition of reactants is varied to obtain different information about the compounds being tested. For example, test compounds that interfere with the interaction between the target gene products and the binding partners, e.g., by competition, are identified by conducting the reaction in the presence of the test substance; i.e., by adding the test substance to the reaction mixture prior to or simultaneously with the target gene protein and an interacting cellular or extracellular binding partner. Alternatively, test compounds that disrupt preformed complexes, e.g. compounds with higher binding constants that displace one of the components from the complex, are tested by adding the test compound to the reaction mixture after complexes have been formed. The various formats are described briefly below.

In a heterogeneous assay system, either the target gene protein or the interactive cellular or extracellular binding partner, is anchored onto a solid surface, while the non-anchored species is labeled, either directly or indirectly. In practice, microtiter plates are conveniently utilized. The anchored species is immobilized either by non-covalent or covalent attachment. Non-covalent attachment is accomplished simply by coating the solid surface with a solution of the target gene product or binding partner and drying the coated surface. Alternatively, an immobilized antibody specific for the species to be anchored is used to anchor the species to the solid surface. The surfaces can be prepared in advance and stored.

In order to conduct the assay, the partner of the immobilized species is exposed to the coated surface with or without the test compound. After the reaction is complete, unreacted components are removed (e.g., by washing) and any complexes formed will remain immobilized on the solid surface. The detection of complexes anchored on the solid surface is accomplished in a number of ways. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, is directly labeled or indirectly labeled with a labeled anti-Ig antibody). Depending upon the order of addition of reaction components, test compounds which inhibit complex formation or which disrupt preformed complexes are detected.

Alternatively, the reaction is conducted in a liquid phase in the presence or absence of the test compound, the reaction products separated from unreacted components, and complexes detected; e.g., using an immobilized antibody specific for one of the binding components to anchor any complexes formed in solution, and a second, labeled antibody specific for the other partner to detect anchored complexes. Again, depending upon the order of addition of reactants to the liquid phase, test compounds which inhibit complex or which disrupt preformed complexes are identified.

In an alternate embodiment of the invention, a homogeneous assay can be used. In this approach, a preformed complex of the target gene protein and the interacting cellular or extracellular binding partner is prepared in which either the target gene product or its binding partner is labeled, but the signal generated by the label is quenched due to complex formation (see, e.g., U.S. Pat. No. 4,109,496 by Rubenstein which utilizes this approach for immunoassays). The addition of a test substance that competes with and displaces one of the species from the preformed complex results in the generation of a signal above background. In this way, test substances which disrupt target gene protein/cellular or extracellular binding partner interaction are identified.

In a particular embodiment, the target gene product is prepared for immobilization using recombinant DNA techniques described above. For example, the target gene coding region is fused to a glutathione-S-transferase (GST) gene using a fusion vector, such as pGEX-5X-1, in such a manner that its binding activity is maintained in the resulting fusion protein. The interactive cellular or extracellular binding partner is purified and used to raise a monoclonal antibody, using methods routinely practiced in the art and as described above. This antibody is labeled with the radioactive isotope ¹²⁵I, for example, by methods routinely practiced in the art. In a heterogeneous assay, e.g., the GST-target gene fusion protein is anchored to glutathione-agarose beads. The interactive cellular or extracellular binding partner is then added in the presence or absence of the test compound in a manner that allows interaction and binding to occur. At the end of the reaction period, unbound material can be washed away, and the labeled monoclonal antibody is added to the system and allowed to bind to the complexed components. The interaction between the target gene protein and the interactive cellular or extracellular binding partner is detected by measuring the amount of radioactivity that remains associated with the glutathione-agarose beads. A successful inhibition of the interaction by the test compound results in a decrease in measured radioactivity.

Alternatively, the GST-target gene fusion protein and the interactive cellular or extracellular binding partner are mixed together in liquid in the absence of the solid glutathione-agarose beads. The test compound is added either during or after the species are allowed to interact. This mixture is added to the glutathione-agarose beads and unbound material is washed away. Again the extent of inhibition of the target gene product/binding partner interaction is detected by adding the labeled antibody and measuring the radioactivity associated with the beads.

In another embodiment of the invention, these same techniques are employed using peptide fragments that correspond to the binding domains of the target gene product and/or the interactive cellular or extracellular binding partner (in cases where the binding partner is a protein), in place of one or both of the full length proteins. Any number of methods routinely practiced in the art are used to identify and isolate the binding sites. These methods include, but are not limited to, mutagenesis of the gene encoding one of the proteins and screening for disruption of binding in a co-immunoprecipitation assay. Compensating mutations in the gene encoding the second species in the complex are then selected. Sequence analysis of the genes encoding the respective proteins reveals the mutations that correspond to the region of the protein involved in interactive binding. Alternatively, one protein is anchored to a solid surface using methods described above, and allowed to interact with and bind to its labeled binding partner, which has been treated with a proteolytic enzyme, such as trypsin. After washing, a short, labeled peptide comprising the binding domain remains associated with the solid material, and can be isolated and identified by amino acid sequencing. Also, once the gene coding for the cellular or extracellular binding partner is obtained, short gene segments are engineered to express peptide fragments of the protein, which are tested for binding activity and purified or synthesized.

For example, and not by way of limitation, a target gene product is anchored to a solid material as described, above, by making a GST-target gene fusion protein and allowing it to bind to glutathione agarose beads. The interactive cellular or extracellular binding partner is labeled with a radioactive isotope, such as ³⁵S, and cleaved with a proteolytic enzyme such as trypsin. Cleavage products are added to the anchored GST-target gene fusion protein and allowed to bind. After washing away unbound peptides, labeled bound material, representing the cellular or extracellular binding partner binding domain, is eluted, purified, and analyzed for amino acid sequence by well known methods. Peptides so identified are produced synthetically or fused to appropriate facilitative proteins using well known recombinant DNA technology.

### 5.4.1.2 Screening a Combinatorial Chemical library

In one embodiment of the present invention, the proteins encoded by the fungal genes identified using the methods of the present invention are isolated and expressed. These recombinant proteins are then used as targets in assays to screen libraries of compounds for potential drug candidates. The generation of chemical libraries is well known in the art. For example, combinatorial chemistry is used to generate a library of compounds to be screened in the assays described herein. A combinatorial chemical library is a collection of diverse chemical compounds generated by either chemical synthesis or biological synthesis by combining a number of chemical “building block” reagents. For example, a linear combinatorial chemical library such as a polypeptide library is formed by combining amino acids in every possible combination to yield peptides of a given length. Millions of chemical compounds theoretically can be synthesized through such combinatorial mixings of chemical building blocks. For example, one commentator observed that the systematic, combinatorial mixing of 100 interchangeable chemical building blocks results in the theoretical synthesis of 100 million tetrameric compounds or 10 billion pentameric compounds. (Gallop et al., “Applications of Combinatorial Technologies to Drug Discovery, Background and Peptide Combinatorial Libraries,” Journal of Medicinal Chemistry, Vol. 37, No. 9, 1233-1250 (1994). Other chemical libraries known to those in the art may also be used, including natural product libraries.

Once generated, combinatorial libraries are screened for compounds that possess desirable biological properties. For example, compounds which may be useful as drugs or to develop drugs would likely have the ability to bind to the target protein identified, expressed and purified as discussed above. Further, if the identified target protein is an enzyme, candidate compounds would likely interfere with the enzymatic properties of the target protein. For example, the enzymatic function of a target protein may be to serve as a protease, nuclease, phosphatase, dehydrogenase, transporter protein, transcriptional enzyme, replication component, and any other type of enzyme known or unknown. Thus, the present invention contemplates using the protein products described above to screen combinatorial chemical libraries.

In some embodiments of the present invention, the biochemical activity of the protein, as well as the chemical structure of a substrate on which the protein acts is known. In other embodiments of the present invention, the biochemical activity of the target protein is unknown and the target protein has no known substrates.

In some embodiments of the present invention, libraries of compounds are screened to identify compounds that function as inhibitors of the target gene product. First, a library of small molecules is generated using methods of combinatorial library formation well known in the art. U.S. Pat. Nos. 5,463,564 and 5,574,656, to Agrafiotis, et al., entitled “System and Method of Automatically Generating Chemical Compounds with Desired Properties,” the disclosures of which are incorporated herein by reference in their entireties, are two such teachings. Then the library compounds are screened to identify those compounds that possess desired structural and functional properties. U.S. Pat. No. 5,684,711, the disclosure of which is incorporated herein by reference in its entirety, also discusses a method for screening libraries.

To illustrate the screening process, the target gene product, an enzyme, and chemical compounds of the library are combined and permitted to interact with one another. A labeled substrate is added to the incubation. The label on the substrate is such that a detectable signal is emitted from metabolized substrate molecules. The emission of this signal permits one to measure the effect of the combinatorial library compounds on the enzymatic activity of target enzymes by comparing it to the signal emitted in the absence of combinatorial library compounds. The characteristics of each library compound are encoded so that compounds demonstrating activity against the enzyme can be analyzed and features common to the various compounds identified can be isolated and combined into future iterations of libraries.

Once a library of compounds is screened, subsequent libraries are generated using those chemical building blocks that possess the features shown in the first round of screen to have activity against the target enzyme. Using this method, subsequent iterations of candidate compounds will possess more and more of those structural and functional features required to inhibit the function of the target enzyme, until a group of enzyme inhibitors with high specificity for the enzyme can be found. These compounds can then be further tested for their safety and efficacy as antibiotics for use in mammals.

It will be readily appreciated that this particular screening methodology is exemplary only. Other methods are well known to those skilled in the art. For example, a wide variety of screening techniques are known for a large number of naturally-occurring targets when the biochemical function of the target protein is known. For example, some techniques involve the generation and use of small peptides to probe and analyze target proteins both biochemically and genetically in order to identify and develop drug leads. Such techniques include the methods described in PCT publications No. WO9935494, WO9819162, WO9954728, the disclosures of which are incorporated herein by reference in their entireties.

Similar methods may be used to identify compounds which inhibit the activity of proteins from organisms other than Candida albicans which are homologous to the Candida albicans target proteins described herein. For example, the proteins may be from animal fugal pathogens such as Aspergillus fumigatus, Aspergillus niger, Aspergillus flavis, Candida tropicalis, Candida parapsilopsis, Candida krusei, Cryptococcus neoformans, Coccidioides immitis, Exophalia dermatiditis, Fusarium oxysporum, Histoplasma capsulatum, Phneumocystis carinii, Trichosporon beigelii, Rhizopus arrhizus, Mucor rouxii, Rhizomucorpusillus, or Absidia corymbigera, or the plant fungal pathogens, such as Botrytis cinerea, Erysiphe graminis, Magnaporthe grisea, Puccinia recodita, Septoria triticii, Tilletia controversa, Ustilago maydis, or any species falling within the genera of any of the above species. In some embodiments, the proteins are from an organism other than Saccharomyces cerevisiae.

### 5.4.1.3 In vitro Enzyme Assays

Aspergillus fumigatus strains are used to develop in vitro assays for biochemical activities shown to be essential to cell viability, e.g., by homology to known essential genes of Candida albicans. A number of such essential genes identified by sequence analysis of the Aspergillus fumigatus genome display statistically significant similarity to biochemically characterized gene products from other organisms. For example, based on amino acid sequence similarity, a number of essential and fungal specific genes listed in Table 1 are predicted to possess known biochemical activities.

Therefore, a number of well characterized standard in vitro biochemical assays (e.g., DNA binding, RNA processing, GTP binding and hydrolysis, and phosphorylation) are readily adapted for these validated drug targets. Alternatively, novel assays are developed using biochemical information pertaining to validated drug targets within the Aspergillus fumigatus sequenced gene collection. Any assays known in the art for enzymes with similar biochemical activities (e.g., mechanism of action, class of substrate) are adapted for screening for inhibitors of the enzymes encoded by these essential Aspergillus fumigatus genes.

The present invention also provides cell extracts useful in establishing in vitro assays for suitable biochemical targets. For example, in an embodiment of the present invention, conditional-expression Aspergillus fumigatus mutant strains are grown either under constitutive expression conditions or transcription repression conditions to either overproduce or deplete a particular gene product. Cellular extracts resulting from strains incubated under these two conditions are compared with extracts prepared from identically-grown wild type strains. These extracts are then used for the rapid evaluation of targets using existing in vitro assays or new assays directed toward novel gene products, without having to purify the gene product. Such a whole cell extract approach to in vitro assay development is typically necessary for targets involved in cell wall biosynthetic pathways (e. g. (1,3)-β-glucan synthesis or chitin synthesis) which involve multiple gene products that transit the secretory pathway before receiving essential post-translational modifications required for their functional activity. Conditional-expression Aspergillus fumigatus mutant strains for conditional expression of target genes involved in these, or other cell wall pathways (e. g. (1,6)-β-glucan synthesis) enable in vitro assays to be performed directly in Aspergillus fumigatus.

### 5.4.2 Cell-based Screening Assays

Current cell-based assays used to identify or to characterize compounds for drug discovery and development frequently depend on detecting the ability of a test compound to modulate the activity of a target molecule located within a cell or located on the surface of a cell. Most often such target molecules are proteins such as enzymes, receptors and the like. However, target molecules also include other molecules such as DNAs, lipids, carbohydrates and RNAs including messenger RNAs, ribosomal RNAs, tRNAs and the like. A number of highly sensitive cell-based assay methods are available to those of skill in the art to detect binding and interaction of test compounds with specific target molecules. However, these methods are generally not highly effective when the test compound binds to or otherwise interacts with its target molecule with moderate or low affinity. In addition, the target molecule may not be readily accessible to a test compound in solution, such as when the target molecule is located inside the cell or within a cellular compartment such as the periplasm of a bacterial cell. Thus, current cell-based assay methods are limited in that they are not effective in identifying or characterizing compounds that interact with their targets with moderate to low affinity or compounds that interact with targets that are not readily accessible.

The cell-based assay methods of the present invention have substantial advantages over current cell-based assays. These advantages derive from the use of sensitized cells in which the level or activity of at least one gene product required for fungal survival, growth, proliferation, virulence, or pathogenicity (the target molecule) has been specifically reduced to the point where the presence or absence of its function becomes a rate-determining step for fungal survival, growth, proliferation, virulence, or pathogenicity. Such sensitized cells become much more sensitive to compounds that are active against the affected target molecule. For example, sensitized cells are obtained by growing a conditional-expression Aspergillus fumigatus mutant strain in the presence of a concentration of inducer or repressor which provides a level of a gene product required for fungal growth, survival, proliferation, virulence, or pathogenicity such that the presence or absence of its function becomes a rate-determining step for fungal growth, survival, proliferation, virulence, or pathogenicity. Thus, cell-based assays of the present invention are capable of detecting compounds exhibiting low or moderate potency against the target molecule of interest because such compounds are substantially more potent on sensitized cells than on non-sensitized cells. The effect may be such that a test compound may be two to several times more potent, at least 10 times more potent, at least 20 times more potent, at least 50 times more potent, at least 100 times more potent, at least 1000 times more potent, or even more than 1000 times more potent when tested on the sensitized cells as compared to the non-sensitized cells.

Due in part to the increased appearance of antibiotic resistance in pathogenic microorganisms and to the significant side-effects associated with some currently used antibiotics, novel antibiotics acting at new targets are highly sought after in the art. Yet, another limitation in the current art related to cell-based assays is the problem of repeatedly identifying hits against the same kinds of target molecules in the same limited set of biological pathways. This may occur when compounds acting at such new targets are discarded, ignored or fail to be detected because compounds acting at the “old” targets are encountered more frequently and are more potent than compounds acting at the new targets. As a result, the majority of antibiotics in use currently interact with a relatively small number of target molecules within an even more limited set of biological pathways.

The use of sensitized cells of the current invention provides a solution to the above problems in two ways. First, desired compounds acting at a target of interest, whether a new target or a previously known but poorly exploited target, can now be detected above the “noise” of compounds acting at the “old” targets due to the specific and substantial increase in potency of such desired compounds when tested on the sensitized cells of the current invention. Second, the methods used to sensitize cells to compounds acting at a target of interest may also sensitize these cells to compounds acting at other target molecules within the same biological pathway. For example, expression of a gene encoding a ribosomal protein at a level such that the function of the ribosomal protein becomes rate limiting for fungal growth, survival, proliferation, virulence, or pathogenicity is expected to sensitize the cell to compounds acting at that ribosomal protein to compounds acting at any of the ribosomal components (proteins or rRNA) or even to compounds acting at any target which is part of the protein synthesis pathway. Thus an important advantage of the present invention is the ability to reveal new targets and pathways that were previously not readily accessible to drug discovery methods.

Sensitized cells of the present invention are prepared by reducing the activity or level of a target molecule. The target molecule may be a gene product, such as an RNA or polypeptide produced from the nucleic acids required for fungal growth, survival, proliferation, virulence, or pathogenicity described herein. In addition, the target may be an RNA or polypeptide in the same biological pathway as the nucleic acids required for fungal growth, survival, proliferation, virulence, or pathogenicity as described herein. Such biological pathways include, but are not limited to, enzymatic, biochemical and metabolic pathways as well as pathways involved in the production of cellular structures such as the cell membrane.

Current methods employed in the arts of medicinal and combinatorial chemistries are able to make use of structure-activity relationship information derived from testing compounds in various biological assays including direct binding assays and cell-based assays. Occasionally compounds are directly identified in such assays that are sufficiently potent to be developed as drugs. More often, initial hit compounds exhibit moderate or low potency. Once a hit compound is identified with low or moderate potency, directed libraries of compounds are synthesized and tested in order to identify more potent leads. Generally these directed libraries are combinatorial chemical libraries consisting of compounds with structures related to the hit compound but containing systematic variations including additions, subtractions and substitutions of various structural features. When tested for activity against the target molecule, structural features are identified that either alone or in combination with other features enhance or reduce activity. This information is used to design subsequent directed libraries containing compounds with enhanced activity against the target molecule. After one or several iterations of this process, compounds with substantially increased activity against the target molecule are identified and may be further developed as drugs. This process is facilitated by use of the sensitized cells of the present invention since compounds acting at the selected targets exhibit increased potency in such cell-based assays, thus; more compounds can now be characterized providing more useful information than would be obtained otherwise.

Thus, it is now possible using cell-based assays of the present invention to identify or characterize compounds that previously would not have been readily identified or characterized including compounds that act at targets that previously were not readily exploited using cell-based assays. The process of evolving potent drug leads from initial hit compounds is also substantially improved by the cell-based assays of the present invention because, for the same number of test compounds, more structure-function relationship information is likely to be revealed.

The method of sensitizing a cell entails selecting a suitable gene. A suitable gene is one whose expression is required for the growth, survival, proliferation, virulence, or pathogenicity of the cell to be sensitized. The next step is to obtain a cell in which the level or activity of the target can be reduced to a level where it is rate limiting for growth, survival, proliferation, virulence or pathogenicity. For example, the cell may be a conditional-expression Aspergillus fumigatus mutant strain in which the selected gene is under the control of a regulatable promoter. The amount of RNA transcribed from the selected gene is limited by varying the concentration of an inducer or repressor which acts on the regulatable promoter, thereby varying the activity of the promoter driving transcription of the RNA. Thus, cells are sensitized by exposing them to an inducer or repressor concentration that results in an RNA level such that the function of the selected gene product becomes rate limiting for fungal growth, survival, proliferation, virulence, or pathogenicity.

In one embodiment of the cell-based assays, conditional-expression Aspergillus fumigatus mutant strains, in which the sequences required for fungal survival, growth, proliferation, virulence, or pathogenicity of Aspergillus fumigatus described herein are under the control of a regulatable promoter, are grown in the presence of a concentration of inducer or repressor which causes the function of the gene products encoded by these sequences to be rate limiting for fungal growth, survival, proliferation, virulence, or pathogenicity. To achieve that goal, a growth inhibition dose curve of inducer or repressor is calculated by plotting various doses of inducer or repressor against the corresponding growth inhibition caused by the limited levels of the gene product required for fungal proliferation. From this dose-response curve, conditions providing various growth rates, from 1 to 100% as compared to inducer or repressor-free growth, can be determined. For example, if the regulatable promoter is repressed by tetracycline, the conditional-expression Aspergillus fumigatus mutant strain may be grown in the presence of varying levels of tetracyline. Similarly, inducible promoters may be used. In this case, the conditional-expression Aspergillus fumigatus mutant strains are grown in the presence of varying concentrations of inducer. For example, the highest concentration of the inducer or repressor that does not reduce the growth rate significantly can be estimated from the dose-response curve. Cellular proliferation can be monitored by growth medium turbidity via OD measurements. In another example, the concentration of inducer or repressor that reduces growth by 25% can be predicted from the dose-response curve. In still another example, a concentration of inducer or repressor that reduces growth by 50% can be calculated from the dose-response curve. Additional parameters such as colony forming units (cfu) are also used to measure cellular growth, survival and/or viability.

In another embodiment of the present invention, an individual haploid strain may similarly be used as the basis for detection of an antifungal or therapeutic agent. In this embodiment, the test organism (e.g. Cryptococcus neoformans, Magnaportha grisea or any other haploid organisms represented in Table 2) is a strain constructed by modifying the single allele of the target gene in one step by recombination with a promoter replacement fragment comprising a heterologous regulatable promoter, such that the expression of the gene is conditionally regulated by the heterologous promoter. Such individual sensitized haploid cells are used in whole cell-based assay methods to identify compounds displaying a preferential activity against the affected target.

In various embodiments, the conditional-expression Aspergillus fumigatus mutant strain is grown under a first set of conditions where the heterologous promoter is expressed at a relatively low level (i.e. partially repressed) and the extent of growth determined. This experiment is repeated in the presence of a test compound and a second measurement of growth obtained. The extent of growth in the presence and in the absence of the test compound are then compared to provide a first indicator value. Two further experiments are performed, using non-repressing growth conditions where the target gene is expressed at substantially higher levels than in the first set of conditions. The extent of growth is determined in the presence and absence of the test compound under the second set of conditions to obtain a second indicator value. The first and second indicator values are then compared. If the indicator values are essentially the same, the data suggest that the test compound does not inhibit the test target. However, if the two indicator values are substantially different, the data indicates that the level of expression of the target gene product may determine the degree of inhibition by the test compound and, therefore, it is likely that the gene product is the target of that test compound. Whole-cell assays comprising collections or subsets of multiple sensitized strains may also be screened, for example, in a series of 96-well, 384-well, or even 1586-well microtiter plates, with each well containing individual strains sensitized to identify compounds displaying a preferential activity against each affected target comprising a target set or subset selected from, but not limited to the group consisting of fungal-specific, pathogen-specific, desired biochemical-function, human-homolog, cellular localization, and signal transduction cascade target sets.

Cells to be assayed are exposed to the above-determined concentrations of inducer or repressor. The presence of the inducer or repressor at this sub-lethal concentration reduces the amount of the proliferation-required gene product to the lowest amount in the cell that will support growth. Cells grown in the presence of this concentration of inducer or repressor are therefore specifically more sensitive to inhibitors of the proliferation-required protein or RNA of interest as well as to inhibitors of proteins or RNAs in the same biological pathway as the proliferation-required protein or RNA of interest but not specifically more sensitive to inhibitors of unrelated proteins or RNAs.

Cells pretreated with sub-inhibitory concentrations of inducer or repressor, which therefore contain a reduced amount of proliferation-required target gene product, are used to screen for compounds that reduce cell growth. The sub-lethal concentration of inducer or repressor may be any concentration consistent with the intended use of the assay to identify candidate compounds to which the cells are more sensitive than are control cells in which this gene product is not rate-limiting. For example, the sub-lethal concentration of the inducer or repressor may be such that growth inhibition is at least about 5%, at least about 8%, at least about 10%, at least about 20%, at least about 30%, at least about 40%, at least about 50%, at least about 60% at least about 75%, at least 80%, at least 90%, at least 95% or more than 95%. Cells which are pre-sensitized using the preceding method are more sensitive to inhibitors of the target protein because these cells contain less target protein to inhibit than wild-type cells.

It will be appreciated that similar methods may be used to identify compounds which inhibit virulence or pathogenicity. In such methods, the virulence or pathogenicity of cells exposed to the candidate compound which express rate limiting levels of a gene product involved in virulence or pathogenicity is compared to the virulence or pathogenicity of cells exposed to the candidate compound in which the levels of the gene product are not rate limiting. Virulence or pathogenicity may be measured using the techniques described herein.

In another embodiment of the cell-based assays of the present invention, the level or activity of a gene product required for fungal growth, survival, proliferation, virulence, or pathogenicity is reduced using a mutation, such as a temperature sensitive mutation, in the sequence required for fungal growth, survival, proliferation, virulence, or pathogenicity and an inducer or repressor level which, in conjunction with the temperature sensitive mutation, provides levels of the gene product required for fungal growth, survival, proliferation, virulence, or pathogenicity which are rate limiting for proliferation. Growing the cells at an intermediate temperature between the permissive and restrictive temperatures of the temperature sensitive mutant where the mutation is in a gene required for fungal growth, survival, proliferation, virulence, or pathogenicity produces cells with reduced activity of the gene product required for growth, survival, proliferation, virulence, or pathogenicity. The concentration of inducer or repressor is chosen so as to further reduces the activity of the gene product required for fungal growth, survival, proliferation, virulence, or pathogenicity. Drugs that may not have been found using either the temperature sensitive mutation or the inducer or repressor alone may be identified by determining whether cells in which expression of the nucleic acid encoding the proliferation-required gene product has been reduced and which are grown at a temperature between the permissive temperature and the restrictive temperature are substantially more sensitive to a test compound than cells in which expression of the gene product required for fungal growth, survival, proliferation, virulence, or pathogenicity has not been reduced and which are grown at a permissive temperature. Also drugs found previously from either the use of the inducer or repressor alone or the temperature sensitive mutation alone may have a different sensitivity profile when used in cells combining the two approaches, and that sensitivity profile may indicate a more specific action of the drug in inhibiting one or more activities of the gene product.

Temperature sensitive mutations may be located at different sites within a gene and may lie within different domains of the protein. For example, the dnaB gene of Escherichia coli encodes the replication fork DNA helicase. DnaB has several domains, including domains for oligomerization, ATP hydrolysis, DNA binding, interaction with primase, interaction with DnaC, and interaction with DnaA. Temperature sensitive mutations in different domains of DnaB confer different phenotypes at the restrictive temperature, which include either an abrupt stop or a slow stop in DNA replication either with or without DNA breakdown (Wechsler, J. A. and Gross, J. D. 1971 Escherichia coli mutants temperature-sensitive for DNA synthesis. Mol. Gen. Genetics 113:273-284) and termination of growth or cell death. Thus, temperature sensitive mutations in different domains of the protein may be used in conjunction with conditional-expression Aspergillus fumigatus mutant strains in which expression of the protein is under the control of a regulatable promoter.

It will be appreciated that the above method may be performed with any mutation which reduces but does not eliminate the activity or level of the gene product which is required for fungal growth, survival, proliferation, virulence, or pathogenicity.

When screening for antimicrobial agents against a gene product required for fungal growth, survival, proliferation, virulence, or pathogenicity, growth inhibition, virulence or pathogenicity of cells containing a limiting amount of that gene product can be assayed. Growth inhibition can be measured by directly comparing the amount of growth, measured by the optical density of the culture relative to uninoculated growth medium, between an experimental sample and a control sample. Alternative methods for assaying cell proliferation include measuring green fluorescent protein (GFP) reporter construct emissions, various enzymatic activity assays, and other methods well known in the art. Virulence and pathogenicity may be measured using the techniques described herein.

It will be appreciated that the above method may be performed in solid phase, liquid phase, a combination of the two preceding media, or in vivo. For example, cells grown on nutrient agar containing the inducer or repressor which acts on the regulatable promoter used to express the proliferation required gene product may be exposed to compounds spotted onto the agar surface. A compound's effect may be judged from the diameter of the resulting killing zone, the area around the compound application point in which cells do not grow. Multiple compounds may be transferred to agar plates and simultaneously tested using automated and semi-automated equipment including but not restricted to multi-channel pipettes (for example the Beckman Multimek) and multi-channel spotters (for example the Genomic Solutions Flexys). In this way multiple plates and thousands to millions of compounds may be tested per day.

The compounds are also tested entirely in liquid phase using microtiter plates as described below. Liquid phase screening may be performed in microtiter plates containing 96, 384, 1536 or more wells per microtiter plate to screen multiple plates and thousands to millions of compounds per day. Automated and semi-automated equipment are used for addition of reagents (for example cells and compounds) and for determination of cell density.

The compounds are also tested in vivo using the methods described herein.

It will be appreciated that each of the above cell-based assays may be used to identify compounds which inhibit the activity of gene products from organisms other than Aspergillus fumigatus which are homologous to the Aspergillus fumigatus gene products described herein. For example, the target gene products may be from animal fugal pathogens such as Aspergillus niger, Aspergillus flavis, Candida tropicalis, Candida albicans, Candida parapsilopsis, Candida krusei, Cryptococcus neoformans, Coccidioides immitis, Exophalia dermatiditis, Fusarium oxysporum, Histoplasma capsulatum, Phneumocystis carinii, Trichosporon beigelii, Rhizopus arrhizus, Mucor rouxii, Rhizomucor pusillus, or Absidia corymbigera, or the plant fungal pathogens, such as Botrytis cinerea, Erysiphe graminis, Magnaporthe grisea, Puccinia recodita, Septoria triticii, Tilletia controversa, Ustilago maydis, or any species falling within the genera of any of the above species. In some embodiments, the gene products are from an organism other than Saccharomyces cerevisiae.

### 5.4.2.1 Cell-Based Assays Using Conditional-expression Aspergillus fumigatus Mutant Strains

Conditional-expression Aspergillus fumigatus mutant strains in which a gene required for fungal survival, growth, proliferation, virulence, or pathogenicity is placed under the control of a regulatable promoter are constructed using the methods described herein. For the purposes of the present example, the regulatable promoter may be the tetracycline regulated promoter described herein, but it will be appreciated that any regulatable promoter may be used.

In one embodiment of the present invention, an individual conditional-expression Aspergillus fumigatus mutant strain is used as the basis for detection of a therapeutic agent active against a diploid pathogenic fungal cell. In this embodiment, the test organism is a conditional-expression Aspergillus fumigatus mutant strain having a gene that has been modified, by recombination, to place the gene under the controlled expression of a heterologous promoter. This test conditional-expression Aspergillus fumigatus mutant strain is then grown under a first set of conditions where the heterologous promoter is expressed at a relatively low level (“repressing”) and the extent of growth determined. This measurement may be carried out using any appropriate standard known to those skilled in the art including optical density, wet weight of pelleted cells, total cell count, viable count, DNA content, and the like. This experiment is repeated in the presence of a test compound and a second measurement of growth obtained. The extent of growth in the presence and in the absence of the test compound, which can conveniently be expressed in terms of indicator values, are then compared. A dissimilarity in the extent of growth or indicator values provides an indication that the test compound may interact with the target essential gene product.

To gain more information, two further experiments are performed, using a second set of “non-repressing” growth conditions where the essential gene, under the control of the heterologous promoter, is expressed at a level substantially higher than in the first set of conditions described above. The extent of growth or indicator values is determined in the presence and absence of the test compound under this second set of conditions. The extent of growth or indicator values in the presence and in the absence of the test compound are then compared. A dissimilarity in the extent of growth or indicator values provides an indication that the test compound may interact with the target essential gene product.

Furthermore, the extent of growth in the first and in the second set of growth conditions can also be compared. If the extent of growth is essentially the same, the data suggest that the test compound does not inhibit the gene product encoded by the modified gene carried by the conditional-expression Aspergillus fumigatus mutant strain tested. However, if the extent of growth are substantially different, the data indicate that the level of expression of the subject gene product may determine the degree of inhibition by the test compound and, therefore, it is likely that the subject gene product is the target of that test compound.

Although each conditional-expression Aspergillus fumigatus mutant strain can be tested individually, it will be more efficient to screen entire sets or subsets of a conditional-expression Aspergillus fumigatus mutant strain collection at one time. Therefore in one aspect of this invention, arrays may be established, for example in a series of 96-well microtiter plates, with each well containing a single conditional-expression Aspergillus fumigatus mutant strain. In one representative, but not limiting approach, four microtiter plates are used, comprising two pairs where the growth medium in one pair supports greater expression of the heterologous promoter controlling the remaining active allele in each strain, than the medium in the other pair of plates. One member of each pair is supplemented with a compound to be tested and measurements of growth of each conditional-expression Aspergillus fumigatus mutant strain is determined using standard procedures to provide indicator values for each isolate tested. The collection of conditional-expression Aspergillus fumigatus mutant strains used in such a method for screening for therapeutic agents may comprise a subset of conditional-expression Aspergillus fumigatus mutant strains selected from, but not limited to the group consisting of fungal-specific, pathogen-specific, desired biochemical-function, human-homolog, cellular localization, and signal transduction cascade target sets.

The conditional-expression Aspergillus fumigatus mutant strains are grown in medium comprising a range of tetracycline concentrations to obtain the growth inhibitory dose-response curve for each strain. First, seed cultures of the conditional-expression Aspergillus fumigatus mutant strains are grown in the appropriate medium. Subsequently, aliquots of the seed cultures are diluted into medium containing varying concentrations of tetracycline. For example, the conditional-expression Aspergillus fumigatus mutant strains may be grown in duplicate cultures containing two-fold serial dilutions of tetracycline. Additionally, control cells are grown in duplicate without tetracycline. The control cultures are started from equal amounts of cells derived from the same initial seed culture of a conditional-expression Aspergillus fumigatus mutant strain of interest. The cells are grown for an appropriate period of time and the extent of growth is determined using any appropriate technique. For example, the extent of growth may be determined by measuring the optical density of the cultures. When the control culture reaches mid-log phase the percent growth (relative to the control culture) for each of the tetracycline containing cultures is plotted against the log concentrations of tetracycline to produce a growth inhibitory dose response curve for tetracycline. The concentration of tetracycline that inhibits cell growth to 50% (IC₅₀) as compared to the 0 mM tetracyline control (0% growth inhibition) is then calculated from the curve. Alternative methods of measuring growth are also contemplated. Examples of these methods include measurements of proteins, the expression of which is engineered into the cells being tested and can readily be measured. Examples of such proteins include green fluorescent protein (GFP) and various enzymes.

Cells are pretreated with the selected concentration of tetracycline and then used to test the sensitivity of cell populations to candidate compounds. For example, the cells may be pretreated with a concentration of tetracycline which inhibits growth by at least about 5%, at least about 8%, at least about 10%, at least about 20%, at least about 30%, at least about 40%, at least about 50%, at least about 60% at least about 75%, at least 80%, at least 90%, at least 95% or more than 95%. The cells are then contacted with the candidate compound and growth of the cells in tetracycline containing medium is compared to growth of the control cells in medium which lacks tetracycline to determine whether the candidate compound inhibits growth of the sensitized cells (i.e. the cells grown in the presence of tetracycline). For example, the growth of the cells in tetracycline containing medium may be compared to the growth of the cells in medium lacking tetracycline to determine whether the candidate compound inhibits the growth of the sensitized cells (i.e. the cells grown in the presence of tetracyline) to a greater extent than the candidate compound inhibits the growth of cells grown in the absence of tetracycline. For example, if a significant difference in growth is observed between the sensitized cells (i.e. the cells grown in the presence of tetracycline) and the non-sensitized cells (i.e. the cells grown in the absence of tetracycline), the candidate compound may be used to inhibit the proliferation of the organism or may be further optimized to identify compounds which have an even greater ability to inhibit the growth, survival, or proliferation of the organism.

Similarly, the virulence or pathogenicity of cells exposed to a candidate compound which express a rate limiting amount of a gene product required for virulence or pathogenicity may be compared to the virulence or pathogenicity of cells exposed to the candidate compound in which the level of expression of the gene product required for virulence or pathogenicity is not rate limiting. In such methods, test animals are challenged with the conditional-expression Aspergillus fumigatus mutant strain and fed a diet containing the desired amount of tetracycline and the candidate compound. Thus, the conditional-expression Aspergillus fumigatus mutant strain infecting the test animals expresses a rate limiting amount of a gene product required for virulence or pathogenicity (i.e. the conditional-expression Aspergillus fumigatus mutant cells in the test animals are sensitized). Control animals are challenged with the conditional-expression Aspergillus fumigatus mutant strain and are fed a diet containing the candidate compound but lacking tetracycline. The virulence or pathogenicity of the conditional-expression Aspergillus fumigatus mutant strain in the test animals is compared to that in the control animals. For example, the virulence or pathogenicity of the conditional-expression Aspergillus fumigatus mutant strain in the test animals may be compared to that in the control animals to determine whether the candidate compound inhibits the virulence or pathogenicity of the sensitized conditional-expression Aspergillus fumigatus mutant cells (i.e. the cells in the animals whose diet included tetracyline) to a greater extent than the candidate compound inhibits the growth of the conditional-expression Aspergillus fumigatus mutant cells in animals whose diet lacked tetracycline. For example, if a significant difference in growth is observed between the sensitized conditional-expression Aspergillus fumigatus mutant cells (i.e. the cells in animals whose diet included tetracycline) and the non-sensitized cells (i.e. the conditional-expression Aspergillus fumigatus mutant cells animals whose diet did not include tetracycline), the candidate compound may be used to inhibit the virulence or pathogenicity of the organism or may be further optimized to identify compounds which have an even greater ability to inhibit the virulence or pathogenicity of the organism. Virulence or pathogenicity may be measured using the techniques described therein.

It will be appreciated that the above cell-based assays may be used to identify compounds which inhibit the activity of gene products from organisms other than Aspergillus fumigatus which are homologous to the Aspergillus fumigatus gene products described herein. For example, the gene products may be from animal fugal pathogens such as Aspergillus niger, Aspergillus flavis, Candida tropicalis, Candida parapsilopsis, Candida krusei, Cryptococcus neoformans, Coccidioides immitis, Exophalia dermatiditis, Fusarium oxysporum, Histoplasma capsulatum, Phneumocystis carinii, Trichosporon beigelii, Rhizopus arrhizus, Mucor rouxii, Rhizomucor pusillus, or Absidia corymbigera, or the plant fungal pathogens, such as Botrytis cinerea, Erysiphe graminis, Magnaporthe grisea, Puccinia recodita, Septoria triticii, Tilletia controversa, Ustilago maydis, or any species falling within the genera of any of the above species. In some embodiments, the gene products are from an organism other than Saccharomyces cerevisiae or Candida albicans.

In many of the fungal plant pathogens where homologous target genes are identified, standard genetic manipulation methods that are known to those of skill in the art, e.g., transformation and homologous recombination, are applicable. Non-limiting examples of recombinant gene expression systems include the following: F. oxysporum panC promoter induced by steroidal glycoalkaloid alpha-tomatine (Perez-Espinosa et al., Mol Genet Genomics (2001) 265(5):922-9); Ustilago maydis hsp70-like gene promoter in a high-copy number autonomously replicating expression vector (Keon et al., Antisense Nucleic Acid Drug Dev (1999), 9(1):101-4); Cochliobolus heterostrophus transient and stable gene expression systems using P1 or GPD 1 (glyceraldehyde 3 phosphate dehydrogenase) promoter of C. heterostrophus or GUS or hygromycin B phosphotransferase gene (hph) of E. coli (Monke et al., Mol Gen Genet (1993) 241(1-2):73-80); Rhynchosporium secalis (barley leaf scald fungus) transformed to hygromycin-B and phleomycin resistance using the hph gene from E. coli and the ble gene from Streptoalloteichus hindustanus under the control of Aspergillus nidulans promoter and terminator sequences, plasmid DNA introduced into fungal protoplasts by PEG/CaCl₂ treatment (Rohe et al., Curr Genet (1996), 29(6): 587-90). Pathogens of banana and plantain (Musa spp.) Mycosphaerella fijiensis and Mycosphaerella musicola, and Mycosphaerella eumusae can be transformed as taught in Balint-Kurti et al., FEMS Microbiol Lett (2001), 195(1): 9-15. Gibberella pulicaris (Fusarium sambucinum) a trichothecene-producing plant pathogen can be transformed with three different vectors: cosHyg1, pUCH1, and pDH25, all of which carry hph (encoding hygromycin B phosphotransferase) as the selectable marker (Salch et al., Curr Genet (1993), 23(4): 343-50). Leptosphaeria maculans, a fungal pathogen of Brassica spp.can be transformed with the vector pAN8-1, encoding phleomycin resistance; protoplasts can be retransformed using the partially homologous vector, pAN7-1 which encodes hygromycin B resistance. Farmnan et al., Mol Gen Genet (1992) 231(2):243-7. Cryphonectria parasitica; targeted disruption of enpg-1 of this chestnut blight fungus was accomplished by homologous recombination with a cloned copy of the hph gene of Escherichia coli inserted into exon 1, see Gao et al., Appl Environ Microbiol (1996), 62(6):1984-90.

Another example, Glomerella cingulataf sp. phaseoli (Gcp) was transformed using either of two selectable markers: the amdS+gene of Aspergillus nidulans, which encodes acetamidase and permits growth on acetamide as the sole nitrogen source and the hygBR gene of Escherichia coli which permits growth in the presence of the antibiotic Hy. The amdS+gene functioned in Gcp under control of A. nidulans regulatory signals and hygBR was expressed after fusion to a promoter from Cochliobolus heterostrophus, another filamentous ascomycete. Protoplasts to be transformed were generated with the digestive enzyme complex Novozym 234 and then were exposed to plasmid DNA in the presence of 10 mM CaCl₂ and polyethylene glycol. Transformation occurred by integration of single or multiple copies of either the amdS+ or hygBR plasmid into the fungal genome. (Rodriquez et al., Gene (1987), 54(1):73-81); integration vectors for homologous recombination; deletion studies demonstrated that 505 bp (the minimum length of homologous promoter DNA analysed which was still capable of promoter function) was sufficient to target integration events. Homologous integration of the vector resulted in duplication of the gdpA promoter region. (Rikkerink et al., Curr Genet (1994), 25(3): 202-8).

The cell-based assay described above may also be used to identify the biological pathway in which a nucleic acid required for fungal proliferation, virulence or pathogenicity or the gene product of such a nucleic acid lies. In such methods, cells expressing a rate limiting level of a target nucleic acid required for fungal proliferation, virulence or pathogenicity and control cells in which expression of the target nucleic acid is not rate limiting are contacted with a panel of antibiotics known to act in various pathways. If the antibiotic acts in the pathway in which the target nucleic acid or its gene product lies, cells in which expression of target nucleic acid is rate limiting will be more sensitive to the antibiotic than cells in which expression of the target nucleic acid is not rate limiting.

As a control, the results of the assay may be confirmed by contacting a panel of cells in which the levels of many different genes required for proliferation, virulence or pathogenicity, including the target gene, is rate limiting. If the antibiotic is acting specifically, heightened sensitivity to the antibiotic will be observed only in the cells in which the target gene is rate limiting (or cells in which genes in the same pathway as the target gene is rate limiting) but will not be observed generally in which a gene product required for proliferation, virulence or pathogenicity is rate limiting.

It will be appreciated that the above method for identifying the biological pathway in which a nucleic acid required for proliferation, virulence or pathogenicity lies may be applied to nucleic acids from organisms other than Aspergillus fumigatus which are homologous to the Aspergillus fumigatus nucleic acids described herein. For example, the nucleic acids may be from animal fugal pathogens such as Aspergillus niger, Aspergillus flavis, Candida tropicalis, Candida albicans, Candida parapsilopsis, Candida krusei, Cryptococcus neoformans, Coccidioides immitis, Exophalia dermatiditis, Fusarium oxysporum, Histoplasma capsulatum, Phneumocystis carinii, Trichosporon beigelii, Rhizopus arrhizus, Mucor rouxii, Rhizomucorpusillus, or Absidia corymbigera, or the plant fungal pathogens, such as Botrytis cinerea, Erysiphe graminis, Magnaporthe grisea, Puccinia recodita, Septoria triticii, Tilletia controversa, Ustilago maydis, or any species falling within the genera of any of the above species. In some embodiments, the nucleic acids are from an organism other than Saccharomyces cerevisiae.

Similarly, the above method may be used to determine the pathway on which a test compound, such as a test antibiotic acts. A panel of cells, each of which expresses a rate limiting amount of a gene product required for fungal survival, growth, proliferation, virulence or pathogenicity where the gene product lies in a known pathway, is contacted with a compound for which it is desired to determine the pathway on which it acts. The sensitivity of the panel of cells to the test compound is determined in cells in which expression of the nucleic acid encoding the gene product required for proliferation, virulence or pathogenicity is at a rate limiting level and in control cells in which expression of the gene product required for proliferation, virulence or pathogenicity is not at a rate limiting level. If the test compound acts on the pathway in which a particular gene product required for proliferation, virulence, or pathogenicity lies, cells in which expression of that particular gene product is at a rate limiting level will be more sensitive to the compound than the cells in which gene products in other pathways are at a rate limiting level. In addition, control cells in which expression of the particular gene required for fungal proliferation, virulence or pathogenicity is not rate limiting will not exhibit heightened sensitivity to the compound. In this way, the pathway on which the test compound acts may be determined.

It will be appreciated that the above method for determining the pathway on which a test compound acts may be applied to organisms other than Aspergillus fumigatus by using panels of cells in which the activity or level of gene products which are homologous to the Aspergillus fumigatus gene products described herein is rate limiting. For example, the gene products may be from animal fugal pathogens such as Aspergillus niger, Aspergillus flavis, Candida tropicalis, Candida parapsilopsis, Candida krusei, Cryptococcus neoformans, Coccidioides immitis, Exophalia dermatiditis, Fusarium oxysporum, Histoplasma capsulatum, Pneumocystis carinii, Trichosporon beigelii, Rhizopus arrhizus, Mucor rouxii, Rhizomucorpusillus, or Absidia corymbigera, or the plant fungal pathogens, such as Botrytis cinerea, Erysiphe graminis, Magnaporthe grisea, Puccinia recodita, Septoria triticii, Tilletia controversa, Ustilago maydis, or any species falling within the genera of any of the above species. In some embodiments, the gene products are from an organism other than Saccharomyces cerevisiae or Candida albicans.

One skilled in the art will appreciate that further optimization of the assay conditions, such as the concentration of inducer or repressor used to produce rate limiting levels of a gene product required for fungal proliferation, virulence or pathogenicity and/or the growth conditions used for the assay (for example incubation temperature and medium components) may further increase the selectivity and/or magnitude of the antibiotic sensitization exhibited.

It will be appreciated that the above methods for identifying the pathway in which a gene required for growth, survival, proliferation, virulence or pathogenicity lies or the pathway on which an antibiotic acts may be performed using organisms other than Aspergillus fumigatus in which gene products homologous to the Aspergillus fumigatus gene products described herein are rate limiting. For example, the gene products may be from animal fugal pathogens such as Aspergillus niger, Aspergillus flavis, Candida albicans, Candida tropicalis, Candida parapsilopsis, Candida krusei, Cryptococcus neoformans, Coccidioides immitis, Exophalia dermatiditis, Fusarium oxysporum, Histoplasma capsulatum, Pneumocystis carinii, Trichosporon beigelii, Rhizopus arrhizus, Mucor rouxii, Rhizomucor pusillus, or Absidia corymbigera, or the plant fungal pathogens, such as Botrytis cinerea, Erysiphe graminis, Magnaporthe grisea, Puccinia recodita, Septoria triticii, Tilletia controversa, Ustilago maydis, or any species falling within the genera of any of the above species. In some embodiments, the gene products are from an organism other than Saccharomyces cerevisiae.

Furthermore, as discussed above, panels of conditional-expression Aspergillus fumigatus mutant strains may be used to characterize the point of intervention of any compound affecting an essential biological pathway including antibiotics with no known mechanism of action.

Another embodiment of the present invention is a method for determining the pathway against which a test antibiotic compound is active, in which the activity of proteins or nucleic acids involved in pathways required for fungal growth, survival, proliferation, virulence or pathogenicity is reduced by contacting cells with a sub-lethal concentration of a known antibiotic which acts against the protein or nucleic acid. The method is similar to those described above for determining which pathway a test antibiotic acts against, except that rather than reducing the activity or level of a gene product required for fungal proliferation, virulence or pathogenicity by expressing the gene product at a rate limiting amount in a conditional-expression Aspergillus fumigatus mutant strain, the activity or level of the gene product is reduced using a sub-lethal level of a known antibiotic which acts against the gene product.

Growth inhibition resulting from the presence of sub-lethal concentration of the known antibiotic may be at least about 5%, at least about 8%, at least about 10%, at least about 20%, at least about 30%, at least about 40%, at least about 50%, at least about 60%, or at least about 75%, at least 80%, at least 90%, at least 95% or more than 95%.

Alternatively, the sub-lethal concentration of the known antibiotic may be determined by measuring the activity of the target proliferation-required gene product rather than by measuring growth inhibition.

Cells are contacted with a combination of each member of a panel of known antibiotics at a sub-lethal level and varying concentrations of the test antibiotic. As a control, the cells are contacted with varying concentrations of fine test antibiotic alone. The IC₅₀ of the test antibiotic in the presence and absence of the known antibiotic is determined. If the IC₅₀s in the presence and absence of the known drug are substantially similar, then the test drug and the known drug act on different pathways. If the IC₅₀s are substantially different, then the test drug and the known drug act on the same pathway.

Similar methods may be performed using known antibiotics which act on a gene product homologous to the Aspergillus fumigatus sequences described herein. The homolgous gene product may be from animal fugal pathogens such as Aspergillus niger, Aspergillus flavis, Candida tropicalis, Candida parapsilopsis, Candida krusei, Cryptococcus neoformans, Coccidioides immitis, Exophalia dermatiditis, Fusarium oxysporum, Histoplasma capsulatum, Pneumocystis carinii, Trichosporon beigelii, Rhizopus arrhizus, Mucor rouxii, Rhizomucorpusillus, or Absidia corymbigera, or the plant fungal pathogens, such as Botrytis cinerea, Erysiphe graminis, Magnaporthe grisea, Puccinia recodita, Septoria triticii, Tilletia controversa, Ustilago maydis, or any species falling within the genera of any of the above species. In some embodiments, the gene products are from an organism other than Saccharomyces cerevisae or Candida albicans.

Another embodiment of the present invention is a method for identifying a candidate compound for use as an antibiotic in which the activity of target proteins or nucleic acids involved in pathways required for fungal proliferation, virulence or pathogenicity is reduced by contacting cells with a sub-lethal concentration of a known antibiotic which acts against the target protein or nucleic acid. The method is similar to those described above for identifying candidate compounds for use as antibiotics except that rather than reducing the activity or level of a gene product required for proliferation, virulence or pathogenicity using conditional-expression Aspergillus fumigatus mutant strains which express a rate limiting level of the gene product, the activity or level of the gene product is reduced using a sub-lethal level of a known antibiotic which acts against the proliferation-required gene product.

The growth inhibition from the sub-lethal concentration of the known antibiotic may be at least about 5%, at least about 8%, at least about 10%, at least about 20%, at least about 30%, at least about 40%, at least about 50%, at least about 60%, or at least about 75%, or more.

Alternatively, the sub-lethal concentration of the known antibiotic may be determined by measuring the activity of the target proliferation-required gene product rather than by measuring growth inhibition.

In order to characterize test compounds of interest, cells are contacted with a panel of known antibiotics at a sub-lethal level and one or more concentrations of the test compound. As a control, the cells are contacted with the same concentrations of the test compound alone. The IC₅₀ of the test compound in the presence and absence of the known antibiotic is determined. If the IC₅₀ of the test compound is substantially different in the presence and absence of the known drug then the test compound is a good candidate for use as an antibiotic. As discussed above, once a candidate compound is identified using the above methods its structure may be optimized using standard techniques such as combinatorial chemistry.

Similar methods may be performed using known antibiotics which act on a gene product homologous to the Aspergillus fumigatus sequences described herein. The homolgous gene product may be from animal fugal pathogens such as Aspergillus niger, Aspergillus flavis, Candida albicans, Candida tropicalis, Candida parapsilopsis, Candida krusei, Cryptococcus neoformans, Coccidioides immitis, Exophalia dermatiditis, Fusarium oxysporum, Histoplasma capsulatum, Pneumocystis carinii, Trichosporon beigelii, Rhizopus arrhizus, Mucor rouxii, Rhizomucor pusillus, or Absidia corymbigera, or the plant fungal pathogens, such as Botrytis cinerea, Erysiphe graminis, Magnaporthe grisea, Puccinia recodita, Septoria triticii, Tilletia controversa, Ustilago maydis, or any species falling within the genera of any of the above species. In some embodiments, the gene products are from an organism other than Saccharomyces cerevisiae.

In another embodiment of the present invention, all potential drug targets of a pathogen could be screened simultaneously against a library of compounds using, for example a 96 well microtiter plate format, where growth, measured by optical density or pellet size after centrifugation, may be determined for each well. A genomic approach to drug screening eliminates reliance upon potentially arbitrary and artificial criteria used in evaluating which target to screen and instead allows all potential targets to be screened. This approach not only offers the possibility of identifying specific compounds which inhibit a preferred process (e. g. cell wall biosynthetic gene products) but also the possibility of identifying all fungicidal compounds within that library and linking them to their cognate cellular targets.

In still another embodiment of the present invention, conditional-expression Aspergillus fumigatus mutant strains could be screened to identify synthetic lethal mutations, and thereby uncover a potentially novel class of drug targets of significant therapeutic value. For example two separate genes may encode homologous proteins that participate in a common and essential cellular function, where the essential nature of this function will only become apparent upon inactivation of both family members. Accordingly, examination of the null phenotype of each gene separately would not reveal the essential nature of the combined gene products, and consequently, this potential drug target would not be identified. Provided the gene products are highly homologous to one another, compounds found to inhibit one family member are likely to inhibit the other and are therefore predicted to approximate the synthetic growth inhibition demonstrated genetically. In other cases however, synthetic lethality may uncover seemingly unrelated (and often nonessential) processes, which when combined produce a synergistic growth impairment (cell death). For example, although disruption of the S. cerevisiae gene RVS161 does not present any discemable vegetative growth phenotype in yeast carrying this single mutation, at least 9 other genes are known to display a synthetic lethal effect when combined with inactivation of RVS161. These genes participate in processes ranging from cytoskeletal assembly and endocytosis, to signal transduction and lipid metabolism and identifies multiple avenues to pursuing a combination drug target strategy. A directed approach to uncovering synthetic lethal interactions with essential and nonessential drug targets is now performed where a conditional-expression Aspergillus fumigatus mutant strain is identified as displaying an enhanced sensitivity to the tested compound, not because it expresses a reduced level of activity for the drug target, but because its mutation is synthetically lethal in combination with inhibition of a second drug target. Discerning whether the compound specifically inhibits the drug target in the sensitized conditional-expression Aspergillus fumigatus mutant strain may be achieved by screening the entire conditional-expression Aspergillus fumigatus mutant strain set for additional mutant strains displaying equal or greater sensitivity to the compound, followed by genetic characterization of a double mutant strain demonstrating synthetic lethality between the two mutations.

### 5.4.2.2 Screening for Non-antifungal Therapeutic Agents With Conditional-expression Aspergillus fumigatus Mutant Strains

The biochemical similarity existing between pathogenic fungi and the mammalian hosts they infect limits the range of clinically useful antimycotic compounds. However, this similarity can be exploited using a conditional-expression Aspergillus fumigatus mutant strain collection to facilitate the discovery of therapeutics that are not used as antimycotics, but are useful for treatment a wide-range of diseases, such as cancer, inflammation, etc.

In this embodiment of the invention, fungal genes that are homologous to disease-causing genes in an animal or plant, are selected and conditional-expression Aspergillus fumigatus mutant strains of this set of genes are used for identification of compounds that display potent and specific bioactivity towards the products of these genes, and therefore have potential medicinal value for the treatment of diseases. Essential and non-essential genes and the corresponding conditional-expression Aspergillus fumigatus mutant strains carrying modified genes are useful in this embodiment of the invention. It has been predicted that as many as 40% of the genes found within the Aspergillus fumigatus genome share human functional homologs. It has also been predicted that as many as 1% of human genes are involved in human diseases and therefore may serve as potential drug targets. Accordingly, many genes within the conditional-expression Aspergillus fumigatus mutant strain collection are homologs to disease-causing human genes and compounds that specifically inactivate individual members of this gene set may in fact have alternative therapeutic value. The invention provides a pluralities of conditional-expression Aspergillus fumigatus mutant strains in which the modified alleles are fungal genes that share sequence, structural and/or functional similarities to genes that are associated with one or more diseases of the animal or plant.

For example, much of the signal transduction machinery that promotes cell cycle progression and is often perturbed in a variety of cancers is conserved in fungi. Many of these genes encode for cyclins, cyclin-dependent kinases (CDK), CDK inhibitors, phosphatases, and transcription factors that are both structurally and functionally related. As a result, compounds found to display specificity towards any of these functional classes of proteins could be evaluated by secondary screens to test for potential anticancer activity. However, cytotoxic compounds identified in this way need not act on cancer causing targets to display therapeutic potential. For example the taxol family of anti-cancer compounds, which hold promise as therapeutics for breast and ovarian cancers, bind tubulin and promote microtubule assembly, thereby disrupting normal microtubule dynamics. Yeast tubulin displays similar sensitivity to taxol, suggesting that additional compounds affecting other fundamental cellular processes shared between yeast and man could similarly be identified and assessed for antitumor activity.

The phenomenon of pathogenesis extends far beyond the taxonomic borders of microbes and ultimately reflects the underlying physiology. In many ways, the phenomenon of cancer is analogous to the process of pathogenesis by an opportunistic pathogen such as Aspergillus fumigatus. Both are non-infectious diseases caused by either the body's own cells, or microbes from its natural fauna. These cells grow in a manner unchecked by the immune system and in both cases disease manifests itself by colonization of vital organs and eventual tissue damage resulting in death. Effective drug-based treatment is also elusive for both diseases primarily because the causative agent in both cases is highly related to the host.

In fact, a number of successful therapeutic drugs affecting processes unrelated to cancer have also been discovered through anti-fungal drug screening programs. One clinically-important class of compounds includes the immunosuppressant molecules rapamycin, cyclosporin A, and FK506, which inhibit conserved signal transduction components. Cyclosporin A and FK506, form distinct drug-prolyl isomerase complexes (CyPA− Cyclosporin A and FKBP12-FK506 respectively) which bind and inactivate the regulatory subunit of the calcium and calmodulin-dependent phosphatase, calcineurin. Rapamycin also complexes with FKBP12, but this drug-protein complex also binds to the TOR family of phosphatidylinositol kinases to inhibit translation and cell cycle progression. In each case, both the mechanism of drug action, and the drug targets themselves are highly conserved from yeast to humans.

The identification of Aspergillus fumigatus drug targets, and grouping the targets into essential-gene, fungal-specific, and pathogen-specific target sets provide the basis for the development of whole-cell screens for compounds that interact with and inhibit individual members of any of these targets. Therefore, similar analyses can be used to identify other sets of conditional-expression Aspergillus fumigatus mutant strains having modified allelic pairs of genes encoding drug targets with other specific common functions or attributes. For example, conditional-expression Aspergillus fumigatus mutant strain subsets can be established which comprise gene targets that are highly homologous to human genes, or gene targets that display a common biochemical function, enzymatic activity, or that are involved in carbon compound catabolism, bosynthesis, transport of molecules (transporter activity), cellular localization, signal transduction cascades, cell cycle control, cell adhesion, transcription, translation, DNA replication, etc.

### 5.4.2.3 Target Gene Dosage-Based Whole Cell Assays

Experiments involving modulating the expression levels of the encoding gene to reveal phenotypes from which gene function may be inferred can be carried out in a pathogenic fungus, such as Aspergillus fumigatus, using the strains and methods of the present invention. The principle of drug-target-level variation in drug screening involves modulating the expression level of a drug target to identify specific drug resistance or drug sensitivity phenotypes, thereby linking a drug target to a particular compound. Often, these phenotypes are indicative of the target gene encoding the bona fide drug target of this compound. In examples where this is not the case, the candidate target gene may nonetheless provide important insight into the true target gene that is functioning either in a pathway or process related to that inhibited by the compound (e.g. producing synthetic phenotype), or instead functioning as a drug resistance mechanism associated with the identified compound.

The expression level of a given gene product is also elevated by cloning the gene into a plasmid vector that is maintained at multiple copies in the cell. Overexpression of the encoding gene is also achieved by fusing the corresponding open reading frame of the gene product to a more powerful promoter carried on a multicopy plasmid. Using these strategies, a number of overexpression screens have been successfully employed in Saccharomyces cerevisiae to discover novel compounds that interact with characterized drug targets as well as to identify the protein targets bound by existing therapeutic compounds.

The conditional-expression Aspergillus fumigatus mutant strain collection of the invention are not only useful in target validation under repressing conditions, but are also useful as a collection of strains overexpressing these same validated drug targets under nonrepressing conditions for whole cell assay development and drug screening.

Variation in the level of expression of a target gene product in a conditional-expression Aspergillus fumigatus mutant strain is also used to explore resistance to antimycotic compounds. Resistance to existing antifungal therapeutic agents reflects both the limited number of antifungal drugs available and the alarming dependence and reliance clinicians have in prescribing them. For example, dependence on azole-based compounds such as fluconazole for the treatment of fungal infections, has dramatically undermined the clinical therapeutic value for this compound. The conditional-expression Aspergillus fumigatus mutant strain collection is used to combat fluconazole resistance by identifying gene products that interact with the cellular target of fluconazole. Such products are used to identify drug targets which, when inactivated in concert with fluconazole, provide a synergistic effect and thereby overcome resistance to fluconazole seen when this compound is used alone. This is accomplished, for example, by using the conditional-expression Aspergillus fumigatus mutant strain collection to overexpress genes that enhance drug resistance. Such genes include novel or known plasma membrane exporters including ATP-binding cassette (ABC) transporters and multidrug resistance (MDR) efflux pumps, pleiotropic drug resistance (PDR) transcription factors, and protein kinases and phosphatases. Alternatively, genes specifically displaying a differential drug sensitivity are identified by screening conditional-expression Aspergillus fumigatus mutant strains expressing reduced levels (e.g., by threshold expression via the Aspergillus niger Pgla A promoter in the presence of xylose) of individual members of the target set. Identifying such genes provides important clues to drug resistance mechanisms that could be targeted for drug-based inactivation to enhance the efficacy of existing antifungal therapeutics.

In another aspect of the present invention, overexpression of the target gene for whole cell assay purposes is supported with promoters other than the tetracycline promoter system. (see Sections 5.3.1, and 6.2). For example, the Aspergillus niger Pgla A promoter is used to overexpress Aspergillus fumigatus drug targets genes. In Saccharomyces cerevisiae, the PGK1 promoter is known to provide strong constitutive expression in the presence of glucose. See, Guthrie, C., and G. R. Fink. 1991. Guide to yeast genetics and molecular biology. Methods Enzymol. 194:373-398.

In another aspect of the present invention, intermediate expression levels of individual drug targets within the conditional-expression Aspergillus fumigatus mutant strain collection may be engineered to provide strains tailored for the development of unique whole cell assays. In this embodiment of the invention, conditional-expression Aspergillus fumigatus mutant strains are grown in a medium containing a tetracycline concentration determined to provide only a partial repression of transcription. Under these conditions, it is possible to maintain an expression level between that of the constitutively expressed overproducing strain and that of wild type strain, as well as levels of expression lower than that of the wild-type strain. That is, it is possible to titrate the level of expression to the minimum required for cell viability. By repressing gene expression to this critical state, novel phenotypes, resembling those produced by a partial loss of function mutation (i.e. phenocopies of hypomorphic mutants) may be produced and offer additional target expression levels applicable for whole cell assay development and drug screening. Repressing expression of the remaining allele of an essential gene to the threshold level required for viability, therefore will provide a strain with enhanced sensitivity toward compounds active against this essential gene product.

Variation in the level of expression of a target gene product in a conditional-expression Aspergillus fumigatus mutant strain is also used to explore resistance to antimycotic compounds. Resistance to existing antifungal therapeutic agents reflects both the limited number of antifungal drugs available and the alarming dependence and reliance clinicians have in prescribing them. For example, dependence on azole-based compounds such as fluconazole for the treatment of fingal infections, has dramatically undermined the clinical therapeutic value for this compound. The conditional-expression Aspergillus fumigatus mutant strain collection is used to combat fluconazole resistance by identifying gene products that interact with the cellular target of fluconazole. Such products are used to identify drug targets which, when inactivated in concert with fluconazole, provide a synergistic effect and thereby overcome resistance to fluconazole seen when this compound is used alone. This is accomplished, for example, by using the conditional-expression Aspergillus fumigatus mutant strain collection to overexpress genes that enhance drug resistance. Such genes include novel or known plasma membrane exporters including ATP-binding cassette (ABC) transporters and multidrug resistance (MDR) efflux pumps, pleiotropic drug resistance (PDR) transcription factors, and protein kinases and phosphatases. Alternatively, genes specifically displaying a differential drug sensitivity are identified by screening conditional-expression Aspergillus fumigatus mutant strains expressing reduced levels (either by haploinsufficiency or threshold expression via the tetracycline promoter) individual members of the target set. Identifying such genes provides important clues to drug resistance mechanisms that could be targeted for drug-based inactivation to enhance the efficacy of existing antifungal therapeutics.

In another aspect of the present invention, overexpression of the target gene for whole cell assay purposes is supported with promoters other than the tetracycline promoter system. (see Sections 5.3.1, and 6.2). For example, the Aspergillus niger Pgla A promoter is used to overexpress Aspergillus fumigatus drug targets genes. In Saccharomyces cerevisiae, the PGK1 promoter is known to provide strong constitutive expression in the presence of glucose. See, Guthrie, C., and G. R. Fink. 1991. Guide to yeast genetics and molecular biology. Methods Enzymol. 194:373-398.

In another aspect of the present invention, intermediate expression levels of individual drug targets within the conditional-expression Aspergillus fumigatus mutant strain collection may be engineered to provide strains tailored for the development of unique whole cell assays. In this embodiment of the invention, conditional-expression Aspergillus fumigatus mutant strains are grown in a medium containing a tetracycline concentration determined to provide only a partial repression of transcription. Under these conditions, it is possible to maintain an expression level between that of the constitutively expressed overproducing strain and that of wild type strain, as well as levels of expression lower than that of the wild-type strain. That is, it is possible to titrate the level of expression to the minimum required for cell viability. By repressing gene expression to this critical state, novel phenotypes, resembling those produced by a partial loss of function mutation (i.e. phenocopies of hypomorphic mutants) may be produced and offer additional target expression levels applicable for whole cell assay development and drug screening. Repressing expression of the remaining allele of an essential gene to the threshold level required for viability, therefore will provide a strain with enhanced sensitivity toward compounds active against this essential gene product.

### 5.5.2.4 Uses of Tagged Strains

In still another aspect of the present invention, one or more unique oligonucleotide sequence tags or “bar codes” are incorporated into individual mutant strains included within a heterozygous strain collection of validated targets. In certain preferred embodiments, two unique sequence tags are incorporated into each conditional-expression Aspergillus fumigatus mutant strain. The presence of these sequence tags enables an alternative whole cell assay approach to drug screening. Multiple target strains may be screened simultaneously in a mixed population (rather than separately) to identify phenotypes between a particular drug target and its inhibitory agent.

Large-scale parallel analyses are performed using mixed populations of the entire bar coded heterozygous essential strain collection target set and comparing the relative representation of individual strains within a mixed population prior to and after growth in the presence of a compound. Drug-dependent depletion or overrepresentation of a unique bar-coded strain is determined by PCR-amplifying and fluorescently labeling all bar codes within the mixed population and hybridizing the resulting PCR products to an array of complementary oligonucleotides. In preferred embodiments, two sequences tags are incorporated within each conditional-expression Aspergillus fumigatus mutant strain and, therefore, two signals are generated by hybridization with the array of complementary oligonucleotides. Use of at least two sequence tags therefore provides a more precise determination of the representation of each conditional-expression Aspergillus fumigatus mutant strain present in the population. Differential representation between bar coded strains indicates gene-specific hypersensitivity or resistance and suggests the corresponding gene product may represent the molecular target of the compound tested.

In one specific embodiment, the mutant strains are conditional-expression Aspergillus fumigatus mutant strains, and each of the conditional-expression Aspergillus fumigatus mutant strains of the set comprises at least one, and preferably two unique molecular tags, which, generally, are incorporated within the promoter-replacement cassette used to place the target gene under the control of a heterologous, conditionally-expressed promoter. Each molecular tag is flanked by primer sequences which are common to all members of the set being tested. Growth is carried out in repressive and non-repressive media, in the presence and absence of the compound to be tested. The relative growth of each strain is assessed by carrying out simultaneous PCR amplification of the entire collection of embedded sequence tags.

In one non-limiting aspect of the present invention, the PCR amplification is performed in an asymmetric manner with fluorescent primers and the resulting single stranded nucleic acid product hybridized to an oligonucleotide array fixed to a surface and comprises the entire corresponding set of complementary sequences. Analysis of the level of each fluorescent molecular tag sequence is then determined to estimate the relative amount of growth of GRACE strain of the set, in those media, in the presence and absence of the compound tested.

Therefore, for each conditional-expression Aspergillus fumigatus mutant strain of the set tested, there could be, in one non-limiting example of this method, four values for the level of the corresponding molecular tag found within the surviving population. They would correspond to cell growth under repressing and non-repressing conditions, both in the presence and absence of the compound being tested. Comparison of growth in the presence and absence of the test compound provides a value or “indicator” for each set of growth media; that is, an indicator derived under repressing and non-repressing conditions. Again, comparison of the two indicator values will reveal if the test compound is active against the gene product expressed by the modified allelic gene pair carried by that specific member of the conditional-expression Aspergillus fumigatus mutant strain set tested.

In still another aspect of the present invention, each potential drug target gene in this heterozygous tagged or bar-coded collection, may be overexpressed by subsequently introducing either the Tet promoter or another strong, constitutively expressed promoter (e. g. CaACT1, CaADH1 and CaPGK1) upstream of the remaining non-disrupted allele. These constructions allow a further increase in the dosage of the encoded target gene product of individual essential genes to be used in mixed-population drug susceptibility studies. Although overexpression may itself disrupt the normal growth rate of numerous members of the population, reliable comparisons could still be made between mock and drug-treated mixed cultures to identify compound-specific growth differences.

In Saccharomyces cerevisiae, the molecular drug targets of several well-characterized compounds including 3-amino-triazol, benomyl, tunicamycin and fluconazole were identified by a similar approach. In that study, bar-coded strains bearing heterozygous mutations in HIS3, TUB1, ALG7, and ERG11, (i.e. the respective drug targets to the compounds listed above) displayed significantly greater sensitivity when challenged with their respective compound than other heterozygote bar-coded strains when grown together in a mixed population.

In another aspect of the present invention, screens for antifungal compounds can be carried out using complex mixtures of compounds that comprise at least one compound active against the target strain. Tagging or bar-coding the conditional-expression Aspergillus fumigatus mutant strain collection facilitates a number of large scale analyses necessary to identify gene sets as well as evaluate and ultimately evaluate individual targets within particular gene sets. For example, mixed-population drug screening using a bar-coded conditional-expression Aspergillus fumigatus mutant strain collection effectively functions as a comprehensive whole cell assay. Minimal amounts of a complex compound library are sufficient to identify compounds that act on individual essential target genes within the collection. This is done without the need to array the collection. Also, strong predictions as to the ‘richness’ of any particular compound library could be made before committing to it in drug screening. It becomes possible then to assess whether, for example, a carbohydrate-based chemical library possesses greater fungicidal activity than a natural product or synthetic compound library. Particularly potent compounds within any complex library of molecules can be immediately identified and evaluated according to the priority of targets and assays available for drug screening. Alternatively, the invention provides applying this information to developing “tailored” screens, in which only those targets which were demonstrated to be inactivated in mixed population experiments by a particular compound library would be included in subsequent array-formatted screens.

Traditionally, drug discovery programs have relied on an individual or a limited set of validated drug targets. The preceding examples emphasize that such an approach is no longer necessary and that high throughput target evaluation and drug screening are now possible. However, a directed approach based on selecting individual targets may still be preferred depending on the expertise, interest, strategy, or budget of a drug discovery program.

### 5.4.3 Target Evaluation in an Animal Model System

Currently, validation of an essential drug target is demonstrated by examining the effect of gene inactivation under standard laboratory conditions. Putative drug target genes deemed nonessential under standard laboratory conditions may be examined within an animal model, for example, by testing the pathogenicity of a strain homozygous for a deletion in the target gene versus wild type. However, essential drug targets are precluded from animal model studies. Therefore, the most desirable drug targets are omitted from the most pertinent conditions to their target evaluation.

In an embodiment of the invention, conditional expression, provided by the conditional-expression Aspergillus fumigatus mutant essential strain collection, overcomes this longstanding limitation to target validation within a host environment. Animal studies can be performed using mice inoculated with conditional-expression Aspergillus fumigatus mutant essential strains and examining the effect of gene inactivation by conditional expression. For examples of mouse models of Aspergillosis, see, for example Matsumoto et al. (2000) Antimicrob. Agents and Chemother 44 (3): 619-21; Brown et al. (2000) Mol. Microbiol. 36 (6): 1371-80; Bowman et al. (2001) Antimicrob. Agents and Chemother 45 (12): 3347481; and Dannaoui et al. (1999) J Med Microbiol 48 (12): 1087-93. In a preferred embodiment of the invention, the effect on mice injected with a lethal inoculum of a conditional-expression Aspergillus fumigatus mutant essential strain could be determined depending on whether the mice were provided with an appropriate concentration of tetracycline to inactivate expression of a drug target gene. The lack of expression of a gene demonstrated to be essential under laboratory conditions can thus be correlated with prevention of a terminal Aspergillus fumigatus infection. In this type of experiment, only mice “treated” with tetracycline-supplemented water, are predicted to survive infection because inactivation of the target gene has killed the conditional-expression Aspergillus fumigatus mutant strain pathogen within the host.

In yet another embodiment of the invention, conditional expression could be achieved using a temperature-responsive promoter to regulate expression of the target gene or a temperature sensitive allele of a particular drug target, such that the gene is functional at 30° C. but inactivated within the normal body temperature of the mouse.

The conditional-expression Aspergillus fumigatus mutant strain collection or a desired subset thereof is also well suited for evaluating acquired resistance/suppression or distinguishing between fungicidal/fungistatic phenotypes for an inactivated drug target within an animal model system. In this embodiment of the invention, conditional-expression Aspergillus fumigatus mutant strains repressed for expression of different essential drug target genes would be inoculated into mice raised on tetracycline-supplemented water. Each of the conditional-expression Aspergillus fumigatus mutant strains would then be compared according to the frequency of death associated with the different mice populations they infected. It is expected that the majority of infected mice will remain healthy due to fungal cell death caused by tetracycline-dependent inactivation of the essential gene in the conditional-expression Aspergillus fumigatus mutant strain. However, a conditional-expression Aspergillus fumigatus mutant strain harboring a drug target more likely to develop extragenic suppressors because it is a fungistatic target rather than fungicidal one, or suppressed by an alternative physiological process active within a host environment, can be identified by the higher incidence of lethal infections detected in mice infected with this particular strain. By this method, it is possible to evaluate/rank the likelihood that individual drug target genes may develop resistance within the host environment.

### 5.4.4 Rational Design of Binding Compounds

Compounds identified via assays such as those described herein can be useful, for example, for inhibiting the growth of the infectious agent and/or ameliorating the symptoms of an infection. Compounds can include, but are not limited to, other cellular proteins. Binding compounds can also include, but are not limited to, peptides such as, for example, soluble peptides, comprising, for example, extracellular portions of target gene product transmembrane receptors, and members of random peptide libraries (see, e.g., Lam et al., 1991, Nature 354:82-84; Houghten et al., 1991, Nature 354:84-86) made of D-and/or L-configuration amino acids, rationally-designed antipeptide peptides, (see e.g., Hurby et al., Application of Synthetic Peptides: Antisense Peptides,” In Synthetic Peptides, A User's Guide, W. H. Freeman, NY (1992), pp. 289-307), antibodies (including, but not limited to polyclonal, monoclonal, human, humanized, anti-idiotypic, chimeric or single chain antibodies, and FAb, F(ab′)₂ and FAb expression library fragments, and epitope-binding fragments thereof), and small organic or inorganic molecules. In the case of receptor-type target molecules, such compounds can include organic molecules (e.g., peptidomimetics) that bind to the ECD and either mimic the activity triggered by the natural ligand (i.e., agonists); as well as peptides, antibodies or fragments thereof, and other organic compounds that mimic the ECD (or a portion thereof) and bind to a “neutralize” natural ligand.

Computer modeling and searching technologies permit identification of compounds, or the improvement of already identified compounds, that can modulate target gene expression or activity. Having identified such a compound or composition, the active sites or regions are preferably identified. In the case of compounds affecting receptor molecules, such active sites might typically be ligand binding sites, such as the interaction domains of ligand with receptor itself. The active site is identified using methods known in the art including, for example, from the amino acid sequences of peptides, from the nucleotide sequences of nucleic acids, or from study of complexes of the relevant compound or composition with its natural ligand. In the latter case, chemical or X-ray crystallographic methods are used to find the active site by finding where on the factor the complexed ligand is found.

The three-dimensional geometric structure of the active site is then preferably determined. This is done by known methods, including X-ray crystallography, which determines a complete molecular structure. Solid or liquid phase NMR is also used to determine certain intra-molecular distances within the active site and/or in the ligand binding complex. Other experimental methods of structure determination known to those of skill in the art, are also used to obtain partial or complete geometric structures. The geometric structures are measured with a complexed ligand, natural or artificial, which increases the accuracy of the active site structure determined. Methods of computer based numerical modeling are used to complete the structure (e.g., in embodiments wherein an incomplete or insufficiently accurate structure is determined) or to improve its accuracy.

Finally, having determined the structure of the active site, either experimentally, by modeling, or by a combination, candidate modulating compounds are identified by searching databases containing compounds along with information on their molecular structure. Such a search seeks compounds having structures that match the determined active site structure and that interact with the groups defining the active site. Such a search can be manual, but is preferably computer assisted. These compounds found from this search are potential target or pathway gene product modulating compounds.

In general, the method is based on determining the three-dimensional structure of the polypeptide encoded by each essential gene, e.g., using X-ray crystallography or NMR, and using the coordinates of the determined structure in computer-assisted modeling programs to identify compounds that bind to and/or modulate the activity or expression level of encoded polypeptide. Thus, the method employs three basic steps: 1) the generation of high-purity crystals of the encoded recombinant (or endogenous) polypeptide for analysis; 2) determination of the three-dimensional structure of the polypeptide; and, 3) the use of computer-assisted “docking” programs to analyze the molecular interaction of compound structure and the polypeptide (i.e., drug screening).

General methods for performing each of the above steps are described below and are also well known to those of skill in the art. Any method known to those of skill in the art, including those described herein, may be employed for generating the three-dimensional structure for each identified essential gene product and its use in the drug-screening assays.

The products of the Aspergillus fumigatus essential genes identified herein are used as molecular targets for rational drug design. In one embodiment, the three-dimensional structure of the product of the essential gene is determined using X-ray crystallography and the resulting crystallographic data are used in in silico drug screening assays to identify agents that are capable of binding to and modulating the amount or activity of the essential gene product.

Under special conditions, molecules condense from solution into a highly-ordered crystalline lattice, which is defined by a unit cell, the smallest repeating volume of the crystalline array. The contents of such a cell can interact with and diffract certain electromagnetic and particle waves (e.g., X-rays, neutron beams, electron beams etc.). Due to the symmetry of the lattice, the diffracted waves interact to create a diffraction pattern. By measuring the diffraction pattern, crystallographers attempt to reconstruct the three-dimensional structure of the atoms in the crystal.

A crystal lattice is defined by the symmetry of its unit cell and any structural motifs the unit cell contains. For example, there are 230 possible symmetry groups for an arbitrary crystal lattice, while the unit cell of the crystal lattice group may have an arbitrary dimension that depends on the molecules making up the lattice. Biological macromolecules, however, have asymmetric centers and are limited to 65 of the 230 symmetry groups. See Cantor et al., Biophysical Chemistry, Vol. 11, W. H. Freeman & Company (1980), which is incorporated herein by reference in its entirety.

A crystal lattice interacts with electromagnetic or particle waves, such as X-rays or electron beams respectively, that have a wavelength with the same order of magnitude as the spacing between atoms in the unit cell. The diffracted waves are measured as an array of spots on a detection surface positioned adjacent to the crystal. Each spot has a three-dimensional position, hkl, and an intensity, I (hkl), both of which are used to reconstruct the three-dimensional electron density of the crystal with the so-called Electron Density Equation. The Electron Density Equation states that the three-dimensional electron density of the unit cell is the Fourier transform of the structure factors. Thus, in theory, if the structure factors are known for a sufficient number of spots in the detection space, then the three-dimensional electron density of the unit cell could be calculated using the Electron Density Equation.

Another aspect of the present invention comprises a method of using a crystal of the present invention and/or a dataset comprising the three-dimensional coordinates obtained from the crystal in a drug-screening assay. The present invention further provides the novel agents (modulators or drugs) that are identified by the method of the present invention, along with the method of using agents (modulators or drugs) identified by a method of the present invention, for inhibiting the activity of or modulating the amount of an essential gene product.

This method of drug screening relies on structure based drug design. In this case, the three dimensional structure of product of the essential gene is determined and potential agonists and/or potential antagonists are designed with the aid of computer modeling (Bugg et al., Scientific American, Dec.:92-98 (1993); West et al., TIPS, 16:67-74 (1995); Dunbrack et al., Folding & Design, 2:27-42 (1997)). However, heretofore the three-dimensional structure of the product of the essential genes identified herein has remained unknown. Therefore, there is a need for obtaining a crystal of these gene products with sufficient quality to allow high quality crystallographic data to be obtained. Furthermore there is a need for the determination of the three-dimensional structure of such crystals. Finally, there is a need for procedures for related structural based drug design predicated on such crystallographic data.

Computer analysis may be performed with one or more of the computer programs including: QUANTA, CHARMM, FlexX, INSIGHT, SYBYL, MACROMODEL and ICM (Dunbrack et al., Folding & Design, 2:27-42 (1997)). In a further embodiment of this aspect of the invention, an initial drug-screening assay is performed using the three-dimensional structure so obtained, preferably along with a docking computer program. Such computer modeling can be performed with one or more Docking programs such as DOC, FlexX, GRAM and AUTO DOCK (Dunbrack et al., Folding & Design, 2:27-42 (1997)).

It should be understood that for each drug screening assay provided herein, a number of iterative cycles of any or all of the steps may be performed to optimize the selection. The drug screening assays of the present invention may use any of a number of means for determining the interaction between an agent or drug and an Aspergillus fumigatus essential gene product.

In one such assay, a drug can be specifically designed to bind to an essential gene of the present invention through NMR based methodology, (Shuker et al., Science 274:1531-1534 (1996) hereby incorporated by reference herein in its entirety). NMR Spectroscopy and Structure Calculations: NMR spectra were recorded at 23° C. using Varian Unity Plus 500 and unity 600 spectrometers, each equipped with a pulsed-field gradient triple resonance probe as analyzed as described in Bagby et al., (Cell 82:857-867 (1995)) hereby incorporated by reference in its entirely. Sequential resonance assignments of backbone ¹H, ¹⁵N, and ¹³C atoms were made using a combination of triple resonance experiments similar to those previously described (Bagby et al., Biochemistry, 33:2409-2421 (1994)), except with enhanced sensitivity (Muhandiram and Kay, J. Magn. Reson., 103: 203-216 (1994)) and minimal H₂O saturation (Kay et al., J. Magn. Reson., 109:129-133 (1994)). Side chain ¹H and ¹³C assignments were made using HCCH-TOCSY (Bax et al., J. Magn. Reson., 87:620-627 (1990)) experiments with mixing times of 8 ms and 16 ms in solution and were not included in structure calculations. Nuclear Overhauser effect (NOE) cross peaks in two-dimensional ¹H-¹H NOE spectroscopy (NOESY), three-dimensional ¹⁵N-edited NOESY-HSQC (Zhang et al., J. Biomol, NMR, 4:845-858 (1994)) and three-dimensional simultaneous acquisition ¹⁵N/¹³C-edited NOE (Pascal et al., J. Magn. Reson., 103:197-201 (1994)) spectra were obtained with 100 ms NOE mixing times. Standard pseudo-atom distance corrections (Wuthrich et al., J. Mol. Biol., 169:949-961 (1983)) were incorporated to account for center averaging. An additional 0.5 Å was added to the upper limits for distances involving methyl groups (Wagner et al., J. Mol. Biol., 196:611-639 (1987); Clore et al., Biochemistry, 26:8012-8023 (1987)).

The structures are calculated using a simulated annealing protocol (Nilges et al, In computational Aspects of the Study of Biological Macromolecules by Nuclear Magnetic Resonance Spectroscopy, J. C. Hoch, F. M. Poulsen, and C. Redfield, eds., New York: Plenum Press, pp. 451-455 (1991) within X-PLOR (Brunger, X-PLOR Manual, Version 3.1, New Haven, Conn.: Department of Molecular Biophysics and Biochemistry, Yale University (1993) using the previously described strategy (Bagby et al., Structure, 2:107-122 (1994)). Interhelical angels were calculated using an in-house program written by K. Yap. Accessible surface areas were calculated using the program Naccess, available from Prof. J. Thornton, University College, London.

Any method known to those of skill in the art, including those set forth below, may be employed to prepare high-purity crystals. For example, crystals of the product of the identified essential gene can be grown by a number of techniques including batch crystallization, vapor diffusion (either by sitting drop or hanging drop) and by microdialysis. Seeding of the crystals in some instances is required to obtain X-ray quality crystals. Standard micro and/or macro seeding of crystals may therefore be used. Exemplified below is the hanging-drop vapor diffusion procedure. Hanging drops of an essential gene product (2.5 μl, 10 mg/ml) in 20 mM Tris, pH 8.0, 100 mM NaCl are mixed with an equal amount of reservoir buffer containing 2.7-3.2 M sodium formate and 100 mM Tris buffer, pH 8.0, and kept at 4° C. Crystal showers may appear after 1-2 days with large single crystals growing to full size (0.3×0.3×0.15 mm3) within 2-3 weeks. Crystals are harvested in 3.5 M sodium formate and 100 mM Tris buffer, pH 8.0 and cryoprotected in 3.5 M sodium formate, 100 mM Tris buffer, pH 8.0, 10% (w/v) sucrose, and 10% (v/v) ethylene glycol before flash freezing in liquid propane. Once a crystal of the present invention is grown, X-ray diffraction data can be collected.

Therefore, any person with skill in the art of protein crystallization having the present teachings and without undue experimentation could crystallize a large number of alternative forms of the essential gene products from a variety of different organisms, or polypeptides having conservative substitutions in their amino acid sequence.

Once the three-dimensional structure of a crystal comprising an essential gene product is determined, a potential modulator of its activity can be examined through the use of computer modeling using a docking program such as GRAM, DOCK, FlexX or AUTODOCK (Dunbrack et al., 1997, supra), to identify potential modulators. This procedure can include computer fitting of potential modulators to the polypeptide or fragments thereof to ascertain how well the shape and the chemical structure of the potential modulator will bind. Computer programs are employed to estimate the attraction, repulsion, and steric hindrance of the two binding partners (e.g., the essential gene product and a potential modulator). Generally the tighter the fit, the lower the steric hindrances, and the greater the attractive forces, the more potent the potential modulator since these properties are consistent with a tighter binding constant. Furthermore, the more specificity in the design of a potential drug the more likely that the drug will not interact as well with other proteins. This will minimize potential side-effects due to unwanted interactions with other proteins.

Compound and compound analogs can be systematically modified by computer modeling programs until one or more promising potential analogs is identified. In addition systematic modification of selected analogs can then be systematically modified by computer modeling programs until one or more potential analogs are identified. Such analysis has been shown to be effective in the development of HIV protease inhibitors (Lam et al., Science 263:380-384 (1994); Wlodawer et al., Ann. Rev. Biochem. 62:543-585 (1993); Appelt, Perspectives in Drug Discovery and Design 1:23-48 (1993); Erickson, Perspectives in Drug Discovery and Design 1: 109-128 (1993)). Alternatively a potential modulator could be obtained by initially screening a random peptide library produced by recombinant bacteriophage for example, (Scott and Smith, Science, 249:386-390 (1990); Cwirla et al., Proc. Natl. Acad. Sci., 87:6378-6382 (1990); Devlin et al., Science, 249:404-406 (1990)). A peptide selected in this manner would then be systematically modified by computer modeling programs as described above.

Alternatively, these methods are used to identify improved modulating compounds from an already known modulating compound or ligand. The composition of the known compound is modified and the structural effects of modification are determined using the experimental and computer modeling methods described above applied to the new composition. The altered structure is then compared to the active site structure of the compound to determine if an improved fit or interaction results. In this manner systematic variations in composition, such as by varying side groups, are quickly evaluated to obtain modified modulating compounds or ligands of improved specificity or activity.

Further experimental and computer modeling methods useful to identify modulating compounds based upon identification of the active sites of target or pathway gene or gene products and related transduction and transcription factors are apparent to those of skill in the art.

There are a number of articles that review the art of computer modeling of drugs that interact with specific proteins, including the following: Rotivinen et al., 1988, Acta Pharmaceutical Fennica 97:159-166; Ripka, (June 16, 1988), New Scientist 54-57; McKinaly and Rossmann, 1989, Annu. Rev. Pharmacol. Toxiciol. 29:111-122; Perry and Davies, OSAR: Quantitative Structure-Activity Relationships in Drug Design pp. 189-193 (Alan R. Liss, Inc. 1989); Lewis and Dean, 1989 Proc. R. Soc. Lond. 236:125-140 and 1-162; and, with respect to a model receptor for nucleic acid components, Askew et al., 1989, J. Am. Chem. Soc. 111:1082-1090.

Although generally described above with reference to design and generation of compounds which could alter binding, one could also screen libraries of known compounds, including natural products or synthetic chemicals, as well as other biologically active materials, including proteins, for compounds which are inhibitors or activators.

### 5.5 Transcriptional Profiling

### 5.5.1 Analysis of Gene Expression

Gene expression profiling techniques are important tools for the identification of suitable biochemical targets, as well as for the determination of the mode of action of known compounds. Large scale sequencing of the Aspergillus fumigatus genome and development of nucleic acid microarrays incorporating this information, will enable genome-wide gene expression analyses to be carried out with this diploid pathogenic fungus. Therefore, the present invention provides methods for obtaining the transcriptional response profiles for both essential and virulence/pathogenicity genes of Aspergillus fumigatus. Conditional expression of essential genes serves to delineate, for example, regulatory interactions valuable for the design of drug screening programs focused upon Aspergillus fumigatus.

In an embodiment of the present invention, the conditional-expression Aspergillus fumigatus mutant strain collection is used for the analysis of expression of essential genes within this pathogen. One particularly powerful application of such a strain collection involves the construction of a comprehensive transcriptional profile database for the entire essential gene set or a desired subset of essential genes within a pathogen. Such a database is used to compare the response profile characteristic of lead antimycotic compounds with the profile obtained with new anti-fungal compounds to distinguish those with similar from those with distinct modes of action. Matching (or even partially overlapping) the transcriptional response profiles determined after treatment of the strain with the lead compound with that obtained with a particular essential target gene under repressing conditions, is used to identity the target and possible mode of action of the drug.

Gene expression analysis of essential genes also permits the biological function and regulation of those genes to be examined within the pathogen, and this information is incorporated within a drug screening program. For example, transcriptional profiling of essential drug targets in Aspergillus fumigatus permits the identification of novel drug targets which participate in the same cellular process or pathway uncovered for the existing drug target and which could not otherwise be identified without direct experimentation within the pathogen. These include genes not only unique to the pathogen but also broad-range gene classes possessing a distinct function or subject to different regulation in the pathogen. Furthermore, pathogen-specific pathways may be uncovered and exploited for the first time.

In another aspect of the present invention, the gene expression profile of conditional-expression Aspergillus fumigatus mutant strains under nonrepressing or induced conditions is established to evaluate the overexpression response profile for one or more drug targets. For example, overexpression of genes functioning in signal transduction pathways often display unregulated activation of the pathway under such conditions. Moreover, several signaling pathways have been demonstrated to function in the pathogenesis process. Transcriptional response profiles generated by overexpressing conditional-expression Aspergillus fumigatus mutant strains provide information concerning the set of genes regulated by such pathways; any of which may potentially serve an essential role in pathogenesis and therefore representing promising drug targets. Furthermore, analysis of the expression profile may reveal one or more genes whose expression is critical to the subsequent expression of an entire regulatory cascade. Accordingly, these genes are particularly important targets for drug discovery and mutants carrying the corresponding modified allelic pair of genes form the basis of a mechanism-of-action based screening assays. Presently such an approach is not possible. Current drug discovery practices result in an exceedingly large number of “candidate” compounds and little understanding of their mode of action. A transcriptional response database comprising both gene shut-off and overexpression profiles generated using the conditional-expression Aspergillus fumigatus mutant strain collection offers a solution to this drug discovery bottleneck by 1) determining the transcriptional response or profile resulting from an antifungal's inhibition of a wild type strain, and 2) comparing this response to the transcriptional profiles resulting from inactivation or overexpression of drug targets comprising the conditional-expression Aspergillus fumigatus mutant strain collection.

Matching or significantly correlating transcriptional profiles resulting from both genetic alteration of a drug target and chemical/compound inhibition of wild type cells provides evidence linking the compound to its cellular drug target and suggests its mechanism of action.

Accordingly, the invention provides a method for evaluating a compound against a target gene product encoded by a nucleotide sequence comprising one of SEQ ID NOs: 2001-2594, as well as the gene product encoded by genomic SEQ ID NOs: 1-594 and 1001-1594, as expressed by Aspergillus fumigatus, said method comprising the steps of (a) contacting wild type diploid fungal cells or control cells with the compound and generating a first transcription profile; (b) determining the transcription profile of mutant fungal cells, such as a conditional-expression Aspergillus fumigatus mutant strain, which have been cultured under conditions wherein the second allele of the target gene is substantially underexpressed, not expressed or overexpressed and generating a second transcription profile for the cultured cells; and comparing the first transcription profile with the second transcription profile to identify similarities in the profiles. For comparisons, similarities of profiles can be expressed as an indicator value; and the higher the indicator value, the more desirable is the compound.

### 5.5.2 Identification of Secondary Targets

Methods are described herein for the identification of secondary targets. “Secondary target,” as used herein, refers to a gene whose gene product exhibits the ability to interact with target gene products involved in the growth and/or survival of an organism (i.e., target essential gene products), under a set of defined conditions, or in the pathogenic mechanism of the organism, (i.e., target virulence gene products) during infection of a host.

Any method suitable for detecting protein-protein interactions can be employed for identifying secondary target gene products by identifying interactions between gene products and target gene products. Such known gene products can be cellular or extracellular proteins. Those gene products which interact with such known gene products represent secondary target gene products and the genes which encode them represent secondary targets.

Among the traditional methods employed are co-immunoprecipitation, crosslinking and co-purification through gradients or chromatographic columns. Utilizing procedures such as these allows for the identification of secondary target gene products. Once identified, a secondary target gene product is used, in conjunction with standard techniques, to identify its corresponding secondary target. For example, at least a portion of the amino acid sequence of the secondary target gene product is ascertained using techniques well known to those of skill in the art, such as via the Edman degradation technique (see, e.g., Creighton, 1983, “Proteins: Structures and Molecular Principles,” W. H. Freeman & Co., N.Y., pp.34-49). The amino acid sequence obtained can be used as a guide for the generation of oligonucleotide mixtures that can be used to screen for secondary target gene sequences. Screening can be accomplished, for example, by standard hybridization or PCR techniques. Techniques for the generation of oligonucleotide mixtures and for screening are well-known. (See, e.g., Ausubel, supra., and PCR Protocols: A Guide to Methods and Applications, 1990, Innis, M. et al., eds. Academic Press, Inc., New York).

Additionally, methods are employed which result in the simultaneous identification of secondary targets which encode proteins interacting with a protein involved in the growth and/or survival of an organism under a set of defined conditions, or in the pathogenic mechanism of the organism during infection of a host. These methods include, for example, probing expression libraries with labeled primary target gene protein known or suggested to be involved in or critical to these mechanisms, using this protein in a manner similar to the well known technique of antibody probing of λgt11 phage libraries.

One method which detects protein interactions in vivo, the two-hybrid system, is described in detail for illustration purposes only and not by way of limitation. One version of this system has been described (Chien et al, 1991, Proc. Natl. Acad. Sci. USA, 88:9578-9582) and is commercially available from Clontech (Palo Alto, Calif.).

Briefly, utilizing such a system, plasmids are constructed that encode two hybrid proteins: one consists of the DNA-binding domain of a transcription activator protein fused to a known protein, in this case, a protein known to be involved in growth of the organism, or in pathogenicity, and the other consists of the activator protein's activation domain fused to an unknown protein that is encoded by a cDNA which has been recombined into this plasmid as part of a cDNA library. The plasmids are transformed into a strain of the yeast Saccharomyces cerevisiae that contains a reporter gene (e.g., lacZ) whose regulatory region contains the transcription activator's binding sites. Either hybrid protein alone cannot activate transcription of the reporter gene, the DNA-binding domain hybrid cannot because it does not provide activation function, and the activation domain hybrid cannot because it cannot localize to the activator's binding sites. Interaction of the two hybrid proteins reconstitutes the functional activator protein and results in expression of the reporter gene, which is detected by an assay for the reporter gene product.

The two-hybrid system or related methodology is used to screen activation domain libraries for proteins that interact with a known “bait” gene product. By way of example, and not by way of limitation, target essential gene products and target virulence gene products are used as the bait gene products. Total genomic or cDNA sequences encoding the target essential gene product, target virulence gene product, or portions thereof, are fused to the DNA encoding an activation domain. This library and a plasmid encoding a hybrid of the bait gene product fused to the DNA-binding domain are cotransformed into a yeast reporter strain, and the resulting transformants are screened for those that express the reporter gene. For example, and not by way of limitation, the bait gene is cloned into a vector such that it is translationally fused to the DNA encoding the DNA-binding domain of the GAL4 protein. These colonies are purified and the library plasmids responsible for reporter gene expression are isolated. DNA sequencing is then used to identify the proteins encoded by the library plasmids.

A cDNA library of the cell line from which proteins that interact with bait gene product are to be detected is made using methods routinely practiced in the art. According to the particular system described herein, for example, the cDNA fragments are inserted into a vector such that they are translationally fused to the activation domain of GAL4. This library is co-transformed along with the bait gene-GAL4 fusion plasmid into a yeast strain which contains a lacZ gene driven by a promoter which contains GAL4 activation sequence. A cDNA encoded protein, fused to GAL4 activation domain, that interacts with bait gene product reconstitutes an active GAL4 protein and thereby drive expression of the lacZ gene. Colonies which express lacZ are detected by their blue color in the presence of X-gal. The cDNA can then be purified from these strains, and used to produce and isolate the bait gene-interacting protein using techniques routinely practiced in the art.

Once a secondary target has been identified and isolated, it is further characterized and used in drug discovery by the methods of the invention.

### 5.5.3 Use of Gene Expression Arrays

To carry out profiling, gene expression arrays and microarrays can be employed. Gene expression arrays are high density arrays of DNA samples deposited at specific locations on a glass surface, silicon, nylon membrane, or the like. Such arrays are used by researchers to quantify relative gene expression under different conditions. An example of this technology is found in U.S. Pat. No. 5,807,522, which is hereby incorporated by reference.

It is possible to study the expression of substantially all of the genes in the genome of a particular microbial organism using a single array. For example, the arrays may consist of 12×24 cm nylon filters containing PCR products corresponding to ORFs from Aspergillus fumigatus. 10 ngs of each PCR product are spotted every 1.5 mm on the filter. Single stranded labeled cDNAs are prepared for hybridization to the array (no second strand synthesis or amplification step is done) and placed in contact with the filter. Thus the labeled cDNAs are of “antisense” orientation. Quantitative analysis is done using a phosphorimager.

Hybridization of cDNA made from a sample of total cell mRNA to such an array followed by detection of binding by one or more of various techniques known to those in the art provides a signal at each location on the array to which cDNA hybridized. The intensity of the hybridization signal obtained at each location in the array thus reflects the amount of mRNA for that specific gene that was present in the sample. Comparing the results obtained for mRNA isolated from cells grown under different conditions thus allows for a comparison of the relative amount of expression of each individual gene during growth under the different conditions.

Gene expression arrays are used to analyze the total mRNA expression pattern at various time points after reduction in the level or activity of a gene product required for fungal proliferation, virulence or pathogenicity. Reduction of the level or activity of the gene product is accomplished by growing a conditional-expression Aspergillus fumigatus mutant strain under conditions in which the product of the nucleic acid linked to the regulatable promoter is rate limiting for fungal growth, survival, proliferation, virulence or pathogenicity or by contacting the cells with an agent which reduces the level or activity of the target gene product. Analysis of the expression pattern indicated by hybridization to the array provides information on other genes whose expression is influenced by reduction in the level or activity of the gene product. For example, levels of other mRNAs may be observed to increase, decrease or stay the same following reduction in the level or activity of the gene product required for growth, survival, proliferation, virulence or pathogenicity. Thus, the mRNA expression pattern observed following reduction in the level or activity of a gene product required for growth, survival, proliferation, virulence or pathogenicity identifies other nucleic acids required for growth, survival, proliferation, virulence or pathogenicity. In addition, the mRNA expression patterns observed when the fungi are exposed to candidate drug compounds or known antibiotics are compared to those observed when the level or activity of a gene product required for fungal growth, survival, proliferation, virulence or pathogenicity is reduced. If the mRNA expression pattern observed with the candidate drug compound is similar to that observed when the level of the gene product is reduced, the drug compound is a promising therapeutic candidate. Thus, the assay is useful in assisting in the selection of promising candidate drug compounds for use in drug development.

In cases where the source of nucleic acid deposited on the array and the source of the nucleic acid being hybridized to the array are from two different microorganisms, gene expression identify homologous genes in the two microorganisms.

### 5.6 Proteomics Assays

In another embodiment of the present invention, and in much the same way that the conditional-expression Aspergillus fumigatus mutant strain collection enables transcriptional profiling within a pathogen, a conditional-expression Aspergillus fumigatus mutant strain collection provides an invaluable resource for the analysis of the expressed protein complement of a genome. By evaluating the overall protein expression by members of a conditional-expression Aspergillus fumigatus mutant strain collection under repressing and non-repressing growth conditions, a correlation between the pattern of protein expression of a cell can be made with the non-expression or the level of expression of an essential gene. Accordingly, the invention provides a pattern of expression of a set of proteins in a conditional-expression Aspergillus fumigatus mutant strain as determined by methods well known in the art for establishing a protein expression pattern, such as two-dimensional gel electrophoresis. A plurality of protein expression patterns will be generated for a conditional-expression Aspergillus fumigatus mutant strain when the strain is cultured under different conditions and different levels of expression of one of the modified allele.

In yet another embodiment, defined genetic mutations can be constructed to create strains exhibiting protein expression profiles comparable to those observed upon treatment of the strain with a previously uncharacterized compound. In this way, it is possible to distinguish between antimycotic compounds that act on multiple targets in a complicated manner from other potential lead compounds that act on unique fungal-specific targets and whose mode of action can be determined.

Evaluation of the full complement of proteins expressed within a cell depends upon definitive identification of all protein species detectable on two-dimensional polyacrylamide gels or by other separation techniques. However, a significant fraction of these proteins are of lower abundance and fall below the threshold level required for positive identification by peptide sequencing or mass spectrometry. Nevertheless, these “orphan” proteins are detectable using an analysis of protein expression by individual conditional-expression Aspergillus fumigatus mutant strains. Conditional expression of low abundance gene products facilitates their positive identification by comparing protein profiles of conditional-expression Aspergillus fumigatus mutant strains under repressing versus nonrepressing or overexpression conditions. In some cases, a more complex protein profile results because of changes of steady state levels for multiple proteins, which is caused indirectly by manipulating the low abundance gene in question. Overexpression of individual targets within the conditional-expression Aspergillus fumigatus mutant strain collection can also directly aid orphan protein identification by providing sufficient material for peptide sequencing or mass spectrometry.

In various embodiments, the present invention provides a method of quantitative analysis of the expressed protein complement of a diploid pathogenic fungal cell: a first protein expression profile is developed for a control diploid pathogenic fungus, which has two, unmodified alleles for the target gene. Mutants of the control strain, in which one allele of the target gene is inactivated, for example, in a conditional-expression Aspergillus fumigatus mutant strain, by insertion by or replacement with a disruption cassette, is generated. The other allele is modified such that expression of that second allele is under the control of a heterologous regulated promoter. A second protein expression profile is developed for this mutant fungus, under conditions where the second allele is substantially overexpressed as compared to the expression of the two alleles of the gene in the control strain. Similarly, if desired, a third protein expression profile is developed, under conditions where the second allele is substantially underexpressed as compared to the expression of the two alleles of the gene in the control strain. The first protein expression profile is then compared with the second expression profile, and if applicable, a third protein expression profile to identify an expressed protein detected at a higher level in the second profile, and if applicable, at a lower level in the third profile, as compared to the level in first profile.

Accordingly, the invention provides a method for evaluating a compound against a target gene product encoded by a nucleotide sequence comprising one of SEQ ID NOs: 2001-2594 and 7001-7603, as well as the gene product encoded by genomic SEQ ID NOs: 1-594, 5001-5603,1001-1594, and 6001-6603, as expressed by Aspergillus fumigatus, said method comprising the steps of (a) contacting wild type diploid fungal cells or control cells with the compound and generating a first protein expression profile; (b) determining the protein expression profile of mutant diploid fungal cells, such as a conditional-expression Aspergillus fumigatus mutant strain, which have been cultured under conditions wherein the second allele of the target gene is substantially underexpressed, not expressed or overexpressed and generating a second protein expression profile for the cultured cells; and comparing the first protein expression profile with the second protein expression profile to identify similarities in the profiles. For comparisons, similarities of profiles can be expressed as an indicator value; and the higher the indicator value, the more desirable is the compound.

### 5.7 Pharmaceutical Compositions And Uses Thereof

Compounds including nucleic acid molecules that are identified by the methods of the invention as described herein can be administered to a subject at therapeutically effective doses to treat or prevent infections by a pathogenic organism, such as Aspergillus fumigatus. Depending on the target, the compounds may also be useful for treatment of a non-infectious disease in a subject, such as but not limited to, cancer. A therapeutically effective dose refers to that amount of a compound (including nucleic acid molecules) sufficient to result in a healthful benefit in the treated subject. Typically, but not so limited, the compounds act by reducing the activity or level of a gene product encoded by a nucleic acid comprising a sequence selected from the group consisting of SEQ ID NOs: 2001-2594 and 7001-7603, as well as the gene product encoded by genomic SEQ ID NOs: 1-594, 5001-5603, 1001-1594, 6001-6603, as expressed by Aspergillus fumigatus. The subject to be treated can be a plant, a vertebrate, a mammal, an avian, or a human. These compounds can also be used for preventing or containing contamination of an object by Aspergillus fumigatus, or used for preventing or inhibiting formation on a surface of a biofilm comprising Aspergillus fumigatus. Biofilm comprising Aspergillus fumigatus are found on surfaces of medical devices, such as but not limited to surgical tools, implanted devices, catheters and stents.

### 5.7.1 Effective Dose

Toxicity and therapeutic efficacy of compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography. A useful dosage can range from 0.001 mg/kg body weight to 10 mg/kg body weight.

### 5.7.2 Formulations and Use

Pharmaceutical compositions for use in accordance with the present invention can be formulated in conventional manner using one or more physiologically acceptable carriers or excipients.

Thus, the compounds and their physiologically acceptable salts and solvents can be formulated for administration by inhalation or insufflation (either through the mouth or the nose) or oral, buccal, parenteral or rectal administration.

For oral administration, the pharmaceutical compositions can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Preparations for oral administration can be suitably formulated to give controlled release of the active compound.

For buccal administration the compositions can take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g. gelatin for use in an inhaler or insufflator can be formulated containing a powder mix of the compound and a suitable powder base such as lactose or starch.

The compounds can be formulated for parenteral administration (i.e., intravenous or intramuscular) by injection, via, for example, bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

The compounds can also be formulated in rectal compositions such as suppositories or retention enemas, e.g., containing conventional suppository bases such as cocoa butter or other glycerides.

In addition to the formulations described previously, the compounds can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

### 6. EXAMPLES

### 6.1 Isolation of Genomic DNA from Aspergillus fumigatus

Genomic DNA was isolated from Aspergillus fumigatus strain CEA10 using a commercially available isolation kit (DNEasy Plant Mini Kit, Qiagen, Inc.) according to the manufacturer's instructions with the following minor modifications. Briefly, mycelia were cultured by collecting spores from a confluent plate using a wet inoculating loop and the scraped spores touched to the surface of culture medium placed in a 24 well culture dish. The spores were swirled in the medium to ensure even growth and the dish was incubated without shaking for about 14 to 16 hours at 37° C. The mycelia grow on the surface at the air-medium interface.

The mycelia were harvested using a sterile toothpick and placed between sterile paper towels. The mycelia were squeezed to remove excess liquid and the harvested mycelia were allowed to dry for 5-10 minutes. The semi-dry mycelia were placed into Bio101 Homogenizing Matrix tubes using a sterile toothpick. To each tube, 400 μl of lysis buffer (Buffer AP1) was added and the tubes were placed into the Bio101 FastPrep Apparatus (Qbiogene), run at a speed setting of 5 for 30 seconds, and then subjected to centrifugation in a microfuge for two minutes at maximum speed at 4° C.

The supernatant containing the genomic DNA was transferred to a sterile 1.5 ml tube, 4 μl of 100 mg/mL solution of RNase was added to each tube, and the tubes were incubated for 10 minutes at 65° C. Approximately 130 μl of protein precipitation buffer (Buffer AP2) was added, the tubes mixed and incubated for about 5 minutes on ice. The supernatant was applied to the supplied QIAshredder spin column (lilac) sitting in a 2 ml collection tube and subjected to centrifugation in a microfuge for 2 min at maximum speed. The flow-through fraction was transferred to a sterile tube without disturbing the cell-debris pellet, 0.5 volume of DNA precipitation buffer (Buffer AP3) and 1 volume of ethanol (96-100%) were added to the cleared supernatant and the tubes mixed by inverting a couple times. The supernatant was applied in 650 μl aliquots, including any precipitate that may have formed, to the supplied DNeasy mini-spin column sitting in a 2 ml collection tube (supplied). The column was subjected to centrifugation in a microfuge for 1 minute at >8000 rpm and flow-through and the collection tube were discarded. The DNEasy column was placed in the supplied 2 ml collection tube, 500 μl of wash buffer (Buffer AW) was added and the DNeasy column was subjected to centrifugation in a microfuge at >8000 rpm for about 1 minute. The flow-through was discarded and the genomic DNA was eluted twice by the addition of 100 μl of a preheated (56° C.-65° C.) elution buffer (Buffer AE). The above-described protocol typically results in ˜50-100 ng of genomic DNA/μl (approximately 200 μl elution volume).

### 6.2 Promoter Replacement and Conditional Expression of the AfHIS3 Gene

The following example demonstrates that promoter replacement and conditional expression of an Aspergillus fumigatus gene is achievable by homologous recombination using a linear promoter replacement cassette.

### 6.2.1 Preparation of the AfHIS3 Promoter Replacement Cassette

The AfHIS3 gene encodes imidazoleglycerol-phosphate dehydratase that is essential for growth of Aspergillus fumigatus in minimal medium lacking exogenous histidine. The promoter of the AfHIS3 gene was replaced with a regulatable, heterologous promoter using a linear promoter replacement cassette. The promoter replacement cassette was designed to integrate into the genome by homologous recombination between regions of nucleotide sequence identity flanking the AfHIS3 promoter. Proper integration of the cassette results in deletion of the AfHIS3 promoter and introduction of the Aspergillus niger glucoamylase promoter, PglaA, which is functional in Aspergillus fumigatus. The cassette also contains a gene encoding a selectable marker, the Aspergillus niger pyrG gene, for selection and easy identification of integrative transformants.

The nucleotide sequence of the AfHIS3 gene, including flanking 5′ and 3′ untranslated sequences, is set forth in SEQ ID NO.: 4001. Based on this genomic sequence, a promoter replacement cassette (SEQ ID NO.: 4002) was constructed from three separate nucleic acid fragments. The first fragment comprising nucleotide sequences upstream of the AfHIS3 promoter was obtained by PCR amplification using genomic Aspergillus fumigatus CEA10 DNA as the template. Oligonucleotide primers (SEQ ID NO.: 4003 and 4004) were designed to amplify a nucleic acid fragment comprising nucleotides 1 to 195 of SEQ ID NO.: 4001. The upstream primer (SEQ ID NO.: 4003) corresponds to nucleotides 1 to 20 of SEQ ID NOS.: 4001 and 4002. The downstream primer (SEQ ID NO.: 4003) contains two separate regions of sequence identity; nucleotides 27 to 46 are complementary to the AfHIS3 promoter (nucleotides 175 to 195 of SEQ ID NO.: 4001) and nucleotides 1-26 are complementary to the 5′-end of the Aspergillus niger pyrG gene fragment (nucleotides 196 to 221 of SEQ ID NO.: 4002). To amplify the fragment, each primer was added at a final concentration of 0.4 μM to 10 ng of genomic Aspergillus fumigatus CEA10 in 50 μl total volume of amplification buffer using a commercially available kit (pfu Turbo Hot Start Kit, Stratagene, La Jolla, Calif.) and reactions were performed according to the manufacturers instructions. The resulting 221 bp fragment was purified from an agarose gel using a Qiagen MinElute PCR Purification Kit (Qiagen, Inc.) according to the manufacturer's instructions.

The second nucleic acid fragment containing the Aspergillus niger pyrG gene and PglaA promoter was obtained by PCR amplification using a derivative of plasmid pGUS64 (Verdoes et al., Gene 145:179-187 (1994)) containing a wild type pyrG gene, as the template. Oligonucleotide primers (SEQ ID NOS.: 4005 and 4006) were designed to amplify a nucleic acid fragment containing nucleotides 196 to 3915 of SEQ ID NO.: 4002. The upstream primer (SEQ ID NO.: 4005) corresponds to nucleotides 196 to 215 of SEQ ID NO.: 70, and the downstream primer (SEQ ID NO.: 4006) is complementary to nucleotides 3897 to 3917 of SEQ ID NO.: 4002. To amplify the fragment, each primer was added at a final concentration of 0.4 μM to 10 ng of pDXT5 in 50 μl total volume of amplification buffer using a commercially available kit (pfu Turbo Hot Start Kit, Stratagene, La Jolla, Calif.) and reactions were performed according to the manufacturer's instructions. The resulting 3,722 bp fragment was purified using a Qiagen MinElute PCR Purification Kit (Qiagen, Inc.) according to the manufacturer's instructions.

The third nucleic acid fragment containing nucleotide sequences beginning with and downstream of the ATG start codon of the AFHIS3 gene was obtained by PCR amplification using genomic Aspergillus fumigatus CEA10 DNA as the template. Oligonucleotide primers (SEQ ID NOS.: 4007 and 4008) were designed to amplify a nucleic acid fragment comprising nucleotides 3916 to 4202 of SEQ ID NO.: 4002. The downstream primer (SEQ ID NO.: 4008) is complementary to nucleotides 4186 to 4205 of SEQ ID NO.: 70. The upstream primer (SEQ ID NO.: 4007) contains two separate regions of sequence identity; nucleotides 1 to 21 correspond to the 3′-end of the pyrG-PglaA fragment (nucleotides 3896 to 3915 of SEQ ID NO.: 4002) and nucleotides 22 to 41 correspond to the first 20 nucleotides of the AfHIS3 coding sequence (nucleotides 3916 to 3935 of SEQ ID NO.: 4002). To amplify the fragment, each primer was added at a final concentration of 0.4 μM to 10 ng of plasmid in 50 μl total volume of amplification buffer using a commercially available kit (pfu Turbo Hot Start Kit, Stratagene, La Jolla, Calif.) and reactions were performed according to the manufacturer's instructions. The resulting 306 bp fragment was purified using a Qiagen MinElute PCR Purification Kit (Qiagen, Inc.) according to the manufacturer's instructions.

The full-length AfHIS3 promoter replacement cassette was constructed from the three separate fragments using three-way PCR. To construct the promoter replacement cassette, 25 ng of each of the first and third nucleic acid fragment PCR products were added to 100 ng of the second nucleic acid fragment (i.e., the pyrG-PglaA fragment) and the sample subjected to PCR amplification. The two nucleic acids comprising nucleotide sequences corresponding to the regions flanking the AfHIS3 promoter (i.e., the first and third nucleic acid fragments) each contain a 5′-overhang comprising a nucleotide sequence complementary to each end of the pyrG-PglaA fragment. Upon denaturation, the nucleotide sequences of the 5′ overhang anneal to the complementary sequences present of the pyrG-pglA fragment generating a short region of double stranded DNA having a free 3′-end that may be extended by DNA polymerase. Annealing of an intermediate PCR product containing two of the three fragments to the third fragment, or all three at once, and subsequent extension results in the production of a full-length product comprising all three nucleic acid fragments. Oligonucleotide primers (SEQ ID NO.: 4003 and SEQ ID NO.: 4008) were added to the reaction mixture at a final concentration of 0.4 μM, which results in the production of the full-length promoter replacement cassette corresponding to nucleotides 1 to 4,205 of SEQ ID NO.: 4002. The nucleotide sequence was verified directly by automated DNA sequencing.

The resulting 4,205 nucleotide promoter replacement cassette contains 195 nucleotides immediately upstream of the AfHIS3 promoter, 3,721 nucleotides containing Aspergillus niger pyrG gene and PglaA promoter placed in operable association with the first 289 nucleotides of the AfHIS3 coding sequence beginning at the ATG start codon.

### 6.2.2 Transformation of Aspergillus fumigatus Protoplasts

### 6.2.2.1 Growth and Harvest of Mycelia

An aliquot of approximate 10⁹ spores of Aspergillus fumigatus CEA10 was inoculated into 250 ml of non-selective medium supplemented with uridine and uracil, e.g., Aspergillus complete medium (ACM), and the culture was incubated with shaking at 250 rpm for about 14 to 16 hours at 30° C. After incubation, the culture is checked under a microscope to determine whether balls of mycelia have formed. If balls of mycelia are not evident, the culture was shifted to 37° C. and incubated for another 2-3 hours to stimulate mycelia ball formation. Approximately 10 transformation procedures can be performed from 250 ml of primary culture.

The mycelia were collected by filtration using a vacuum flask adapted with a sterile, cheesecloth-lined funnel. The collected mycelia were washed with 25 ml of a sterile solution of cold 0.6 M MgSO₄ and the washed mycelia were allowed to dry for about one minute. The mycelia were harvested using a sterile spatula to remove the mycelia from the cheesecloth and placed in a tube. The mass of mycelia should optimally occupy no more than 20% of the volume of the tube for optimal protoplast formation.

### 6.2.2.2 Generation and Collection of Protoplasts

Approximately a 10 ml volume of collected mycelia was placed in a 50 ml conical tube, and a sterile solution of osmotic medium (1.2 M MgSO₄, 10 mM NaPO₄, pH 5.8) is added to the tube to a final volume of 50 ml. The mycelia were dispersed by vortexing for 0.5 to 1 minute. In a separate 2 ml tube, 250 mg of Driselase enzyme (Interspex Products, San Mateo, Calif.) was added to about 1 ml of osmotic medium and placed on ice for 5 minutes. The tube was subjected to brief centrifugation at 14,000×G for 30 seconds to pellet the enzyme starch carrier. Failure to remove the starch carrier may interfere with obtaining protoplasts. The enzyme supernatant was transferred to a sterile tube and 400 mg β-D-glucanase (Interspex Products, San Mateo, Calif.) was added. The enzyme mixture was allowed to dissolve, added to the 50 ml mycelia preparation, and mixed by inverting.

The contents of the tube were poured into 500 ml Erlenmeyer flask and incubated with shaking between 100-125 rpm for 2.5 hours at 30° C. The progress of protoplast formation was examined microscopically at various time intervals until complete. Protoplast formation is typically complete within two hours. The protoplast suspension was dispensed into several 50 ml conical tubes adding no more than 10 ml volume to each tube. The suspension was gently overlaid with an equal volume of sterile Trapping Buffer (0.6 M Sorbitol in 0.1 M Tris-Cl, pH 7.0) being careful not to mix the two layers. The tubes were subjected to centrifugation at 3,000×G in a swinging bucket rotor for 15 minutes. The fuzzy white layer of that forms at the Osmotic medium/Trapping Buffer interface containing the protoplasts was removed using a transfer pipette and the samples were combined.

The combined samples were placed into a plastic centrifuge tube capable of withstanding up to 10,000×g and an equal volume of sterile STC buffer (1.2 M sorbitol, 10 mM CaCl₂ in 10 mM Tris-HCl, pH 7) was added. The protoplasts were pelleted by subjecting the protoplast sample to centrifugation at 8,000×g for 8 minutes at 4° C. The supernatant of the sample was removed taking care not to disturb the pellet. The pellet was gently resuspended in 5 ml STC buffer using a transfer pipette and the protoplasts were pelleted by subjecting the protoplast sample to centrifugation at 8,000×g for 8 minutes at 4° C. The above-described STC buffer wash steps were repeated an additional two times, the protoplasts were combined into a single tube, and resuspended into an appropriate volume for transformation (approximately 100 μl protoplast suspension/transformation reaction).

### 6.2.2.3 Protoplast Transformation

Approximately 2.5 μg of the AfHIS3 promoter replacement cassette was added to 20 μl of STC buffer a round bottom 15 ml Falcon tube (VWR Scientific). Approximately a 100 μl aliquot of protoplast preparation and 50 μl of PEG solution (60% PEG 3350, 10 mM CaCl₂, in 10 mM Tris-HCl, pH7.5) was added and the sample was incubated for 25 minutes at room temperature. After incubation, 1 ml of PEG solution was added, the tube gently rolled to mix the contents and placed on ice for 10 minutes. To each tube, 5 ml STC buffer was added and the solutions mixed completely. The protoplasts in each tube were pelleted by subjecting the protoplast samples to centrifugation at 8,000×g for 8 minutes at 4° C. The supernatant of each sample was removed taking care not to disturb the pellet and each pellet was gently resuspended in 100 μl of STC buffer. The transformation mixture was plated onto selective medium, e.g., Aspergillus minimal medium (e.g., Pontecorvo et al., Adv. Genet. 5:141-238 (1953)) lacking uracil and uridine, supplemented with sorbitol. The plates were incubated at 37° C. for 48 hours and then analyzed.

### 6.2.3 Isolation of a Strain Comprising an AfHIS3 Promoter Replacement

Approximately 15 representative pyrG+ heterokaryon transformants were streaked for isolated single colonies and screened for those colonies capable of growing on minimal medium plates lacking histidine under inducing conditions (i.e., in the presence of maltose) but unable to grow under non-inducing conditions (i.e., in the presence of xylose). Approximately 6 colonies displayed such a phenotype. Because Aspergillus fumigatus is known to integrate DNA via non-homologous recombination, the integrity of the integration event in each transformant was verified by PCR analysis using the Qiagen 2X HotStar Amplification Kit (Qiagen, Inc.). Oligonucleotide primers (SEQ ID Nos: 4003 and 4008) were used in pairwise combination to amplify a nucleic acid molecule comprising a nucleotide sequence spanning the junction region for each transformant. Proper integration of the cassette produces 1,230 bp amplification product whereas amplification of the endogenous AfHIS3 gene results in the production of a 4,689 bp fragment. Those colonies exhibiting the presence of the 1,230 bp fragment were retained for further analysis.

### 6.2.4 Titration Against 3-Aminotriazole

Aspergillus fumigatus is sensitive to the catalase inhibitor 3-aminotriazole (3-AT), which targets the product of the HIS3 gene. The concentration of 3-AT sufficient to inhibit growth of Aspergillus fumigatus is thusly dependent on the amount of HIS3 gene product present in the cells. As such, the regulation of the AfHIS3 gene by the replacement promoter may be demonstrated by varying the growth conditions to differentially express the AfHIS3 gene over a range of expression levels and demonstrating altered sensitivity of the resulting strain to 3-AT.

For instance, culturing the transformed cells containing the integrated PglaA promoter in a medium supplemented with different carbon sources or ratios of carbon sources allows the amount of AfHIS3 gene product to be increased or decreased relative to endogenous levels to generate cells that are more or less sensitive to 3-AT based on the amount of HIS3 gene product. It is known, for example, that transcription from the PglaA promoter is induced in the presence of maltose, repressed in the presence of xylose and intermediate levels of activity are detected from cells grown on glucose. By adjusting the ratio of maltose to xylose in the culture medium, the amount of transcription may be titrated, at least in a step-wise manner, to adjust levels of transcript in the cell. Those cells grown in the presence of maltose (e.g. 2% maltose) exhibited increased resistance in the presence of 3-AT whereas, conversely, cells grown in the presence of xylose (e.g. 1% xylose) exhibited decreased resistance to 3-AT.

### 6.3 Replacement of the Aspergillus fumigatus ALB1 Gene by Homologous Recombination

The following example demonstrates that deletion of coding sequence of an Aspergillus fumigatus gene and replacement with a gene encoding a selectable marker is achievable by homologous recombination using a linear gene replacement cassette. In this example transcription initiated from the pyrG marker gene is in the same direction as transcription of the AfALB 1 gene.

### 6.3.1 Preparation of the AfALB1 Gene Replacement Cassette

The ALB1 gene of Aspergillus fumigatus encodes a polyketide synthase involved in conidia coloration. For instance, particular mutations identified in the coding sequence of the ALB1 gene result in the production of white conidia, rather than green, which can be readily measured by visual examination. The AfALB1 gene replacement cassette was designed to integrate into the genome by homologous recombination between regions of nucleotide sequence identity flanking the AfALB I gene. Proper integration of the cassette results in deletion of the AfALB1 gene and introduction of the Aspergillus niger pyrG gene (e.g., see, Verdoes et al., Gene 145:179-187 (1994)) which may be used for selection and easy identification of integrative transformants.

The nucleotide sequence of the AfALB1 gene, including flanking 5′ and 3′ untranslated sequences, is set forth in SEQ ID NO.: 4009. Based on the genomic sequence, an AfALB1 gene replacement cassette (SEQ ID NO.: 4010) was constructed from three separate nucleic acid fragments. The first fragment comprising nucleotide sequences upstream of the AfALB1 gene was obtained by PCR amplification using genomic Aspergillus fumigatus CEA10 DNA as the template. Oligonucleotide primers (SEQ ID NOS.: 4011 and 4012) were designed to amplify a nucleic acid fragment comprising nucleotides 1 to 570 of SEQ ID NO.: 4010. The upstream primer (SEQ ID NO.: 4011) corresponds to nucleotides 1 to 20 of SEQ ID NO 4010 (nucleotides 449 to 468 of SEQ ID NO 77). The downstream primer (SEQ ID NO.: 4012) contains two separate regions of sequence identity; nucleotides 21 to 40 are complementary to the nucleotide sequences upstream of the AfALB1 coding sequence (nucleotides 551 to 570 of SEQ ID NO.: 4010) and nucleotides 1-20 are complementary to the 5′-end of the Aspergillus niger pyrG gene fragment (nucleotides 571 to 590 of SEQ ID NO.: 4010). To amplify the fragment, each primer was added at a final concentration of 0.4 μM to 10 ng of genomic Aspergillus fumigatus CEA10 in 50 μl total volume of amplification buffer using a commercially available kit (pfu Turbo Hot Start Kit, Stratagene, La Jolla, Calif.) and reactions were performed according to the manufacturer's instructions. The resulting 590 bp fragment was purified from an agarose gel using a Qiagen MinElute PCR Purification Kit (Qiagen, Inc.) according to the manufacturer's instructions.

The second nucleic acid fragment containing the Aspergillus niger pyrG gene was obtained by PCR amplification using a derivative of plasmid pGUS64 (Verdoes et al., Gene 145:179-187 (1994)) containing a wild type pyrG gene, as the template. Oligonucleotide primers (SEQ ID NOS.: 4013 and 4014) were designed to amplify a nucleic acid fragment containing nucleotides 571 to 2,776 of SEQ ID NO.: 4010. The upstream primer (SEQ ID NO.: 4013) corresponds to nucleotides 571 to 590 of SEQ ID NO.: 78, and the downstream primer (SEQ ID NO.: 4014) is complementary to nucleotides 2757 to 2776 of SEQ ID NO.: 4010. To amplify the fragment, each primer was added at a final concentration of 0.4 μM to 10 ng of plasmid in 50 μl total volume of amplification buffer using a commercially available kit (pfu Turbo Hot Start Kit, Stratagene, La Jolla, Calif.) and reactions were performed according to the manufacturer's instructions. The resulting 2,206 bp fragment was purified using a Qiagen MinElute PCR Purification Kit (Qiagen, Inc.) according to the manufacturer's instructions.

The third nucleic acid fragment containing nucleotide sequences downstream of the AFALB1 gene was obtained by PCR amplification using genomic Aspergillus fumigatus

CEA10 DNA as the template. Oligonucleotide primers (SEQ ID NOS.: 4015 and 4016) were designed to amplify a nucleic acid fragment comprising nucleotides 2,757 to 3,481 of SEQ ID NO.: 4010. The downstream primer (SEQ ID NO.: 4016) is complementary to nucleotides 3,461 to 3,481 of SEQ ID NO.: 4010. The upstream primer (SEQ ID NO.: 4015) contains two separate regions of sequence identity; nucleotides 1 to 20 correspond to the 3′-end of the pyrG fragment (nucleotides 2,757 to 2,776 of SEQ ID NO.: 4010) and nucleotides 21 to 36 correspond to nucleotides 2,777 to 2,792 of SEQ ID NO.: 4010). To amplify the fragment, each primer was added at a final concentration of 0.4 μM to 10 ng of plasmid in 50 μl total volume of amplification buffer using a commercially available kit (pfu Turbo Hot Start Kit, Stratagene, La Jolla, Calif.) and reactions were performed according to the manufacturer's instructions. The resulting 725 bp fragment was purified using a Qiagen MinElute PCR Purification Kit (Qiagen, Inc.) according to the manufacturer's instructions.

The full-length AFALB1 gene replacement cassette was constructed from the three separate fragments using three-way PCR. To construct the gene replacement cassette, 25 ng of each of the first and third nucleic acid fragments (i.e., flanking AfALB1 sequences) were added to 100 ng of the second nucleic acid fragment (i.e., the pyrG fragment) and the sample subjected to PCR amplification. The two nucleic acids comprising nucleotide sequences corresponding to the regions flanking the AfALB1 gene (i.e., the first and third nucleic acid fragments) each contain a 5′ overhang comprising a nucleotide sequence complementary to each end of the pyrG fragment. Upon denaturation, the nucleotide sequences of the 5′ overhang anneal to the complementary sequences present of the pyrG fragment generating a short region of double stranded DNA having a free 3′-end that may be extended by DNA polymerase. Annealing of an intermediate PCR product containing two of the three fragments to the third fragment, or all three at once, and subsequent extension results in the production of a full-length product comprising all three nucleic acid fragments. Oligonucleotide primers (SEQ ID NO.: 4011 and SEQ ID NO.: 4016) were added to the reaction mixture at a final concentration of 0.4 μM, which results in the production of the full-length, 3,481 bp gene replacement cassette of SEQ ID NO.: 4010. The nucleotide sequence was verified directly by automated DNA sequencing.

### 6.3.2 Transformation of Aspergillus fumigatus Protoplasts and Transformant Identification

Aspergillus fumigatus protoplasts were prepared from mycelia according to the methods outlined above in Section 6.2, and approximately 2.5 ng of the AfALB1 gene replacement cassette was used to transform the protoplasts essentially as described above. The protoplasts were plated onto selective medium (Aspergillus minimal medium lacking uracil and uridine) and cultured at 37° C. until mycelial transformants appeared. Isolated heterokaryon transformants were streaked for isolate colonies and the conidia were visually examined. Those colonies that produced only white condia were retained for further analysis. The presence of the replaced ALB1 gene was confirmed by PCR amplification using primers that span the junction regions.

### 6.4 Replacement of the Aspergillus fumigatus PYROA Gene by Homologous Recombination

The following example provides further demonstration that the coding sequence of an Aspergillus fumigatus gene may be deleted and replaced with a gene encoding a selectable marker is achievable by homologous recombination using a linear gene replacement cassette. In this example transcription initiated from the pyrG marker gene is in the opposite (antisense) direction to transcription of the AfPYROA gene.

### 6.4.1 Preparation of the PYROA Gene Replacement Cassette

The PYROA gene is required for pyridoxine synthesis and is required indirectly for resistance to photosensitizers (Osmani et al., J Biol Chem. 13;274(33):23565-9 (1999)). Mutations in the coding sequence of the PYROA gene result in the production of pyridoxine auxotrophs. The AFPYROA gene replacement cassette was designed to integrate into the genome by homologous recombination between regions of nucleotide sequence identity flanking the AfPYROA gene. Proper integration of the cassette results in deletion of the AfPYROA gene and introduction of the Aspergillus niger pyrG gene (e.g., see, Verdoes et al., Gene 145:179-187 (1994)) which may be used for selection and easy identification of integrative transformants.

The nucleotide sequence of the AFPYROA gene, including flanking 5′ and 3′ untranslated sequences, is set forth in SEQ ID NO.: 4019. The nucleotide sequence of the contiguous coding sequence is set forth in SEQ ID NO.: 4017, and the deduced amino acid sequence is set forth in SEQ ID NO.: 4018. Based on the genomic sequence, an AfPYROA gene replacement cassette (SEQ ID NO.: 4020) was constructed from three separate nucleic acid fragments. The first fragment comprising nucleotide sequences upstream of the AFPYROA gene was obtained by PCR amplification using genomic Aspergillus fumigatus CEA10 DNA as the template. Oligonucleotide primers (SEQ ID NOS.: 4021 and 4022) were designed to amplify a nucleic acid fragment comprising nucleotides 1 to 576 of SEQ ID NO.: 4020. The upstream primer (SEQ ID NO.: 4021) corresponds to nucleotides 1 to 20 of SEQ ID NOS.: 4020 (nucleotides 568 to 587 of SEQ ID NO.: 4019). The downstream primer (SEQ ID NO.: 4022) contains two separate regions of sequence identity; nucleotides 21 to 39 are complementary to the nucleotide sequences upstream of the AfPYROA coding sequence (nucleotides 538 to 557 of SEQ ID NO.: 4020) and nucleotides 1-21 are complementary to the 3′-end of the Aspergillus niger pyrG gene fragment (nucleotides 558 to 576 of SEQ ID NO.: 4020). To amplify the fragment, each primer was added at a final concentration of 0.4 μM to 10 ng of genomic Aspergillus fumigatus CEA 10 in 50 μl total volume of amplification buffer using a commercially available kit (pfu Turbo Hot Start Kit, Stratagene, La Jolla, Calif.) and reactions were performed according to the manufacturer's instructions. The resulting 576 bp fragment was purified from an agarose gel using a Qiagen MinElute PCR Purification Kit (Qiagen, Inc.) according to the manufacturer's instructions.

The second nucleic acid fragment containing the Aspergillus niger pyrG gene was obtained by PCR amplification using a derivative of plasmid pGUS64 (Verdoes et al., Gene 145:179-187 (1994)) containing a wild type pyrG gene, as the template. Oligonucleotide primers (SEQ ID NOS.: 4013 and 4014) were designed to amplify a nucleic acid fragment containing nucleotides 558 to 2,762 of SEQ ID NO.: 4020. The upstream primer (SEQ ID NO.: 4014) corresponds to nucleotides 558 to 577 of SEQ ID NO.: 4020, and the downstream primer (SEQ ID NO.: 4013) is complementary to nucleotides 2,743 to 2,762 of SEQ ID NO.: 4020. To amplify the fragment, each primer was added at a final concentration of 0.4 μM to 10 ng of plasmid in 50 μl total volume of amplification buffer using a commercially available kit (pfu Turbo Hot Start Kit, Stratagene, La Jolla, Calif.) and reactions were performed according to the manufacturer's instructions. The resulting 2,204 bp fragment was purified using a Qiagen MinElute PCR Purification Kit (Qiagen, Inc.) according to the manufacturer's instructions.

The third nucleic acid fragment containing nucleotide sequences downstream of the AfPYROA gene was obtained by PCR amplification using genomic Aspergillus fumigatus CEA10 DNA as the template. Oligonucleotide primers (SEQ ID NOS.: 4023 and 4024) were designed to amplify a nucleic acid fragment comprising nucleotides 2,803 to 4,343 of SEQ ID NO.: 4020. The downstream primer (SEQ ID NO.: 4024) is complementary to nucleotides 4,324 to 4,343 of SEQ ID NO.: 4020. The upstream primer (SEQ ID NO.: 4023) contains two separate regions of sequence identity; nucleotides 1 to 16 correspond to the 5′-end of the pyrG fragment (nucleotides 2,803 to 2,818 of SEQ ID NO.: 4020) and nucleotides 17 to 40 correspond to nucleotides downstream of the AfPYROA coding sequence (nucleotides 2,819 to 2,842 of SEQ ID NO.: 4020). To amplify the fragment, each primer was added at a final concentration of 0.4 μM to 10 ng of plasmid in 50 μl total volume of amplification buffer using a commercially available kit (pfu Turbo Hot Start Kit, Stratagene, La Jolla, Calif.) and reactions were performed according to the manufacturer's instructions. The resulting 1,541 bp fragment was purified using a Qiagen MinElute PCR Purification Kit (Qiagen, Inc.) according to the manufacturer's instructions.

The full-length AfPYROA gene replacement cassette was constructed from the three separate fragments using three-way PCR. To construct the gene replacement cassette, 25 ng of each of the first and third nucleic acid fragments (i.e., flanking AfPYROA sequences) were added to 100 ng of the second nucleic acid fragment (i.e., the pyrG fragment) and the sample subjected to PCR amplification. The two nucleic acids comprising nucleotide sequences corresponding to the regions flanking the AfPYROA gene (i.e., the first and third nucleic acid fragments) each contain a 5′-overhang comprising a nucleotide sequence complementary in each end of the pyrG fragment. Upon denaturation, the nucleotide sequences of the 5′-overhang anneal to the complementary sequences present of the pyrG fragment generating a short region of double stranded DNA having a free 3′-end that may be extended by DNA polymerase. Annealing of an intermediate PCR product containing two of the three fragments to the third fragment, or all three at once, and subsequent extension results in the production of a full-length product comprising all three nucleic acid fragments. Oligonucleotide primers (SEQ ID NO.: 4021 and SEQ ID NO.: 4024) were added to the reaction mixture at a final concentration of 0.4 μM which results in the production of the full-length, 4,343 bp gene replacement cassette of SEQ ID NO.: 4020. The nucleotide sequence was verified directly by automated DNA sequencing.

### 6.4.2 Transformation of Aspergillus fumigatus Protoplasts and Transformant Identification

Aspergillus fumigatus protoplasts were prepared from mycelia according to the methods outlined above in Section 6.2, and approximately 2.5 ng of the AfPYROA gene replacement cassette was used to transform the protoplasts essentially as described above. The protoplasts were plated onto selective medium (Aspergillus minimal medium lacking uracil and uridine) and cultured at 37° C. until mycelial transformants appeared. Isolated heterokaryon transformants were streaked for isolate colonies on medium containing exogenous pyridoxine and those colonies that grew in the presence, but not the absence, of pyridoxine were retained for further analysis. The presence of the replaced PYROA gene was confirmed by PCR amplification using primers that span the junction regions.

### 6.5 Identification and Nucleotide Sequence of a Homolog of the AfERG11 Gene (AfERG11α), AfERG11β

The Aspergillus fumigatus ERG11 gene has been cloned and its nucleotide sequence has been determined (e.g., American Type Culture Collection Accession No. 36607; SEQ ID NO.: 4025). The amino acid sequence of the AfERG11 gene shares 58% identity to the pathogenic fungus Candida albicans. The deduced amino acid sequence of AfERG11 is set forth in SEQ ID NO.: 4026.

The nucleotide sequence of an Aspergillus fumigatus gene sharing a high degree of identity to the nucleotide sequence of the Aspergillus fumigatus ERG11 gene is set forth in SEQ I) NO.: 4027. A nucleotide sequence comparison of the identified AfERG11 gene and the AfERG11β homolog gene revealed the ERG11 homolog is 58% identical to a 522 nucleotide region of the Aspergillus fumigatus ERG11 gene. The amino acid sequence of AfERG11β is 63.9% identity over a stretch of 482 amino acids from amino acid position about 20 to about amino acid 500 of SEQ ID NO.: 4028. The amino acid sequence shares approximately the same degree of sequence identity to the AfERG11 gene when compared to the Candida albicans ERG11 gene.

A comparison of the nucleotide sequence of the ERG11 genes of Candida albicans and Neurospora crassa against their respective genome failed to identify any corresponding homologs in these organisms. It has been previously demonstrated that Aspergillus fumigatus is relatively resistant to antifungal azole compounds. It is known in other organisms that the target of such azole compounds is the product of the ERG11 gene. Thus, the presence of an additional gene having a similar, but different, nucleotide/amino acid sequence may contribute to the observed resistance thereby providing an excellent target for drug discovery for identifying agents that inhibit the expression or activity of AfERG11 as well as the homolog of AfERG11β.

Aspergillus fumigatus strains were constructed in which either the AfERG11 gene or the AfERG11β gene had been “knocked out.” Each of these strains was compared to the wild-type Aspergillus fumigatus strain CEA 10 with respect to its sensitivity, as analyzed on agar gradient plates, to two representative azole compounds, ketoconazole and itraconazole. The data indicated that the wild-type strain, CEA 10 is more resistant to both ketoconazole and itraconazole than either knockout Aspergillus fumigatus strain. Moreover, it was also apparent that the AfERG11β knockout strain is more resistant to both ketoconazole and itraconazole than is the AfERG11α knockout strain, demonstrating that the gene products of AfERG11β and AfERG11α are differentially sensitive to azole compounds.

Therefore, it appears that the gene product of AfERG11β complements the function of AfERG11 gene product and that the azole compounds have a differential inhibitory effect on the AfERG11 and AfERG11β gene products. Regulated expression, under repressing conditions, or deletion of either the AfERG11 or the AfERG11β gene have provided modified Aspergillus fumigatus strains displaying differential sensitivity to azole compounds and, therefore, are suitable for screening for compounds active against the biosynthetic step encoded by AfERG11 and/or AfERG11β in Aspergillus fumigatus. In addition, Candida albicans mutants which lack the native CaERG11 gene but comprises one and/or both of the AfERG11 paralogs can be created. Due to a difference in codon usage, the nucleotide sequence of AfERG11 and Aferg11β may have to be modified for expression in C. albicans. Such C. albicans mutants can be useful in a screen for compounds that display an inhibitory activity towards the Aspergillus fumigatus gene products.

### 6.6 Identification and Determination of the Nucleotide Sequence of the AfALG7 Gene

In other organisms it has been demonstrated that the ALG genes function in the dolichol pathway in the synthesis of the lipid-linked oligosaccharide precursor for protein N-glycosylation. Increasing evidence suggests a role for these genes in the cell cycle (Kukuruzinska et al., Biochim Biophys Acta 1999 Jan 6;1426(2):359-72). The first gene in the pathway is ALG7, which encodes dolichol-P-dependent N-acetylglucosamine-1-P transferase. The nucleotide sequence of the portion of the Aspergillus fumigatus genome that encodes the corresponding AfALG7 gene is set forth in SEQ ID NO.: 4029. The nucleotide sequence of the coding region and the deduced amino acid sequence derived therefrom are set forth in SEQ ID NOS.: 4030 and 4031, respectively. In other organisms, it has been shown that the product of the ALG7 gene is the target of tunic amycin. Hence, the encoded polypeptide of AfALG7 is of great interest with respect to its use in drug discovery assays to develop novel antifungal compounds effective against Aspergillus fumigatus.

### 6.7 Identification and Determination of the Nucleotide Sequence of the AfAAD14 Gene

The genes encoding an aryl-alcohol dehydrogenase that is involved in isoprenoid biosynthesis have been identified from Arabidopsis and Escherichia coli (e.g., see WO 99/53071 and EP 1033405). The nucleotide sequence of the portion of the Aspergillus fumigatus genome that encodes the corresponding AfAAD14 gene is set forth in SEQ ID NO.: 4032. The nucleotide sequence of the coding region and the deduced amino acid sequence derived therefrom are set forth in SEQ ID NOS.: 4033 and 4034, respectively. The encoded protein may be used in drug discovery assays to identify compounds that inhibit its activity.

### 6.8 Identification of a Target Pathway

A target pathway is a genetic or biochemical pathway wherein one or more of the components of the pathway (e.g., enzymes, signaling molecules, etc) is a drug target as determined by the methods of the invention.

### 6.8.1 Preparation of Stocks of Conditional-expression Aspergillus fumigatus Mutant Strains for Assay

To provide a consistent source of cells to screen, frozen stocks of host conditional-expression Aspergillus fumigatus mutant strains are prepared using standard microbiological techniques. For example, a single clone of the microorganism can be isolated by streaking out a sample of the original stock onto an agar plate containing nutrients for cell growth and an antibiotic for which the conditional-expression Aspergillus fumigatus mutant strain contains a gene which confers resistance. After overnight growth an isolated colony is picked from the plate with a sterile needle and transferred to an appropriate liquid growth medium containing the antibiotic to which the conditional-expression Aspergillus fumigatus mutant strain is resistant. The cells are incubated under appropriate growth conditions to yield a culture in exponential growth. Cells are frozen using standard techniques.

### 6.8.2 Growth of Conditional-expression Aspergillus fumigatus Mutant Strains for Use in the Assay

Prior to performing an assay, a stock vial is removed from the freezer, rapidly thawed and a loop of culture is streaked out on an agar plate containing nutrients for cell growth and an antibiotic for which the conditional-expression Aspergillus fumigatus mutant strain contains a gene which confers resistance. After overnight growth, randomly chosen, isolated colonies are transferred from the plate (sterile inoculum loop) to a sterile tube containing medium containing the antibiotic to which the conditional-expression Aspergillus fumigatus mutant strain contains a gene which confers resistance. After vigorous mixing to form a homogeneous cell suspension, the optical density of the suspension is measured and if necessary an aliquot of the suspension is diluted into a second tube of medium plus antibiotic. The culture is then incubated until the cells reach an optical density suitable for use in the assay.

### 6.8.3 Selection of Medium to be Used in Assay

Two-fold dilution series of the inducer or repressor for the regulatable promoter which is linked to the gene required for the fungal proliferation, virulence or pathogenicity of the conditional-expression Aspergillus fumigatus mutant strain are generated in culture medium containing the appropriate antibiotic for which the conditional-expression Aspergillus fumigatus mutant strain contains a gene which confers resistance. Several medium are tested side by side and three to four wells are used to evaluate the effects of the inducer or repressor at each concentration in each media. Equal volumes of test media-inducer or repressor and conditional-expression Aspergillus fumigatus mutant strain cells are added to the wells of a 384 well microtiter plate and mixed. The cells are prepared as described above and diluted in the appropriate medium containing the test antibiotic immediately prior to addition to the microtiter plate wells. For a control, cells are also added to several wells of each medium that do not contain inducer or repressor. Cell growth is monitored continuously by incubation by monitoring the optical density of the wells. The percent inhibition of growth produced by each concentration of inducer or repressor is calculated by comparing the rates of logarithmic growth against that exhibited by cells growing in medium without inducer or repressor. The medium yielding greatest sensitivity to inducer or repressor is selected for use in the assays described below.

### 6.8.4 Measurement of Test Antibiotic Sensitivity in Conditional-expression Aspergillus fumigatus Mutant Strains in which the Level of the Target Gene Product is not Rate Limiting

Two-fold dilution series of antibiotics of known mechanism of action are generated in the culture medium selected for further assay development that has been supplemented with the antibiotic used to maintain the conditional-expression Aspergillus fumigatus mutant strain. A panel of test antibiotics known to act on different pathways is tested side by side with three to four wells being used to evaluate the effect of a test antibiotic on cell growth at each concentration. Equal volumes of test antibiotic and cells are added to the wells of a 384 well microtiter plate and mixed. Cells are prepared as described above using the medium selected for assay development supplemented with the antibiotic required to maintain the conditional-expression Aspergillus fumigatus mutant strain and are diluted in identical medium immediately prior to addition to the microtiter plate wells. For a control, cells are also added to several wells that lack antibiotic, but contain the solvent used to dissolve the antibiotics. Cell growth is monitored continuously by incubation in a microtiter plate reader monitoring the optical density of the wells. The percent inhibition of growth produced by each concentration of antibiotic is calculated by comparing the rates of logarithmic growth against that exhibited by cells growing in medium without antibiotic. A plot of percent inhibition against log [antibiotic concentration] allows extrapolation of an IC₅₀ value for each antibiotic.

### 6.8.5 Measurement of Test Antibiotic Sensitivity in the Conditional-expression Aspergillus fumigatus Mutant Strains in which the Level of the Target Gene Product is Rate Limiting

The culture medium selected for use in the assay is supplemented with inducer or repressor at concentrations shown to inhibit cell growth by a desired amount as described above, as well as the antibiotic used to maintain the conditional-expression Aspergillus fumigatus mutant strain. Two fold dilution series of the panel of test antibiotics used above are generated in each of these media. Several antibiotics are tested side by side in each medium with three to four wells being used to evaluate the effects of an antibiotic on cell growth at each concentration. Equal volumes of test antibiotic and cells are added to the wells of a 384 well microtiter plate and mixed. Cells are prepared as described above using the medium selected for use in the assay supplemented with the antibiotic required to maintain the conditional-expression Aspergillus fumigatus mutant strain. The cells are diluted 1:100 into two aliquots of identical medium containing concentrations of inducer that have been shown to inhibit cell growth by the desired amount and incubated under appropriate growth conditions. Immediately prior to addition to the microtiter plate wells, the cultures are adjusted to an appropriate optical density by dilution into warm sterile medium supplemented with identical concentrations of the inducer and antibiotic used to maintain the conditional-expression Aspergillus fumigatus mutant strain. For a control, cells are also added to several wells that contain solvent used to dissolve test antibiotics but which contain no antibiotic. Cell growth is monitored continuously by incubation under suitable growth conditions in a microtiter plate reader monitoring the optical density of the wells. The percent inhibition of growth produced by each concentration of antibiotic is calculated by comparing the rates of logarithmic growth against that exhibited by cells growing in medium without antibiotic. A plot of percent inhibition against log [antibiotic concentration] allows extrapolation of an IC₅₀ value for each antibiotic.

### 6.8.6 Determining the Specificity of the Test Antibiotics

A comparison of the IC₅₀s generated by antibiotics of known mechanism of action under conditions in which the level of the gene product required for fungal proliferation, virulence or pathogenicity is rate limiting or is not rate limiting allows the pathway in which a gene product required for fungal proliferation, virulence or pathogenicity lies to be identified. If cells expressing a rate limiting level of a gene product required for fungal proliferation, virulence or pathogenicity are selectively sensitive to an antibiotic acting via a particular pathway, then the gene product encoded by the gene linked to the regulatable promoter in the conditional-expression Aspergillus fumigatus mutant strain is involved in the pathway on which the antibiotic acts.

### 6.8.7 Identification of Pathway in which a Test Antibiotic Acts

As discussed above, the cell-based assay may also be used to determine the pathway against which a test antibiotic acts. In such an analysis, the pathways against in which the gene under the control of the regulatable promoter in each member of a panel of conditional-expression Aspergillus fumigatus mutant strains lies is identified as described above. A panel of cells, each containing a regulatable promoter which directs transcription of a proliferation, virulence or pathogenicity-required nucleic acid which lies in a known biological pathway required for fungal proliferation, virulence or pathogenicity, is contacted with a test antibiotic for which it is desired to determine the pathway on which it acts under conditions in which the gene product of the nucleic acid is rate limiting or is not rate limiting. If heightened sensitivity is observed in cells in which the gene product is rate limiting for a gene product which lies in a particular pathway but not in cells expressing rate limiting levels of gene products which lie in other pathways, then the test antibiotic acts against the pathway for which heightened sensitivity was observed.

### 6.9 Sequence Analysis of cDNAs Corresponding to Aspergillus fumigatus Essential Genes Disclosed Herein

Total RNA was isolated from wild type Aspergillus fumigatus strain, CEA 10, that had been grown in ACM complete medium for 24 hours. Myclia were collected and total RNA isolated, generally according to the manufacturer's instructions using a commercially available kit (RNeasy Plant Mini Kit, Catalog no. 79004 Qiagen Inc., Ontario, Canada). Generally, the mycelial pellet was frozen in liquid nitrogen, ground two powder using a mortar and pestle, and lysed in a buffer containing guanidine hydrochloride and β-mercaptoethanol. The lysate was passed through a QIAshredder column to remove cell debris and homogenize the sample. The clarified lysate was applied to a silica gel membrane, washed, dried, and finally eluted with RNase-free water.

Total RNA was then treated with DNase to remove any contaminating genomic DNA. The DNase activity was in turn removed using commercially-available DNase Inactivation Reagent according to the manufacturer's instructions (RNAqueous™-4PCR Kit, Catalog No. 1914, Ambion Inc., Austin, Tex.).

First strand cDNA synthesis was carried out using an avian RNase H⁻ reverse transcriptase, oligo dT primers, reagents, and conditions generally according to the manufacturer's instructions (ThermoScript™ RT-PCR System, Catalog No. 11146-016, Invitrogen, Carlsbad, Calif.).

PCR amplification of the cDNA product was carried out using forward primers designed to hybridize upstream of the initiation codon and reverse primers designed to hybridize downstream of the translation termination codon. For each gene analyzed, generally three forward and two reverse primers were employed in six separate PCR amplification reactions, which correspond to each pairwise combination of forward and reverse primers. A typical PCR amplification program was as follows: (1) 94° C., 2 minutes; (2) 35 cycles of: 94° C. 30 sec., 60° C. 30 sec., 72° C. 2 min., and (3) final extension at 72° C. for 10 minutes. An aliquot of each of the PCR amplification reactions was analyzed by agarose gele electrophoresis to determine which reaction yielded the longest PCR product. That reaction product was then isolated after applying the corresponding PCR reaction to a silica gel membrane spin column. Contaminants were washed through the membrane in high-salt buffers, and the double-stranded PCR product isolated in a low-salt buffer using reagents, materials, and procedures generally as recommended by the manufacturer (Concert™ Rapid PCR Purification System, Cat. 11458-015, Invitrogen, Carlsbad, Calif.). The isolated PCR product was then sequenced using methods, reagents, and equipment well known in the art. Using these methods, the nucleotide sequence of the cDNA derived from each of a number of Aspergillus fumigatus essential genes has been determined.

### 6.10 Promoter Replacement and Conditional Expression of the AfErg 8 Gene

The following example demonstrates that promoter replacement and conditional expression of an essential Aspergillus fumigatus gene is achievable by homologous recombination using a linear promoter replacement cassette.

### 6.10.1 Preparation of the AfErg 8 Promoter Replacement Cassette

The promoter of the AfErg 8 gene was replaced with a regulatable, heterologous promoter using a linear promoter replacement cassette. The promoter replacement cassette was designed to integrate into the genome by homologous recombination between regions of nucleotide sequence identity flanking the AfErg 8 promoter. Proper integration of the cassette results in deletion of the AfErg 8 promoter and introduction of the Aspergillus niger glucoamylase promoter, PglaA, which is functional in Aspergillus fumigatus. The cassette also contains a gene encoding a selectable marker, the Aspergillus niger pyrg gene, for selection and easy identification of integrative transformants.

The nucleotide sequence of the AfErg 8 gene, including flanking 5′ and 3′ untranslated sequences, is set forth in SEQ ID NO.: 406. Based on this genomic sequence, a promoter replacement cassette (SEQ ID NO.: 4038) was constructed from three separate nucleic acid fragments. The first fragment comprising nucleotide sequences upstream of the AfErg 8 promoter was obtained by PCR amplification using genomic Aspergillus fumigatus CEA10 DNA as the template. Oligonucleotide primers were designed to amplify a nucleic acid fragment corresponding to the initial segment of SEQ ID NO.: 4038. The upstream primer used corresponds to the first ˜20 nucleotides of SEQ ID NOS.: 4038. The downstream primer contained two separate regions of sequence identity; the 3′-terminal portion of the downstream primer was corresponded to a sequence in the AfErg 8 while the 5′-terminal portion of this downstream primer was complementary to the 5′-end of the Aspergillus niger pyrG gene fragment (SEQ ID NO.: 4002). To amplify the fragment, each primer was added an aliquot of genomic Aspergillus fumigatus CEA10 in amplification buffer using a commercially available kit (pfu Turbo Hot Start Kit, Stratagene, La Jolla, Calif.) and reactions were performed according to the manufacturer's instructions. The resulting fragment was purified from an agarose gel using a Qiagen MinElute PCR Purification Kit (Qiagen, Inc., Valencia Calif.) according to the manufacturer's instructions.

The second nucleic acid fragment containing the Aspergillus niger pyrG gene and PglaA promoter was obtained by PCR amplification using a derivative of plasmid pGUS64 (Verdoes et al.., Gene 145:179-187 (1994)) containing a wild type pyrG gene, as the template. Oligonucleotide primers (SEQ ID NOS.: 4005 and 4006) were designed to amplify a nucleic acid fragment containing nucleotides 196 to 3915 of SEQ ID NO.: 4002. The upstream primer (SEQ ID NO.: 4005) corresponds to nucleotides 196 to 215 of SEQ ID NO.: 70, and the downstream primer (SEQ ID NO.: 4006) is complementary to nucleotides 3897 to 3917 of SEQ ID NO.: 4002. To amplify the fragment, each primer was added at a final concentration of 0.4 μM to 10 ng of pDXT5 in 50 μl total volume of amplification buffer using a commercially available kit (pfu Turbo Hot Start Kit, Stratagene, La Jolla, Calif.) and reactions were performed according to the manufacturer's instructions. The resulting 3,722 bp fragment was purified using a Qiagen MinElute PCR Purification Kit (Qiagen, Inc.) according to the manufacturer's instructions.

The third nucleic acid fragment containing nucleotide sequences beginning with and downstream of the ATG start codon of the AfErg 8 gene was obtained by PCR amplification using genomic Aspergillus fumigatus CEA10 DNA as the template. Oligonucleotide primers were designed to amplify a nucleic acid fragment comprising the downstream portion of SEQ ID NO: 4038. The upstream primer contains two separate regions of sequence identity; the 5′-end corresponded to the 3′-end of the pyrG-PglaA fragment and the 3′-end corresponded to the amino-terminal coding sequence of the AfErg 8 gene. To amplify the fragment, each primer was added to genomic Aspergillus fumigatus CEA10 DNA in amplification buffer using a commercially available kit (pfu Turbo Hot Start Kit, Stratagene, La Jolla, Calif.) and reactions were performed according to the manufacturer's instructions. The resulting fragment was purified using a Qiagen MinElute PCR Purification Kit (Qiagen, Inc.) according to the manufacturer's instructions.

The full-length AfErg 8 promoter replacement cassette was constructed from the three separate fragments using three-way PCR. To construct the promoter replacement cassette, 25 ng of each of the first and third nucleic acid fragment PCR products were added to 100 ng of the second nucleic acid fragment (i.e., the pyrG-PglaA fragment) and the sample subjected to PCR amplification. The two nucleic acids comprising nucleotide sequences corresponding to the regions flanking the AfErg 8 promoter (i.e., the first and third nucleic acid fragments) each contain a 5′-overhang comprising a nucleotide sequence complementary to each end of the pyrG-PglaA fragment. Upon denaturation, the nucleotide sequences of the 5′ overhang anneal to the complementary sequences present of the pyrG-pglA fragment generating a short region of double stranded DNA having a free 3′-end that may be extended by DNA polymerase. Annealing of an intermediate PCR product containing two of the three fragments to the third fragment, or all three at once, and subsequent extension results in the production of a full-length product comprising all three nucleic acid fragments.

The resulting 5158 nucleotide promoter replacement cassette contains ˜640 nucleotides immediately upstream of the AfErg 8 promoter, ˜3,800 nucleotides containing Aspergillus niger pyrG gene and PglaA promoter placed in operable association with the first ˜700 nucleotides of the Erg 8 coding sequence beginning at the ATG start codon.

### 6.10.2 Conditional Expression of the Aspergillus fumigatus Essential Gene AfErg 8

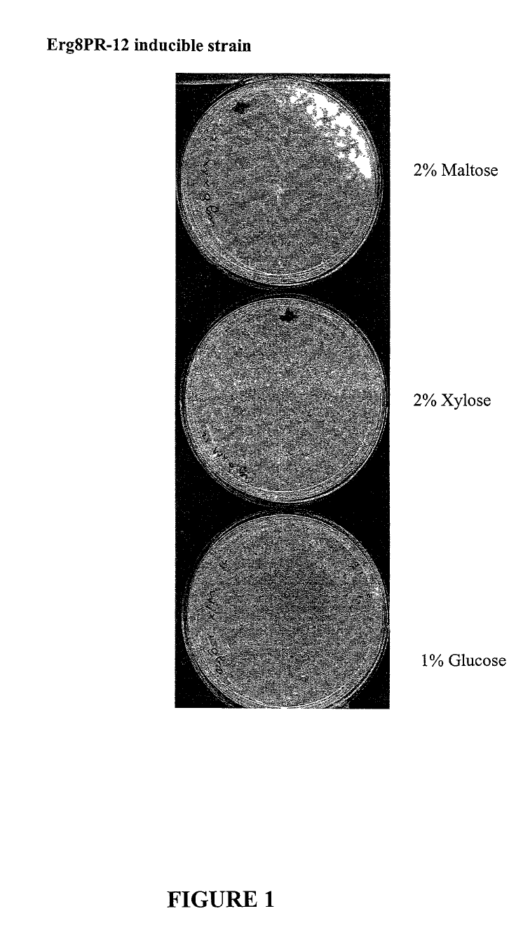

Transformants of Aspergillus fumigatus CEA10, with AfErg 8 expressed under the control of the PglaA promoter were streaked onto selective, minimal media supplemented with either 2% maltose, 2% xylose, or 1% glucose as the carbon source. As noted above, transcription from the PglaA promoter is 100-fold greater in the presence of maltose than in the presence of xylose. As demonstrated in FIG. 1, the Aspergillus fumigatus CEA10 transformant having AfErg 8 under the control of the PglaA promoter grows well on the maltose-supplemented medium. In contrast, suppression of AfErg 8 transcription in the presence of xylose leads to the inhibition of growth of this Aspergillus fumigatus CEA10 transformant, demonstrating the essentiality of the AfErg 8 gene.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

Various references are cited herein, the disclosures of which are incorporated by reference in their entireties.

What is claimed is:

1. A purified or isolated nucleic acid molecule comprising a nucleotide sequence encoding a gene product, wherein said gene product consists essentially of an amino acid sequence of one of SEQ ID NO: 3001-3594 or 8001-8603.

2. The nucleic acid molecule of claim 1, wherein said nucleotide sequence is one of SEQ ID NO: 2001-2594 or 7001-7603.

3. A nucleic acid molecule comprising a fragment of one of SEQ ID NO: 1-594, 5001-5603,1001-1594, or 6001-6603, said fragment selected from the group consisting of fragments comprising at least 10, at least 20, at least 25, at least 30, at least 50 and at least 100 consecutive nucleotides of one of SEQ ID NO: 1-594, 5001-5603,1001-1594, or 6001-6603.

4. A nucleic acid molecule comprising a nucleotide sequence that hybridizes under stringent condition to a second nucleic acid molecule consisting of (a) a nucleotide sequence selected from the group consisting of one of SEQ ID NO: 1-594, 5001-5603,1001-1594, or 6001-6603, or (b) a nucleotide sequence that encodes a polypeptide consisting of an amino acid sequence selected from the group consisting of one of SEQ ID NO: 3001-3594 or 8001-8603;
wherein said stringent condition comprises hybridization to filter-bound DNA in 6×sodium chloride/sodium citrate (SSC) at about 45° C. followed by one or more washes in 0.2×SSC/0.1% SDS at about 50-65° C.

5. A purified or isolated nucleic acid molecule obtained from an organism other than Candida albicans or Saccharomyces cerevisiae comprising a nucleotide sequence having at least 30% identity to a sequence selected from the group consisting of SEQ ID NO: 1-594, 5001-5603,1001-1594, or 6001-6603; fragments comprising at least 25 consecutive nucleotides of SEQ ID NO: 1-594, 5001-5603,1001-1594, or 6001-6603; the sequences complementary to SEQ ID NO: 1-594, 5001-5603,1001-1594, or 6001-6603; and the sequences complementary to fragments comprising at least 25 consecutive nucleotides of SEQ ID NO: 1-594, 5001-5603,1001-1594, or 6001-6603, as determined using BLASTN version 2.0 with default parameters.

6. A vector comprising a promoter operably linked to the nucleic acid molecule of claim 1, 2, 3, 4, or 5.

7. The vector of claim 6, wherein said promoter is regulatable.

8. A host cell containing the vector of claim 7.

9. A purified or isolated polypeptide comprising an amino acid sequence selected from the group consisting of one of SEQ ID NO: 3001-3594, or 8001-8603.

10. A purified or isolated polypeptide obtained from an organism other than Candida albicans or Saccharomyces cerevisiae comprising an amino acid sequence having at least 30% similarity to an amino acid sequence selected from the group consisting of one of SEQ ID NO: 3001-3594 or 8001-8603, as determined using FASTA version 3.0t78 with the default parameters.

11. A fusion protein comprising a fragment of a first polypeptide fused to a second polypeptide, said fragment consisting of at least 6 consecutive residues of an amino acid sequence selected from one of SEQ ID NO: 3001-3594 or 8001-8603.

12. A method of producing a polypeptide, said method comprises introducing into a cell, a vector comprising a promoter operably linked to a nucleotide sequence encoding a polypeptide consisting of an amino acid sequence selected from the group consisting of one of SEQ ID NO: 3001-3594 or 8001-8603; and culturing the cell such that the nucleotide sequence is expressed.

13. A method of producing a polypeptide, said method comprising providing a cell which comprises a heterologous promoter operably linked to a nucleotide sequence encoding a polypeptide consisting of an amino acid sequence selected from the group consisting of one of SEQ ID NO: 3001-3594 or 8001-8603; and culturing the cell such that the nucleotide sequence is expressed.

14. A method for identifying a compound which modulates the activity of a gene product encoded by a nucleic acid comprising a nucleotide sequence selected from the group consisting of one of SEQ ID NO: 2001-2594 or 7001-7603, said method comprising:
(a) contacting said gene product with a compound; and
(b) determining whether said compound modulates the activity of said gene product.

15. The method of claim 14, wherein the activity of the gene product is inhibited.

16. The method of claim 14, wherein said gene product is a polypeptide and said activity is selected from the group consisting of an enzymatic activity, carbon compound catabolism activity, a biosynthetic activity, a transporter activity, a transcriptional activity, a translational activity, a signal transduction activity, a DNA replication activity, and a cell division activity.

17. A method of eliciting an immune response in an animal, comprising introducing into the animal a composition comprising an isolated polypeptide, the amino acid sequence of which comprises at least 6 consecutive residues of one of SEQ ID NO: 3001-3594 or 8001-8603.

18. An isolated strain of Aspergillus fumigatus wherein the gene comprising a nucleotide sequence selected from the group consisting of one of SEQ ID NO: 1-594, 5001-5603,1001-1594, 6001-6603, 2001-2594, and 7001-7603, is inactive or placed under the control of a heterologous promoter.

19. An isolated strain of Aspergillus fumigatus comprising a nucleic acid molecule comprising a nucleotide sequence selected from one of SEQ ID NO: 1-594, 5001-5603,1001-1594, 6001-6603, 2001-2594, and 7001-7603, under the control of a heterologous promoter.

20. The strain of claim 18 or 19, wherein said heterologous promoter is regulatable.

21. A method of identifying a compound or binding partner that binds to a polypeptide comprising an amino acid sequence selected from the group consisting of one of SEQ ID NO: 3001-3594 or 8001-8603, or a fragment thereof said method comprising:
(a) contacting the polypeptide or fragment thereof with a plurality of compounds or a preparation comprising one or more binding partners; and
(b) identifying a compound or binding partner that binds to the polypeptide or fragment thereof.

22. A method for identifying a compound having the ability to inhibit growth or proliferation of Aspergillus fumigatus, said method comprising the steps of:
(a) reducing the level or activity of a gene product encoded by a nucleic acid selected from the group consisting of SEQ ID NO: 2001-2594 or 7001-7603, in a Aspergillus fumigatus cell relative to a wild type cell, wherein said reduced level is not lethal to said cell;
(b) contacting said cell with a compound; and
(c) determining whether said compound inhibits the growth or proliferation of said cell.

23. The method of claim 22, wherein said step of reducing the level or activity of said gene product comprises transcribing a nucleotide sequence encoding said gene product from a regulatable promoter under conditions in which said gene product is expressed at said reduced level.

24. The method of claim 23, wherein said gene product is a polypeptide comprising a sequence selected from the group consisting of polypeptides encoded by SEQ ID NO: 3001-3594 or 8001-8603.

25. A method for inhibiting growth or proliferation of Aspergillus fumigatus cells comprising contacting the cells with a compound that (i) reduce the level of or inhibit the activity of a nucleotide sequence selected from the group consisting of SEQ ID NO: 1-594, 5001-5603,1001-1594, 6001-6603, 2001-2594, and 7001-7603, or (ii) reduce the level of or inhibit the activity of a gene product encoded by a nucleotide sequence selected from the group consisting of SEQ ID NO: 1-594, 5001-5603,1001-1594, 6001-6603, 2001-2594, and 7001-7603,.

26. The method of claim 25, wherein said gene product is a polypeptide comprising an amino acid sequence selected from the group consisting of polypeptides encoded by SEQ ID NO: 3001-3594 or 8001-8603.

27. The method of claim 25, wherein the compound is an antibody, a fragment of an antibody, an antisense nucleic acid molecule, or a ribozyme.

28. A method for manufacturing an antimycotic compound comprising the steps of:
(a) screening a plurality of candidate compounds to identify a compound that reduces the activity or level of a gene product encoded by a nucleotide sequence selected from the group consisting of SEQ ID NO: 1-594, 5001-5603, 1001-1594, 6001-6603, 2001-2594, and 7001-7603; and
(b) manufacturing the compound so identified.

29. The method of claim 28, wherein said gene product is a polypeptide comprising an amino acid sequence selected from the group consisting of polypeptides encoded by SEQ ID NO: 1-594, 5001-5603, 1001-1594, 6001-6603, 2001-2594, and 7001-7603.

30. A method for treating an infection of a subject by Aspergillus fumigatus comprising administering a pharmaceutical composition comprising a therapeutically effective amount of a compound that reduces the activity or level of a gene product encoded by a nucleic acid comprising a sequence selected from the group consisting of SEQ ID NO: 1-594, 5001-5603, 1001-1594, 6001-6603, 2001-2594, and 7001-7603, and a pharmaceutically acceptable carrier, to said subject.

31. The method of claim 30, wherein the compound is an antibody, a fragment of an antibody, an antisense nucleic acid molecule, or a ribozyme.

32. A method for preventing or containing contamination of an object by Aspergillus fumigatus comprising contacting the object with a composition comprising an effective amount of a compound that reduces the activity or level of a gene product encoded by a nucleic acid comprising a sequence selected from the group consisting of SEQ ID NO: 1-594, 5001-5603, 1001-1594, 6001-6603, 2001-2594, and 7001-7603.

33. A pharmaceutical composition comprising a therapeutically effective amount of an agent which reduces the activity or level of a gene product encoded by a nucleic acid selected from the group consisting of SEQ ID NO: 1-594, 5001-5603, 1001-1594, 6001-6603, 2001-2594, and 7001-7603, in a pharmaceutically acceptable carrier.

34. The method of claim 30, wherein said subject is selected from the group consisting of a plant, a vertebrate, a mammal, an avian, and a human.

35. An antibody preparation which binds the polypeptide of claim 9 or 10.

36. The antibody preparation of claim 35 which comprises a monoclonal antibody.

37. A method for evaluating a compound against a target gene product encoded by a nucleotide sequence comprising one of SEQ ID NO: 1-594, 5001-5603, 1001-1594, 6001-6603, 2001-2594, and 7001-7603, said method comprising the steps of:
(a) contacting wild type fungal cells with the compound and generating a first protein expression profile;
(b) determining the protein expression profile of the fungal cells of claim 18 or 19, which have been cultured under conditions wherein the target gene is substantially underexpressed, not expressed or overexpressed and generating a second protein expression profile for the cultured cells; and
(c) comparing the first protein expression profile with the second protein expression profile to identify similarities in the profiles.

38. A collection of Aspergillus fumigatus strains of claim 18 or 19, wherein the cells of each strain further comprises one or more molecular tags each of about 20 nucleotides, wherein the sequences of each tag in a cell is unique to the strain of cells.

39. The collection of claim 38, wherein the molecular tag(s) is disposed within the gene disruption cassette.

40. An isolated strain of Aspergillus fumigatus of claim 18 or 19, wherein the cells of said strain further comprises one or more molecular tags each of about 20 nucleotides, wherein the sequence of each tag in said cells is unique to said strain of cells.

41. A nucleic acid molecule microarray comprising a plurality of nucleic acid molecules, wherein each nucleic acid molecule comprises a nucleotide sequence that is hybridizable to a target nucleotide sequence selected from the group consisting of SEQ ID NO: 1-594, 5001-5603, 1001-1594, 6001-6603, 2001-2594, and 7001-7603.

42. A computer or a computer readable medium that comprises at least one nucleotide sequence selected from the group consisting of SEQ ID NO.: 1-594, 5001-5603, 1001-1594, 6001-6603, 2001-2594, and 7001-7603, or at least one amino acid sequence selected from the group consisting of SEQ ID NO.: 3001-3594 and 8001-8603.

43. A method assisted by a computer for identifying a putatively essential gene of a fungus, comprising detecting sequence homology between a fungal nucleotide sequence or fungal amino acid sequence with at least one nucleotide sequence selected from the group consisting of SEQ ID NO.: 1-594, 5001-5603, 1001-1594, 6001-6603, 2001-2594, and 7001-7603, or at least one amino acid sequence selected from the group consisting of SEQ ID NO.: 3001-3594 and 8001-8603.
